(19) United States
(12) Patent Application Publication
(10) Pub. No.: US 2024/0051931 A1
(43) Pub. Date: Feb. 15, 2024

(54) HDAC INHIBITORS

(71) Applicant: University of Pittsburgh - Of the Commonwealth System of Higher Education, Pittsburgh, PA (US)

(72) Inventors: Peter Wipf, Pittsburgh, PA (US); Donna M. Huryn, Allentown, PA (US); Matthew G. LaPorte, Pittsburgh, PA (US); Leila Terrab, Pittsburgh, PA (US); Michael James Houghton, Niskayuna, NY (US); Andrea Topacio, Pittsburgh, PA (US); Taber Sarah Maskrey, Pittsburgh, PA (US); Tyler Kristufek, Pittsburgh, PA (US); Uygar Sozer, Long Island, NY (US); Desirae Lynn Crocker, Jeannette, PA (US); Sipak Joyasawal, Pittsburgh, PA (US); Alyssa Thornton, Washington, DC (US); Shikha Singh Chauhan, State College, PA (US); Mary Liang, Pittsburgh, PA (US); Prema Iyer, Carnegie, PA (US); Jagannath Panda, Pittsburgh, PA (US)

(73) Assignee: University of Pittsburgh - Of the Commonwealth System of Higher Education, Pittsburgh, PA (US)

(21) Appl. No.: 18/039,941

(22) PCT Filed: Dec. 2, 2021
(86) PCT No.: PCT/US2021/061611

§ 371 Date: Jun. 1, 2023

Related U.S. Application Data
(60) Provisional application No. 63/121,119, filed on Dec. 3, 2020.

(51) Int. Cl. C07D 311/66; C07D 405/12; C07D 265/36; C07D 405/04

(52) U.S. Cl. CPC C07D 311/66; C07D 405/12; C07D 265/36; C07D 405/04

(57) ABSTRACT

The present disclosure is directed to chromane compounds, chromane compounds demonstrating HDAC inhibition, and pharmaceutical compositions thereof. Additional embodiments include methods of using the chromane compounds. For example, the disclosure includes methods of inhibiting histone acetylation in a cell, comprising contacting the cell with a chromane compound of the disclosure. Additional embodiments include methods of treating a disease capable of treatment by inhibition of histone acetylation in a patient in need thereof, comprising administering a chromane compound of the disclosure.

### CROSS-REFERENCE TO RELATED APPLICATIONS

This application claims the benefit of priority to U.S. Provisional Patent Application Ser. No. 63/121,119, filed on Dec. 3, 2020, the entire disclosure of which is hereby incorporated by reference herein.

### STATEMENT REGARDING FEDERALLY SPONSORED RESEARCH OR DEVELOPMENT

This invention was made with government support under HHSN261200800001E awarded by the NIH. The government has certain rights in the invention.

### BACKGROUND

Tumor initiation and progression is regulated by epigenetic processes. Among these regulatory pathways, histone acetylation has been well studied. Histone deacetylases (HDACs) are one of the enzyme families that control the protein acetylation status. Specifically, they are involved in the removal of an acetyl group from lysine residues. In addition, HDACs epigenetically regulate histone tail, chromatin conformation, protein-DNA interaction, and transcription. To date, 18 different human HDACs have been identified, which can be further categorized into four classes based on their similarities to yeast HDACs: class I, class II, class III, and class IV. Class I, II, and IV are similar in function as Zn²⁺-dependent enzymes and share a homologous catalytic core for acetyl-lysine amide bond hydrolysis. Class III HDACs are distinct both in structure and enzymatic activity based on their dependence on the cofactor nicotinamide adenine dinucleotide (NAD⁺).

Class I HDACs include HDACs 1, 2, 3, and 8. This class of HDAC enzymes is homologous to the yeast RPD3 protein. They are typically present in the nucleus, with the exception of HDAC 8, which can be found in both nucleus and cytoplasm. These enzymes are expressed ubiquitously in various human tissues and are vital in cell proliferation, differentiation, and cell cycle progression. HDAC 1, 2, and 3 make up the components of multi-protein complexes which are important to transcriptional repression. HDAC 8 differs from the rest of the class I HDACs since it is mainly limited to specific tissues and exhibits deacetylase activity independent from other cofactors.

Class II HDACs include HDACs 4, 5, 6, 7, 9, and 10. This group is closely related to the yeast HDA1 protein. They are typically located both in the nucleus and cytoplasm. This class also has both histone and non-histone proteins targets. Unlike class I, these enzymes are tissue specific. They are involved in differentiation, and by triggering transcriptional repression, play a role in the development of skeletal, cardiac, smooth muscle, bone, immune system, vascular system, and brain. Class II HDACs can be further subdivided into class IIa (HDACs 4, 5, 7, and 9) and class IIb (HDACs 6 and 10), which are based on the presence or absence, respectively, of a double catalytic domain. Class IIa enzymes show poor deacetylase activities unless operating alongside class I HDACs. Class IIb enzymes are similar in structure. HDAC 6 is particularly unique, as it has two independent catalytic domains and can deacetylate α-tubulin in vitro and in vivo. Because of this, HDAC 6 plays a vital role in cytoskeleton regulation and its associated mediated processes.

Class III HDACs include the sirtuin family: sirt1-7. Sirtuins can be found in the nucleus, cytoplasm, and mitochondria. The wide expression of sirtuins in the cells makes them suitable for a multitude of biological functions, ranging from aging, DNA repair, regulation of oxidative stress, and regulation of the metabolism. Sirtuins demonstrate two roles-one which has a pro-oncogenic effect and another which involves a tumor suppressor function in carcinomas.

Class IV contains a single isoform, HDAC 11. This enzyme can be found in the nucleus and cytoplasm. It shares the same catalytic domain of both class I and class II HDACs. Not much has been published yet about the specific role of this class IV HDAC, so its purpose remains uncertain; however, HDAC 11 has been found to be involved in Hodgkin lymphoma.

HDACs are an integral part of the regulation of multiple processes of life, ranging from gene expressions to protein activities. It has been noted that a high expression of HDACs is commonly found in several types of cancers. Thus, HDAC inhibitors (“HDACis”) have great potential for the therapy of human cancers. Most HDACis follow the common pharmacophore models—consisting of a cap part, a zinc binding group (ZBG), and a linker part connecting the ZBG and cap part. There is typically an aromatic ring or a heteroaromatic hydrophobic moiety in the cap part which helps mediate the interaction with the amino acids at the rim of the HDAC enzyme. This is mainly responsible for the selectivities among HDAC isoforms. The ZBG acts as the chelating group for the zinc ion in the active site of HDACs. Thus, modification of the ZBG can lead to a change in potency of HDACis.

Since class I, II, and IV HDACs are all dependent on a Zn²⁺ ion, they are susceptible to inhibitors that occupy the catalytic core of the Zn-binding site. HDACis can be categorized into five main classes based on the structure of their ZBG: hydroxamic acids, carboxylic acids, benzamides, cyclic peptides, ketones and others. HDACis containing hydroxamates are currently the most broadly investigated and most potent structural class. Most hydroxamates tend to be pan-HDACis, while benzamides have increased selectivity for class I HDACs.

Four HDACis have been approved by the FDA for the treatment of hematologic malignancies: Vorinostat (SAHA), Belinostat (PXD101), Panobinostat (LBH589), and Romidepsin (FK2228).

The present HDAC inhibitors mainly belong to pan-inhibitors with adverse side effects, but researchers hypothesize that isoform-selective HDACis could lead to a better therapeutic index and fewer unfavorable side effects. Through modifications of the cap region and linker, the development of selective HDAC inhibitors has made great advances to date.

A need exists for improved HDAC inhibitors, with a good selectivity profile. It is against this background that a need arose to develop the embodiments described herein.

### SUMMARY OF THE DISCLOSED SUBJECT MATTER

The present technology is directed to chromane compounds, chromane compounds demonstrating HDAC inhibition, and methods of using the same.

Certain embodiments include a compound represented by a structure of Formula (II):

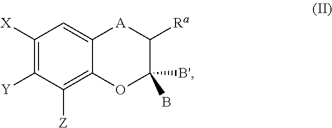

or a pharmaceutically acceptable salt, solvate, or prodrug thereof, wherein: A is CR′R′, CR′OH, CR′OCH₃, or NR′; each R′ is independently H or alkyl; R^{a} is H or alkyl; one of B and B′ is H or alkyl and the other is C(O)Q or B and B′ together form a cycloalkyl or heterocycle that is substituted by C(O)Q or (CH₂)₁₋₂C(O)Q; Q is NHOH; X is H, halo, or R¹; Y is H, halo, or R¹; Z is H, halo, or R¹; each R¹ is independently D-E-G,
where D is a bond, —O—, —NR—, —OCONR—, —OCO—, —NRSO₂—, —NRCO—, —NRSO₂NR—, —NRCOO—, —NRCONR—, or —NRC(NR)NR—; E is absent or is selected from an optionally substituted C₁-C₆ alkyl, C₁-C₆ alkenyl, C₁-C₆ alkynyl, or alkoxy; and G is H, an optionally substituted aryl, an optionally substituted heteroaryl,

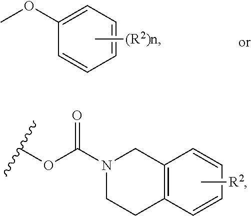

where each R² is independently selected from optionally substituted alkyl, alkoxy, aryl, halo, and SF₅; each R is independently H or C₁-C₃ alkyl; and n is 0, 1, 2, or 3; wherein at least one of X, Y and Z is not H. In some embodiments, A is CH₂. In some embodiments, B is C(O)Q and B′ is H. In some embodiments, X is R¹, and is represented by the following:

### G

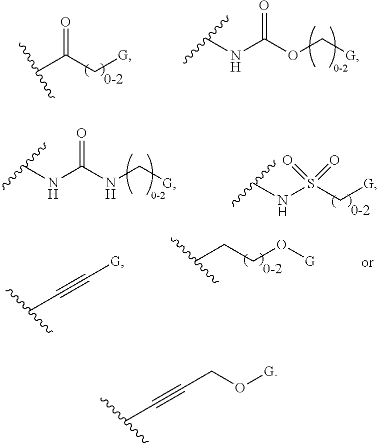

In some embodiments, G is

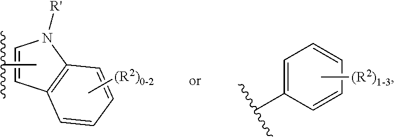

where each R² is independently selected from optionally substituted alkyl, alkoxy, aryl, halo, SF₅, or two adjacent R² form a ring. In some embodiments, Z is R¹, and is represented by the following:

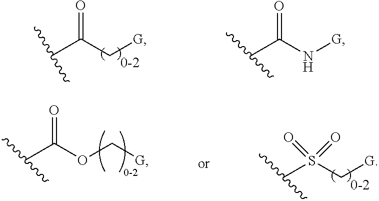

In some embodiments, G is

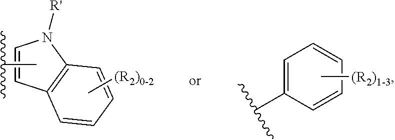

where R² is selected from optionally substituted alkyl, alkoxy, aryl, halo, SF₅, or two adjacent R² form a ring. In some embodiments, Y is G or

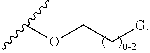

In some embodiments, G is

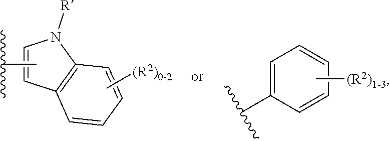

where each R² is independently selected from optionally substituted alkyl, alkoxy, aryl, halo, SF₅, or two adjacent R² form a ring. In some embodiments, G is phenyl optionally substituted with 1 to 5 substituents independently selected from F, Cl, CH₃, CF₃, OMe, SF₅, and phenyl. In some embodiments, G is heteroaryl optionally substituted with 1 to 5 substituents independently selected from F, Cl, CH₃, CF₃, OMe, and SF₅. In some embodiments, E is absent. In some embodiments, R^{a} is H. In some embodiments, each R² is independently selected from F, Cl, CH₃, CF₃, OMe, and SF₅. In some embodiments, R′ is H or CH₃. In some embodiments, R is H. In some embodiments, B and B′ together form a heterocycle having the following structure:

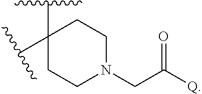

In some embodiments, B and B′ are

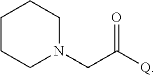

Additional embodiments include a compound selected from Table 1, or compound 50b, compound 50d, or compound 50e, or a pharmaceutically acceptable salt, solvate, or prodrug thereof. Additional embodiments include a compound selected from Table 1, or a pharmaceutically acceptable salt, solvate, or prodrug thereof. Additional embodiments include a pharmaceutical composition comprising a compound of any one of the above embodiments and a pharmaceutically acceptable excipient.

Additional embodiments include a method of inhibiting histone acetylation in a cell, comprising contacting the cell with a compound of any one of the above embodiments, or a composition comprising a compound of any one of the above embodiments.

Additional embodiments include a method of treating a disease capable of treatment by inhibition of histone acetylation in a patient in need thereof, comprising administering a compound or composition of any one of claims 1 to 12. In some embodiments, the disease is a malignancy. In some embodiments, the malignancy is a hematologic malignancy. In some embodiments, the disease is an infectious disease.

Both the foregoing summary and the following detailed description are exemplary and explanatory. They are intended to provide further details of the disclosure, but are not to be construed as limiting. Other objects, advantages, and novel features will be readily apparent to those skilled in the art from the following detailed description of the disclosure.

### DETAILED DESCRIPTION

### I. Compounds of the Disclosure

The present disclosure provides chromane compounds and chromane compounds demonstrating HDAC inhibition, and methods of using the same.

In an aspect is provided a compound represented by a structure of Formula (II):

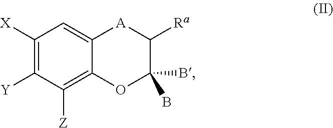

or a pharmaceutically acceptable salt, solvate, or prodrug thereof, wherein:
A is CR′R′, CR′OH, or NR′;
each R′ is independently H or alkyl;
R^{a} is H or alkyl;
one of B and B′ is H or alkyl and the other is C(O)Q or B and B′ together form a cycloalkyl or heterocycle that is substituted by C(O)Q or (CH₂)₁₋₂C(O)Q;
Q is NHOH;
X is H, halo, or R¹;
Y is H, halo, or R¹;
Z is H, halo, or R¹;
each R¹ is independently D-E-G,
where D is a bond, —O—, —NR—, —OCONR—, —OCO—, —NRSO₂—, —NRCO—, —NRSO₂NR—, —NRCOO—, —NRCONR—, or —NRC(NR)NR—;
E is absent or is selected from a optionally substituted C₁-C₆ alkyl, C₁-C₆ alkenyl, C₁-C₆ alkynyl, or alkoxy; and
   G is H, an optionally substituted aryl, an optionally substituted heteroaryl,

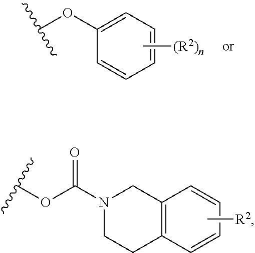

where each R² is independently selected from optionally substituted alkyl, alkoxy, aryl, halo, and SF₅;
each R is independently H or C₁-C₃ alkyl; and
n is 0, 1, 2, or 3; and
wherein at least one of X, Y and Z is not H.

In another aspect, compounds of the present disclosure include those represented by Formula (I):

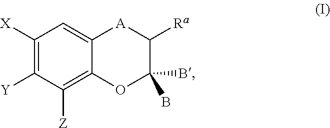

or a pharmaceutically acceptable salt, solvate, or prodrug thereof, wherein:
A is CR′R′, CR′OH, or NR′;
R′ is H or alkyl;
R^{a} is H or alkyl;
one of B and B′ is H or alkyl and the other is C(O)Q or B and B′ together form a cycloalkyl or heterocycle that is substituted by C(O)Q or (CH₂)₁₋₂C(O)Q;
Q is NHOH;
X is H, halo, or R¹;
Y is H, halo, or R¹;
Z is H, halo, or R¹;
R¹ is D-E-G,
where D is a bond, —O—, —NR—, —OCONR—, —OCO—, —NRSO₂—, —NRCO—, —NRSO₂NR—, —NRCOO—, —NRCONR—, or —NRC(NR)NR—;
E is absent or is selected from a C₁-C₆ alkane, C₁-C₆ alkene, C₁-C₆ alkyne, or alkoxy; and
   G is an optionally substituted aryl or

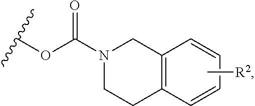

where R² is selected from optionally substituted alkyl, alkoxy, aryl, halo, SF₅,
wherein at least one of X, Y and Z is not H.

In some embodiments, A is CH₂. In other embodiments, A is CHOH. In some embodiments, A is CHOCH₃. In other embodiments, A is NH or N-alkyl. In some embodiments, A is NCH₃.

In some embodiments, B is C(O)Q and B′ is H. In some embodiments, B and B′ together form a cycloalkyl that is substituted by C(O)Q or (CH₂)₁₋₂C(O)Q. In some embodiments, B and B′ together form a heterocycle that is substituted by C(O)Q or (CH₂)₁₋₂C(O)Q. In some embodiments, B and B′ together form a heterocycle having the following structure

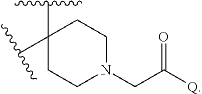

In other embodiments, B and B′ are

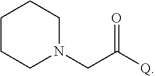

In some embodiments, X is H. In some embodiments, X is halo. In some embodiments, X is Cl.

In some embodiments, X is R¹, and is represented by the following:

### G

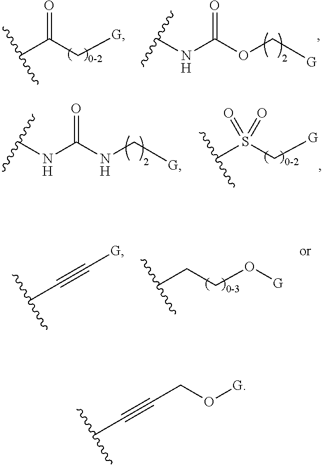

In some embodiments, G is

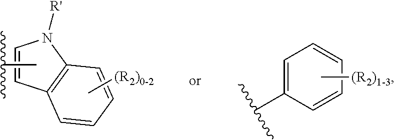

where R² is selected from optionally substituted alkyl, alkoxy, aryl, halo, SF₅, or two adjacent R² form a ring. In some embodiments, G is

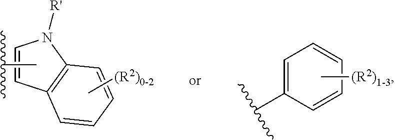

where each R² is independently selected from optionally substituted alkyl, alkoxy, aryl, halo, SF₅, or two adjacent R² form a ring.

In some embodiments, Z is H. In some embodiments, Z is CH₃.

In some embodiments, Z is R¹, and is represented by the following:

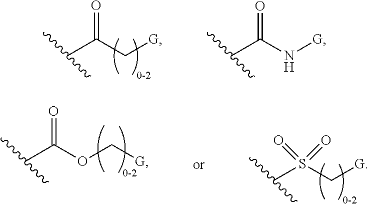

In some embodiments, G is

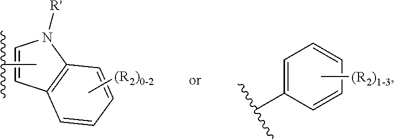

where R² is selected from optionally substituted alkyl, alkoxy, aryl, halo, SF₅, or two adjacent R² form a ring. In some embodiments, G is

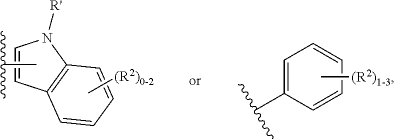

where each R² is independently selected from optionally substituted alkyl, alkoxy, aryl, halo, SF₅, or two adjacent R² form a ring.

In some embodiments, Y is H.

In some embodiments, Y is G or

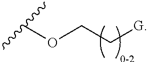

In some embodiments, G is

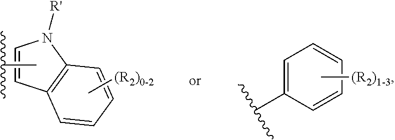

where R² is selected from optionally substituted alkyl, alkoxy, aryl, halo, SF₅, or two adjacent R² form a ring. In some embodiments, G is

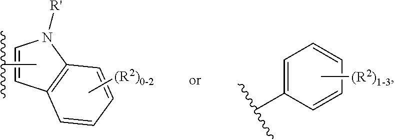

where each R² is selected from optionally substituted alkyl, alkoxy, aryl, halo, SF₅, or two adjacent R² form a ring.

In some embodiments, G is H. In some embodiments, G is optionally substituted aryl. In some embodiments, G is phenyl optionally substituted with 1 to 5 substituents independently selected from F, Cl, CH₃, CF₃, OMe, SF₅, and phenyl. In some embodiments, G is unsubstituted phenyl. In some embodiments, G is unsubstituted naphthyl. In some embodiments, G is naphthyl optionally substituted with 1 to 5 substituents independently selected from F, Cl, CH₃, CF₃, OMe, and SF₅. In some embodiments, G is optionally substituted heteroaryl. In some embodiments, G is heteroaryl optionally substituted with 1 to 5 substituents independently selected from F, Cl, CH₃, CF₃, OMe, and SF₅. In some embodiments, G is indolyl optionally substituted with 1 to 5 substituents independently selected from F, Cl, CH₃, CF₃, OMe, and SF₅. In some embodiments, G is indolyl substituted with CH₃.

In some embodiments, X and Y are H and Z is R¹. In some embodiments, X is R¹ and Y and Z are H.

In some embodiments, D is a bond. In some embodiments, D is —O—. In some embodiments, D is —NRSO₂—. In some embodiments, D is —NRCO—. In some embodiments, D is —NRCOO—. In some embodiments, D is —NRCONR—. In some embodiments, D is —NRC(NR)NR—.

In some embodiments, E is absent. In some embodiments, E is C₁-C₆ alkyl optionally substituted with 1-6 halogens. In some embodiments, E is C₁-C₆ alkyl optionally substituted with CF₃. In some embodiments, E is —CH₂—. In some embodiments, E is C₁-C₆ alkenyl. In some embodiments, E is C₁-C₆ alkynyl. In some embodiments, E is alkoxy.

In some embodiments, D is —O— and E is absent.

In some embodiments, R^{a} is H. In some embodiments, R^{a} is CH₃.

In some embodiments, each R² is independently C₁-C₆ alkyl, optionally substituted with 1 to 6 halogens. In some embodiments, each R² is independently selected from F, Cl, CH₃, CF₃, OMe, and SF₅. In some embodiments, R² is unsubstituted phenyl.

In some embodiments, R′ is H. In some embodiments, R′ is CH₃.

In some embodiments, R is H. In some embodiments, R is CH₃.

In some embodiments, n is 0. In some embodiments, n is 1. In some embodiments, n is 2. In some embodiments, n is 3.

In some embodiments, the compound is represented by a structure of Formula (IIa):

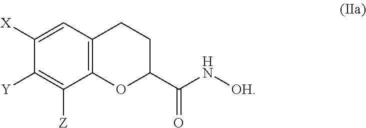

In some embodiments, the compound is represented by a structure of Formula (IIb):

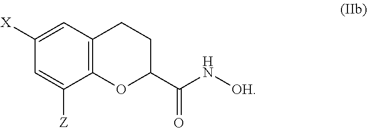

Other embodiments include a compound selected from Table 1, or compound 50b, compound 50d, or compound 50e, or a pharmaceutically acceptable salt, solvate, or prodrug thereof. Other embodiments include a compound selected from Table 1, or a pharmaceutically acceptable salt, solvate, or prodrug thereof.

TABLE 1

| Compound | Structure |
|---|---|
| 1-1 | 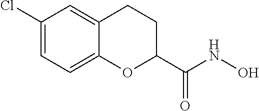 [SMILES: O=C(NO)C1CCc2cc(Cl)ccc2O1] |
| 1-2 | 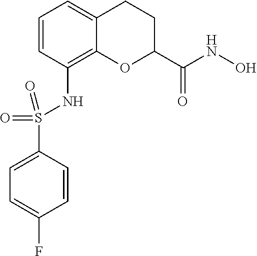 [SMILES: O=C(NO)C1CCc2cccc(NS(=O)(=O)c3ccc(F)cc3)c2O1] |
| 1-3 | 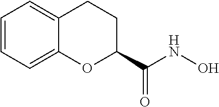 [SMILES: O=C(NO)[C@@H]1CCc2ccccc2O1] |
| 1-4 | 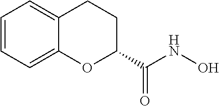 [SMILES: O=C(NO)[C@H]1CCc2ccccc2O1] |
| 1-5 | 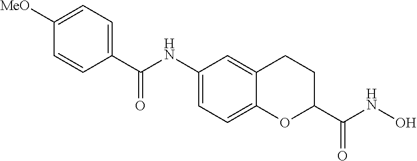 [SMILES: COc1ccc(C(=O)Nc2ccc3c(c2)CCC(C(=O)NO)O3)cc1] |
| 1-6 | 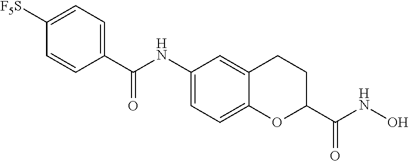 |
| 1-7 | 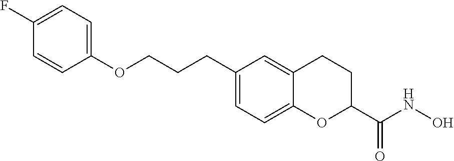 [SMILES: O=C(NO)C1CCc2cc(CCCOc3ccc(F)cc3)ccc2O1] |
| 1-8 | 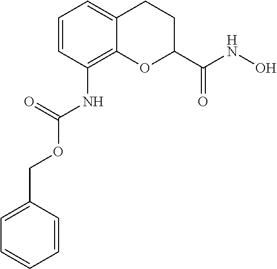 [SMILES: O=C(Nc1cccc2c1OC(C(=O)NO)CC2)OCc1ccccc1] |
| 1-9 | 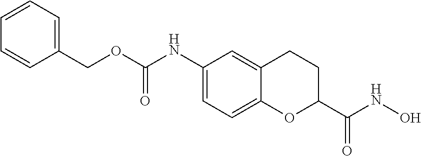 [SMILES: O=C(Nc1ccc2c(c1)CCC(C(=O)NO)O2)OCc1ccccc1] |
| 1-10 | 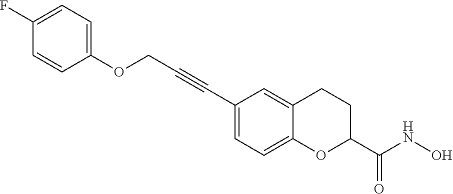 [SMILES: O=C(NO)C1CCc2cc(C#CCOc3ccc(F)cc3)ccc2O1] |
| 1-11 | 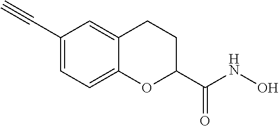 [SMILES: C#Cc1ccc2c(c1)CCC(C(=O)NO)O2] |
| 1-12 | 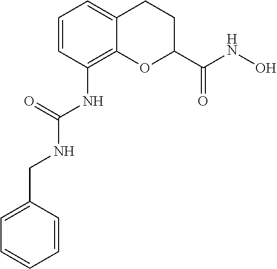 [SMILES: O=C(NCc1ccccc1)Nc1cccc2c1OC(C(=O)NO)CC2] |
| 1-13 | 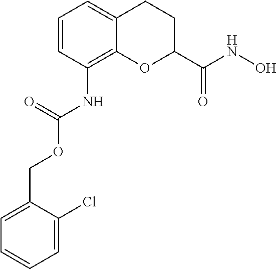 [SMILES: O=C(Nc1cccc2c1OC(C(=O)NO)CC2)OCc1ccccc1Cl] |
| 1-14 | 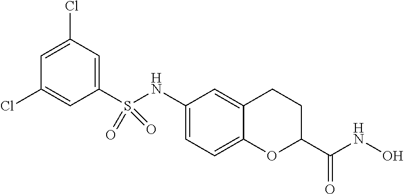 [SMILES: O=C(NO)C1CCc2cc(NS(=O)(=O)c3cc(Cl)cc(Cl)c3)ccc2O1] |
| 1-15 | 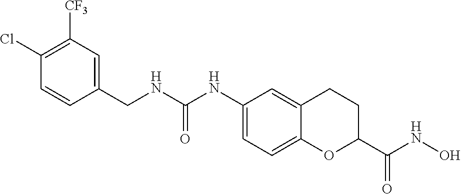 [SMILES: O=C(NCc1ccc(Cl)c(C(F)(F)F)c1)Nc1ccc2c(c1)CCC(C(=O)NO)O2] |
| 1-16 | 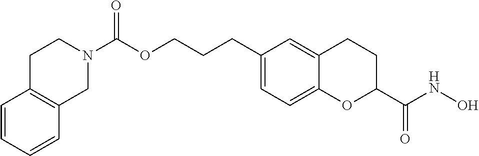 [SMILES: O=C(NO)C1CCc2cc(CCCOC(=O)N3CCc4ccccc4C3)ccc2O1] |
| 1-17 | 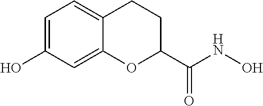 [SMILES: O=C(NO)C1CCc2ccc(O)cc2O1] |
| 1-18 | 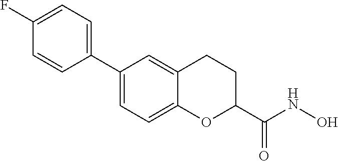 [SMILES: O=C(NO)C1CCc2cc(-c3ccc(F)cc3)ccc2O1] |
| 1-19 | 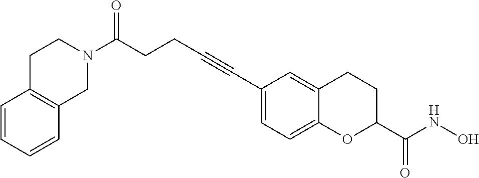 [SMILES: O=C(NO)C1CCc2cc(C#CCCC(=O)N3CCc4ccccc4C3)ccc2O1] |
| 1-20 | 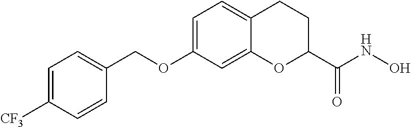 [SMILES: O=C(NO)C1CCc2ccc(OCc3ccc(C(F)(F)F)cc3)cc2O1] |
| 1-21 | 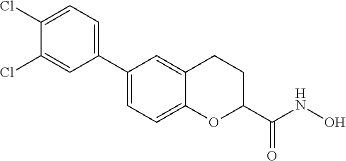 [SMILES: O=C(NO)C1CCc2cc(-c3ccc(Cl)c(Cl)c3)ccc2O1] |
| 1-22 | 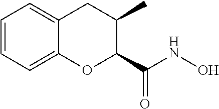 |
| 1-23 | 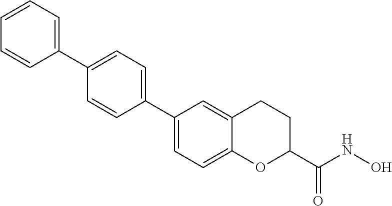 [SMILES: O=C(NO)C1CCc2cc(-c3ccc(-c4ccccc4)cc3)ccc2O1] |
| 1-24 | 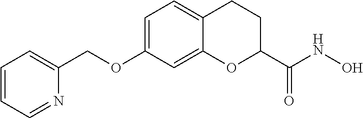 [SMILES: O=C(NO)C1CCc2ccc(OCc3ccccn3)cc2O1] |
| 1-25 | 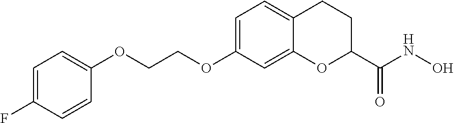 [SMILES: O=C(NO)C1CCc2ccc(OCCOc3ccc(F)cc3)cc2O1] |
| 1-26 | 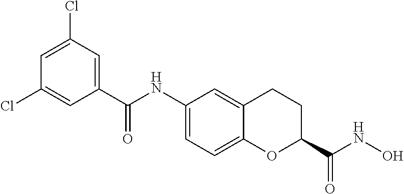 [SMILES: O=C(Nc1ccc2c(c1)CC[C@@H](C(=O)NO)O2)c1cc(Cl)cc(Cl)c1] |
| 1-27 | 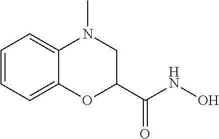 |
| 1-28 | 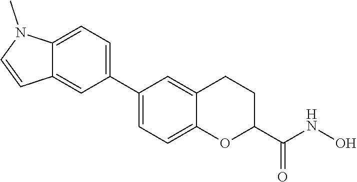 [SMILES: Cn1ccc2cc(-c3ccc4c(c3)CCC(C(=O)NO)O4)ccc21] |
| 1-29 | 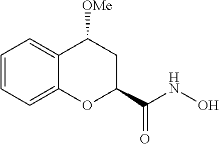 [SMILES: CO[C@@H]1C[C@@H](C(=O)NO)Oc2ccccc21] |
| 1-30 | 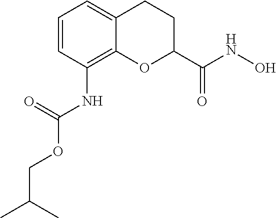 [SMILES: CC(C)COC(=O)Nc1cccc2c1OC(C(=O)NO)CC2] |
| 1-31 | 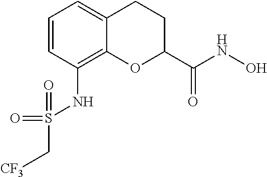 [SMILES: O=C(NO)C1CCc2cccc(NS(=O)(=O)CC(F)(F)F)c2O1] |
| 1-32 | 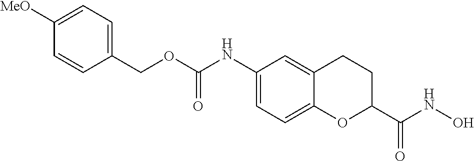 [SMILES: COc1ccc(COC(=O)Nc2ccc3c(c2)CCC(C(=O)NO)O3)cc1] |
| 1-33 | 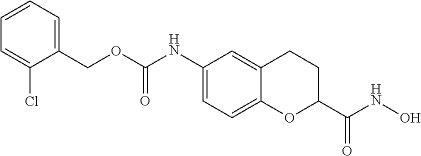 [SMILES: O=C(Nc1ccc2c(c1)CCC(C(=O)NO)O2)OCc1ccccc1Cl] |
| 1-34 | 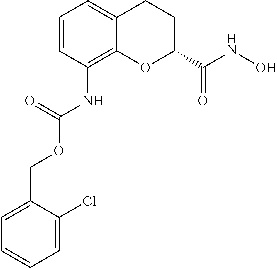 [SMILES: O=C(Nc1cccc2c1O[C@@H](C(=O)NO)CC2)OCc1ccccc1Cl] |
| 1-35 | 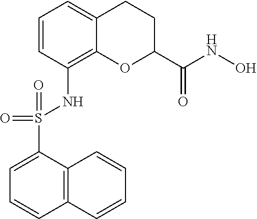 [SMILES: O=C(NO)C1CCc2cccc(NS(=O)(=O)c3cccc4ccccc34)c2O1] |
| 1-36 | 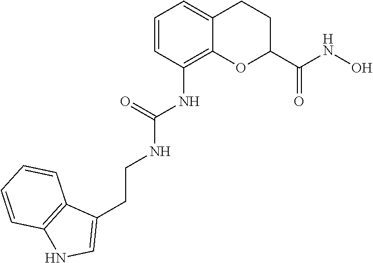 [SMILES: O=C(NCCc1c[nH]c2ccccc12)Nc1cccc2c1OC(C(=O)NO)CC2] |
| 1-37 | 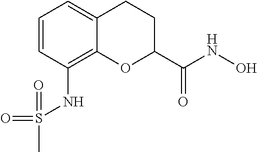 [SMILES: CS(=O)(=O)Nc1cccc2c1OC(C(=O)NO)CC2] |
| 1-38 | 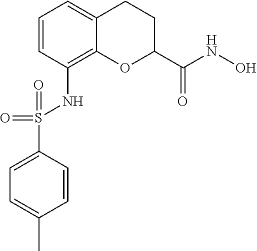 [SMILES: Cc1ccc(S(=O)(=O)Nc2cccc3c2OC(C(=O)NO)CC3)cc1] |
| 1-39 | 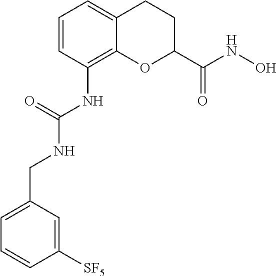 |
| 1-40 | 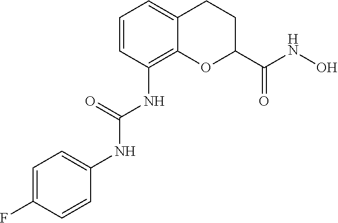 [SMILES: O=C(Nc1ccc(F)cc1)Nc1cccc2c1OC(C(=O)NO)CC2] |
| 1-41 | 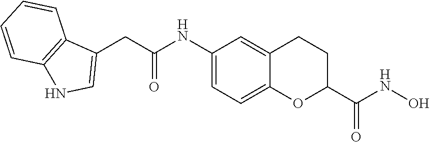 [SMILES: O=C(Cc1c[nH]c2ccccc12)Nc1ccc2c(c1)CCC(C(=O)NO)O2] |
| 1-42 | 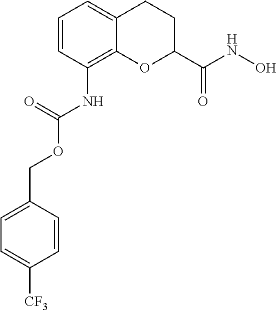 [SMILES: O=C(Nc1cccc2c1OC(C(=O)NO)CC2)OCc1ccc(C(F)(F)F)cc1] |
| 1-43 | 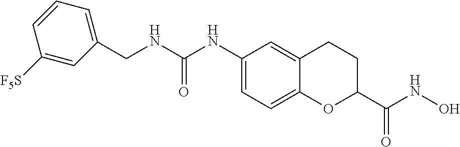 |
| 1-44 | 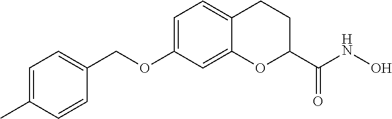 [SMILES: Cc1ccc(COc2ccc3c(c2)OC(C(=O)NO)CC3)cc1] |
| 1-45 | 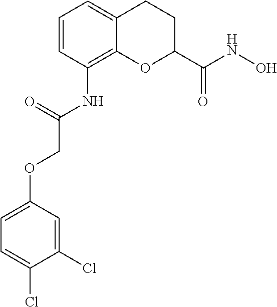 [SMILES: O=C(COc1ccc(Cl)c(Cl)c1)Nc1cccc2c1OC(C(=O)NO)CC2] |
| 1-46 | 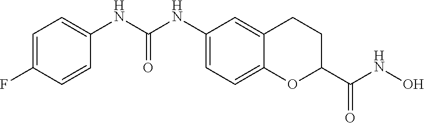 [SMILES: O=C(Nc1ccc(F)cc1)Nc1ccc2c(c1)CCC(C(=O)NO)O2] |
| 1-47 | 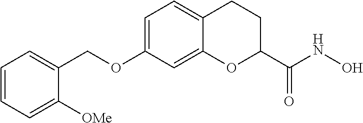 [SMILES: COc1ccccc1COc1ccc2c(c1)OC(C(=O)NO)CC2] |
| 1-48 | 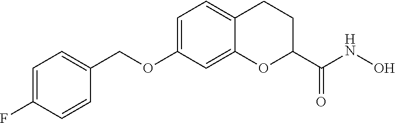 [SMILES: O=C(NO)C1CCc2ccc(OCc3ccc(F)cc3)cc2O1] |
| 1-49 | 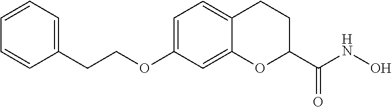 [SMILES: O=C(NO)C1CCc2ccc(OCCc3ccccc3)cc2O1] |
| 1-50 | 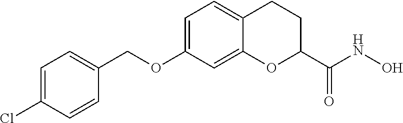 [SMILES: O=C(NO)C1CCc2ccc(OCc3ccc(Cl)cc3)cc2O1] |
| 1-51 | 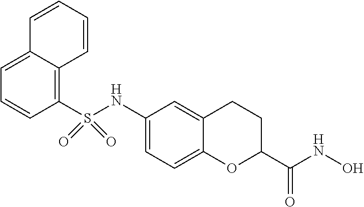 [SMILES: O=C(NO)C1CCc2cc(NS(=O)(=O)c3cccc4ccccc34)ccc2O1] |
| 1-52 | 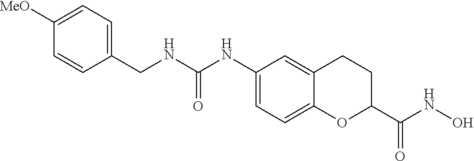 [SMILES: COc1ccc(CNC(=O)Nc2ccc3c(c2)CCC(C(=O)NO)O3)cc1] |
| 1-53 | 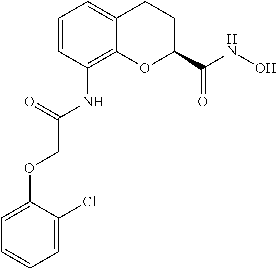 [SMILES: O=C(COc1ccccc1Cl)Nc1cccc2c1O[C@H](C(=O)NO)CC2] |
| 1-54 | 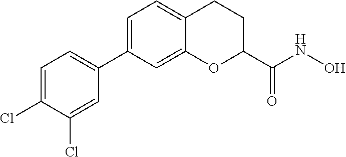 [SMILES: O=C(NO)C1CCc2ccc(-c3ccc(Cl)c(Cl)c3)cc2O1] |
| 1-55 | 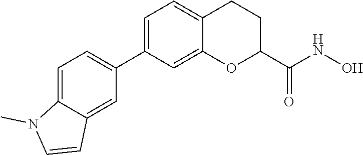 [SMILES: Cn1ccc2cc(-c3ccc4c(c3)OC(C(=O)NO)CC4)ccc21] |
| 1-56 | 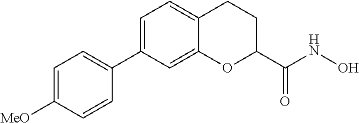 [SMILES: COc1ccc(-c2ccc3c(c2)OC(C(=O)NO)CC3)cc1] |
| 1-57 | 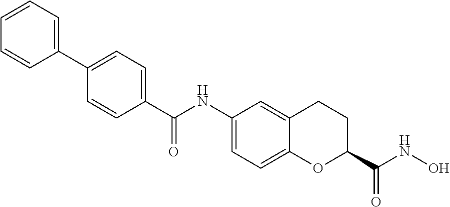 [SMILES: O=C(Nc1ccc2c(c1)CC[C@@H](C(=O)NO)O2)c1ccc(-c2ccccc2)cc1] |
| 1-58 | 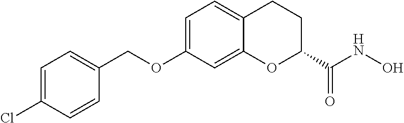 [SMILES: O=C(NO)[C@H]1CCc2ccc(OCc3ccc(Cl)cc3)cc2O1] |
| 1-59 | 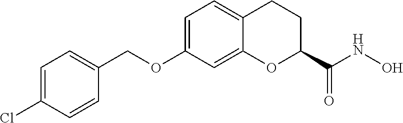 [SMILES: O=C(NO)[C@@H]1CCc2ccc(OCc3ccc(Cl)cc3)cc2O1] |
| 1-60 | 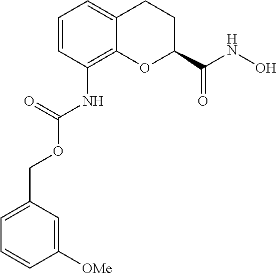 [SMILES: COc1cccc(COC(=O)Nc2cccc3c2O[C@H](C(=O)NO)CC3)c1] |
| 1-61 | 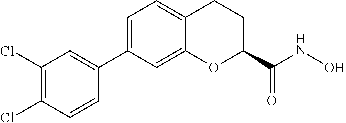 [SMILES: O=C(NO)[C@@H]1CCc2ccc(-c3ccc(Cl)c(Cl)c3)cc2O1] |
| 1-62 | 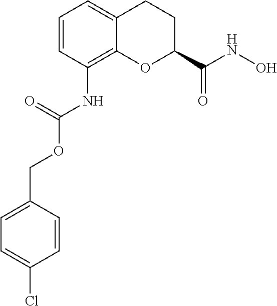 [SMILES: O=C(Nc1cccc2c1O[C@H](C(=O)NO)CC2)OCc1ccc(Cl)cc1] |
| 1-63 | 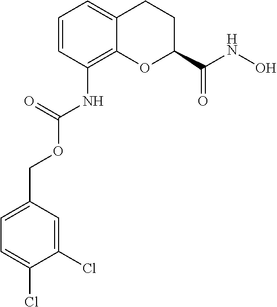 [SMILES: O=C(Nc1cccc2c1O[C@H](C(=O)NO)CC2)OCc1ccc(Cl)c(Cl)c1] |
| 1-64 | 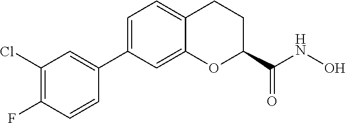 [SMILES: O=C(NO)[C@@H]1CCc2ccc(-c3ccc(F)c(Cl)c3)cc2O1] |
| 1-65 | 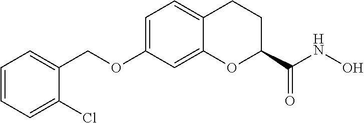 [SMILES: O=C(NO)[C@@H]1CCc2ccc(OCc3ccccc3Cl)cc2O1] |
| 1-66 | 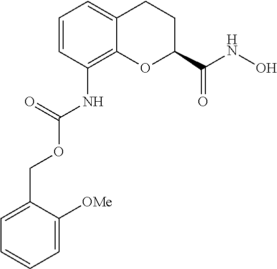 [SMILES: COc1ccccc1COC(=O)Nc1cccc2c1O[C@H](C(=O)NO)CC2] |

### II. Pharmaceutical Formulations

For oral administration, liquid or solid dose formulations may be used. Some non-limiting examples of oral dosage formulations include tablets, gelatin capsules, pills, troches, elixirs, suspensions, syrups, wafers, chewing gum and the like. The compounds can be mixed with a suitable pharmaceutical carrier (vehicle) or excipient as understood by practitioners in the art. Non-limiting examples of carriers and excipients include starch, milk, sugar, certain types of clay, gelatin, lactic acid, stearic acid or salts thereof, including magnesium or calcium stearate, talc, vegetable fats or oils, gums and glycols.

For systemic, intracerebroventricular, intrathecal, topical, intranasal, subcutaneous, or transdermal administration, formulations of the compounds useful in the methods of the present technology may utilize conventional diluents, carriers, or excipients etc., which are known in the art to be employed to deliver the compounds. For example, the formulations may comprise one or more of the following: a stabilizer, a surfactant (such as a nonionic, ionic, anionic, cationic, or zwitterionic surfactant), and optionally a salt and/or a buffering agent. The compound may be delivered in the form of a solution or in a reconstituted lyophilized form.

In some embodiments, the stabilizer may, for example, be an amino acid, such as for instance, glycine or an oligosaccharide, such as for example, sucrose, tetralose, lactose or a dextran. Alternatively, the stabilizer may be a sugar alcohol, such as for instance, mannitol, sorbitol, xylitol, or a combination thereof. In some embodiments, the stabilizer or combination of stabilizers constitutes from about 0.1% to about 10% by weight of the formulation, or any percentage in between these two values.

In some embodiments, the surfactant is a nonionic surfactant, such as a polysorbate. Some examples of suitable surfactants include polysorbates (e.g., Tween20, Tween80); a polyethylene glycol or a polyoxyethylene polyoxypropylene glycol, such as Pluronic F-68 from about 0.001% (w/v) to about 10% (w/v).

A salt or buffering agent may be any salt or buffering agent, such as sodium chloride, or sodium/potassium phosphate, respectively. In certain embodiments, the buffering agent maintains the pH of the pharmaceutical composition in the range of about 5.5 to about 7.5. The salt and/or buffering agent is also useful to maintain the osmolality at a level suitable for administration to a human or an animal. In some embodiments, the salt or buffering agent is present at a roughly isotonic concentration of about 150 mM to about 300 mM.

The formulations of the compounds useful in the methods of the present technology may additionally comprise one or more conventional additives. Some non-limiting examples of such additives include a solubilizer such as glycerol; an antioxidant such as benzalkonium chloride (a mixture of quaternary ammonium compounds, known as “quats”), benzyl alcohol, chloretone or chlorobutanol; anaesthetic agent such as a morphine derivative; or an isotonic agent etc., such as described above. As a further precaution against oxidation or other spoilage, the pharmaceutical compositions may be stored under nitrogen gas in vials sealed with impermeable stoppers.

The mammal can be any mammal, including farm animals, such as sheep, pigs, cows, and horses; pet animals, such as dogs and cats; laboratory animals, such as rats, mice and rabbits. In some embodiments, the mammal is a human.

### III. Methods of Treatment

In some embodiments, the compounds of the present disclosure may be used to inhibit histone acetylation in a cell or a subject.

One aspect of the present technology includes methods of inhibiting histone acetylation in a subject in need thereof. In some embodiments, the treatment of the subject in need thereof comprises administering a compound of the present disclosure (e.g., a compound of Formula (I)) to the subject. In some embodiments, the subject is suffering from, suspected as having, or at risk of having a disease capable of treatments by inhibition of histone acetylation, such as malignancies (e.g., a hematologic malignancy). In some embodiments, the subject is suffering from, suspected as having, or at risk of having a cancer that is responsive to HDAC inhibition, such as Pancreatic, Esophageal squamous cell carcinoma (ESCC), Multiple myeloma, Prostate carcinoma, Gastric cancer, Leukemia, breast, Liver cancer, ovarian cancer, non-Hodgkin lymphoma and Neuroblastoma. In some embodiments, the subject is suffering from, suspected as having, or at risk of having an infectious disease that is responsive to HDAC inhibition, such as malaria (e.g., P. falciparum infections), HIV, pnumenoia, C. albicans infections.

The compound may be included in a pharmaceutical formulation, such as those disclosed herein, and may be administered in any pharmaceutically acceptable manner, including methods of administration described herein.

The compounds useful in the methods of the present technology are administered to a mammal in an amount effective in inhibiting histone acetylation. The therapeutically effective amount can be determined by methods known in the art.

An effective amount of a compound useful in the methods of the present technology, for example in a pharmaceutical composition, may be administered to a mammal in need thereof by any of a number of well-known methods for administering pharmaceutical compounds. The compound may be administered systemically or locally. In one embodiment, the compound is administered intravenously. For example, the compounds useful in the methods of the present technology may be administered via rapid intravenous bolus injection. In some embodiments, the compound is administered as a constant rate intravenous infusion. The compound may also be administered orally, topically, intranasally, intramuscularly, subcutaneously, or transdermally. Other routes of administration include intracerebroventricularly or intrathecally. Intracerebroventiculatly refers to administration into the ventricular system of the brain. Intrathecally refers to administration into the space under the arachnoid membrane of the spinal cord.

The compounds useful in the methods of the present technology may also be administered to mammals by sustained or controlled release, as is known in the art. Sustained release administration is a method of drug delivery to achieve a certain level of the drug over a particular period of time. The level typically is measured by serum or plasma concentration.

In one preferred embodiment, the compounds are administered orally. In one preferred embodiment, the compounds are administered intravenously. In one preferred embodiment, the compounds are administered at less than about 1 gram per day. In other embodiments of the present technology, the compounds are administered at less than about 10, at less than about 9, at less than about 8, at less than about 7, at less than about 6, at less than about 5, at less than about 4, at less than about 3, at less than about 2, at less than about 1, at less than about 0.9, at less than about 0.8, at less than about 0.7, at less than about 0.6, at less than about 0.5, at less than about 0.4, at less than about 0.3, at less than about 0.2, or at less than about 0.1 grams per day, or any amount in between these values.

### IV. Definitions

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Certain ranges are presented herein with numerical values being preceded by the term “about”. The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Generally, reference to a certain element such as hydrogen or H is meant to include all isotopes of that element. For example, if a group is defined to include hydrogen or H, it also includes deuterium and tritium. Hence, isotopically labeled compounds are within the scope of the invention. In some embodiments, one or more of the H in Formulae (I) or (I′) or (II) is replaced with a deuterium.

In general, “substituted” refers to an organic group (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. The present disclosure is understood to include embodiments where, for instance a “substituted alkyl” optionally contains one or more alkene and/or alkyne. A substituted group will be substituted with one or more substituents, unless otherwise specified. In some embodiments, a substituted group is substituted with 1, 2, 3, 4, 5, or 6 substituents.

Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; aryl groups; heteroaryl groups; cycloalkyl groups; heterocyclyl groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

Substituted ring groups such as substituted cycloalkyl, aryl, heterocycle and heteroaryl groups also include rings and fused ring systems in which a bond to a hydrogen atom is replaced with a bond to a carbon atom. Therefore, substituted cycloalkyl, aryl, heterocycle and heteroaryl groups may also be substituted with substituted or unsubstituted alkyl, alkenyl, and alkynyl groups as defined below.

Alkyl groups include straight chain and branched alkyl groups having from 1 to about 20 carbon atoms, and typically from 1 to 12 carbons or, in some embodiments, from 1 to 8, 1 to 6, or 1 to 4 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, and isopentyl groups. Representative substituted alkyl groups may be substituted one or more times with substituents such as those listed herein. As stated above, the present disclosure is understood to include embodiments where, for instance a “substituted alkyl” optionally contains one or more alkene and/or alkyne.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include mono-, bicyclic and polycyclic ring systems, such as, for example bridged cycloalkyl groups as described below, and fused rings, such as, but not limited to, decalinyl, and the like. In some embodiments, polycyclic cycloalkyl groups have three rings. Substituted cycloalkyl groups may be substituted one or more times with non-hydrogen and non-carbon groups as defined above.

However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4-2,5- or 2,6-di-substituted cyclohexyl groups, which may be substituted with substituents such as those listed above. In some embodiments, a cycloalkyl group has one or more alkene bonds, but is not aromatic.

Aryl groups are cyclic aromatic hydrocarbons that do not contain heteroatoms. Aryl groups include monocyclic, bicyclic and polycyclic ring systems. Thus, aryl groups include, but are not limited to, phenyl, azulenyl, heptalenyl, biphenylenyl, indacenyl, fluorenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, and in others from 6 to 12 or even 6-10 carbon atoms in the ring portions of the groups. Although the phrase “aryl groups” includes groups containing fused rings, such as fused aromatic-aliphatic ring systems (e.g., indanyl, tetrahydronaphthyl, and the like), it does not include aryl groups that have other groups, such as alkyl or halo groups, bonded to one of the ring members. Rather, groups such as tolyl are referred to as substituted aryl groups. Representative substituted aryl groups may be mono-substituted or substituted more than once. For example, monosubstituted aryl groups include, but are not limited to, 2-, 3-, 4-, 5-, or 6-substituted phenyl or naphthyl groups, which may be substituted with substituents such as those listed above.

Heterocycle groups include aromatic (also referred to as heteroaryl) and non-aromatic ring compounds containing 3 or more ring members, of which one or more is a heteroatom such as, but not limited to, N, O, S or B. In some embodiments, heterocycle groups include 3 to 20 ring members, whereas other such groups have 3 to 6, 3 to 10, 3 to 12, or 3 to 15 ring members. Heterocycle groups encompass unsaturated, partially saturated and saturated ring systems, such as, for example, imidazolyl, imidazolinyl and imidazolidinyl groups. The phrase “heterocycle group” includes fused ring species including those comprising fused aromatic and non-aromatic groups, such as, for example, benzotriazolyl, 2,3-dihydrobenzo[1,4]dioxinyl, and benzo[1,3]dioxolyl. The phrase also includes bridged polycyclic ring systems containing a heteroatom such as, but not limited to, quinuclidyl. However, the phrase does not include heterocycle groups that have other groups, such as alkyl, oxo or halo groups, bonded to one of the ring members. Rather, these are referred to as “substituted heterocycle groups”. Heterocycle groups include, but are not limited to, aziridinyl, azetidinyl, pyrrolidinyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl, tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocycle groups may be mono-substituted or substituted more than once, such as, but not limited to, pyridyl or morpholinyl groups, which are 2-, 3-, 4-, 5-, or 6-substituted, or disubstituted with various substituents such as those listed above.

Heteroaryl groups are aromatic ring compounds containing 5 or more ring members, of which, one or more is a heteroatom such as, but not limited to, N, O, S or B. Heteroaryl groups include, but are not limited to, groups such as pyrrolyl, pyrazolyl, triazolyl, tetrazolyl, oxazolyl, isoxazolyl, thiazolyl, pyridyl, pyridazinyl, pyrimidinyl, pyrazinyl, thiophenyl, benzothiophenyl, furanyl, benzofuranyl, indolyl, azaindolyl (pyrrolopyridyl), indazolyl, benzimidazolyl, imidazopyridyl (azabenzimidazolyl), pyrazolopyridyl, triazolopyridyl, benzotriazolyl, benzoxazolyl, benzothiazolyl, benzothiadiazolyl, imidazopyridyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups. Although the phrase “heteroaryl groups” includes fused ring compounds such as indolyl and 2,3-dihydro indolyl, the phrase does not include heteroaryl groups that have other groups bonded to one of the ring members, such as alkyl groups. Rather, heteroaryl groups with such substitution are referred to as “substituted heteroaryl groups.” Representative substituted heteroaryl groups may be substituted one or more times with various substituents such as those listed above.

Alkoxy groups are hydroxyl groups (—OH) in which the bond to the hydrogen atom is replaced by a bond to a carbon atom of a substituted or unsubstituted alkyl group as defined above. Examples of linear alkoxy groups include, but are not limited to, methoxy, ethoxy, propoxy, butoxy, pentoxy, hexoxy, and the like. Examples of branched alkoxy groups include but are not limited to isopropoxy, sec-butoxy, tert-butoxy, isopentoxy, isohexoxy, and the like. Examples of cycloalkoxy groups include, but are not limited to, cyclopropyloxy, cyclobutyloxy, cyclopentyloxy, cyclohexyloxy, and the like. Representative substituted alkoxy groups may be substituted one or more times with substituents such as those listed above.

The term “amine” (or “amino”) as used herein refers to —NHR* and —NR*R* groups, wherein R* are independently hydrogen, or a substituted or unsubstituted alkyl, alkenyl, alkynyl, cycloalkyl, aryl, aralkyl, heterocyclylalkyl or heterocycle group as defined herein. In some embodiments, the amine is NH₂, methylamino, dimethylamino, ethylamino, diethylamino, propylamino, isopropylamino, phenylamino, or benzylamino.

The term “amide” refers to a —NR*R*C(O)— group wherein R* each independently refer to a hydrogen, (C₁-C₅)alkyl, or (C₃-C₆)aryl.

Those of skill in the art will appreciate that compounds of the invention may exhibit the phenomena of tautomerism, conformational isomerism, geometric isomerism and/or optical isomerism. As the formula drawings within the specification and claims can represent only one of the possible tautomeric, conformational isomeric, optical isomeric or geometric isomeric forms, it should be understood that the invention encompasses any tautomeric, conformational isomeric, optical isomeric and/or geometric isomeric forms of the compounds having one or more of the utilities described herein, as well as mixtures of these various different forms.

As readily understood by one skilled in the art, a wide variety of functional groups and other structures may exhibit tautomerism, and all tautomers of compounds as described herein are within the scope of the present invention.

Stereoisomers of compounds, also known as “optical isomers,” include all chiral, diastereomeric, and racemic forms of a structure, unless the specific stereochemistry is expressly indicated. Thus, compounds used in the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these are all within the scope of the invention.

By “pharmaceutically acceptable” is meant a material that is not biologically or otherwise undesirable, e.g., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. and Drug administration.

By “patient” is meant any animal for which treatment is desirable. Patients may be mammals, and typically, as used herein, a patient is a human individual.

The term “pharmaceutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the present invention and the like.

The term “solvates” is used in its broadest sense. For example, the term solvates includes hydrates formed when a compound of the present invention contains one or more bound water molecules.

Certain compounds within the scope of the disclosure are derivatives referred to as prodrugs. The expression “prodrug” denotes a derivative of a known direct acting drug, e.g. esters and amides, which derivative has enhanced delivery characteristics and therapeutic value as compared to the drug, and is transformed into the active drug by an enzymatic or chemical process; see Notari, R. E., “Theory and Practice of Prodrug Kinetics,” Methods in Enzymology 112: 309-23 (1985); Bodor, N., “Novel Approaches in Prodrug Design,” Drugs of the Future, 6: 165-82 (1981); and Bundgaard, H., “Design of Prodrugs: Bioreversible-Derivatives for Various Functional Groups and Chemical Entities,” in DESIGN OF PRODRUGS (H. Bundgaard, ed.), Elsevier (1985), Goodman and Gilmans, THE PHARMACOLOGICAL BASIS OF THERAPEUTICS, 8th ed., McGraw-Hill (1992). In some embodiments, the “prodrug” is a compound that generally converts to an active compound of the present disclosure within a physiological environment (e.g., stomach, colon, blood). Pro-drugs include esters, carbonates, carbamates, oximes of active alcohols (and/or acids for esters), amides, carbamates, ureas, oximes, Mannich bases, imines of amines (and/or acids for amides), carbondithianes of active thiols, conjugates of reactive species such as a,b-unsaturated carbonyl derivatives. The selection and synthesis of prodrugs include strategies such as those in: Karaman, R., “Prodrugs design based on inter- and intramolecular chemical processes,” Chem. Biol. Drug Des., 82: 643-668 (2013); Huttunen et al., “Prodrugs—from serendipity to rational design,” Pharmacol. Rev., 63, 750-771 (2011); Blencowe et al., “Self-immolative linkers in polymeric delivery systems,” Polym. Chem., 2: 773-790 (2011); Arpicco et al., “Anticancer prodrugs: An overview of major strategies and recent developments,” Curr. Top. Med. Chem. (Sharjah, United Arab Emirates), 11: 2346-2381 (2011); Tietze et al., “Antibody-directed enzyme prodrug therapy: A promising approach for a selective treatment of cancer based on prodrugs and monoclonal antibodies” Chem. Biol. Drug Des., 74: 205-211 (2009); Simplicio et al., “Prodrugs for amines,” Molecules, 13: 519-547 (2008); Rautio et al., “Prodrugs: Design and clinical applications,” Nat. Rev. Drug Discovery, 7: 255-270 (2008); Lee et al., “Pro-drug and Antedrug: Two Diametrical Approaches in Designing Safer Drugs,” Arch. Pharm. Res., 25: 111-136 (2002); and Lee, Chem. Biol. Drug Des., 82: 643-668 (2013).

### EXAMPLES

The present technology is further illustrated by the following examples, which should not be construed as limiting in any way.

General information. All non-aqueous reactions were carried out under a nitrogen atmosphere in oven- or flame-dried glassware unless otherwise noted. Anhydrous tetrahydrofuran and diethyl ether were distilled from sodium benzophenone ketyl; anhydrous dichloromethane and toluene were distilled from CaH₂; alternatively, the same solvents were obtained from a solvent purification system using alumina columns. All other solvents and reagents were used as obtained from commercial sources without further purification unless otherwise noted. Reactions were monitored via TLC using 250 μm pre-coated silica gel 60 F₂₅₄ plates, which were visualized with 254 nm and/or 365 nm UV light and by staining with KMnO₄ (1.5 g KMnO₄, 10 g K₂CO₃, and 1.25 mL 10% NaOH in 200 mL water), cerium molybdate (0.5 g Ce(NH₄)₂(NO₃)₆, 12 g (NH₄)₆Mo₇O₂₄·4H₂O, and 28 mL conc. H₂SO₄ in 235 mL water), or vanillin (6 g vanillin and 1.5 mL conc. H₂SO₄ in 100 mL EtOH). Flash chromatography was performed with SiliCycle silica gel 60 (230-400 mesh) or with ISCO MPLC. ¹H and ¹³C NMR spectra were recorded on Bruker Avance 300, 400, or 500 MHz spectrometers, using the residual solvent as an internal standard. IR spectra were obtained on a Smiths IdentifyIR or PerkinElmer Spectrum 100. HRMS data were obtained on a Thermo Scientific Exactive HRMS coupled to a Thermo Scientific Accela HPLC system using a 2.1×50 mm 3.5 μm Waters XTerra Cis column eluting with MeCN/H₂O containing 0.1% formic acid. Purity of compounds was assessed using the same HPLC system with either the PDA or an Agilent 385 ELSD. All final screening samples passed QC based on >95% purity by LC/MS/ELSD analysis.

### General Synthetic Methods

The compounds of the present disclosure can be prepared using the following general methods and procedures. The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

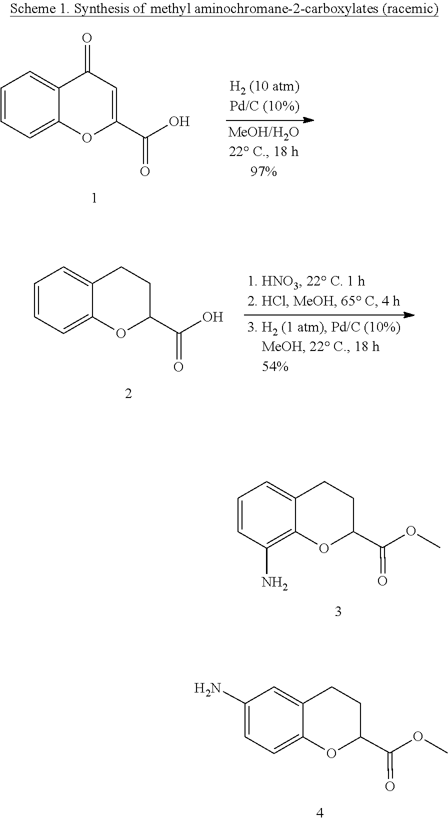

Chromane-2-carboxylic acid (2). In each of three Parr vials, a suspension of chromone-carboxylic acid (2.22 g, 11.7 mmol) in MeOH (15 mL) was added H₂O (0.2 mL) followed by Pd/C (10%, 0.240 g, 0.225 mmol). The mixture was stirred under H₂ (10 bar) using Parr apparatus. After 5 h, the reaction was recharged with H₂ (10 bar). After 16 h, the combined mixtures were filtered through Celite®, rinsed with MeOH, and concentrated to give chromane-2-carboxylic acid (2, 6.19 g, 97%) as a beige-colored solid: ¹H NMR (DMSO-d₆, 300 MHz) δ 13.01 (s, 1H), 7.06 (m, 2H), 6.81 (m, 2H), 4.90 (dd, J=6.5, 3.8 Hz, 0.15H), 4.76 (dd, J=6.3, 3.9 Hz, 0.85H), 2.78 (m, 1H), 2.68-2.58 (m, 1H), 2.20-1.99 (m, 2H); HRMS (ESI+) m/z calcd for C₁₀H₉O₃ [M−H] 177.0546, found 177.0537.

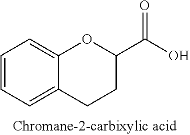 [SMILES: O=C(O)C1CCc2ccccc2O1]

To a mixture of 4-oxo-4H-chromene-2-carboxylic acid (1.07 g, 5.62 mmol) in MeOH (10 mL) and H₂O (0.20 mL) was added Pd/C (10%, 0.124 g). The mixture was subjected to H₂ (60 psi) using Parr apparatus. After 4 h, a few drops of AcOH were added and the reaction was subjected to H₂ (60 psi) using Parr apparatus overnight. The solution was filtered through Celite®, rinsed with MeOH and concentrated. The residue was diluted with CH₂Cl₂ and concentrated to give chromane-2-carboxylic acid (48, 0.974 g, 97%) as an off-white solid that was used without further purification: ¹H NMR (DMSO-d₆, 300 MHz) δ 12.99 (br s, 1H), 7.05-7.03 (m, 2H), 6.81-6.77 (m, 2H), 4.77-4.74 (m, 1H), 2.78-2.75 (m, 1H), 2.73-2.63 (m, 1H), 2.15-2.06 (m, 2H).

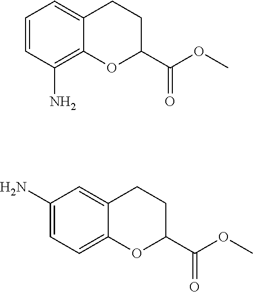

Methyl 8-aminochromane-2-carboxylate (3) and methyl 6-aminochromane-2-carboxylate (4). Nitration: An ice cold solution of nitric acid (20.0 mL, 70%) was treated with 86 (1.09 g, 6.14 mmol) portion wise. The solution turned green after 30-45 min. The mixture was warmed to room temperature after 45 min, and was left to stir for an additional 15 min. The solution was poured into ice, and the mixture was extracted with CHCl₃ (4×). The combined organic layer was concentrated to 200 mL, washed with brine, dried (Na₂SO₄), filtered, and concentrated to give an orange colored residue (1.10 g). Esterification: The residue (1.10 g) was dissolved in MeOH (16 mL) and treated with conc. HCl (3 pipette drops) at room temperature. The mixture was heated at reflux for 4 h. The mixture was concentrated, diluted with EtOAc, washed with satd. NaHCO₃, brine, dried (Na₂SO₄), filtered and concentrated to provide a beige/yellowish powder (970 mg). Reduction: The mixture was diluted with MeOH (15 mL) in a 100 mL RBF. The flask was flushed with N₂, and Pd/C (10%, 0.215 g, 0.199 mmol) was added. The flask was flushed with H₂ for 10 min, and then kept under H₂ (1 atm-balloon) for 18 h. The mixture was filtered through Celite®, rinsed with MeOH, and concentrated. The oil was purified by chromatography on SiO₂ (20-100% EtOAc/hexanes) to give methyl 8-aminochromane-2-carboxylate (3, 0.312, 25%) as a light pink-colored solid and methyl 6-aminochromane-2-carboxylate (4, 0.371, 29%) as an amber oil: 3: mp 68-70° C.; IR (CDCl₃) 3464, 3372, 3031, 2952, 2928, 2851, 1745, 1617, 1485, 1438, 1195, 1095 cm⁻¹; ¹H NMR (CDCl₃₉, 500 MHz): δ 6.69 (app t, J=7.7 Hz, 1H), 6.58-6.56 (m, 1H), 6.45 (dt, J=7.6, 0.7 Hz, 1H), 4.76 (dd, J=7.8, 3.6 Hz, 1H), 3.79 (s, 3H), 3.79 (br s, 2H), 2.81 (1H), 2.75-2.69 (m, 1H), 2.31-2.25 (m, 1H), 2.17 (m, 1H); ¹³C NMR (CDCl₃, 125 MHz): δ 141.3, 135.7, 121.4, 121.0, 118.8, 113.4, 74.0, 52.4, 25.0, 23.4; HRMS (ESI+) m/z calcd for C₁₁H₁₄NO₃[M+H]208.0968, found 208.0968; 4: IR (CDCl₃) 3431, 3358, 3012, 2952, 2850, 1747, 1629, 1498, 1451, 1203 cm⁻¹; ¹H NMR (CDCl₃, 500 MHz): δ 6.76 (d, J=8.6 Hz, 1H), 6.50 (dd, J=8.6, 2.7 Hz, 1H), 6.39 (d, J=2.6 Hz, 1H), 4.65 (dd, J=8.0, 3.3 Hz, 1H), 3.79 (s, 3H), 3.37 (s, 2H), 2.81-2.74 (m, 1H), 2.70-2.65 (m, 1H), 2.28-2.22 (m, H), 2.18-2.10 (m, 1H); ¹³C NMR (CDCl₃, 125 MHz): δ 171.8, 146.7, 140.1, 122.0, 117.7, 115.9, 115.4, 74.0, 52.5, 25.1, 23.8; HRMS (ESI+) m/z calcd for C₁₁H₁₄NO₃ [M+H] 208.0968, found 208.0969.

### Synthesis of Intermediates 1a and 1b

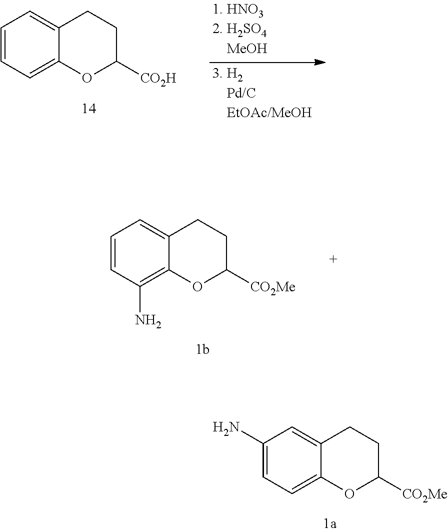 [SMILES: O=C(O)C1CCc2ccccc2O1 ; COC(=O)C1CCc2cccc(N)c2O1 ; COC(=O)C1CCc2cc(N)ccc2O1]

Synthetic intermediates 1a and 1b were prepared as follows according to Scheme above: To an ice-cold solution of nitric acid (45 mL) was added chromane-2-carboxylic acid 14 (3.50 g, 19.6 mmol) portion wise over 2-3 minutes. After 10 min, the dark reaction mixture was warmed to room temperature. After 30 min, the mixture was added to ice and the light green solid was collected by filtration, washed with H₂O, and dried in vacuo (2.9 g, crude). The filtrate was extracted with CH₂Cl₂ (2×). The combined organic layer was washed with brine, dried (Na₂SO₄), filtered and concentrated to give an orange solid residue (2.1 g, crude). This residue was diluted with MeOH (50 mL) and treated with H₂SO₄ (5 small drops) and heated to reflux. After 2 h, the solution was concentrated and the residue was extracted with EtOAc, washed with saturated NaHCO₃, brine, dried (Na₂SO₄), filtered and concentrated to give crude products as an orange solid residue. The light green solid (2.9 g, crude) was diluted with MeOH (50 mL) and treated with H₂SO₄ (5 small drops) and heated to reflux. After 2 h, the mixture was concentrated. The residue was diluted with H₂O and EtOAc, neutralized with NaHCO₃. The mixture was extracted with EtOAc, washed with satd. NaHCO₃, brine, dried (Na₂SO₄), filtered and concentrated to give an orange solid that was combined with material obtained above.

The orange solid residue (6.5 g, crude-combined) was diluted with MeOH (50 mL)/EtOAc (10 mL) evacuated and backflushed with N₂ and then treated with Pd/C (10%, 0.585 g). The mixture was evacuated and backflushed with H₂ (2×) and kept under H₂ (1 atm-balloon). After 18 h, the mixture was filtered through Celite®, rinsed with EtOAc and concentrated. The residue was absorbed onto Celite® and purified by chromatography on SiO₂ (ISCO-Rf, 0-100% EtOAc/hexanes) to give methyl 8-aminochromane-2-carboxylate 1b (1.04 g, 26% 3-steps) as a beige solid and methyl 6-aminochromane-2-carboxylate 1a (1.76 g, 43% 3-steps) as an amberoil.

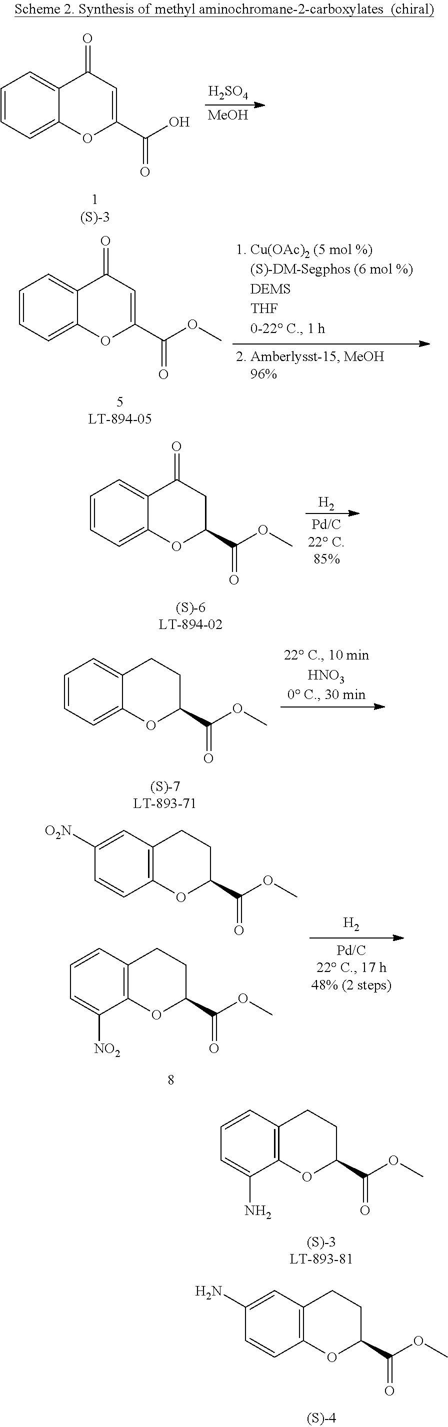

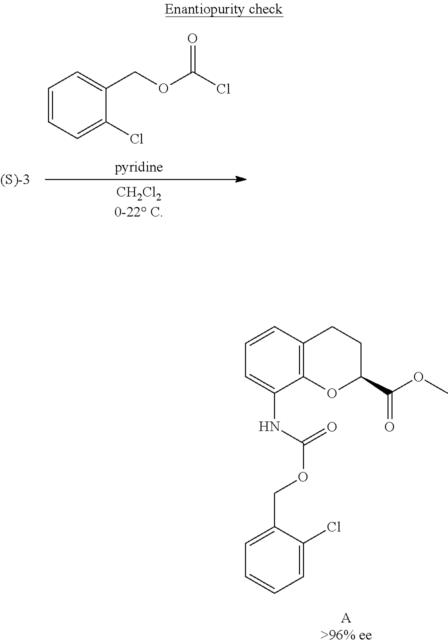 [SMILES: O=C(Cl)OCc1ccccc1Cl ; COC(=O)[C@@H]1CCc2cccc(NC(=O)OCc3ccccc3Cl)c2O1]

Methyl 4-oxo-4H-chromene-2-carboxylate (5). Chromone-2-carboxylic acid (45.0 g, 237 mmol) in methanol (700 mL) was treated with conc. sulfuric acid (5.4 mL) and warmed to 50° C. After 18 h, the mixture was cooled to room temperature, concentrated, transferred to a 2 L flask and diluted with EtOAc and satd. NaHCO₃. The biphasic layer was stirred for 15 min and the layers were separated. The organic layer was washed with brine, dried (Na₂SO₄), filtered, and concentrated to give methyl 4-oxo-4H-chromene-2-carboxylate (5, 43.8 g, 91%) as a light yellow solid: ¹H NMR (CDCl₃, 300 MHz): δ 8.19 (dd, J=8.0, 1.7 Hz, 1H), 7.76-7.71 (m, 1H), 7.59 (dd, J=8.4, 0.7 Hz, 1H), 7.47-7.42 (m, 1H), 7.10 (s, 1H), 4.00 (s, 3H); HRMS (ESI+) m/z calcd for C₁₁H₉O₄ [M+H] 205.0495, found 205.0493.

Methyl (S)-4-oxochromane-2-carboxylate ((S)-6). A bondi-blue solution of Cu(OAc)₂ (Strem, 0.089 g, 0.490 mmol) in freshly distilled THE (20 mL) was stirred under an atmosphere of N₂ until a homogeneous green-blue solution was obtained (ca. 15 min). Neat (S)-DM-Segphos (390 mg, 0.54 mmol) was added and the mixture was stirred for 15 min at room temperature, cooled to 0° C. and treated dropwise with diethoxymethylsilane (990 mg, 7.35 mmol). The reaction mixture was stirred for an additional 30 min at 0° C. with the solution gradually turned yellow. A solution of 5 (1.0 g, 4.90 mmol) in dry THE (10 mL) was added dropwise over 5 min. The solution was stirred at 0° C. for 30 min, and at rt for another 30 min, while it turned light brown. The reaction mixture was cooled to 0° C. and quenched with satd. NH₄Cl (10 mL) under vigorous stirring for 15 min. The reaction mixture was extracted with EtOAc. The organic layer was washed with satd. NaHCO₃, dried (Na₂SO₄), and concentrated. The oily residue was purified by chromatography on SiO₂ (0-25% EtOAc/hexanes) to give a 1:1 mixture of ketone:silyl enol ether (quant.). A solution of the combined ketone/enol ether fractions in MeOH (20 mL) were treated with Amberlyst-15 (100 mg) at room temperature. After 1 h, the reaction mixture was filtered and concentrated to give methyl (S)-4-oxochromane-2-carboxylate ((S)-6, 1.040 g, 96%, 93% purity) as a light yellow, waxy solid: (HPLC analysis >97% ee, ChiralPak AD-H (0.46×25 cm, 0.5 □M, 90/10 hexanes/EtOH): ¹H NMR (CDCl₃, 500 MHz): δ 7.89 (dd, J=7.9, 1.7 Hz, 1H), 7.55-7.71 (m, 1H), 7.11 (d, J=8.4 Hz, 1H), 7.09-7.06 (m, 1H), 5.10 (dd, J=8.4, 6.1 Hz, 1H), 3.82 (s, 3H), 3.07 (d, J=0.5 Hz, 1H), 3.06 (d, J=3.2 Hz, 1H); 13C NMR (CDCl₃, 125 MHz) δ 189.7, 169.3, 160.3, 136.6, 127.1, 122.4, 121.1, 118.3, 75.3, 53.0, 39.7; HRMS (ESI+) m/z calcd for C₁₁H₁₁O₄ [M+H] 207.0652, found 207.0650. C₁₁H₉O₄ [M+H] 205.0495, found 205.0493.

(S)-chromane-2-carboxylate ((S)-7). To a suspension methyl (S)-4-oxochromane-2-carboxylate ((S)-6, 0.094 g, 0.456 mmol) in MeOH (1.20 mL) was added H₂O (0.05 mL) followed by Pd/C (10%, 0.028 g, 0.027 mmol). The mixture was stirred under H₂ (1 atm-balloon). After 40 h, the mixture was filtered through Celite®, rinsed with EtOAc (10 mL), and concentrated to provide methyl (S)-chromane-2-carboxylate ((S)-7, 0.075 g, 85%) as a colorless oil: [α]_{D}=+8.7 (c 2.8, CHCl₃); ¹H NMR (CDCl₃, 300 MHz): δ 7.12 (app t, J=7.1 Hz, 1H), 7.04 (d, J=7.4 Hz, 1H), 6.93 (dd, J=8.2, 0.8 Hz, 1H), 6.87 (dt, J=7.4, 1.2 Hz, 1H), 4.74 (dd, J=7.5, 3.7 Hz, 1H), 3.80 (s, 3H), 2.90-2.70 (m, 2H), 2.34-2.13 (m, 2H).

Methyl (S)-8-aminochromane-2-carboxylate ((S)-3) and methyl (S)-6-aminochromane-2-carboxylate ((S)-4). An ice-cold solution of methyl (S)-chromane-2-carboxylate ((S)-7, 0.107 g, 0.558 mmol) was treated with an ice-cold solution of nitric acid (2.50 mL, 70%) portion wise. The solution turned dark blue after 30 min. The mixture was warmed to room temperature and left to stir for 10 min. The solution was poured into ice, and the color changed to dark green. The aqueous solution was basified with satd. NaHCO₃ (solid) until pH 8-9. The mixture was extracted with CHCl₃ (4×). The combined organic layer was washed with brine, dried (Na₂SO₄), filtered, and concentrated to give an orange solid. The crude mixture was diluted with methanol (5 mL). The flask was flushed with N₂ and Pd/C (10%, 0.054 g, 0.055 mmol) was added. The flask was flushed with H₂ (balloon) for 10 min and then kept under H₂ (1 atm-balloon) for 13 h. The mixture was filtered through Celite®, rinsed with methanol and EtOAc, and concentrated. The oil was purified by chromatography on SiO₂ (18-100% EtOAc/hexanes) to give methyl (S)-8-aminochromane-2-carboxylate ((S)-3, 0.026 g, 23%) as a light pink solid and methyl (S)-6-aminochromane-2-carboxylate ((S)-4, 0.031 g, 26%) as an amber oil: (S)-3: [α]_{D}=−18.1 (c 0.30, CHCl₃); ¹H NMR (CDCl₃, 400 MHz): δ 6.77 (app t, J=7.6 Hz, 1H), 6.57 (d, J=7.0 Hz, 1H), 6.45 (d, J=7.5 Hz, 1H), 4.76 (dd, J=7.8, 3.6 Hz, 1H), 3.94-3.66 (br s, 2H), 2.85-2.68 (m, 2H), 2.31-2.14 (m, 2H); (S)-4: [α]_{D}=+39.0 (c 0.042, CHCl₃); ¹H NMR (CDCl₃, 400 MHz): δ 6.76 (d, J=8.6 Hz, 1H), 6.50 (dd, J=8.6, 2.8 Hz, 1H), 6.40 (d, J=2.8 Hz, 1H), 4.65 (dd, J=8.0, 3.4 Hz, 1H), 3.79 (s, 3H), 3.52-3.28 (br s, 2H), 2.82-2.64 (m, 2H), 2.29-2.09 (m, 2H).

Methyl (S)-8-((((2-chlorobenzyl)oxy)carbonyl)amino)chromane-2-carboxylate (A). 2-Chlorobenzyl carbonochloridate (75%, 0.0450 g, 0.165 mmol) was slowly added dropwise to a solution of methyl (S)-8-aminochromane-2-carboxylate ((S)-3, 0.0206 g, 0.0994 mmol), pyridine (0.040 mL, 0.497 mmol) in anhydrous CH₂Cl₂ (0.4 mL) at 0° C. After 0.5 h, the reaction mixture was extracted with CH₂Cl₂, washed with H₂O, 0.5M KHSO₄, brine, dried (Na₂SO₄), and filtered through a pad of SiO₂ to give methyl (S)-8-((((2-chlorobenzyl)oxy)carbonyl)amino)chromane-2-carboxylate (A, 0.0261 g, 70%): HPLC analysis >96% ee, ChiralPak AD-H (0.46×25 cm, 0.5 μM, 95/5 hexanes/EtOH.

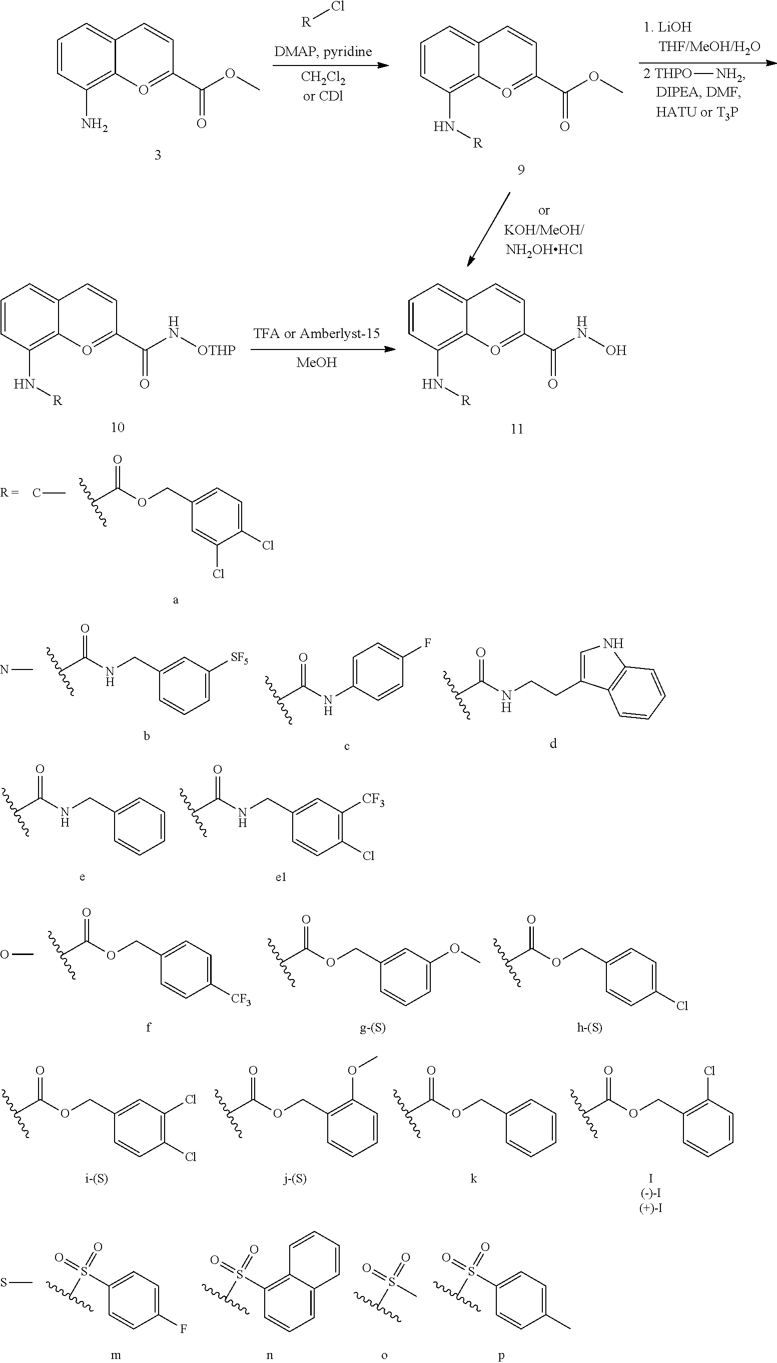

Methyl 8-(2-chloroacetamido)chromane-2-carboxylate (B). To a solution of methyl 8-aminochromane-2-carboxylate (1.30 g, 6.20 mmol) in CH₂Cl₂ (30 mL) was added at Et₃N (1.74 mL, 12.6 mmol) and DMAP (76.0 mg, 0.62 mmol) at 0° C. followed by 2-chloroacetyl chloride (0.64 mL, 7.44 mmol). The reaction mixture was slowly warmed to room temperature. After 16 h, the reaction mixture was extracted with CH₂Cl₂ and washed with H₂O. The aqueous layer was back extracted with CH₂Cl₂ (3×). The combined organic layer was dried (Na₂SO₄), filtered and concentrated. The residue was purified by chromatography on SiO₂ (ISCO-Rf, 20-50% EtOAc/hexanes) to give methyl 8-(2-chloroacetamido)chromane-2-carboxylate (B, 1.00 g, 57%) as a yellow solid: ¹H NMR (CDCl₃, 400 MHz): δ 8.98 (s, 1H), 8.17 (d, J=7.7 Hz, 1H), 6.88 (app t, J=7.8 Hz, 1H), 6.82 (d, J=7.3 Hz, 1H), 4.82 (dd, J=7.8, 3.6 Hz, 1H), 4.19 (q, J_{AB}=15.6 Hz, 2H), 3.80 (s, 3H), 2.91-2.68 (m, 2H), 2.39-2.28 (m, 1H), 2.24-2.12 (m, 1H); ¹³C NMR (CDCl₃, 100 MHz): δ 170.6, 163.6, 142.2, 126.0, 124.6, 121.1, 120.7, 117.7, 74.0, 52.4, 43.1, 24.3, 22.9.

Methyl 8-(2-(3,4-dichlorophenoxy)acetamido)chromane-2-carboxylate (9a). To a solution of 8-(2-chloroacetamido)chromane-2-carboxylate (B, 110 mg, 0.38 mmol) in DMF (3.0 mL) was added K₂CO₃ (97 mg, 0.70 mmol) followed by 3,4-dichlorophenol (75 mg, 0.46 mmol). After 16 h, the reaction mixture was extracted with EtOAc and washed with H₂O. The aqueous layer was back extracted with EtOAc (2×). The combined organic layer was washed with H₂O, brine, dried (Na₂SO₄), filtered and concentrated. The residue was purified by chromatography on SiO₂ (ISCO-Rf, 5-20% EtOAc/hexanes) to give methyl 8-(2-(3,4-dichlorophenoxy)acetamido)chromane-2-carboxylate (9a, 130 mg, 88%) as an off-white solid: ¹H NMR (CDCl₃, 600 MHz): δ 9.02 (s, 1H), 8.22 (d, J=8.1 Hz, 1H), 7.37 (d, J=8.5 Hz, 1H), 7.18 (d, J=3.0 Hz, 1H), 6.91-6.86 (m, 2H), 6.80 (d, J=7.7 Hz, 1H), 4.74 (dd, J=9.1, 3.2 Hz, 1H), 4.56 (s, 2H), 3.82 (s, 3H), 2.86 (ddd, J=16.3, 9.6, 5.5 Hz, 1H), 2.77 (dt, J=16.6, 5.1 Hz, 1H), 2.39-2.33 (m, 1H), 2.12 (ddd, J=13.4, 9.0, 5.7 Hz, 1H); ¹³C NMR (CDCl₃, 150 MHz): δ 170.4, 164.8, 155.9, 142.0, 133.1, 130.8, 125.9, 125.5, 124.3, 121.0, 120.8, 117.7, 117.3, 114.2, 74.0, 67.7, 52.4, 24.5, 23.3.

8-(2-(3,4-Dichlorophenoxy)acetamido)-N-hydroxychromane-2-carboxamide (11a). To an ice-cooled solution of hydroxylamine hydrochloride (577 mg, 8.25 mmol) in methanol (5.0 mL) was added powdered KOH (554 mg, 9.90 mmol) portionwise and the resulting mixture was stirred at room temperature for 1 hour after the addition of KOH was completed. The precipitate was removed by filtration and the filtrate was added dropwise to an ice-cooled solution of methyl 8-(2-(3,4-dichlorophenoxy)acetamido)chromane-2-carboxylate (9a, 130 mg, 0.33 mmol) in methanol (2.0 mL) and THE (2.0 mL). An additional amount of KOH (185 mg, 3.3 mmol) was added to the reaction solution. After 1 h at 0° C., the reaction mixture was treated with 1M HCl to pH 7.0-8.0 and the solid was collected by filtration, washed with H₂O, hexanes and dried in vacuo to give 8-(2-(3,4-dichlorophenoxy)acetamido)-N-hydroxychromane-2-carboxamide (11a, 70 mg, 54%) as an off white solid: ¹H NMR (DMSO-d₆, 600 MHz): δ 10.95 (s 1H), 9.55 (s, 1H), 9.06 (s, 1H), 7.60 (d, J=7.3 Hz, 1H), 7.56 (app t, J=9.2 Hz, 1H), 7.32 (d, J=2.7 Hz, 1H), 7.04 (dd, J=9.1, 2.4 Hz, 1H), 6.89 (d, J=7.9 Hz, 1H), 6.84 (app t, J=7.5 Hz, 1H), 4.81 (app q, J=14.5, 2H), 4.72 (dd, J=7.4, 3.3 Hz, 1H), 2.79 (ddd, J=17.1, 7.5, 5.5 Hz, 1H), 2.66 (dt, J=7.1, 6.1 Hz, 1H), 2.15-2.08 (m, 1H), 2.08-2.01 (m, 1H); ¹³C NMR (DMSO-d₆, 150 MHz): δ 166.4, 166.2, 157.2, 144.3, 131.6, 131.1, 125.8, 125.2, 123.2, 122.5, 121.1, 120.1, 116.8, 115.9, 74.6, 67.5, 23.8, 22.5; HRMS (LCMS ESI+) m/z calcd for C₁₈H₁₅O₅N₂Cl₂ [M−H] 409.0353, found 409.0364.

Methyl 8-(3-(3-(pentafluoro-l⁶-sulfaneyl)benzyl)ureido)chromane-2-carboxylate. To a solution of methyl 8-aminochromane-2-carboxylate (150 mg, 0.72 mmol) in CH₂Cl₂ (2.5 mL) was added carbonyl diimidazole (146 mg, 0.90 mmol) at room temperature. After 16 h, (3-(pentafluoro-l⁶-sulfaneyl)phenyl)methanamine (200 mg, 0.86 mmol) in CH₂Cl₂ (2.0 ml) was added. After 16 h, the reaction mixture was extracted with CH₂Cl₂ and washed with H₂O. The aqueous layer was back extracted with CH₂Cl₂ (3×). The combined organic layer was dried (Na₂SO₄), filtered and concentrated. The residue was purified by chromatography on SiO₂ (ISCO-Rf, 0-50% EtOAc/hexanes) to give methyl 8-(3-(3-(pentafluoro-l⁶-sulfaneyl)benzyl)ureido)chromane-2-carboxylate as an off white solid (200 mg, 60%): ¹H NMR (CDCl₃, 400 MHz): 37.76 (dd, J=8.1, 1.8 Hz, 1H), 7.70 (app t, J=1.9 Hz, 1H), 7.64 (dd, J=8.4, 2.2 Hz, 1H), 7.52 (d, J=7.7 Hz, 1H), 7.41 (app t, J=8.3 Hz, 1H), 6.89 (s, 1H), 6.87 (app t, J=7.9 Hz, 1H), 6.76 (dd, J=7.8, 1.6 Hz, 1H), 5.65 (app t, J=6.0 Hz, 1H), 4.78 (dd, J=7.1, 4.4 Hz, 1H), 4.55 (dd, J=15.5, 5.6 Hz, 1H), 4.45 (dd, J=15.5, 5.6 Hz, 1H), 3.76 (s, 3H), 2.88-2.68 (m, 2H), 2.34-2.14 (m, 2H).

8-(3-(3-(Pentafluoro-l⁶-sulfaneyl)benzyl)ureido)chromane-2-carboxylic acid (10b-int). To a solution of methyl 8-(3-(3-(pentafluoro-l⁶-sulfaneyl)benzyl)ureido)chromane-2-carboxylate (9b, 0.200 g, 0.43 mmol) in MeOH/THF (2.0/1.0 mL) was added 1M LiGH (0.80 mL) at room temperature. After 16 h, the solution was concentrated and the residue was diluted with H₂O, acidified with 1M HCl and extracted with EtOAc (2×). The combined organic layer was washed with brine, dried (Na₂SO₄), filtered and concentrated to give 8-(3-(3-(pentafluoro-l⁶-sulfaneyl)benzyl)ureido)chromane-2-carboxylic acid (10b-int, 0.168 g, contained residual solvent) as a foamy solid that was used without further purification: ¹H NMR (CDCl₃, 400 MHz): δ 11.11 (br s, 0.5H), 8.27 (s, 1H), 7.64 (s, 1H), 7.59 (d, J=7.5 Hz, 1H), 7.42-7.34 (m, 3H), 6.81-6.77 (m, 2H), 6.51 (br s, 0.5H), 4.63 (d, J=7.5 Hz, 1H), 4.42 (s, 2H), 2.83-2.79 (m, 2H), 2.33-2.31 (m, 1H), 2.09-2.06 (m, 1H); HRMS (LCMS ESI+) m/z calcd for C₁₈H₁₈O₄N₂F₅S [M+H]453.0902, found 453.0901.

8-(3-(3-(Pentafluoro-l⁶-sulfaneyl)benzyl)ureido)-N-((tetrahydro-2H-pyran-2-yl)oxy)chromane-2-carboxamide (10b). To a solution of 8-(3-(3-(pentafluoro-l⁶-sulfaneyl)benzyl)ureido)chromane-2-carboxylic acid (0.168 g, 0.33 mmol) in CH₂Cl₂/DMF (1.5 mL/0.1 mL) was added O-(tetrahydro-2H-pyran-2-yl)hydroxylamine (0.065 g, 0.55 mmol) in CH₂Cl₂ (0.5 mL) followed by T3P (0.295 g, 50% EtOAc soln) then DIPEA (0.050 mL, 0.30 mmol) at room temperature. After 20 h, the solution was extracted with CH₂Cl₂, washed with 0.2M HCl, 1M LiCl, H₂O, dried (Na₂SO₄), filtered and concentrated. The solid residue was slurried with Et₂O and collected by filtration and dried in vacuo to give 8-(3-(3-(pentafluoro-l⁶-sulfaneyl)benzyl)ureido)-N-((tetrahydro-2H-pyran-2-yl)oxy)chromane-2-carboxamide 0.148 g, 76%-contained residual solvent) and was used without further purification: ¹H NMR (DMSO-d₆, 400 MHz): 311.42 (s, 0.5H), 11.31 (s, 0.5H), 8.06 (d, J=8.8 Hz, 1H), 7.83 (s, 1H), 7.78-7.72 (m, 1.5H), 7.62-7.58 (m, 2.5H), 7.26-7.21 (m, 1H), 6.77-6.67 (m, 2H), 4.91-4.89 (m, 1H), 4.73 (app t, J=2.7 Hz, 0.5H), 4.68 (dd, J=3.4, 8.1 Hz, 0.5H), 4.40 (d, J=6.0 Hz, 2H), 4.00-3.95 (m, 1H), 3.45-3.42 (m, 1H), 2.77-2.62 (m, 2H), 2.16-1.96 (m, 2H), 1.71-1.63 (m, 3H), 1.52-1.49 (m, 3H); HRMS (LCMS ESI+) m/z calcd for C₂₃H₂₆O₅N₃F₅SNa [M+Na] 574.1406, found 574.1404.

N-Hydroxy-8-(3-(3-(pentafluoro-l⁶-sulfaneyl)benzyl)ureido)chromane-2-carboxamide (11b). To a mixture of 8-(3-(3-(pentafluoro-l⁶-sulfaneyl)benzyl)ureido)-N-((tetrahydro-2H-pyran-2-yl)oxy)chromane-2-carboxamide (0.148 g, 0.254 mmol) in MeOH/THF (1.5 mL/2.0 mL) was added Amberlyst-15 (0.041 g, washed with MeOH) at room temperature. The suspension was stirred at room temperature overnight at which time the reaction became homogeneous. The mixture was filtered through Celite®, rinsed with MeOH then THE and concentrated. The solid residue was slurried with Et₂O and collected by filtration to give N-hydroxy-8-(3-(3-(pentafluoro-l⁶-sulfaneyl)benzyl)ureido)chromane-2-carboxamide (0.097 g, 81%) as an off-white solid: mp 203-204° C.; IR (neat) 3346, 1682, 1645, 1599, 1580, 1480, 1446, 1336, 1260, 1241, 1220, 1108, 1094, 985, 888, 785, 690 cm⁻¹; ¹H NMR (DMSO-d₆, 400 MHz): 310.80 (d, J=1.6 Hz, 1H), 9.05 (d, J=2.0 Hz, 1H), 8.03 (s, 1H), 7.84 (s, 1H), 7.80-7.74 (m, overlapping signals, 2H), 7.62-7.59 (m, 2H), 7.17 (app t, J=6.0 Hz, 1H), 6.75 (app t, J=7.6 Hz, 1H), 6.68 (app t, J=6.4 Hz, 1H), 4.63 (dd, J=2.8, 8.4 Hz, 1H), 4.41 (d, J=6.0 Hz, 2H), 2.80-2.77 (m, 1H), 2.69-2.65 (m, 1H), 2.20-2.17 (m, 1H), 2.16-1.96 (m, 1H); ¹³C NMR (DMSO-d₆, 100 MHz): δ166.2, 155.4, 153.1, 153.0, 152.8, 142.5, 142.4, 131.3, 129.5, 128.0, 124.2, 122.4, 121.7, 120.1, 117.6, 74.6, 42.3, 24.3, 23.0; HRMS (LCMS ESI+) m/z calcd for C₁₈H₁₇O₄N₃F₅S [M−H] 466.0854, found 466.0851.

Methyl 8-(3-(4-fluorophenyl)ureido)chromane-2-carboxylate (9c). To a solution of methyl 8-aminochromane-2-carboxylate (150 mg, 0.72 mmol) in CH₂Cl₂ (2.5 mL) was added carbonyl diimidazole (185 mg, 1.15 mmol) at room temperature. After 16 h, 4-fluoroaniline (95.5 mg, 0.86 mmol) in CH₂Cl₂ (2.0 ml) was added. After 16 h, the reaction mixture was extracted with CH₂Cl₂ and washed with H₂O. The aqueous layer was back extracted with CH₂Cl₂ (3×). The combined organic layer was dried (Na₂SO₄), filtered and concentrated. The residue was purified by chromatography on SiO₂ (ISCO-Rf, 0-50% EtOAc/hexanes) to give methyl 8-(3-(4-fluorophenyl)ureido)chromane-2-carboxylate (190 mg, 77%) as a gray solid: ¹H NMR (CDCl₃, 400 MHz): δ 7.93 (d, J=8.4 Hz, 1H), 7.40-7.33 (m, 3H), 7.25 (s, 1H), 6.99 (t, J=6.7 Hz, 2H), 6.88 (app t, J=6.9 Hz, 1H), 6.76 (d, J=6.6 Hz, 1H), 4.75-4.69 (m, 1H), 3.78 (s, 3H), 2.92-2.72 (m, 2H), 2.35-2.25 (m, 1H), 2.21-2.09 (m, 1H).

8-(3-(4-Fluorophenyl)ureido)chromane-2-carboxylic acid (10c-int). To a solution of methyl 8-(3-(4-fluorophenyl)ureido)chromane-2-carboxylate (0.190 g, 0.55 mmol) in MeOH/THF (2.0 mL/1.0 mL) was added 1M LiGH (0.80 mL) at room temperature. After 16 h, the solution was concentrated and the residue was diluted with H₂O, acidified with 1M HCl, extracted with EtOAc (2×). The combined organic layer was washed with brine, dried (Na₂SO₄), filtered and concentrated to give 8-(3-(4-fluorophenyl)ureido)chromane-2-carboxylic acid (0.128 g, 67%-contained residual solvent) and was used without further purification: ¹H NMR (DMSO-d₆, 400 MHz): δ 13.16 (s, 1H), 9.41 (s, 1H), 8.07 (s, 1H), 7.93 (d, J=8.0 Hz, 1H), 7.47-7.43 (m, 2H), 7.11 (app t, J=8.8 Hz, 2H), 6.76 (app t, J=7.8 Hz, 1H), 6.66 (d, J=6.6 Hz, 1H), 4.99 (app t, J=4.5 Hz, 1H), 2.77-2.71 (m, 1H), 2.65-2.61 (m, 1H), 2.18-2.13 (m, 2H); ¹⁹F NMR (DMSO-d₆, 376 MHz): δ−121.7; HRMS (LCMS ESI+) m/z calcd for C₁₇H₁₆O₄N₂F [M+H] 331.1089, found 331.1089.

8-(3-(4-Fluorophenyl)ureido)-N-((tetrahydro-2H-pyran-2-yl)oxy)chromane-2-carboxamide (10c). To a solution of 8-(3-(4-fluorophenyl)ureido)chromane-2-carboxylic acid (0.125 g, 0.378 mmol) in CH₂Cl₂/DMF (1.5 mL, 0.1 mL) was added O-(tetrahydro-2H-pyran-2-yl)hydroxylamine (0.054 g, 0.46 mmol) in CH₂Cl₂ (0.5 mL) followed by T3P (0.310 g, 50% EtOAc soln) then DIPEA (0.075 mL, 0.45 mmol) at room temperature. After 22 h, the solution was extracted with CH₂Cl₂, washed with 0.2M HCl, 1M LiCl, H₂O, dried (Na₂SO₄), filtered and concentrated. The solid was slurried with Et₂O and collected by filtration and dried in vacuo to give 8-(3-(4-fluorophenyl)ureido)-N-((tetrahydro-2H-pyran-2-yl)oxy)chromane-2-carboxamide (0.135 g, 83%) as a white solid: ¹H NMR (DMSO-d₆, 400 MHz): 311.39 (s, 0.5H), 11.33 (s, 0.5H), 9.22 (s, 1H), 8.12 (d, J=1.6 Hz, 1H), 7.81 (d, J=6.7 Hz, 0.5H), 7.74 (d, J=7.3 Hz, 0.5H), 7.47-7.45 (m, 2H), 7.14-7.09 (m, 2H), 6.79 (app t, J=7.7 Hz, 1H), 6.72 (app t, J=6.4 Hz, 1H), 4.92 (d, J=2.3 Hz, 1H), 4.78-4.71 (m, 1H), 4.01-3.98 (m, 1H), 3.49-3.46 (m, 1H), 2.78-2.65 (m, 2H), 2.12-2.02 (m, 2H), 1.71-1.66 (m, 3H), 1.58-1.51 (m, 3H); ¹⁹F NMR (DMSO-d₆, 376 MHz): δ−121.6; HRMS (LCMS ESI+) m/z calcd for C₂₂H₂₄O₅N₃FNa [M+Na] 452.1592, found 452.1590.

8-(3-(4-Fluorophenyl)ureido)-N-hydroxychromane-2-carboxamide (11c). To a solution of 8-(3-(4-fluorophenyl)ureido)-N-((tetrahydro-2H-pyran-2-yl)oxy)chromane-2-carboxamide (0.135 g, 0.314 mmol) in MeOH/THF (1.0/3.0 mL) was added Amberlyst-15 (0.036 g, washed with MeOH) at room temperature. After 16 h, the mixture was diluted with THE and the solution was transferred to a flask and concentrated. The solid residue was slurried with Et₂O and the white solid was collected by filtration and dried in vacuo at 90° C. to give 8-(3-(4-fluorophenyl)ureido)-N-hydroxychromane-2-carboxamide (0.092 g, 85%): mp 231-232° C.; IR (neat) 3254, 1655, 1618, 1579, 1509, 1452, 1211, 1194, 1095, 975, 828, 752, 705 cm⁻¹; ¹H NMR (DMSO-d₆, 600 MHz): 310.82 (s, 1H), 9.12 (s, 1H), 9.08 (d, J=1.3 Hz, 1H), 8.11 (s, 1H), 7.82 (d, J=7.8 Hz, 1H), 7.49-7.46 (m, 2H), 7.12 (app t, J=8.8 Hz, 2H), 6.79 (app t, J=7.8 Hz, 1H), 6.72 (d, J=7.4 Hz, 1H), 4.68 (dd, J=3.1, 8.2 Hz, 1H), 2.80-2.77 (m, 1H), 2.71-2.68 (m, 1H), 2.19-2.15 (m, 1H), 2.03-1.98 (m, 1H); ¹³C NMR (DMSO-d₆, 150 MHz): 3166.3, 158.1, 156.5, 152.7, 142.4, 136.1, 127.6, 122.7, 121.8, 120.1, 119.84, 119.79, 117.5, 115.4, 115.2, 74.5, 24.3, 22.9; ¹⁹F NMR (DMSO-d₆, 565 MHz): δ−121.5; HRMS (LCMS ESI+) m/z calcd for C₁₇H₁₅O₄N₃F [M−H] 344.1041, found 344.1042.

Methyl 8-(3-(2-(1H-indol-3-yl)ethyl)ureido)chromane-2-carboxylate (9d). To a solution of methyl 8-aminochromane-2-carboxylate (3, 200 mg, 0.965 mmol) in CH₂Cl₂ (3.0 mL) was added carbonyl diimidazole (250 mg, 1.54 mmol), and the resulting mixture was stirred overnight at rt. To this solution was added tryptamine (186 mg, 1.16 mmol). After 6 h, the reaction mixture was extracted with CH₂Cl₂, washed with brine (2×), dried (Na₂SO₄), filtered and concentrated. The residue was purified by chromatography on SiO₂ (4/1, hexanes/EtOAc) to give methyl 8-(3-(2-(1H-indol-3-yl)ethyl)ureido)chromane-2-carboxylate (9d, 0.260 g, 68%) as a white solid. (LCMS ESI+) m/z calcd for C₂₂H₂₄O₄N₃ [M+H] 394.1761, found 394.1759.

Lithium 8-(3-(2-(1H-indol-3-yl)ethyl)ureido)chromane-2-carboxylate (10d-int). A solution of methyl 8-(3-(2-(1H-indol-3-yl)ethyl)ureido)chromane-2-carboxylate (9d, 0.260 g, 0.661 mmol, crude) in THF/MeOH (3.0/1.5 mL) was treated with 1M LiGH (0.73 mL) at room temperature. After 15.5 h, the solution was concentrated and the residue was treated with PhMe and concentrated (3×) and the solid residue was used without further purification. HRMS (ESI+) m/z calcd for C₂₁H₂₂N₃O₄ [M+H] 380.1605, found 380.1609.

8-(3-(2-(1H-Indol-3-yl)ethyl)ureido)-N-hydroxychromane-2-carboxamide. To a solution of lithium 8-(3-(2-(1H-indol-3-yl)ethyl)ureido)chromane-2-carboxylate (10d-int, 254 mg, 0.659 mmol) in DMF (3.5 mL) was added O-(tetrahydro-2H-pyran-2-yl)hydroxylamine (99.2 mg, 0.847 mmol) in DMF (1 mL). To this solution was added T3P (523 mg, 0.822 mmol, 50% EtOAc) and DIPEA (121 mg, 0.939 mmol). After 3 d, the reaction mixture was extracted with EtOAc, washed with satd. NH₄Cl, 1M LiCl, and brine. The organic layer was dried (Na₂SO₄), filtered, and concentrated to give crude product (10d) that was used directly for the next step. To the crude material (10d, 300 mg, 0.626 mmol) in methanol (5 mL) was added Amberlyst-15 (50.0 mg) at room temperature. After 16 h, the reaction mixture was diluted with THE (5 mL), filtered through Celite®, rinsed with THF, and concentrated. The residue was suspended in Et₂O and the solid was collected by filtration and dried in vacuo at 100° C. to give 8-(3-(2-(1H-indol-3-yl)ethyl)ureido)-N-hydroxychromane-2-carboxamide (11d, 0.088 g, 36%) as a beige solid: ¹H NMR (DMSO-d₆, 400 MHz): δ 10.84 (s, 1H), 10.80 (s, 1H), 9.07 (s, 1H), 7.88 (s, 1H), 7.58 (d, J=7.6 Hz, 1H), 7.34 (d, J=8.0 Hz, 1H), 7.18 (d, J=2.0 Hz, 1H), 7.06 (dd, J=6.8 Hz, 1.2 Hz, 1H), 6.97 (dd, J=7.2, 0.4 Hz, 1H), 6.75 (app t, J=7.6 Hz, 1H), 6.66 (d, J=6.8 Hz, 1H), 6.59 (app t, J=5.6 Hz, 1H), 4.62 (dd, J=8.8, 2.8 Hz, 1H), 3.44-3.37 (m, 2H), 2.87 (t, J=3.2 Hz, 2H), 2.80-2.75 (m, 1H), 2.70-2.69 (m, 1H), 2.18 (m, 1H), 1.93 (m, 1H); ¹³C NMR (DMSO-d₆, 100 MHz): δ 166.3, 155.4, 142.4, 136.3, 128.3, 127.2, 122.7, 122.1, 121.6, 120.9, 120.1, 118.4, 118.2, 117.5, 111.8, 111.4, 74.6, 25.9, 24.3, 23.1; HRMS (ESI+) m/z for C₂₁H₂₁O₄N₄, [M−H] calcd 393.1557, found 393.1559.

Methyl 8-(3-benzylureido)chromane-2-carboxylate (9e). To a solution of methyl 8-aminochromane-2-carboxylate (3, 0.200 g, 0.965 mmol) in CH₂Cl₂ (4 mL) was added benzyl isocyanate (0.125 mL, 1.013 mmol) in CH₂Cl₂ (2 mL) dropwise. The reaction mixture was extracted with CH₂Cl₂, washed with 0.25M HCl, brine, dried (MgSO₄), filtered and concentrated to give methyl 8-(3-benzylureido)chromane-2-carboxylate (9e, 0.320 g, 97%) as a white solid: HRMS (ESI+) m/z for C₁₉H₂₁O₄N₂ [M+H] calcd 341.1496, found 341.1495.

Lithium 8-(3-benzylureido)chromane-2-carboxylate (10e-int). To a solution of methyl 8-(3-benzylureido)chromane-2-carboxylate (9e, 0.320 g, 0.940 mmol, crude) in THF/MeOH (3.0/1.5 mL) was added 1M LiGH (0.99 mL) at room temperature. After 15.5 h, the solution was concentrated and the residue was treated with PhMe and concentrated (3×) to give crude lithium 8-(3-benzylureido)chromane-2-carboxylate (10e-int) that was used without further purification: HRMS (ESI+) m/z for C₁₈H₁₉O₄N₂ [M+H] calcd 327.1339, found 327.1336.

8-(3-Benzylureido)-N-((tetrahydro-2H-pyran-2-yl)oxy)chromane-2-carboxamide (10e). A solution of lithium 8-(3-benzylureido)chromane-2-carboxylate (10e-int, crude, 0.050 g, 0.150 mmol) in DMF (1.5 mL) was treated with O-(tetrahydro-2H-pyran-2-yl)hydroxylamine (0.019 g, 0.17 mmol) in DMF (1.0 mL) followed by T3P (0.098 g, 0.17 mmol) and DIPEA (0.03 mL, 0.18 mmol) at room temperature. After 15.5 h, the solution was extracted with EtOAc, washed with 0.25M HCl, 1 M LiCl (3×), brine, dried (Na₂SO₄), filtered and concentrated. The residual oil was purified by chromatography on SiO₂ (2/1, hexanes/EtOAc) to give 8-(3-benzylureido)-N-((tetrahydro-2H-pyran-2-yl)oxy)chromane-2-carboxamide (10e, 0.50 g, 78%) as a white solid.

8-(3-Benzylureido)-N-hydroxychromane-2-carboxamide (11e). To a solution of 8-(3-benzylureido)-N-((tetrahydro-2H-pyran-2-yl)oxy)chromane-2-carboxamide (10e, 0.235 g, 0.552 mmol) in MeOH (4.5 mL) was added Amberlyst-15 (0.024 g, washed with MeOH). After 15 h, the reaction mixture was filtered through Celite® and concentrated. The solid was treated with Et₂O and the solid was collected by filtration to give 8-(3-benzylureido)-N-hydroxychromane-2-carboxamide (11e, 0.117 g, 62%) as a white solid: mp 187-190° C.; ¹³C NMR (DMSO-d₆, 150 MHz): δ166.2, 155.3, 142.2, 140.2, 128.3, 128.2, 127.3, 126.8, 122.2, 121.6, 120.1, 117.3, 74.6, 42.8, 24.3, 23.1; HRMS (ESI+) m/z for C₁₈H₂₀O₄N₃ [M+H] calcd 342.1448, found 342.1445.

8-((((4-(Trifluoromethyl)benzyl)oxy)carbonyl)amino)chromane-2-carboxylic acid (10f-int). To a solution of methyl 8-aminochromane-2-carboxylate (3, 0.195 g, 0.941 mmol) in CH₂Cl₂ (5 mL) was added carbonyl diimidazole (0.191 g, 1.176 mmol) at room temperature. After 2 h, (4-(trifluoromethyl)phenyl)methanol (0.155 mL, 1.13 mmol) was added followed by pyridine (152 mL, 1.88 mmol) at room temperature. After 16 h, the reaction mixture was extracted with CH₂Cl₂, washed with 0.2M HCl (3×), brine, dried (Na₂SO₄), filtered and concentrated. The brown oil was purified by chromatography on SiO₂ (ISCO-Rf, 0-100% EtOAc/hexanes) to give semi-pure product that was used without further purification. The product (0.385 g, crude) in MeOH/THF (5 mL, 3/1) was treated with 1M LiGH (1.2 mL) at room temperature. After 2 h, the reaction mixture was concentrated. The residue was partitioned in hexanes and H₂O. The aqueous layer was cooled to 0° C. and acidified with 1M HCl and extracted with EtOAc (4×). The combined organic layer was washed with brine, dried (Na₂SO₄), filtered and concentrated to give 8-((((4-(trifluoromethyl)benzyl)oxy)carbonyl)amino)chromane-2-carboxylic acid (10f-int, 0.087 g, 23%-3 steps) as a white solid: IR (neat): 3429, 3392, 3284, 3056, 2932, 2845, 1754, 1725, 1700, 1642, 1620, 1593, 1538, 1480, 1454, 1433, 1404, 1371, 1323, 1277, 1239, 1216, 1193, 1158, 1109, 1099, 1066, 1020, 986, 963, 881, 854, 825, 765, 726, 699, 659, 606 cm⁻¹; ¹H NMR (DMSO-d₆, 400 MHz): δ 8.58 (s, 1H), 7.76 (d, J=8.0 Hz, 2H), 7.65 (d, J=8.0 Hz, 2H), 7.56-7.53 (m, 1H), 6.81 (d, J=2.0 Hz, 1H), 6.80 (s, 1H), 5.25 (s, 2H), 4.84 (dd, J=3.6, 6.4 Hz, 1H), 2.84-2.77 (m, 1H), 2.68-2.61 (m, 1H), 2.17-2.13 (m, 1H), 2.03-2.10 (m, 1H).

4-(Trifluoromethyl)benzyl (2-(((tetrahydro-2H-pyran-2-yl)oxy)carbamoyl)chroman-8-yl)carbamate (10f). A solution of 8-((((4-(trifluoromethyl)benzyl)-oxy)carbonyl)amino)chromane-2-carboxylic acid (10f-int, 0.082 g, 0.207 mmol) in DMF/CH₂Cl₂ (0.4 mL, 1/1) was cooled to 0° C. and treated with O-(tetrahydro-2H-pyran-2-yl)hydroxylamine (0.041 g, 0.348 mmol) in CH₂Cl₂ (0.25 mL). To this solution was added T3P (0.221 g, 0.348 mmol, 50% CH₃CN soln) in CH₂Cl₂ (0.25 mL) followed by DIPEA (0.057 mL, 0.328 mmol). The solution was stirred at 0° C. for 10 min., then removed from ice and left to stir at room temperature. After 5 h, the reaction mixture was extracted with CH₂Cl₂, washed with 0.25M HCl (2×), 1M LiCl (3×), H₂O (4×), dried (Na₂SO₄), filtered and concentrated. The yellow-brown residue was purified by chromatography on SiO₂ (ISCO-Rf, 0-20% EtOAc/CH₂Cl₂) to give 4-(trifluoromethyl)benzyl (2-(((tetrahydro-2H-pyran-2-yl)oxy)carbamoyl)chroman-8-yl)carbamate (10f, 0.071 g, 69%) as a yellow oil: IR (neat) 3221, 2944, 2852, 1665, 1617, 1594, 1536, 1479, 1453, 1419, 1322, 1162, 1110, 1064, 1036, 1016, 978, 945, 895, 872, 819, 769, 746, 728, 632 cm⁻¹; ¹H NMR (CDCl₃, 400 MHz): δ 9.95 (s, 0.5H), 9.70 (s, 0.5H), 7.64 (d, J=8.4 Hz, 2H), 7.54 (d, J=7.2 Hz, 2H), 7.52-7.49 (m, 0.5H), 7.45-7.43 (m, 0.5H), 6.91-6.86 (m, 2H), 6.81 (br d, J=14.0 Hz, 1H), 5.27 (s, 2H), 5.03 (app t, J=3.6 Hz, 0.5H), 4.96 (app t, J=3.6 Hz, 0.5H), 4.86-4.78 (m, 1H), 4.00-3.90 (m, 1H), 3.55-3.45 (m, 1H), 2.87-2.71 (m, 2H), 2.47-2.14 (m, 2H), 1.88-1.75 (m, 3H), 1.64-1.50 (m, 3H).

4-(Trifluoromethyl)benzyl (2-(hydroxycarbamoyl)chroman-8-yl)carbamate (11f). To a solution of 4-(trifluoromethyl)benzyl (2-(((tetrahydro-2H-pyran-2-yl)oxy)carbamoyl)chroman-8-yl)carbamate (10f, 0.065 g, 0.131 mmol) in MeOH/CH₂Cl₂ (1 mL, 1/1) was added Amberlyst-15 (0.023 g, washed with MeOH) at room temperature. After 17 h, the reaction mixture was filtered through Celite®, rinsed with MeOH/CH₂Cl₂ (1:1) and concentrated. The solid residue was treated with Et₂O and the solid was collected by filtration and dried in vacuo to give 4-(trifluoromethyl)benzyl (2-(hydroxycarbamoyl)chroman-8-yl)carbamate (11f, 0.049 g, 91%) as a tan solid: mp 102-104° C.; IR (neat) 3209, 2927, 2851, 1663, 1655, 1617, 1593, 1535, 1479, 1450, 1419, 1322, 1220, 1191, 1162, 1109, 1094, 1063, 1016, 974, 884, 856, 821, 768, 726, 697, 630, 608 cm⁻¹; ¹H NMR (DMSO-d₆, 400 MHz): δ 11.13 (s, 1H), 9.24 (s, 1H), 9.00 (d, J=1.2 Hz, 1H), 7.78 (d, J=8.4 Hz, 2H), 7.66 (d, J=8.0 Hz, 2H), 7.56 (d, J=5.2 Hz, 1H), 6.85-6.80 (m, 2H), 5.27 (s, 2H), 4.62 (dd, J=2.4, 8.8 Hz, 1H), 2.88-2.78 (m, 1H), 2.72-2.67 (m, 1H), 2.24-2.21 (m, 1H). 1.94-1.85 (m, 1H); ¹³C NMR (DMSO-d₆, 100 MHz): 166.2, 153.7, 143.6, 141.6, 128.6, 128.4, 128.3, 126.2, 125.6, 125.5, 125.41, 125.4, 125.3, 124.4, 122.9, 122.3, 120.2, 119.4, 75.0, 65.0, 24.3, 23.3; ¹⁹F NMR (DMSO-d₆, 470 MHz): δ−60.9; HRMS (ESI+) m/z for C₁₉H₁₈O₅N₂F₃[M+H] calcd 411.1162, found 411.1160.

3-Methoxybenzyl (4-nitrophenyl) carbonate. To a solution of (3-methoxyphenyl)methanol (0.332 g, 2.40 mmol) in dry THE (1.5 mL) was added pyridine (0.30 mL, 3.7 mmol) at 0° C. The solution was then treated with 4-nitrophenyl carbonochloridate (0.624 g, 3.1 mmol). After 10 min, the mixture was warmed to room temperature. After 2 h, the reaction mixture was extracted with EtOAc, washed with 0.2M HCl, brine, dried (Na₂SO₄), filtered and concentrated. The solid residue was absorbed onto Celite® and purified by chromatography on SiO₂ (ISCO-Rf, 0-100 EtOAc/hexanes) to give 3-methoxybenzyl (4-nitrophenyl) carbonate (0.606 g, 71%, approx. 85% purity) as a light beige solid that was used without further purification: ¹H NMR (CDCl₃, 500 MHz): 38.25 (d, J=9.5 Hz, 2H), 7.37 (d, J=9.0 Hz, 2H), 7.31 (app t, J=7.5 Hz, 1H), 7.01 (d, J=7.5 Hz, 1H), 6.97 (s, 1H), 6.91 (dd, J=2.5, 6.0 Hz, 1H), 5.27 (s, 2H), 3.82 (s, 3H).

Methyl (S)-8-((((3-methoxybenzyl)oxy)carbonyl)amino)chromane-2-carboxylate (9g). To a solution of methyl (S)-8-aminochromane-2-carboxylate ((S)-3, 0.130 g, 0.627 mmol) in DMF (3 mL) was added 3-methoxybenzyl (4-nitrophenyl) carbonate (0.145 g, 0.418 mmol), HOBT (0.068 g, 0.500 mmol), and 4 Å powdered molecular sieves (0.098 g). After 22 h, the mixture was filtered and extracted with EtOAc, washed with H₂O, brine, dried (Na₂SO₄), filtered and concentrated. The residue was purified by chromatography on SiO₂ (ISCO-Rf, 0-50% EtOAc/hexanes) to give methyl (S)-8-((((3-methoxybenzyl)oxy)carbonyl)amino)chromane-2-carboxylate (9g, 0.131 g, 84%-contains residual solvent) as a colorless oil and was taken to the next step without further purification: ¹H NMR (CDCl₃, 400 MHz): δ 7.96 (br s, 1H), 7.31-7.28 (m, overlapping signals, 2H), 7.00 (d, J=7.6 Hz, 1H), 6.97-6.93 (m, 1H), 6.87 (app t, J=2.8 Hz, 2H), 6.72 (d, J=7.2 Hz, 1H), 5.18 (d, J=3.6 Hz, 2H), 4.78 (dd, J=4.0, 7.2 Hz, 1H), 3.83 (s, 3H), 3.78 (s, 3H), 2.80-2.70 (m, 2H), 2.31-2.15 (m, 2H).

(S)-8-((((3-Methoxybenzyl)oxy)carbonyl)amino)chromane-2-carboxylic acid (10g-int). A solution of methyl (S)-8-((((3-methoxybenzyl)oxy)carbonyl)amino)chromane-2-carboxylate (9g (0.127 g, 0.342 mmol) in MeOH/THF (4 mL, 3/1) was treated with 1M LiGH (0.700 mL) at room temperature. After 16 h, the solution was cooled to 0° C. and acidified with 1M HCl to pH 2 and extracted with EtOAc (4×). The combined organic layer was washed with brine, dried (Na₂SO₄), filtered and concentrated to give (S)-8-((((3-methoxybenzyl)oxy)carbonyl)amino)chromane-2-carboxylic acid (10g-int, 0.118 g, 97%) as a white solid that was used without further purification: ¹H NMR (DMSO-d₆, 400 MHz): 13.00 (br s, 1H), 8.46 (s, 1H), 7.57 (d, J=6.8 Hz, 1H), 7.30 (app t, J=8.0 Hz, 1H), 7.00-6.98 (m, overlapping signals, 2H), 6.94-6.89 (m, 1H), 6.83-6.78 (m, overlapping signals, 2H), 5.12 (s, 2H), 4.83 (dd, J=4.0, 6.4 Hz, 1H), 3.76 (s, 3H), 2.83-2.76 (m, 1H), 2.68-2.60 (m, 1H), 2.20-2.12 (m, 1H), 2.09-2.01 (m, 1H).

3-Methoxybenzyl ((2S)-2-(((tetrahydro-2H-pyran-2-yl)oxy)carbamoyl)chroman-8-yl)carbamate (10g). A solution of (S)-8-((((3-methoxybenzyl)oxy)carbonyl)amino)chromane-2-carboxylic acid (10g-int, 0.110 g, 0.308 mmol) in DMF/CH₂Cl₂ (1 mL, 1/1) was cooled to 0° C. and treated with O-(tetrahydro-2H-pyran-2-yl)hydroxylamine (0.072 g, 0.616 mmol) in CH₂Cl₂ (0.3 mL). To this solution was added T3P (0.392 g, 0.616 mmol, 50% CH₃CN soln) in CH₂Cl₂ (0.3 mL) followed by DIPEA (0.107 mL, 0.616 mmol). The solution was stirred at 0° C. for 10 min. then warmed to room temperature. After 2 h, the reaction mixture was extracted with CH₂Cl₂ and washed with H₂O. The aqueous layer was back extracted with CH₂Cl₂ (3×) and the combined organic layer was washed with 0.25M HCl (2×), 1M LiCl (3×), brine, dried (Na₂SO₄), filtered and concentrated. The brown residue was purified by chromatography on SiO₂ (ISCO-Rf, 0-100% EtOAc/hexanes) to give 3-methoxybenzyl ((2S)-2-(((tetrahydro-2H-pyran-2-yl)oxy)carbamoyl)chroman-8-yl)carbamate (10g, 0.119 g, 85%-contains residual solvent) and was used without further purification: ¹H NMR (CDCl₃, 500 MHz): δ 10.05 (s, 0.5H), 9.76 (s, 0.5H), 7.52-7.50 (m, 0.5H), 7.43-7.41 (m, 0.5H), 7.32-7.28 (m, 1H), 7.00 (d, J=7.5 Hz, 1H), 6.98-6.94 (m, 1H), 6.90-6.85 (m, overlapping signals, 3H), 6.77 (s, 0.5H), 6.72 (s, 0.5H), 5.19 (d, J=2.0 Hz, 2H), 5.04 (app t, J=3.0 Hz, 0.5H), 4.98 (app t, J=3.0 Hz, 0.5H), 4.86 (app t, J=5.0 Hz, 0.5H), 4.81 (dd, J=4.0, 6.0 Hz, 0.5H), 4.02-3.92 (m, 1H), 3.82 (s, 3H), 3.57-3.47 (m, 1H), 2.84-2.71 (m, 2H), 2.47-2.30 (m, 1.5H), 2.27-2.13 (m, 1H), 1.88-1.73 (m, 3H), 1.62-1.50 (m, 3H).

Methoxybenzyl (S)-(2-(hydroxycarbamoyl)chroman-8-yl)carbamate (11g). To a solution of 3-methoxybenzyl ((2S)-2-(((tetrahydro-2H-pyran-2-yl)oxy)carbamoyl)chroman-8-yl)carbamate (10g, 0.113 g, 0.247 mmol) in MeOH/CH₂Cl₂ (3 mL 1/1) was added Amberlyst-15 (0.050 g, washed with MeOH) at room temperature. After 15 h, MeOH (1 mL) was added and the solution was heated to 50° C. After 1.5 h, the solution was removed from heat, filtered through Celite® and concentrated. The solid residue was treated with Et₂O and the solid was collected by filtration and dried in vacuo to give 3-methoxybenzyl (S)-(2-(hydroxycarbamoyl)chroman-8-yl)carbamate (11g, 0.054 g, 59%) as a pink solid: mp 71-73° C.; [α]_{D}=−86.5 (c 0.06, DMSO); IR (neat) 3216, 3050, 2933, 2836, 1661, 1614, 1588, 1532, 1489, 1480, 1449, 1323, 1295, 1266, 1217, 1190, 1168, 1092, 1051, 1018, 969, 883, 863, 831, 768, 739, 727, 690, 632 cm⁻¹; ¹H NMR (DMSO-d₆, 600 MHz): δ 11.15 (d, J=1.2 Hz, 1H), 9.17 (s, 1H), 8.99 (d, J=1.8 Hz, 1H), 7.56 (br d, J=6.6 Hz, 1H), 7.31 (app t, J=8.4 Hz, 1H), 7.01-6.99 (m, overlapping signals, 2H), 6.92-6.90 (m, 1H), 6.84-6.79 (m, overlapping signals, 2H), 5.13 (s, 2H), 4.60 (dd, J=3.0, 9.6 Hz, 1H), 3.76 (s, 3H), 2.85-2.80 (m, 1H), 2.71-2.67 (m, 1H), 2.24-2.20 (m, 1H), 1.91-1.85 (m, 1H); ¹³C NMR (DMSO-d₆, 150 MHz): δ 166.2, 159.3, 153.8, 143.6, 138.1, 129.6, 126.3, 124.3, 122.3, 120.3, 120.1, 119.4, 113.7, 113.5, 75.0, 65.8, 55.1, 24.3, 23.3; HRMS (ESI+) m/z for C₁₉H₂₁O₆N₂[M+H] calcd 373.1394, found 373.1393.

Chlorobenzyl (4-nitrophenyl) carbonate. To a solution of 4-chlorobenzyl alcohol (131.17 mg, 0.92 mmol) in dry THE (0.750 mL) was added pyridine (0.125 mL, 1.545 mmol) at 0° C. The solution was treated with 4-nitrophenyl carbonochloridate (0.240 g, 1.191 mmol). After 10 min, the mixture was warmed to room temperature and stirred for two hours. The reaction mixture was extracted with EtOAc, washed with 0.25M HCl (2×), brine (2×), dried (Na₂SO₄), filtered and concentrated. The residue was purified by chromatography on SiO₂ (ISCO-Rf, 0-30% EtOAc/hexanes) to give the 4-chlorobenzyl (4-nitrophenyl) carbonate (0.216 g, 76%) as a white solid: IR (neat) 3114, 3081, 2964, 2925, 2861, 1757, 1614, 1592, 1523, 1489, 1461, 1410, 1385, 1351, 1311, 1261, 1210, 1157, 1109, 1089, 1066, 1046, 1015, 988, 959, 862, 840, 824, 804, 767, 731, 706, 684, 669, 643, 627 cm⁻¹; ¹H NMR (CDCl₃, 600 MHz): δ 8.29-8.26 (m, 2H), 7.39-7.37 (m, overlapping signals, 6H), 5.26 (s, 2H).

Methyl (S)-8-((((4-chlorobenzyl)oxy)carbonyl)amino)chromane-2-carboxylate (9h). To a solution of methyl (S)-8-aminochromane-2-carboxylate ((S)-3, 131 mg, 0.634 mmol) in DMF (2 mL) was added 4-chlorobenzyl (4-nitrophenyl) carbonate (130 mg, 0.423 mmol), HOBT (68.5 mg, 0.507 mmol) and 4 Å powdered molecular sieves (100 mg). After 22 h, the mixture was filtered and extracted with EtOAc, washed with H₂O, brine, dried (Na₂SO₄), filtered and concentrated. The residue was purified by chromatography on SiO₂ (ISCO-Rf, 0-100% EtOAc/hexanes) to give methyl (S)-8-((((4-chlorobenzyl)oxy)carbonyl)amino)chromane-2-carboxylate (9 h, 0.114 g, 72% contains residual solvent) as a colorless oil and was used without further purification: ¹H NMR (CDCl₃, 400 MHz): δ 7.94 (br s, 1H), 7.37-7.33 (m, 4H), 7.30 (br s, 1H), 6.87 (app t, J=8.0 Hz, 1H), 6.73 (d, J=7.6 Hz, 1H), 5.17 (d, J=2.4 Hz, 2H), 4.78 (dd, J=4.0, 7.2 Hz, 1H), 3.78 (s, 3H), 2.82-2.70 (m, 2H), 2.31-2.15 (m, 2H).

(S)-8-((((4-Chlorobenzyl)oxy)carbonyl)amino)chromane-2-carboxylic acid (10 h-int). A solution of methyl (S)-8-((((4-chlorobenzyl)oxy)carbonyl)amino)chromane-2-carboxylate (9 h, 0.110 g, 0.293 mmol) in MeOH/THF (4 mL, 3/1) was treated with 1M LiGH (0.7 mL) at room temperature. After 3 h, the reaction mixture was cooled to 0° C. and acidified with conc. HCl to pH 2. The solution was extracted with EtOAc and washed with H₂O. The aqueous layer was back extracted with EtOAc (5×). The combined organic layer was washed with brine, dried (Na₂SO₄), filtered and concentrated give (S)-8-((((4-chlorobenzyl)oxy)carbonyl)amino)chromane-2-carboxylic acid (10 h-int, 99 mg, 93%) as a white solid: IR (neat) 3391, 3128, 3055, 2028, 2963, 2938, 1740, 1718, 1616, 1592, 1536, 1490, 1481, 1450, 1434, 1410, 1388, 1367, 1351, 1326, 1289, 1249, 1197, 1169, 1106, 1089, 1064, 1043, 1014, 972, 946, 926, 897, 885, 850, 801, 776, 762, 743, 727, 689, 652, 609 cm⁻¹; ¹H NMR (DMSO-d₆, 600 MHz): 38.51 (s, 1H), 7.55 (br s, 1H), 7.47-7.45 (m, 4H), 6.82-6.78 (m, 2H), 5.13 (s, 2H), 4.81 (dd, J=3.6, 6.6 Hz, 1H), 2.81-2.77 (m, 1H), 2.66-2.61 (m, 1H), 2.16-2.14 (m, 1H), 2.07-2.03 (m, 1H).

4-Chlorobenzyl ((2S)-2-(((tetrahydro-2H-pyran-2-yl)oxy)carbamoyl)chroman-8-yl)carbamate (10h). A solution of (S)-8-((((4-chlorobenzyl)oxy)carbonyl)amino)chromane-2-carboxylic acid (10 h-int, 0.097 g, 0.268 mmol) in DMF/CH₂Cl₂ (0.4 mL, 1/1) was cooled to 0° C. and treated with O-(tetrahydro-2H-pyran-2-yl)hydroxylamine (0.063 g, 0.536 mmol) in CH₂Cl₂ (0.4 mL). To this solution was added T3P (0.341 g, 0.536 mmol, 50% CH₃CN soln) in CH₂Cl₂ (0.4 mL) followed by DIPEA (0.094 mL, 0.536 mmol). The solution was stirred at 0° C. for 10 min and warmed to room temperature. After 7 h, the reaction mixture was extracted with CH₂Cl₂ and washed with H₂O. The aqueous layer was back extracted with CH₂Cl₂ (4×). The combined organic layer was washed with 0.25M HCl (2×), 1M LiCl (2×), brine, dried (Na₂SO₄), filtered and concentrated. The residue was purified by chromatography on SiO₂ (ISCO-Rf, 0-100% EtOAc/hexanes) to give 4-chlorobenzyl ((2S)-2-(((tetrahydro-2H-pyran-2-yl)oxy)carbamoyl)chroman-8-yl)carbamate (10 h, 0.115 g, 93%) as a colorless oil: ¹H NMR (CDCl₃, 600 MHz): δ 10.00 (br s, 0.5H), 9.71 (br s, 0.5H), 7.52-7.49 (m, 0.5H), 7.42-7.41 (m, 0.5H), 7.38-7.34 (m, 4H), 6.89-6.85 (m, 2H), 6.76 (br s, 0.5H), 6.71 (br s, 0.5H), 5.17 (d, J=3.0 Hz, 2H), 5.04 (app t, J=2.4 Hz, 0.5H), 4.97 (app t, J=3.0 Hz, 0.5H), 4.85 (app t, J=4.8 Hz, 0.5H), 4.79 (dd, J=4.2, 6.6 Hz, 0.5H), 4.00-3.92 (m, 1H), 3.55-3.46 (m, 1H), 2.86-2.72 (m, 2H), 2.47-2.31 (m, 1H), 2.26-2.14 (m, 1H), 1.88-1.74 (m, 3H), 1.63-1.52 (m, 3H).

4-Chlorobenzyl (S)-(2-(hydroxycarbamoyl)chroman-8-yl)carbamate (11h). A solution of 4-chlorobenzyl ((2S)-2-(((tetrahydro-2H-pyran-2-yl)oxy)carbamoyl)chroman-8-yl)carbamate (10 h (0.115 g, 0.249 mmol) in CH₂Cl₂/MeOH (4 mL, 1/1) was treated with Amberlyst-15 (0.052 g, washed with MeOH). After 24 h, the reaction mixture was filtered through Celite®, washed with MeOH then with CH₂Cl₂/MeOH, and concentrated. The solid residue is treated with Et₂O/hexanes (30:70) and the solid was collected by filtration and dried in vacuo to give 4-chlorobenzyl (S)-(2-(hydroxycarbamoyl)chroman-8-yl)carbamate (11 h, 0.079 g, 85%) as a tan solid: mp 155-157° C.; [α]_{D}=−76.0 (c 0.08, DMSO); IR (neat) 3302, 3066, 2897, 2846, 1678, 1612, 1596, 1527, 1459, 1433, 1410, 1369, 1343, 1331, 1314, 1260, 1238, 1222, 1201, 1183, 1108, 1086, 1060, 1028, 1013, 978, 888, 833, 806, 776, 759, 727, 705, 662, 637 cm⁻¹; ¹H NMR (DMSO-d₆, 600 MHz): δ 11.15 (s, 1H), 9.19 (s, 1H), 9.00 (s, 1H), 7.55 (d, J=7.2 Hz, 1H), 7.48-7.46 (m, 4H), 6.83-6.79 (m, 2H), 5.16 (s, 2H), 4.61 (dd, J=3.0, 9.0 Hz, 1H), 2.85-2.80 (m, 1H), 2.71-2.66 (m, 1H), 2.24-2.20 (m, 1H), 1.91-1.86 (m, 1H); ¹³C NMR (DMSO-d₆, 150 MHz): δ 166.7, 154.2, 144.1, 136.1, 133.2, 130.6, 129.0, 126.8, 124.8, 122.8, 120.6, 119.9, 75.5, 65.6, 24.8, 23.8; HRMS (ESI+) m/z for C₁₈H₁₆O₅N₂Cl [M−H] calcd 375.0742, found 375.0753.

3,4-Dichlorobenzyl (4-nitrophenyl) carbonate. To a solution of 3,4-dichlorobenzyl alcohol (177.02 mg, 0.92 mmol) in dry THE (0.75 mL) was added pyridine (0.125 mL, 1.545 mmol) at 0° C. The solution was treated with 4-nitrophenyl carbonochloridate (0.240 g, 1.191 mmol). After 10 min, the mixture was warmed to room temperature. After 3 h, the reaction mixture was extracted with EtOAc washed with 0.25 M HCl (2×), brine (2×), dried (Na₂SO₄), filtered and concentrated. The residue was purified by chromatography on SiO₂ (ISCO-Rf, 0-100% EtOAc/hexanes) to give 3,4-dichlorobenzyl (4-nitrophenyl) carbonate (0.199 mg, 63%). as a white solid: ¹H NMR (CDCl₃, 600 MHz): δ 8.30-8.28 (m, 2H), 7.56 (d, J=1.8 Hz, 1H), 7.49 (d, J=7.8 Hz, 1H), 7.40-7.38 m, 2H), 7.29 (dd, J=2.4, 8.4 Hz, 1H), 5.24 (s, 2H).

Methyl (S)-8-((((3,4-dichlorobenzyl)oxy)carbonyl)amino)chromane-2-carboxylate (9i). To a mixture of methyl (S)-8-aminochromane-2-carboxylate ((S)-3 (118 mg, 0.570 mmol), HOBT (69 mg, 0.456 mmol), and 4 Å powdered molecular sieves (130 mg) in DMF (0.5 mL) was added 3,4-dichlorobenzyl (4-nitrophenyl) carbonate (130 mg, 0.380 mmol) in anhydrous DMF (0.15 mL) at room temperature. After 17 h, the mixture was filtered and extracted with EtOAc, washed with brine, dried (Na₂SO₄), filtered and concentrated. The residue was purified by chromatography on SiO₂ (ISCO-Rf, 0-100% EtOAc/hexanes) to give methyl (S)-8-((((3,4-dichlorobenzyl)oxy)carbonyl)amino)chromane-2-carboxylate (9i, 0.153 g, 98%-approx 70% purity) as a yellow oil and was used without further purification: ¹H NMR (CDCl₃, 600 MHz): δ 7.92 (br s, 1H), 7.51 (d, J=1.8 Hz, 1H), 7.44 (d, J=7.8 Hz, 1H), 7.26-7.24 (m, 2H), 6.87 (app t, J=8.4 Hz, 1H), 6.73 (dd, J=0.6, 1.2 Hz, 1H), 5.14 (app q, J=12.6 Hz, 2H), 4.80 (dd, J=4.2, 7.2 Hz, 1H), 3.80 (s, 3H), 2.85-2.80 (m, 1H), 2.77-2.72 (m, 1H), 2.31-2.26 (m, 1H), 2.23-2.17 (m, 1H).

(S)-8-((((3,4-Dichlorobenzyl)oxy)carbonyl)amino)chromane-2-carboxylic acid (10i-int). A solution of methyl (S)-8-((((3,4-dichlorobenzyl)oxy)carbonyl)amino)chromane-2-carboxylate (9i, 0.146 g, 0.356 mmol) in MeOH/THF (4 mL, 3/1) was treated with 1M LiGH (0.9 mL) at room temperature. After stirring for 3.5 h, the reaction mixture was concentrated to reduce volume, cooled to 0° C., and acidified with conc. HCl to pH 2. The solution was extracted with EtOAc and washed with H₂O. The aqueous layer was back extracted with EtOAc (4×). The combined organic layer was washed with brine, dried (Na₂SO₄), filtered and concentrated to give (S)-8-((((3,4-dichlorobenzyl)oxy)carbonyl)amino)chromane-2-carboxylic acid (10i-int, 137 mg, 97%, approximate 70% purity) as a white solid and was used without further purification: ¹H NMR (DMSO-d₆, 600 MHz): δ 13.04 (s, 1H), 8.60 (s, 1H), 7.73 (s, 1H), 7.66 (d, J=8.4 Hz, 1H), 7.55 (br s, 1H), 7.43 (dd, J=1.2, 8.4 Hz, 1H), 6.82-6.79 (m, 2H), 5.14 (s, 2H), 4.84 (dd, J=4.2, 6.6 Hz, 1H), 2.82-2.77 (m, 1H), 2.66-2.61 (m, 1H), 2.18-2.13 (m, 1H), 2.09-2.05 (m, 1H).

3,4-Dichlorobenzyl ((2S)-2-(((tetrahydro-2H-pyran-2-yl)oxy)carbamoyl)chroman-8-yl)carbamate (10i). A solution of(S)-8-((((3,4-dichlorobenzyl)oxy)carbonyl)amino)chromane-2-carboxylic acid (10i-int, 0.130 g, 0.328 mmol) in DMF/CH₂Cl₂ (0.6 mL, 1/1) was cooled to 0° C. and treated with O-(tetrahydro-2H-pyran-2-yl)hydroxylamine (0.077 g, 0.656 mmol) in CH₂Cl₂ (0.4 mL). To this solution was added T3P (0.417 g, 0.656 mmol, 50% CH₃CN soln) in CH₂Cl₂ (0.20 mL) followed by DIPEA (0.114 mL, 0.656 mmol). The solution was stirred at 0° C. for 10 min and warmed to room temperature. After 23 h, the reaction mixture was extracted with CH₂Cl₂ and washed with H₂O. The aqueous layer was back extracted with CH₂Cl₂ (4×). The combined organic layer was washed with 0.25 M HCl (2×), 1M LiCl (2×), and brine, dried (Na₂SO₄), filtered and concentrated. The residue obtained was purified by chromatography on SiO₂ (ISCO-Rf, 0-100% EtOAc/hexanes) to give 3,4-dichlorobenzyl ((2S)-2-(((tetrahydro-2H-pyran-2-yl)oxy)carbamoyl)chroman-8-yl)carbamate (10i, 0.125 g, 77%, contains residual solvent) and 1 was used without further purification: ¹H NMR (CDCl₃, 400 MHz): δ 9.99 (br s, 0.5H), 9.69 (br s, 0.5H), 7.54 (dd, J=2.0, 10.8 Hz, 1H), 7.51-7.48 (m, 0.5H), 7.45 (dd, J=1.6, 8.4 Hz, 1H), 7.42-7.39 (m, 0.5H), 7.25 (br s, 1H), 6.91-6.86 (m, 2H), 6.76 (br s, 0.5H), 6.71 (br s, 0.5H), 5.21-5.12 (m, 2H), 5.04 (app t, J=2.8 Hz, 0.5H), 4.97 (app t, J=2.4 Hz, 0.5H), 4.86 (app t, J=4.4 Hz, 0.5H), 4.82 (dd, J=4.4, 6.8 Hz, 0.5H), 4.02-3.91 (m, 1H), 3.57-3.47 (m, 1H), 2.83-2.76 (m, 2H), 2.44-2.30 (m, 1H), 2.27-2.13 (m, 1H), 1.90-1.75 (m, 3H), 1.68-1.52 (m, 3H).

3,4-Dichlorobenzyl (S)-(2-(hydroxycarbamoyl)chroman-8-yl)carbamate (11i). A solution of 3,4-dichlorobenzyl ((2S)-2-(((tetrahydro-2H-pyran-2-yl)oxy)carbamoyl)chroman-8-yl)carbamate (10i, 0.121 g, 0.244 mmol) in CH₂Cl₂/MeOH (3 mL, 1/1) was treated with Amberlyst-15 (0.051 g, washed with MeOH) at room temperature. After 23 h, the solution was filtered through Celite®, washed with MeOH then CH₂Cl₂/MeOH and concentrated. The solid residue was treated with Et₂O/hexanes (40:60) and the solid was collected by filtration and dried in vacuo to give 3,4-dichlorobenzyl (S)-(2-(hydroxycarbamoyl)chroman-8-yl)carbamate (11i, 0.067 g, 50%) as a white solid: [α]_{D}=−71.3 (c 0.11, DMSO); IR (neat) 3297, 3091, 3069, 2897, 1677, 1613, 1595, 1535, 1461, 1434, 1401, 1354, 1343, 1315, 1254, 1225, 1130, 1109, 1086, 1062, 1027, 1012, 978, 889, 838, 788, 775, 762, 714, 704, 687, 638 cm⁻¹; ¹H NMR (DMSO-d₆, 600 MHz): δ 11.13 (s, 1H), 9.22 (s, 1H), 9.01 (s, 1H), 7.73 (s, 1H), 7.68 (d, J=8.4 Hz, 1H), 7.55 (br s, 1H), 7.45 (d, J=7.8 Hz, 1H), 6.84-6.81 (m, 2H), 5.17 (s, 2H), 4.62 (dd, J=2.4, 9.0 Hz, 1H), 2.85-2.80 (m, 1H), 2.71-2.67 (m, 1H), 2.23-2.21 (m, 1H), 1.92-1.87 (m, 1H); ¹³C NMR (DMSO-d₆, 150 MHz): δ 166.6, 154.1, 144.1, 138.3, 131.5, 131.2, 131.1, 130.5, 128.8, 126.7, 124.9, 122.8, 120.6, 119.9, 75.5, 64.9, 40.4, 40.3, 40.1, 40.0, 39.8, 39.7, 39.6, 24.8, 23.7; HRMS (ESI+) m/z for C₁₈H₁₅O₅N₂Cl₂ [M−H] calcd 409.0353, found 409.0357.

2-Methoxybenzyl (4-nitrophenyl) carbonate. To a solution of 2-methoxybenzyl alcohol (0.2 mL, 1.5 mmol), 4-nitrophenyl carbonochloridate (0.373 g, 1.85 mmol) in dry THE (3 mL) was added pyridine (0.242 mL, 3 mmol) at 0° C. After 10 min, the reaction mixture was warmed to room temperature and stirred for 2.5 hours. The reaction mixture was extracted with EtOAc, washed with 0.25M HCl (3×), brine (2×), dried (Na₂SO₄), filtered and concentrated. The residue was purified by chromatography on SiO₂ (ISCO-Rf, 0-20% EtOAc/hexanes) to give 2-methoxybenzyl (4-nitrophenyl) carbonate (0.145 g, 32%) as a white solid: ¹H NMR (CDCl₃, 600 MHz): δ 8.27 (d, J=9.0 Hz, 2H), 7.41-7.36 (m, overlapping signals, 4H), 6.99 (app t, J=7.8 Hz, 1H), 6.94 (d, J=8.4 Hz, 1H), 5.37 (s, 2H), 3.89 (s, 3H).

Methyl (S)-8-((((2-methoxybenzyl)oxy)carbonyl)amino)chromane-2-carboxylate (9j). To a mixture of methyl (S)-8-aminochromane-2-carboxylate ((S)-3, 130 mg, 0.628 mmol), HOBt (77 mg, 0.503 mmol), and 4 Å powdered molecular sieves (130 mg) in DMF (0.5 mL) was added 2-methoxybenzyl (4-nitrophenyl) carbonate (127 mg, 0.419 mmol) in DMF (1.5 mL) at room temperature. After 2.5 d, the reaction mixture was extracted with EtOAc, washed with H₂O, brine, dried (Na₂SO₄), filtered and concentrated. The residue was purified by chromatography on SiO₂ (ISCO-Rf, 0-100% EtOAc/hexanes) to give semi-pure product (0.111 g). The residue was extracted with CH₂Cl₂ and washed with saturated NaHCO₃ and concentrated to give methyl (S)-8-((((2-methoxybenzyl)oxy)carbonyl)amino)chromane-2-carboxylate (9j, 85 mg, 55%-contains residual solvent) as a colorless oil that was used without further purification: ¹H NMR (CDCl₃, 500 MHz): δ 7.99 (br s, 1H), 7.41 (d, J=7.5 Hz, 1H), 7.34-7.30 (m, 2H), 6.97 (app t, J=7.5 Hz, 1H), 6.91 (d, J=8.0 Hz, 1H), 6.87 (app t, J=8.0 Hz, 1H), 6.71 (d, J=7.5 Hz, 1H), 5.27 (s, 2H), 4.77 (dd, J=4.0, 7.0 Hz, 1H), 3.87 (s, 3H), 3.77 (s, 3H), 2.84-2.70 (m, 2H), 2.28-2.18 (m, 2H).

(S)-8-((((2-Methoxybenzyl)oxy)carbonyl)amino)chromane-2-carboxylic acid (10j-int). A solution of methyl (S)-8-((((2-methoxybenzyl)oxy)carbonyl)amino)chromane-2-carboxylate (9j, 0.083 g, 0.223 mmol) in MeOH/THF (2 mL, 3/1) was treated with 1M LiGH (0.6 mL) at room temperature. After 1.5 h, the reaction was concentrated to reduce volume. The reaction mixture was cooled to 0° C. and acidified with conc. HCl to pH 2. The solution was extracted with EtOAc and washed with H₂O. The aqueous layer was back extracted with EtOAc (4×). The combined organic layer was washed with brine, dried (Na₂SO₄), filtered and concentrated to give S)-8-((((2-methoxybenzyl)oxy)carbonyl)amino)chromane-2-carboxylic acid (10j-int, 80 mg, quant.-contains residual solvent) as a solid that was used without further purification: ¹H NMR (DMSO-d₆, 600 MHz): δ 13.03 (br s, 1H), 8.42 (s, 1H), 7.59 (d, J=6.0 Hz, 1H), 7.39 (d, J=6.6 Hz, 1H), 7.35-7.32 (m, 1H), 7.03 (d, J=7.8 Hz, 1H), 6.98-6.92 (m, 1H), 6.82-6.77 (m, 2H), 5.13 (s, 2H), 4.81 (dd, J=3.6, 6.6 Hz, 1H), 3.82 (s, 3H), 2.81-2.76 (m, 1H), 2.66-2.61 (m, 1H), 2.18-2.13 (m, 1H), 2.07-2.04 (m, 1H).

2-Methoxybenzyl ((2S)-2-(((tetrahydro-2H-pyran-2-yl)oxy)carbamoyl)chroman-8-yl)carbamate (10j). A solution of (S)-8-((((2-methoxybenzyl)oxy)carbonyl)amino)chromane-2-carboxylic acid (10j-int, 0.075 g, 0.209 mmol) in DMF/CH₂Cl₂ (0.4 mL, 1/1) was cooled to 0° C. and treated with O-(tetrahydro-2H-pyran-2-yl)hydroxylamine (0.049 g, 0.419 mmol) in CH₂Cl₂ (0.4 mL). To this solution was added T3P (0.267 g, 0.419 mmol, 50% CH₃CN soln) in CH₂Cl₂ (0.4 mL) followed by DIPEA (0.073 mL, 0.419 mmol). The solution was stirred at 0° C. for 10 min and warmed to at room temperature. After 5 h, the reaction mixture was extracted with CH₂Cl₂ and washed with H₂O. The aqueous layer was back extracted with CH₂Cl₂ (4×). The combined organic layer was washed with 0.25M HCl (2×), 1M LiCl (2×), and brine, dried (Na₂SO₄), filtered and concentrated. The residue was purified by chromatography on SiO₂ (ISCO-Rf, 0-100% EtOAc/hexanes) to give 2-methoxybenzyl ((2S)-2-(((tetrahydro-2H-pyran-2-yl)oxy)carbamoyl)chroman-8-yl)carbamate (10j, 0.078 g, 82%-contained residual solvent) as a colorless oil that was used without further purification: ¹H NMR (CDCl₃, 500 MHz): δ 10.07 (s, 0.5H), 9.78 (s, 0.5H), 7.52 (d, J=6.5 Hz, 0.5H), 7.44-7.39 (m, 1H), 7.35-7.31 (m, 1H), 6.97 (app t, J=7.0 Hz, 1H), 6.92-6.90 (m, 1H), 6.89-6.84 (m, 2H), 6.76 (br s, 0.5H), 6.72 (br s, 0.5H), 5.28 (s, 2H), 5.05 (app t, J=4.0 Hz, 0.5H), 4.99 (app t, J=3.0 Hz, 0.5H), 4.86 (app t, J=4.5 Hz, 0.5H), 4.80 (dd, J=4.0, 6.5 Hz, 0.5H), 3.87 (s, 1.5H), 3.86 (s, 1.5H), 3.58-3.47 (m, 1H), 2.86-2.70 (m, 2H), 2.47-2.30 (m, 1H), 2.26-2.11 (m, 1H), 1.87-1.74 (m, 3H), 1.62-1.51 (m, 3H).

2-Methoxybenzyl (S)-(2-(hydroxycarbamoyl)chroman-8-yl)carbamate (11j). A solution of 2-methoxybenzyl ((2S)-2-(((tetrahydro-2H-pyran-2-yl)oxy)carbamoyl)chroman-8-yl)carbamate (10j, 0.076 g, 0.166 mmol) in CH₂Cl₂/MeOH (3 mL, 1/1) was treated with Amberlyst-15 (0.040 g, washed with MeOH) at room temperature. After 21 h, the mixture was warmed to 40° C. for 2 h. The solution was cooled to room temperature, filtered through Celite®, washed with MeOH rinsed with CH₂Cl₂/MeOH, and concentrated. The solid was treated with Et₂O/hexanes (30:70) and the solid was collected by filtration and dried in vacuo to afford 2-methoxybenzyl (S)-(2-(hydroxycarbamoyl)chroman-8-yl)carbamate (11j, 0.038 g, 62%) as a tan solid: [α]_{D}=−58.3 (c 0.14, DMSO); IR (neat) 3218, 2930, 2837, 1664, 1615, 1591, 1533, 1493, 1479, 1439, 1324, 1289, 1217, 1191, 1092, 1060, 1024, 966, 884, 865, 831, 751, 727, 695; ¹H NMR (DMSO-d₆, 600 MHz): δ 11.15 (s, 1H), 9.13 (s, 1H), 8.98 (s, 1H), 7.56 (d, J=7.8 Hz, 1H), 7.40-7.34 (m, overlapping signals, 2H), 7.05 (d, J=8.4 Hz, 1H), 6.97 (app t, J=7.2 Hz, 1 H), 6.83-6.78 (m, 2H), 5.14 (d, J=1.8 Hz, 2H), 4.60 (dd, J=3.0, 9.6 Hz, 1H), 3.82 (s, 3H), 2.86-2.80 (m, 1H), 2.71-2.66 (m, 1H), 2.24-2.19 (m, 1H), 1.91-1.87 (m, 1H); ¹³CNMR (DMSO-d₆, 150 MHz): δ 166.7, 157.7, 154.4, 144.0, 130.4, 130.2, 126.9, 124.7, 124.6, 122.8, 120.7, 120.6, 119.9, 111.4, 75.5, 62.0, 55.9, 24.9, 23.8; HRMS (ESI+) m/z for C₁₉H₁₉O₆N₂ [M−H] calcd 371.1238, found 371.1254.

Methyl 8-(((benzyloxy)carbonyl)amino)chromane-2-carboxylate (9k). A solution of methyl 8-aminochromane-2-carboxylate (3, 0.233 g, 1.110 mmol) in CH₂Cl₂ (8.0 mL) was cooled to 0° C. and treated with pyridine (0.098 mL, 1.22 mmol) followed by benzyl chloroformate (0.174 mL, 1.22 mmol) in CH₂Cl₂ (2 mL). After 10 min, the reaction mixture was warmed to room temperature. After 3 h, the reaction mixture was extracted with CH₂Cl₂ washed with H₂O, 0.1M HCl, and brine. The organic layer was dried (Na₂SO₄), filtered and concentrated to give methyl 8-(((benzyloxy)carbonyl)amino)chromane-2-carboxylate (9k, 0.352 g, 93%) as an off white solid.

Lithium 8-(((benzyloxy)carbonyl)amino)chromane-2-carboxylate (10k-int). To a solution of methyl 8-(((benzyloxy)carbonyl)amino)chromane-2-carboxylate (9k, 0.426 g, 1.25 mmol, crude) in THF/MeOH (3.0/1.5 mL) was added 1M LiGH (1.25 mL) at room temperature. After 15.5 h, the solution was concentrated and the residue was treated with PhMe and concentrated (3×) to give lithium 8-(((benzyloxy)carbonyl)amino)chromane-2-carboxylate that was used without further purification.

Benzyl (2-(((tetrahydro-2H-pyran-2-yl)oxy)carbamoyl)chroman-8-yl)carbamate (10k). A solution of lithium 8-(((benzyloxy)carbonyl)amino)chromane-2-carboxylate (10k-int, 0.369 g, 1.11 mmol, crude) in DMF (3.0 mL) was treated with O-(tetrahydro-2H-pyran-2-yl)hydroxylamine (0.143 g, 1.22 mmol) in DMF (1.5 mL) followed by HATU (0.463 g, 1.22 mmol) and DIPEA (0.22 mL, 1.33 mmol) at room temperature. After 15.5 h, the solution was extracted with EtOAc, washed with 0.25M HCl, 1M LiCl (3×), brine, dried (Na₂SO₄), filtered and concentrated. The residual oil was purified by chromatography on SiO₂ (2/1, hexanes/EtOAc) to give benzyl (2-(((tetrahydro-2H-pyran-2-yl)oxy)carbamoyl)chroman-8-yl)carbamate (10k, 0.148 g, contains residual solvent) as a white solid.

Benzyl (2-(hydroxycarbamoyl)chroman-8-yl)carbamate (11k). To a solution of benzyl (2-(((tetrahydro-2H-pyran-2-yl)oxy)carbamoyl)chroman-8-yl)carbamate (10k, 0.148 g, 0.347 mmol) in MeOH (3.0 mL) was added Amberlyst-15 (0.015 g, washed with MeOH) at room temperature. After 3 h, the reaction mixture was filtered through Celite®, rinsed with MeOH and concentrated. The solid was purified by chromatography on SiO₂ (0-10% MeOH/CH₂Cl₂) to give benzyl (2-(hydroxycarbamoyl)chroman-8-yl)carbamate (11k, 0.042 g, 11%-4 steps) as a white solid: ¹H NMR (acetone-d₆, 600 MHz): δ 10.74 (s, 1H), 8.18 (s, 1H), 8.17 (s, 1H), 7.67 (d, J=7.2 Hz, 1H), 7.46-7.41 (m, 2H), 7.39-7.36 (m, 2H), 7.35 (app t, J=7.2 Hz, 1H), 6.86-6.82 (m, 2H), 5.19 (s, 2H), 4.72 (dd, J=2.4, 7.2 Hz, 1H), 2.82-2.77 (m, 1H), 2.76-2.74 (m, 1H), 2.25-2.21 (m, 1H), 2.16-2.09 (m, 1H); ¹³C NMR (acetone-d₆. 150 MHz): δ 167.5, 154.9, 144.5, 137.7, 129.3, 129.1, 128.9, 127.5, 125.4, 123.4, 121.3, 120.1, 76.1, 67.2, 25.1, 23.8; HRMS (ESI+) m/z for C₁₈H₁₉O₅N₂ [M+H] calcd 343.1288, found 343.1287.

Methyl 8-((((2-chlorobenzyl)oxy)carbonyl)amino)chromane-2-carboxylate (9L). A solution of methyl 8-aminochromane-2-carboxylate (3, 0.200 g, 0.965 mmol) in CH₂Cl₂ (8.0 mL) was cooled to 0° C. and treated with pyridine (0.086 mL, 1.06 mmol) followed by 2-chlorobenzyl chloroformate (0.16 mL, 1.06 mmol) in CH₂Cl₂ (2 mL). After 10 min, the reaction mixture was warmed to room temperature. After 19 h, the reaction mixture was extracted with CH₂Cl₂, washed with H₂O, 0.2M HCl (2×), and brine. The organic layer was dried (Na₂SO₄), filtered and concentrated to give methyl 8-((((2-chlorobenzyl)oxy)carbonyl)amino)chromane-2-carboxylate (9L, 0.104 g, 29%) as a brown solid.

Lithium 8-((((2-chlorobenzyl)oxy)carbonyl)amino)chromane-2-carboxylate (101-int). To a solution of methyl 8-((((2-chlorobenzyl)oxy)carbonyl)amino)chromane-2-carboxylate (9L, 0.104 g, 0.277 mmol) in THF/MeOH (2.0/1.0 mL) was added 1M LiGH (0.30 mL) at room temperature. After 15.5 h, the solution was concentrated and the residue was treated with PhMe and concentrated (3×) to give lithium 8-((((2-chlorobenzyl)oxy)carbonyl)amino)chromane-2-carboxylate that was used without further purification.

2-Chlorobenzyl (2-(((tetrahydro-2H-pyran-2-yl)oxy)carbamoyl)chroman-8-yl)carbamate (101-racemic). A solution of lithium 8-((((2-chlorobenzyl)oxy)carbonyl)amino)chromane-2-carboxylate (10L-int, 0.102 g, 0.278 mmol theoretical) in DMF (2.5 mL) was treated with O-(tetrahydro-2H-pyran-2-yl)hydroxylamine (0.036 g, 0.305 mmol) in DMF (1.5 mL) followed by T3P (0.194 g, 50% EtOAc soln) and DIPEA (0.06 mL, 0.333 mmol) at room temperature. After 3 h, the reaction mixture was extracted with EtOAc, washed with satd. NH₄Cl, 1M LiCi (3×), brine, dried (Na₂SO₄), filtered and concentrated. The residue was purified by chromatography on SiO₂ (hexanes/EtOAc, 2/1) to give 2-chlorobenzyl (2-(((tetrahydro-2H-pyran-2-yl)oxy)carbamoyl)chroman-8-yl)carbamate (10L, 0.080 g) as a white solid: HRMS (ESI+) m/z for C₂₃H₂₅O₆N₂ClNa [M+Na] calcd 483.1293, found 483.1292.

2-Chlorobenzyl (2-(hydroxycarbamoyl)chroman-8-yl)carbamate ((−)-11L). To a solution of 2-chlorobenzyl (2-(((tetrahydro-2H-pyran-2-yl)oxy)carbamoyl)chroman-8-yl)carbamate (10L, 0.080 g, 0.174 mmol) in MeOH (3.0 mL) was added Amberlyst-15 (0.007 g, washed with MeOH). After 3 h, the reaction mixture was filtered through Celite®, rinsed with MeOH and concentrated. The solid was diluted with Et₂O and collected by filtration and washed with hexanes to give 2-chlorobenzyl (2-(hydroxycarbamoyl)chroman-8-yl)carbamate (11L, 0.0271 g, 10%-4 steps) as a white solid: mp 182-188° C.; ¹³C NMR (acetone-d₆, 100 MHz): δ 166.5, 153.8, 143.8, 134.3, 133.2, 130.2, 129.9, 129.4, 127.3, 126.5, 124.7, 122.6, 120.5, 119.4, 75.3, 63.6, 24.2, 22.9; HRMS (ESI+) m/z for C₁₈H₁₈O₅N₂Cl [M+H] calcd 377.0899, found 377.0893.

Methyl (R)-8-((((2-chlorobenzyl)oxy)carbonyl)amino)chromane-2-carboxylate (+9L) To a mixture of methyl 8-((((2-chlorobenzyl)oxy)carbonyl)amino)chromane-2-carboxylate (9L, 1.99 g, 5.29 mmol) in nBuOH (30 mL) and aqueous buffer (35 mL, pH 7.0) was added Amano-Lipase PS (Burkholderia cepacia (8.45 g) at room temperature. The mixture was heated at 45-50° C. After 2.5 d, the mixture was concentrated. The residue was extracted with EtOAc, washed with 0.5M NaOH (2×) with aqueous pH of 8. The combined original organic layer was dried (Na₂SO₄), filtered and concentrated to give a mixture of methyl and butyl esters (1.1 g) that were separated by chromatography on SiO₂ (ISCO-Rf, 0-25% EtOAc/hexanes) to give methyl (R)-8-((((2-chlorobenzyl)oxy)carbonyl)amino)chromane-2-carboxylate (+9L, 0.526 g, 27%, HPLC-ChiralPak AD-H, 5% EtOH/hexanes, 254 nM, indicates >99% ee; e.r.=477/1) as a peach solid and butyl 8-((((2-chlorobenzyl)oxy)carbonyl)amino)chromane-2-carboxylate (0.381 g, 19%, HPLC-ChiralPak AD-H, 5% EtOH/hexanes, 254 nM, indicates e.r.=1.1/1). The original aqueous (basic) was cooled to 0° C. and acidified with 3M HCl. The solution was extracted with EtOAc (2×). The combined organic was washed with brine, dried (Na₂SO₄), filtered and concentrated to give product (0.600 g) as an orange oil that contained some AcOH. The oil was diluted with CH₂Cl₂, washed with brine (2×), dried (Na₂SO₄), filtered and concentrated to give 8-((((2-chlorobenzyl)oxy)carbonyl)amino)chromane-2-carboxylic acid (0.558 g, 29%) as an amber oil. A small portion of this crude acid (0.004 g, 0.011 mmol) in MeOH (2.0 mL) was treated with SOCl₂ (2 drops) at 0° C. The solution was warmed to room temperature then heated at 50° C. for 1 h. The solution was concentrated and the residue was extracted with EtOAc, washed with satd. NaHCO₃, brine, dried (Na₂SO₄), filtered and concentrated. The residual oil was filtered through SiO₂, eluted with EtOAc and concentrated to give (enantioenriched) methyl (S)-8-((((2-chlorobenzyl)oxy)carbonyl)amino)chromane-2-carboxylate (AA, HPLC-ChiralPak AD-H, 5% EtOH/hexanes, 254 nM, indicates e.r.=3/1). (R)-8-((((2-chlorobenzyl)oxy)carbonyl)amino)chromane-2-carboxylate (+9L): [α]_{D}=+36.9 (c 0.36, CH₂Cl₂); ¹H NMR (CDCl₃, 400 MHz): δ7.94 (s, 1H), 7.47-7.45 (m, 1H), 7.38-7.35 (m, 1H), 7.32-7.30 (m, 1H), 7.25-7.23 (m, 2H), 6.84 (app t, J=7.8 Hz, 1H), 6.69 (d, J=7.3 Hz, 1H), 5.30 (d, J=3.0 Hz, 2H), 4.76 (dd, J=4.0, 6.5 Hz, 1H), 3.75 (s, 3H), 2.78-2.71 (m, 2H), 2.27-2.14 (m, 2H).

Methyl (S)-8-((((2-chlorobenzyl)oxy)carbonyl)amino)chromane-2-carboxylate (−9L). To a solution of enantioenriched (S)-8-((((2-chlorobenzyl)oxy)carbonyl)amino)chromane-2-carboxylic acid (0.558 g, 1.54 mmol) in CH₂Cl₂/MeOH (5 mL, 0.2 mL) was added (trimethylsilyl)diazomethane (1.9 mL, 2.0M hexanes) at 0° C. After 5 min, the solution was warmed to room temperature. After 5 min, the yellow solution was cooled to 0° C. and treated with AcOH (few drops until yellow color removed). The solution was extracted with CH₂Cl₂, washed with satd. NaHCO₃, brine, dried (Na₂SO₄), filtered and concentrated. The crude oil was purified by chromatography on SiO₂ (ISCO-Rf, 0-100% EtOAc/hexanes) to give (enantioenriched) methyl (S)-8-((((2-chlorobenzyl)oxy)carbonyl)amino)chromane-2-carboxylate (AA, 0.418 g, 72%) as a light yellow oil: HPLC ChiralPak AD-H, 95/5 hexanes/EtOH, 254 nM, e.r.=3/1). The mixture was separated by semi-preparative HPLC (Chiralpak AD-H, 2 cm×25 cm, 5 μM, 97/3 hexanes/EtOH, 254 nM) to give methyl (S)-8-((((2-chlorobenzyl)oxy)carbonyl)amino)chromane-2-carboxylate (−9L, 174 mg, HPLC-ChiralPak AD-H, 5% EtOH/hexanes, 254 nM, indicates e.r.=332/1) as a white solid: [α]_{D}=−41.1 (c 0.33, CH₂Cl₂); ¹H NMR (CDCl₃, 500 MHz): 37.97 (s, 1H), 7.50-7.48 (m, 1H), 7.41-7.39 (m, 1H), 7.35-7.33 (m, 1H), 7.28-7.26 (m, 2H), 6.88 (app t, J 7.5 Hz, 1H), 6.73 (d, J=7.5 Hz, 1H), 5.33 (d, J=4.0 Hz, 2H), 4.79 (dd, J=3.5, 7.0 Hz, 1H), 3.78 (s, 3H), 2.85-2.71 (m, 2H), 2.31-2.21 (m, 2H).

(R)-8-((((2-Chlorobenzyl)oxy)carbonyl)amino)chromane-2-carboxylic acid ((+)-101-int). A solution of methyl(R)-8-((((2-chlorobenzyl)oxy)carbonyl)amino)chromane-2-carboxylate (+9L, 0.150 g, 0.400 mmol) in MeOH/THF (2.0 mL, 1/1) was treated with 1M LiGH (0.45 mL) at room temperature. After 5 h, the solution was concentrated. The residue was diluted with H₂O, acidified with 1M HCl and extracted with EtOAc (2×). The combined organic layer was washed with brine, dried (Na₂SO₄), filtered and concentrated to give (R)-8-((((2-chlorobenzyl)oxy)carbonyl)amino)chromane-2-carboxylic acid (+101-int, 0.140 g, 97%) as a light pink solid: mp 119-121° C.; [α]_{D}=+45.1 (c 0.57, CH₂Cl₂); IR (neat) 3363, 3117, 1758, 1685, 1618, 1547, 1458, 1437, 1334, 1258, 1240, 1151, 1101, 1038, 976, 802, 775, 744, 728 cm⁻¹; ¹H NMR (DMSO-d₆, 600 MHz): δ8.56 (s, 1H), 7.60-7.57 (m, 2H), 7.50 (dd, J=3.6, 5.6 Hz, 1H), 7.40-7.38 (m, 2H), 6.82-6.79 (m, 2H), 5.22 (s, 2H), 4.82 (dd, J=3.9, 6.5 Hz, 1H), 2.81-2.77 (m, 1H), 2.67-2.62 (m, 1H), 2.17-2.14 (m, 1H), 2.07-2.04 (m, 1H); ¹³C NMR (DMSO-d₆, 150 MHz): δ171.6, 153.2, 134.0, 132.4, 130.0, 129.9, 129.3, 128.1, 127.4, 127.0, 126.2, 124.2, 121.9, 119.9, 73.2, 63.3, 23.7, 22.3; HRMS (LCMS ESI+) m/z calcd for C₁₈H₁₅O₅NCl [M−H] 360.0633, found 360.0644.

2-Chlorobenzyl ((2R)-2-(((tetrahydro-2H-pyran-2-yl)oxy)carbamoyl)chroman-8-yl)carbamate (10L-chiral). To a solution of (R)-8-((((2-chlorobenzyl)oxy)carbonyl)amino)chromane-2-carboxylic acid (+101-int, 0.140 g, 0.386 mmol) in DMF/CH₂Cl₂ (0.2/1.0 mL) was added O-(tetrahydro-2H-pyran-2-yl)hydroxylamine (0.062 g, 0.53 mmol) in CH₂Cl₂ (0.5 mL). The solution was cooled to 0° C. and treated with T3P (0.382 g, 0.60 mmol) followed by DIPEA (0.09 mL, 0.54 mmol). The reaction mixture was slowly warmed to room temperature. After 4.5 h, the reaction was extracted with CH₂Cl₂, washed with 0.2M HCl, 1M LiCl, H₂O, dried (Na₂SO₄), filtered and concentrated. The residual oil was purified by chromatography on SiO₂ (ISCO-Rf, 0-100% EtOAc/hexanes) to give 2-chlorobenzyl ((2R)-2-(((tetrahydro-2H-pyran-2-yl)oxy)carbamoyl)chroman-8-yl)carbamate (+10L, 0.144 g, 73%, contains residual solvent) as a light amber foam: ¹H NMR (CDCl₃, 600 MHz): 310.18 (s, 0.5H), 9.98 (s, 0.5H), 7.54-7.46 (m, overlapping signals, 2H), 7.42-7.40 (m, 1H), 7.30-7.29 (m, 2H), 7.6 (s, 0.5H), 7.01 (s, 0.5H), 6.89-6.87 (m, 2H), 5.35-5.34 (m, 2H), 5.05 (s, 0.5H), 4.98 (s, 0.5 H), 4.82 (app t, J=4.2 Hz, 0.5H), 4.77 (app t, J=5.3 Hz, 0.5H), 4.01-3.97 (m, 1H), 3.58 (d, J=11.3 Hz, 0.5H), 3.50 (d, J=11.3 Hz, 0.5H), 2.81-2.76 (m, 2H), 2.42-2.39 (m, 0.5H), 2.30-2.27 (m, 1H), 2.19-2.17 (m, 0.5H), 1.87-1.73 (m, 3H), 1.63-1.54 (m, 3H); HRMS (LCMS ESI+) m/z calcd for C₂₃H₂₅O₆N₂ClNa [M+Na] 483.1293, found 483.1292.

2-Chlorobenzyl (R)-(2-(hydroxycarbamoyl)chroman-8-yl)carbamate (11L). To a solution of 2-chlorobenzyl ((2R)-2-(((tetrahydro-2H-pyran-2-yl)oxy)carbamoyl)chroman-8-yl)carbamate (+10L, 0.140 g, 0.303 mmol) in MeOH (2.0 mL) was added Amberlyst-15 (0.036 g, washed with MeOH) at room temperature. After 22 h, the mixture was filtered through Celite®, rinsed with MeOH and concentrated. The residue was slurried with Et₂O/hexanes (1/1) and the solid was collected by filtration and dried in vacuo @ 90° C. to give 2-chlorobenzyl (R)-(2-(hydroxycarbamoyl)chroman-8-yl)carbamate (+11L, 0.086 g, 75%) as an off-white solid: mp 173-175° C.; [α]_{D}=+90.4 (c 0.29, DMSO); IR (neat) 3269, 1704, 1646, 1525, 1454, 1325, 1237, 1216, 1198, 1085, 1062, 1053, 973, 829, 779, 755, 677 cm⁻¹; ¹H NMR (DMSO-d₆, 400 MHz): δ11.15 (s, 1H), 9.24 (s, 1H), 9.00 (s, 1H), 7.60-7.51 (m, 3H), 7.42-7.39 (m, 2H), 6.84-6.79 (m, 2H), 5.25 (s, 2H), 4.61 (dd, J=2.6, 9.2 Hz, 1H), 2.85-2.79 (m, 1H), 2.71-2.65 (m, 1H), 2.23-2.20 (m, 1H), 1.91-1.86 (m, 1H); ¹³C NMR (DMSO-d₆, 100 MHz): δ166.1, 153.6, 143.6, 133.9, 132.7, 130.5, 130.1, 129.4, 127.4, 126.2, 124.4, 122.3, 120.1, 119.4, 75.0, 63.4, 24.3, 23.3; HRMS (LCMS ESI+) m/z calcd for C₁₈H₁₈O₅N₂Cl [M+H] 377.0899, found 377.0898.

(S)-8-((((2-Chlorobenzyl)oxy)carbonyl)amino)chromane-2-carboxylic acid (101-int).

A solution of methyl (S)-8-((((2-chlorobenzyl)oxy)carbonyl)amino)chromane-2-carboxylate (0.094 g, 0.25 mmol) in MeOH/THF (1.0/1.0 mL) was treated with 1M LiGH (0.3 mL) at room temperature. After 2 h, the solution was concentrated, diluted with H₂O, acidified with 1M HCl and extracted with EtOAc (2×). The combined organic layer was washed with brine, dried (Na₂SO₄), filtered and concentrated to give (S)-8-((((2-chlorobenzyl)oxy)carbonyl)amino)chromane-2-carboxylic acid as an off-white solid (101-int, 0.088 g, 97%): [α]_{D}=−49.9 (c 0.64, CH₂Cl₂); ¹H NMR (DMSO-d₆, 500 MHz): 312.99 (s, 1H), 8.56 (s, 1H), 7.60-7.56 (m, overlapping signals, 2H), 7.50 (dd, J=3.7, 5.7 Hz, 1H), 7.41-7.38 (m, 2H), 6.82-6.78 (m, 2H), 5.22 (s, 2H), 4.83 (dd, J=3.9, 6.6 Hz, 1H), 2.81-2.77 (m, 1H), 2.65-2.61 (m, 1H), 2.18-2.12 (m, 1H), 2.09-2.02 (m, 1H).

2-Chlorobenzyl ((2S)-2-(((tetrahydro-2H-pyran-2-yl)oxy)carbamoyl)chroman-8-yl)carbamate (101-chiral). To a solution of (S)-8-((((2-chlorobenzyl)oxy)carbonyl)amino)chromane-2-carboxylic acid (0.085 g, 0.23 mmol) in DMF/CH₂Cl₂ (0.1/1.0 mL) was added O-(tetrahydro-2H-pyran-2-yl)hydroxylamine (0.042 g, 0.36 mmol) in CH₂Cl₂ (0.5 mL). The solution was cooled to 0° C. and treated with T3P (0.218 g, 50% EtOAc soln) followed by DIPEA (0.05 mL, 0.30 mmol). The reaction mixture was slowly warmed to room temperature. After 4.5 h, the reaction was extracted with CH₂Cl₂, washed with 0.2M HCl, 1M LiCl, H₂O, dried (Na₂SO₄), filtered and concentrated. The residual oil was purified by chromatography on SiO₂ (ISCO-Rf, 0-100% EtOAc/hexanes) to give 2-chlorobenzyl ((2S)-2-(((tetrahydro-2H-pyran-2-yl)oxy)carbamoyl)chroman-8-yl)carbamate (101-chiral, 0.097 g, 90%) as a foamy solid: ¹H NMR (CDCl₃, 500 MHz): δ10.14 (s, 0.5H), 9.93 (s, 0.5H), 7.50-7.41 (m, overlapping signals, 2H), 7.39-7.37 (m, 1H), 7.28-7.25 (m, 1H), 7.00 (s, 0.5H), 6.95 (s, 0.5H), 6.87-6.84 (m, 2H), 5.33-5.31 (m, 2H), 5.03 (s, 0.5H), 4.96 (s, 0.5H), 4.80 (app t, J=4.6 Hz, 0.5H), 4.74 (app t, J=5.7 Hz, 0.5H), 3.99-3.92 (m, 1H), 3.56 (d, J=11.3 Hz, 0.5H), 3.47 (d, J=11.3 Hz, 0.5H), 2.80-2.72 (m, 2H), 2.41-2.37 (m, 0.5H), 2.27-2.23 (m, 1H), 2.16-2.12 (m, 0.5H), 1.83-1.72 (m, 3H), 1.59-1.49 (m, 3H); HRMS (LCMS ESI+) m/z calcd for C₂₃H₂₅O₆N₂ClNa [M+Na] 483.1293, found 483.1291.

2-Chlorobenzyl (S)-(2-(hydroxycarbamoyl)chroman-8-yl)carbamate (−11L). A solution of 2-chlorobenzyl ((2S)-2-(((tetrahydro-2H-pyran-2-yl)oxy)carbamoyl)chroman-8-yl)carbamate (101-chiral, 0.097 g, 0.21 mmol) in MeOH (2.0 mL) was treated with Amberlyst-15 (0.056 g, washed with MeOH) at room temperature. After 17 h, the mixture was filtered through Celite®, rinsed with MeOH and concentrated. The residue was slurried with Et₂O and the solid was collected by filtration and dried in vacuo at 90° C. to give 2-chlorobenzyl (S)-(2-(hydroxycarbamoyl)chroman-8-yl)carbamate (−11L, 0.059 g, 74%) as a light pink solid: mp 176-178° C.; [α]_{D}=−90.4 (c 0.26, DMSO); ¹H NMR (DMSO-d₆, 500 MHz): δ11.13 (d, J=1.1 Hz, 1H), 9.23 (s, 1H), 8.98 (d, J=1.5 Hz, 1H), 7.59-7.55 (m, overlapping signals, 2H), 7.53-7.51 (m, 1H), 7.42-7.39 (m, 2H), 6.84-6.79 (m, 2H), 5.25 (s, 2H), 4.62 (dd, J=2.7, 9.2 Hz, 1H), 2.85-2.80 (m, 1H), 2.72-2.66 (m, 1H), 2.24-2.19 (m, 1H), 1.93-1.85 (m, 1H); ¹³C NMR (DMSO-d₆, 125 MHz): δ166.1, 153.5, 143.6, 133.9, 132.7, 130.4, 130.0, 129.3, 127.3, 126.2, 124.3, 122.2, 120.1, 1194, 74.9, 63.3, 24.2, 23.2; HRMS (LCMS ESI+) m/z calcd for C₁₈H₁₈O₅N₂Cl [M+H]377.0899, found 377.0897.

Methyl 8-((4-fluorophenyl)sulfonamido)chromane-2-carboxylate (9m). A solution of methyl 8-aminochromane-2-carboxylate (3, 0.099 g, 0.48 mmol) in CH₂Cl₂ (2.0 mL) was treated with pyridine (0.10 mL, 1.2 mmol) followed by 4-fluorobenzenesulfonyl chloride (0.102 g, 0.52 mmol) at room temperature. After 22 h, the solution was extracted with CH₂Cl₂, washed with 0.5M HCl, brine, dried (Na₂SO₄), filtered and concentrated to give methyl 8-((4-fluorophenyl)sulfonamido)chromane-2-carboxylate (9m, 0.152 g, 87%) as an amber foamy solid that was used without further purification: IR (neat) 3259, 1741, 1590, 1489, 1474, 1342, 1328, 1293, 1209, 1164, 1153, 997, 837, 743, 667 cm⁻¹; ¹H NMR (CDCl₃, 500 MHz): δ7.77 (dd, J=5.0, 9.0 Hz, 2H), 7.33 (dd, J=2.5, 7.5 Hz, 1H), 7.14 (s, 1H), 7.02 (app t, J=8.5 Hz, 2H), 6.76 (dd, J=8.0, 15.5 Hz, 2H), 4.49 (dd, J=3.5, 7.5 Hz, 1H), 3.73 (s, 3H), 2.70-2.66 (m, 1H), 2.62-2.58 (m, 1H), 2.11-2.08 (m, 1H), 2.07-1.95 (m, 1H); ¹³C NMR (CDCl₃, 150 MHz): δ 170.2, 165.8, 164.1, 143.7, 135.02, 135.01, 130.1, 125.8, 124.6, 121.6, 120.6, 120.0, 115.8, 115.6, 73.7, 52.3, 24.0, 22.6; ¹⁹F NMR (CDCl₃, 376 MHz): δ−105.2; HRMS (ESI+) m/z calcd for C₁₇H₁₇O₅NFS [M+H] 366.0806, found 366.0804.

Lithium 8-((4-fluorophenyl)sulfonamido)chromane-2-carboxylate (10m-int). A solution of methyl 8-((4-fluorophenyl)sulfonamido)chromane-2-carboxylate (9m, 0.152 g, 0.42 mmol) in THE (1.0 mL) and MeOH (2.0 mL) was treated with 1M LiGH (0.60 mL) at room temperature. After 2 h, the solution was concentrated and the residue was treated with PhMe and concentrated (2×) to give lithium 8-((4-fluorophenyl)sulfonamido)chromane-2-carboxylate (10m-int, crude) that was used without further purification: HRMS (LCMS ESI+) m/z calcd for C₁₆H₁₄O₅NFSNa [M+Na] 374.0469, found 374.0466.

8-((4-Fluorophenyl)sulfonamido)-N-((tetrahydro-2H-pyran-2-yl)oxy)chromane-2-carboxamide (10m). A solution of lithium 8-((4-fluorophenyl)sulfonamido)chromane-2-carboxylate (10m-int, crude, 0.149 g, 0.42 mmol) in DMF (3.0 mL) was treated with 0-(tetrahydro-2H-pyran-2-yl)hydroxylamine (0.053 g, 0.45 mmol) in DMF (1.0 mL) followed by HATU (0.201 g, 0.52 mmol) and DIPEA (0.10 mL, 0.60 mmol). After 2 d, the solution was extracted with EtOAc, washed with 0.5M HCl, brine, dried (Na₂SO₄), filtered and concentrated. The residue was purified by chromatography on SiO₂ (ISCO-Rf, 0-100% EtOAc/hexanes) to give 8-((4-fluorophenyl)sulfonamido)-N-((tetrahydro-2H-pyran-2-yl)oxy)chromane-2-carboxamide (10m, 0.128 g, 68%) as a white foamy solid: IR (neat) 3167, 1647, 1590, 1471, 1324, 1216, 1167, 1154, 1089, 1034, 991, 894, 873, 836, 818, 745, 674 cm⁻¹; ¹H NMR (CDCl₃, 500 MHz): δ 10.35 (s, 0.5H), 10.31 (s, 0.5H), 8.05 (s, 0.5H), 7.95 (s, 0.5H), 7.81-7.77 (m, 2H), 7.13-7.07 (m, 3H), 6.90-6.88 (m, 1H), 6.82-6.79 (m, 1H), 5.06 (s, 0.5H), 5.04 (s, 0.5H), 4.23-4.19 (m, 1H), 4.08-4.03 (m, 1H), 3.76-3.59 (m, 0.5H), 3.53-3.51 (m, 0.5H), 2.76-2.68 (m, 2H), 2.36-2.31 (m, 1H), 1.96-1.88 (m, 1H), 1.77-1.70 (m, 3H), 1.59-1.43 (m, 3H); HRMS (ESI+) m/z calcd for C₂₁H₂₃O₆N₂FSNa [M+Na] 473.1153, found 473.1153.

8-((4-Fluorophenyl)sulfonamido)-N-hydroxychromane-2-carboxamide (11m). A solution of 8-((4-fluorophenyl)sulfonamido)-N-((tetrahydro-2H-pyran-2-yl)oxy)chromane-2-carboxamide (10m, 0.128 g, 0.28 mmol) in MeOH (4.0 mL) was treated with TFA (1.5 mL) at room temperature. After about 10 min, a white precipitate formed. After 2 h, the mixture was concentrated. The solid residue was slurried with Et₂O and the white solid was collected by filtration, dried in vacuo to give 8-((4-fluorophenyl)sulfonamido)-N-hydroxychromane-2-carboxamide (11m, 0.090 g, 86%) as a white solid: mp 237-239° C.; IR (neat) 3272, 3103, 1634, 1588, 1492, 1325, 1228, 1217, 1142, 1094, 1085, 1057, 995, 848, 819, 798, 756, 710 cm⁻¹; ¹H NMR (DMSO-d₆, 600 MHz): δ10.92 (s, 1H), 9.69 (s, 1H), 9.05 (s, 1H), 7.65 (dd, J=5.4, 9.0 Hz, 2H), 7.30 (app t, J=9.0 Hz, 2H), 7.13 (d, J=7.8 Hz, 1H), 6.90 (d, J=7.8 Hz, 1H), 6.83 (app t, J=7.8 Hz, 1H), 3.82 (dd, J=1.8, 10.2 Hz, 1H), 2.74-2.69 (m, 1H), 2.62-2.59 (m, 1H), 2.13-2.10 (m, 1H), 1.62-1.55 (m, 1H); ¹³C NMR (DMSO-d₆, 150 MHz): δ165.5, 165.0, 163.3, 146.0, 136.04, 136.02, 129.7, 129.6, 127.3, 124.1, 123.9, 122.7, 120.5, 115.9, 115.8, 74.9, 24.5, 23.4; ¹⁹F NMR (DMSO-d₆, 376 MHz): δ−106.3; HRMS (ESI+) m/z calcd for C₁₆H₁₆O₅N₂FS [M+H] 367.0758, found 367.0757.

Methyl 8-(naphthalene-1-sulfonamido)chromane-2-carboxylate (9n). To a solution of methyl 8-aminochromane-2-carboxylate (3, 0.201 g, 0.969 mmol) in dry THE (2.0 mL) was added triethylamine (0.35 mL, 2.47 mmol) and DMAP (0.024 g, 0.19 mmol) followed by 1-naphthalenesulfonyl chloride (0.441 g, 1.93 mmol) at room temperature. After 17 h, the reaction was heated at 40° C. After 24 h, the reaction mixture was extracted with EtOAc and washed with 1M HCl. The aqueous layer was back extracted with EtOAc (3×) and the combined organic layer was washed with brine, dried (Na₂SO₄), filtered and concentrated. The residue was purified by chromatography on SiO₂ (ISCO-Rf, 0-30% EtOAc/hexanes) to give methyl 8-(naphthalene-1-sulfonamido)chromane-2-carboxylate (9n, 0.185 g, 48%) as a light brown foamy solid.

8-(Naphthalene-1-sulfonamido)chromane-2-carboxylic acid (10n-int). A solution of methyl 8-(naphthalene-1-sulfonamido)chromane-2-carboxylate (9n, 0.130 g, 0.327 mmol) in MeOH/THF (3 mL, 3/1) was treated with 1M LiGH (0.4 mL) at room temperature. After 3.5 h, the solution was cooled to 0° C., acidified with 1M HCl, and extracted with EtOAc (4×). The combined organic layer was dried (Na₂SO₄), filtered and concentrated to give 8-(naphthalene-1-sulfonamido)chromane-2-carboxylic acid (10n-int, 0.103 g, 82%, approx. 85% purity) as a white solid: IR (neat) 3251, 3050, 2926, 2849, 1720, 1592, 1473, 1399, 1321, 1200, 1158, 1131, 1097, 993, 896, 862, 829, 803, 767, 742, 676, 630 cm⁻¹; ¹H NMR (DMSO-d₆, 400 MHz): δ12.76 (br s, 1H), 9.71 (br s, 1H), 8.70-8.65 (m, 1H), 8.18 (d, J=8.0 Hz, 1H), 8.10 (dd, J=1.2, 7.6 Hz, 1H), 8.07-8.03 (m, 1H), 7.66-7.60 (m, overlapping signals, 2H), 7.55 (dd, J=7.2, 8.0 Hz, 1H), 7.04 (dd, J=1.6, 7.6 Hz, 1H), 6.76 (dd, J=1.6, 8.0 Hz, 1H), 6.70 (app t, J=8.0 Hz, 1H), 4.04 (dd, J=4.4, 6.4 Hz, 1H), 1.79-1.73 (m, 2H).

8-(Naphthalene-1-sulfonamido)-N-((tetrahydro-2H-pyran-2-yl)oxy)chromane-2-carboxamide (10n). A solution of 8-(naphthalene-1-sulfonamido)chromane-2-carboxylic acid (10n-int, 0.099 g, 0.26 mmol) in DMF/CH₂Cl₂ (0.4 mL, 1/1) was cooled to 0° C. and treated with O-(tetrahydro-2H-pyran-2-yl)hydroxylamine (0.035 g, 0.30 mmol) in CH₂Cl₂ (0.2 mL). To this solution was added T3P (0.197 g, 0.310 mmol, 50% CH₃CN soln) in CH₂Cl₂ (0.2 mL) followed by DIPEA (0.050 mL, 0.28 mmol). The solution was stirred at 0° C. for 10 min and then warmed to room temperature. After 1 h, the reaction mixture was extracted with CH₂Cl₂, washed with 0.25M HCl (2×), 1M LiCl (2×), H₂O (3×), dried (Na₂SO₄), filtered and concentrated. The residue was purified by chromatography on SiO₂ (ISCO-Rf, 0-100% EtOAc/CH₂Cl₂) to give 8-(naphthalene-1-sulfonamido)-N-((tetrahydro-2H-pyran-2-yl)oxy)chromane-2-carboxamide (10n, 0.049 g, 40%) as a white solid: IR (neat) 3164, 2951, 2920, 2853, 1648, 1592, 1504, 1467, 1431, 1320, 1262, 1245, 1212, 1202, 1161, 1135, 1087, 1064, 1035, 987, 936, 886, 873, 818, 805, 769, 744, 682, 653, 632 cm⁻¹; ¹H NMR (DMSO-d₆, 400 MHz): δ11.41 (s, 0.5H), 11.38 (s, 0.5H), 9.96 (d, J=7.2 Hz, 1H), 8.58 (dd, J=8.4, 14.4 Hz, 1H), 8.15 (d, J=8.0 Hz, 1H), 8.10-7.99 (m, 2H), 7.65-7.49 (m, overlapping signals, 3H), 7.20-7.14 (m, 1H), 6.80-6.72 (m, overlapping signals, 2H), 4.95 (br s, 0.5H), 4.85 (br s, 0.5H), 4.16-4.00 (m, 1H), 3.65-3.50 (m, 1H), 1.90-1.49 (m, overlapping signals, 8H).

N-Hydroxy-8-(naphthalene-1-sulfonamido)chromane-2-carboxamide (11n). A solution of 8-(naphthalene-1-sulfonamido)-N-((tetrahydro-2H-pyran-2-yl)oxy)chromane-2-carboxamide (10n, 0.045 g, 0.093 mmol) in MeOH/CH₂Cl₂ (1 mL, 1/1) was treated with Amberlyst-15 (0.026 g, washed with MeOH) at room temperature. After 26 h, the reaction mixture was filtered through Celite®, rinsed with MeOH/CH₂Cl₂ (1:1), and concentrated. The solid residue was treated with Et₂O and the solid was collected by filtration and dried in vacuo to give N-hydroxy-8-(naphthalene-1-sulfonamido)chromane-2-carboxamide (11n, 0.035 g, 95%) as a tan solid: mp 219-221° C.; IR (neat) 3282, 3114, 3057, 3009, 2996, 2900, 2843, 1633, 1593, 1528, 1505, 1488, 1471, 1427, 1389, 1356, 1321, 1299, 1285, 1265, 1250, 1218, 1203, 1155, 1129, 1101, 1086, 1053, 1035, 990, 959, 903, 888, 827, 799, 756, 704, 645, 628 cm⁻¹; ¹H NMR (DMSO-d₆, 400 MHz): δ10.98 (s, 1H), 9.98 (s, 1H), 9.05 (s, 1H), 8.66-8.60 (m, 1H), 8.15 (d, J=8.4 Hz, 1H), 8.07-7.99 (m, 2H), 7.65-7.56 (m, 2H), 7.52 (app t, J=8.0 Hz, 1H), 7.18-7.12 (m, 1H), 6.74 (d, J=5.2 Hz, 2H), 1.96-1.87 (m, 1H), 1.56-1.45 (m, 1H); ¹³C NMR (DMSO-d₆, 100 MHz): δ165.5, 145.3, 134.5, 134.1, 133.5, 129.5, 128.6, 127.5, 127.4, 126.7, 126.3, 124.53, 124.47, 124.2, 122.5, 121.6, 120.3, 74.7, 24.4, 23.2; HRMS (ESI+) m/z calcd for C₂₀H₁₉O₅N₂S [M+H] 399.1009, found 399.1002.

Methyl 8-(methylsulfonamido)chromane-2-carboxylate (9o). A solution of methyl 8-aminochromane-2-carboxylate (3, 150 mg, 0.72 mmol) in CH₂Cl₂ (3.0 mL) was cooled to 0° C. and treated with pyridine (143 mg, 1.80 mmol) followed by methanesulfonyl chloride (92 mg, 0.79 mmol). The reaction mixture was slowly warmed to room temperature. After 16 h, the reaction mixture was extracted with EtOAc and washed with H₂O. The aqueous layer was back extracted with EtOAc (3×). The combined organic layer was dried (Na₂SO₄), filtered and concentrated to give methyl 8-(methylsulfonamido)chromane-2-carboxylate (9o, 250 mg, crude) as a yellow oil that was used without further purification: ¹H NMR (CDCl₃, 400 MHz): δ7.41-7.35 (m, 1H), 6.91-6.85 (m, 2H), 6.74 (br s, 1H), 4.86 (dd, J=6.1, 4.4 Hz, 1H), 3.77 (s, 3H), 3.09 (s, 3H), 2.87-2.67 (m, 2H), 2.35-2.18 (m, 2H).

8-(Methylsulfonamido)chromane-2-carboxylic acid (10o-int). A solution of methyl 8-(methylsulfonamido)chromane-2-carboxylate (9o, crude, 0.724 mmol) in MeOH/THF (3.5 mL, 3/1) was treated with 1M LiGH (0.800 mL) at room temperature. After 18 h, the solution was cooled to 0° C. and acidified with 1M HCl and extracted with EtOAc (3×). The combined organic layer was washed with brine (2×), dried (Na₂SO₄), filtered and concentrated to give a brown solid (0.182 g). The crude brown solid was diluted with EtOAc/H₂O, and 1M NaOH was added dropwise until pH 9. The solution was extracted with EtOAc and the organic layer was subsequently washed with H₂O (2×), NaOH/H₂O (pH 10, 3×). The aqueous layers were combined, acidified with 1M HCl to pH 2, then extracted with EtOAc (3×). The organic layer was dried (Na₂SO₄), filtered and concentrated to give 8-(methylsulfonamido)chromane-2-carboxylic acid (10o-int, 0.150 g, 77%-contained residual solvents) as a brown solid that was used without further purification: ¹H NMR (DMSO-d₆, 400 MHz): 313.06 (br s, 1H), 8.80 (app t, J=4.8 Hz, 1H), 7.07 (d, J=8.0 Hz, 1H), 6.94 (d, J=6.8 Hz, 1H), 6.82 (app t, J=7.6 Hz, 1H), 4.86 (dd, J=4.0, 6.8 Hz, 1H), 2.98 (s, 3H), 2.89-2.79 (m, 1H), 2.70-2.62 (m, 1H), 2.22-2.14 (m, 1H), 2.12-2.03 (m, 1H).

8-(Methylsulfonamido)-N-((tetrahydro-2H-pyran-2-yl)oxy)chromane-2-carboxamide (100). A solution of 8-(methylsulfonamido)chromane-2-carboxylic acid (10o-int, 0.145 g, 0.534 mmol) in DMF/CH₂Cl₂ (0.5 mL, 2/3) was cooled to 0° C. and treated with 0-(tetrahydro-2H-pyran-2-yl)hydroxylamine (0.076 g, 0.649 mmol) in CH₂Cl₂ (0.2 mL). To this solution was added T3P (0.420 g, 0.660 mmol, 50% wt. CH₃CN soln) in CH₂Cl₂ (0.3 mL) followed by DIPEA (0.100 mL, 0.574 mmol), and CH₂Cl₂ (0.5 mL). The solution was stirred at 0° C. for 10 min. and then warmed to room temperature. After 2 h, the reaction mixture was extracted with CH₂Cl₂, washed with 0.25M HCl (2×), 1M LiCl (3×), H₂O (3×), dried (Na₂SO₄), filtered and concentrated. The residue was purified by chromatography on SiO₂ (ISCO-Rf, 0-30% EtOAc/CH₂Cl₂) to give 8-(methylsulfonamido)-N-((tetrahydro-2H-pyran-2-yl)oxy)chromane-2-carboxamide (10o, 0.110 g, 56%) as a white solid: IR (neat) 3230, 3013, 2938, 2852, 1658, 1611, 1591, 1473, 1441, 1399, 1319, 1264, 1245, 1205, 1151, 1113, 1097, 1052, 1033, 991, 969, 944, 893, 872, 816 cm⁻¹; ¹H NMR (DMSO-d₆, 400 MHz): δ11.45 (s, 1H), 9.11 (s, 0.5H), 9.09 (s, 0.5H), 7.13-7.09 (m, 1H), 6.96 (dd, J=1.6, 7.6 Hz, 1H), 6.86 (app t, J=8.0 Hz, 1H), 4.91 (app t, J=2.8 Hz, 0.5H), 4.86 (app t, J=2.8 Hz, 0.5H), 4.72-4.67 (m, 1H), 4.05-3.99 (m, 1H), 3.53-3.47 (m, 1H), 2.92 (s, 1.5H), 2.91 (s, 1.5H), 2.88-2.82 (m, 1H), 2.76-2.70 (m, 1H), 2.33-2.24 (m, 1H), 1.91-1.83 (m, 1H), 1.74-1.72 (m, 2H), 1.66-1.58 (m, 1H), 1.54-1.51 (m, 3H).

N-Hydroxy-8-(methylsulfonamido)chromane-2-carboxamide (11o). A solution of 8-(methylsulfonamido)-N-((tetrahydro-2H-pyran-2-yl)oxy)chromane-2-carboxamide (10o, 0.093 g, 0.25 mmol) in MeOH (1.5 mL) was treated with Amberlyst-15 (0.045 g, washed with MeOH) at room temperature. After 18 h, the reaction mixture was filtered through Celite®, rinsed with MeOH, and concentrated. The solid was treated with Et₂O and the solid was collected by filtration and dried in vacuo to give N-hydroxy-8-(methylsulfonamido)chromane-2-carboxamide (11o, 0.058 g, 81%) as a pink-tan solid: mp 181-183° C.; ¹H NMR (DMSO-d₆, 400 MHz): δ11.08 (s, 1H), 9.16 (s, 1H), 9.04 (br s, 1H), 7.13 (dd, J=0.8, 7.6 Hz, 1H), 6.95 (d, J=6.4 Hz, 1H), 6.86 (app t, J=7.6 Hz, 1H), 4.65 (dd, J=2.4, 10.0 Hz, 1H), 2.91 (s, 3H), 2.90-2.84 (m, 1H), 2.75 (app t, J=4.4 Hz, 0.5H), 2.71 (app t, J=5.2 Hz, 0.5H), 2.33-2.28 (m, 1H), 1.88-1.78 (m, 1H); ¹³C NMR (DMSO-d₆, 150 MHz): δ166.1, 145.8, 126.7 125.1, 123.1, 123.0, 120.5, 75.1, 24.5, 23.6; HRMS (ESI+) m/z calcd for C₁₁H₁₅O₅N₂S [M+H] 287.0696, found 287.0698.

Methyl 8-((4-methylphenyl)sulfonamido)chromane-2-carboxylate (9p). A solution of methyl 8-aminochromane-2-carboxylate (3, 0.150 g, 0.724 mmol) in CH₂Cl₂ (3 mL) was treated with pyridine (0.175 mL, 2.171 mmol) followed by 4-methylbenzenesulfonyl chloride (0.152 g, 0.796 mmol) at room temperature. After 22 h, the solution was extracted with CH₂Cl₂, washed with 0.5M HCl (2×), brine (2×), dried (Na₂SO₄), filtered and concentrated to give methyl 8-((4-methylphenyl)sulfonamido)chromane-2-carboxylate (9p, 0.238 g, 91%) as a brown residue: ¹H NMR (CDCl₃, 400 MHz): δ 7.68 (d, J=8.4 Hz, 2H), 7.35 (dd, J=1.6, 7.6 Hz, 1H), 7.19 (d, J=8.0 Hz, 2H), 7.02 (s, 1H), 6.79 (app t, J=7.6 Hz, 1H), 6.74 (dd, J=2.0, 8.0 Hz, 1H), 4.49 (dd, J=3.6, 7.6 Hz, 1H), 3.77 (s, 3H), 2.75-2.60 (m, 2H), 2.36 (s, 3H), 2.16-2.09 (m, 2H).

8-((4-Methylphenyl)sulfonamido)chromane-2-carboxylic acid (10p-int). A solution of methyl 8-((4-methylphenyl)sulfonamido)chromane-2-carboxylate (9p, 0.262 g, 0.724 mmol) in MeOH/THF (3.5 mL, 3/1) was treated with 1M LiGH (0.72 mL) at room temperature. After 18 h, the solution was cooled to 0° C. and acidified with 1M HCl, extracted with EtOAc (3×). The combined organic layer was washed with brine (2×), dried (Na₂SO₄), filtered and concentrated to give 8-((4-methylphenyl)sulfonamido)chromane-2-carboxylic acid (10p-int, 0.223 g, 89%) as a brown solid: IR (neat) 3504, 3244, 3103, 3063, 3039, 2926, 2852, 2688, 1728, 1612, 1595, 1474, 1446, 1398, 1338, 1323, 1305, 1269, 1241, 1210, 1155, 1089, 1055, 1040, 1018, 993, 904, 866, 850, 832, 812, 775, 740, 703, 659, 604 cm⁻¹; ¹H NMR (DMSO-d₆, 400 MHz): δ7.64 (d, J=8.4 Hz, 2H), 7.28 (d, J=7.6 Hz, 2H), 7.06 (dd, J=2.0, 8.0 Hz, 1H), 6.80 (dd, J=1.6, 7.6 Hz, 1H), 6.73 (app t, J=8.0 Hz, 1H), 4.42 (dd, J=4.0, 7.6 Hz, 1H), 2.74-2.67 (m, 1H), 2.60-2.52 (m, 1H), 2.33 (s, 3H), 2.04-1.97 (m, 1H), 1.91-1.85 (m, 1H).

8-((4-Methylphenyl)sulfonamido)-N-((tetrahydro-2H-pyran-2-yl)oxy)chromane-2-carboxamide (10p). A solution of 8-((4-methylphenyl)sulfonamido)chromane-2-carboxylic acid (0.195 g, 0.561 mmol) in DMF/CH₂Cl₂ (0.6 mL, 1/1) was cooled to 0° C. and treated with 0-(tetrahydro-2H-pyran-2-yl)hydroxylamine (0.079 g, 0.674 mmol) in CH₂Cl₂ (0.3 mL). To this solution was added T3P (0.429 g, 0.674 mmol, 50% CH₃CN soln) in CH₂Cl₂ (0.3 mL) followed by DIPEA (0.107 mL, 0.617 mmol). The solution was stirred at 0° C. for 10 min. and warmed to room temperature. After 2.5 h, the reaction mixture was extracted with CH₂Cl₂, washed with 0.25N HCl (2×), 1M LiCl (3×), H₂O (4×), dried (Na₂SO₄), filtered and concentrated. The residue was purified by chromatography on SiO₂ (ISCO-Rf, 0-100% EtOAc/CH₂Cl₂) to give 8-((4-methylphenyl)sulfonamido)-N-((tetrahydro-2H-pyran-2-yl)oxy)chromane-2-carboxamide (10p, 0.106 g, 42%) as a white solid: ¹H NMR (DMSO-d₆, 400 MHz): δ11.44 (s, 0.5H), 11.36 (s, 0.5H), 9.59 (s, 0.5H), 9.54 (s, 0.5H), 7.52-7.48 (m, 2H), 7.25-7.22 (m, 2H), 7.13 (d, J=8.4 Hz, 1H), 6.88-6.79 (m, overlapping signals, 2H), 4.99 (br s, 0.5H), 4.92 (br s, 0.5H), 4.14-4.09 (m, 1H), 3.81-3.69 (m, 1H), 3.63-3.54 (m, 2H), 2.30 (s, 3H), 2.11-2.08 (m, 1H), 1.80-1.56 (m, 8H).

N-Hydroxy-8-((4-methylphenyl)sulfonamido)chromane-2-carboxamide (11p). A solution of 8-((4-methylphenyl)sulfonamido)-N-((tetrahydro-2H-pyran-2-yl)oxy)chromane-2-carboxamide (10p, 0.103 g, 0.231 mmol) in MeOH (1.4 mL) was treated with Amberlyst-15 (0.041 g, washed with MeOH) at room temperature. After 16 h, the reaction mixture was diluted with MeOH/CH₂Cl₂ and the solution was transferred into a flask. The solution was concentrated and the solid residue was treated with Et₂O and the solid was collected by filtration and dried in vacuo to give N-hydroxy-8-((4-methylphenyl)sulfonamido)chromane-2-carboxamide (11p, 0.061 g, 73%) as a white solid powder: mp 235-237° C.; IR (neat) 3235, 3146, 3039, 2984, 2930, 1632, 1595, 1540, 1478, 1447, 1421, 1354, 1321, 1293, 1283, 1264, 1248, 1211, 1174, 1142, 1102, 1087, 1056, 1033, 1017, 991, 963, 915, 900, 887, 867, 846, 833, 822, 799, 773, 749, 707, 652, 605 cm⁻¹; ¹H NMR (DMSO-d₆, 400 MHz): δ10.98 (d, J=1.6 Hz, 1H), 9.60 (s, 1H), 9.05 (d, J=1.6 Hz, 1H), 7.51 (d, J=8.4 Hz, 2H), 7.27 (d, J=8.0 Hz, 2H), 7.11 (dd, J=2.0, 8.0 Hz, 1H), 6.85 (dd, J=1.6, 7.6 Hz, 1H), 6.80 (app t, J=7.6 Hz, 1H), 3.81 (dd, J=2.4, 10.4 Hz, 1H), 2.75-2.66 (m, 1H), 2.63-2.57 (m, 1H), 2.31 (s, 3H), 2.15-2.10 (m, 1H), 1.64-1.58 (m, 1H); ¹³C NMR (DMSO-d₆, 100 MHz): δ165.6, 145.7, 143.0, 136.8, 129.2, 126.8, 126.7, 124.6, 122.9, 122.6, 120.4, 75.0, 24.6, 23.5, 20.9; HRMS (ESI+) m/z calcd for C₁₇H₁₉O₅N₂S [M+H] 363.1009, found 363.1007.

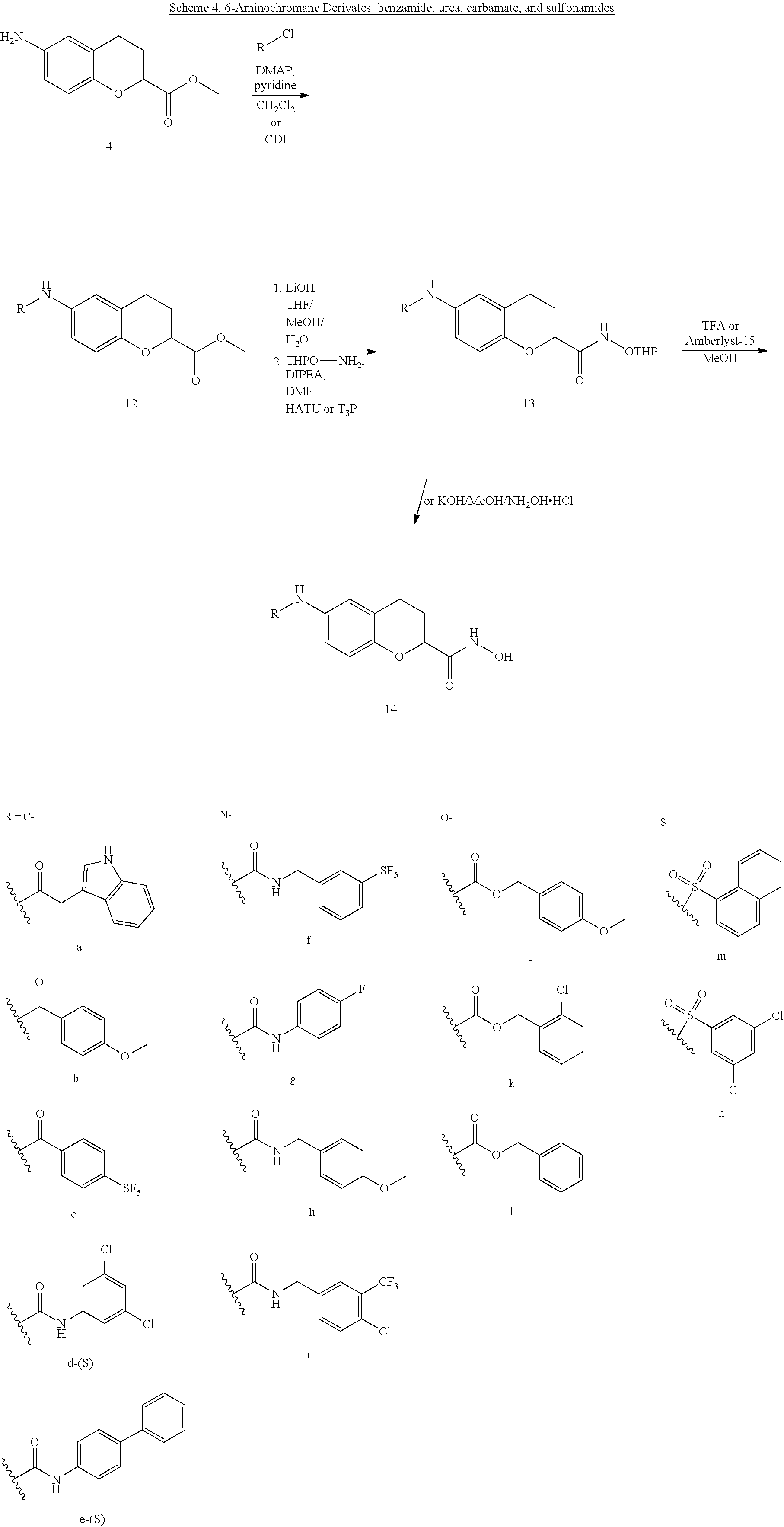

Methyl 6-(2-(1H-indol-3-yl)acetamido)chromane-2-carboxylate (12a). To a solution of methyl 6-aminochromane-2-carboxylate (4, 207 mg, 1.00 mmol) in DMF (3.0 mL) was added 2-(1H-indol-3-yl)acetic acid (161 mg, 1.00 mmol), HATU (475 mg, 1.25 mmol) and Et₃N (0.27 mL, 2.00 mmol) at rt. After 16 h, the reaction mixture was extracted with EtOAc and washed with H₂O. The aqueous layer was back extracted with EtOAc (3×). The combined organic layer was dried (Na₂SO₄), filtered and concentrated. The residue was purified by chromatography on SiO₂ (ISCO-Rf, 5-50% EtOAc/hexanes to give methyl 6-(2-(1H-indol-3-yl)acetamido)chromane-2-carboxylate (12, 210 mg, 60%) as an off-white solid: ¹H NMR (CDCl₃, 400 MHz): δ 8.96 (s, 1H), 7.63 (s, 1H), 7.54 (d, J=7.8 Hz, 1H), 7.30 (d, J=8.2 Hz, 1H), 7.19-7.13 (m, 2H), 7.08 (ddd, J=7.8, 7.0, 0.9 Hz, 1H), 7.00 (d, J=2.5 Hz, 1H), 6.86 (dd, J=8.8, 2.5 Hz, 1H), 6.72 (d, J=8.7 Hz, 1H), 4.62 (dd, J=7.4, 3.3 Hz, 1H), 3.79 (s, 2H), 3.73 (s, 3H), 2.68-2.50 (m, 2H), 2.19-2.09 (m, 1H), 2.08-1.98 (m, 1H); ¹³C NMR (CDCl₃, 100 MHz): δ 171.3, 170.1, 150.0, 136.3, 130.6, 126.7, 124.1, 122.2, 121.8, 121.3, 120.3, 119.8, 118.3, 116.7, 111.5, 107.9, 73.5, 34.0, 24.2, 23.1, 14.0.

6-(2-(1H-Indol-3-yl)acetamido)-N-hydroxychromane-2-carboxamide (14a). To an ice-cooled solution of hydroxylamine hydrochloride (555 mg, 7.93 mmol) in methanol (5.0 mL) was added powdered KOH (520 mg, 9.30 mmol) portion wise. The solution was warmed to room temperature. After 1 h, the precipitate was removed by filtration and the filtrate was added dropwise to an ice-cooled solution of methyl 6-(2-(1H-indol-3-yl)acetamido)chromane-2-carboxylate (12a, 114 mg, 0.31 mmol) in methanol (5 mL). An additional amount of KOH (174 mg, 3.10 mmol) was added to the reaction solution. After 2 h, 1M HCl was slowly added to pH 7 and the solid was collected by filtration, washed with H₂O followed by hexanes and dried in vacuo to give 6-(2-(1H-indol-3-yl)acetamido)-N-hydroxychromane-2-carboxamide (14a, 70.0 mg, 61%) as an off-white solid: ¹H NMR (DMSO-d₆, 400 MHz): δ 10.89 (s, 1H), 10.78 (br s, 1H), 9.87 (s, 1H), 9.05 (br s, 1H), 7.59 (d, J=7.8 Hz, 1H), 7.36-7.32 (m, 2H), 7.27-7.21 (m, 2H), 7.06 (ddd, J=8.4, 7.0, 1.3 Hz, 1H), 6.97 (ddd, J=7.9, 7.1, 1.0 Hz, 1H), 6.93 (d, J=8.6 Hz, 1H), 4.44 (dd, J=9.1, 2.9 Hz, 1H), 3.66 (s, 2H), 2.81-2.56 (m, 2H), 2.14-2.02 (m, 1H), 2.00-1.83 (m, 1H); ¹³C NMR (DMSO-d₆, 100 MHz): δ 169.1, 166.5, 149.2, 136.1, 132.3, 127.2, 123.8, 121.6, 120.9, 120.2, 118.7, 118.3, 116.4, 114.9, 114.8, 111.3, 108.7, 92.9, 73.8, 33.7, 24.3, 23.2; HRMS (ESI+) m/z calcd for C₂₀H₂₀O₄N₃ [M+H] 366.1448, found 366.1446.

Methyl 6-(4-methoxybenzamido)chromane-2-carboxylate (12b). A 0° C. solution of the aniline in DCM (0.48 M, 1.77 mL, 0.850 mmol) was treated with the acid chloride (0.145g, 0.852 mmol), DMAP (0.0397 g, 0.325 mmol) and pyridine (0.100 mL, 1.24 mmol). After 5 min, the solution was warmed to room temperature, and was left to stir under N₂ for 4 h, and more DMAP (0.0420 g, 0.344 mmol) and aniline (0.48 M, 0.2 mL, 0.0960 mmol) were added. After 20 h, the mixture was diluted with DCM (30 mL), washed with 0.5 M HCl (1×30 mL), brine (lx), dried (Na₂SO₄), filtered, and concentrated. 1H NMR shows incomplete conversion. The reaction mixture was saponified.

Saponification:

The crude mixture was diluted with THF (3.0 mL) and Methanol (1.5 mL). LiOH monohydrate (0.0661 g, 1.58 mmol) in H₂O (3.0 mL) was added to the mixture, which was left to stir for 6 h. The solution was concentrated and the residue was azeotroped with PhMe (2×20 mL). Coupling:

The crude residue was dissolved in DMF (4 mL) and treated with the amine (0.189 g, 1.61 mmol), HATU (0.470 g, 1.24 mmol), and DIPEA (0.300 mL, 1.72 mmol). The mixture was stirred under N₂, and after 17 h, was diluted with Ethyl acetate (50 mL), washed with 0.5 M HCl (50 mL), BRINE (50 mL), dried (Na₂SO₄), filtered, and concentrated. The residue was purified by column chromatography (on SiO₂, conditions:2:1 hex:EtOAc->1:1 hex:EtOAc) to give back the methyl ester (0.122 g, 0.358 mmol, 42.00%).

Lithium 6-(4-methoxybenzamido)chromane-2-carboxylate (13b-int). To a solution of methyl 6-(4-methoxybenzamido)chromane-2-carboxylate (12b, 0.120 g, 0.35 mmol) in THF/MeOH (3.0/1.5 mL) was added 0.25M LiOH (1.46 mL) at room temperature. After 2.5 h, the solution was concentrated and the residue was treated with PhMe and concentrated (2×) to give lithium 6-(4-methoxybenzamido)chromane-2-carboxylate (13b-int, crude) that was used without further purification.

6-(4-Methoxybenzamido)-N-((tetrahydro-2H-pyran-2-yl)oxy)chromane-2-carboxamide (13b). A solution of lithium 6-(4-methoxybenzamido)chromane-2-carboxylate (13b-int, 0.175 g, 0.37 mmol, crude) in DMF (2.5 mL) was treated with O-(tetrahydro-2H-pyran-2-yl)hydroxylamine (0.045 g, 0.39 mmol) in DMF (1.2 mL) followed by HATU (0.147 g, 0.39 mmol) and DIPEA (0.07 mL, 0.44 mmol) at room temperature. After 15.5 h, the solution was extracted with EtOAc, washed with 0.5M HCl, brine, dried (Na₂SO₄), filtered and concentrated. The residual oil was purified by chromatography on SiO₂ (2/1, hexanes:EtOAc) to give 6-(4-methoxybenzamido)-N-((tetrahydro-2H-pyran-2-yl)oxy)chromane-2-carboxamide (13b, 0.117 g, 75%) as a white solid.

N-Hydroxy-6-(4-methoxybenzamido)chromane-2-carboxamide (14b). To a solution of 6-(4-methoxybenzamido)-N-((tetrahydro-2H-pyran-2-yl)oxy)chromane-2-carboxamide (13b, 0.131 g, 0.307 mmol) in MeOH (2.5 mL) was added TFA (0.68 mL, 9.21 mmol) at room temperature. After 4 h the mixture was concentrated. The residue was slurried with Et₂O and the solid was collected by filtration, washed with Et₂O and dried in vacuo to give N-hydroxy-6-(4-methoxybenzamido)chromane-2-carboxamide (14b, 0.0479 g, 46%) as a white solid: IR (neat) 3288, 1637, 1605, 1526, 1506, 1420, 1279, 1256, 1214, 1180, 1075, 1022, 906, 895, 872, 849, 823, 765, 737, 662 cm⁻¹; ¹³C NMR (DMSO-d₆, 150 MHz): δ166.5, 164.4, 161.7, 149.5, 132.2, 129.5, 129.3, 127.0, 121.7, 121.5, 120.1, 116.4, 113.6, 113.5, 73.8, 55.4, 23.3, 20.8; HRMS (ESI+) m/z calcd for C₁₈H₁₉O₅N₂ [M+H] 343.1288, found 343.1286.

Methyl 6-(4-(pentafluoro-%⁶-sulfaneyl)benzamido)chromane-2-carboxylate (12c). A solution of methyl 6-aminochromane-2-carboxylate (4, 0.124 g, 0.603 mmol) in CH₂Cl₂ (10 mL) was cooled to 0° C. and treated with pyridine (0.073 mL, 0.91 mmol), DMAP (0.025 g, 0.21 mmol) followed by 4-(pentafluoro-l⁶-sulfaneyl)benzoyl chloride (0.229 g, 0.724 mmol). After 5 min, the solution was warmed to room temperature. After 24 h, the reaction mixture was extracted with CH₂Cl₂, washed with 0.1M HCl, brine, dried (Na₂SO₄), filtered and concentrated. The residue was purified by chromatography on SiO₂ (hexanes/EtOAc, 2/1) to give methyl 6-(4-(pentafluoro-λ⁶-sulfaneyl)benzamido)chromane-2-carboxylate (12c, 0.260 g, 97%) as a white solid.

Lithium 6-(4-(pentafluoro-l⁶-sulfaneyl)benzamido)chromane-2-carboxylate (13c-int). To a solution of methyl 6-(4-(pentafluoro-)⁶-sulfaneyl)benzamido)chromane-2-carboxylate (12c, 0.260 g, 0.60 mmol) in THF/MeOH (3.0/1.5 mL) was added 1M LiGH (0.63 mL) at room temperature. After 48 h, the solution was concentrated and the residue was diluted with PhMe and concentrated (2×) to give lithium 6-(4-(pentafluoro-l⁶-sulfaneyl)benzamido)chromane-2-carboxylate (13c-int) that was used without further purification.

6-(4-(Pentafluoro-%⁶-sulfaneyl)benzamido)-N-((tetrahydro-2H-pyran-2-yl)oxy)chromane-2-carboxamide (13c). A solution of lithium 6-(4-(pentafluoro-l⁶-sulfaneyl)benzamido)chromane-2-carboxylate (13c-int, 0.256 g, 0.60 mmol, crude) in DMF (3.0 mL) was treated with O-(tetrahydro-2H-pyran-2-yl)hydroxylamine (0.077 g, 0.66 mmol) in DMF (1.5 mL) followed by HATU (0.249 g, 0.66 mmol) and DIPEA (0.12 mL, 0.72 mmol) at room temperature. After 15.5 h, the reaction mixture was extracted with EtOAc, washed with brine, 1M LiCi (2×), dried (Na₂SO₄), filtered and concentrated. The residual oil was purified by chromatography on SiO₂ (hexanes/EtOAc, 2:1) to give 6-(4-(pentafluoro-λ⁶-sulfaneyl)benzamido)-N-((tetrahydro-2H-pyran-2-yl)oxy)chromane-2-carboxamide (13c, 0.176 g, 57%) as a white solid.

N-Hydroxy-6-(4-(pentafluoro-λ⁶-sulfaneyl)benzamido)chromane-2-carboxamide (14c). A solution of 6-(4-(pentafluoro-λ⁶-sulfaneyl)benzamido)-N-((tetrahydro-2H-pyran-2-yl)oxy)chromane-2-carboxamide (13c, 0.173 g, 0.331 mmol) in MeOH (2.0 mL) was treated with TFA (0.74 mL, 9.93 mmol) at room temperature. After 2 h the mixture was concentrated. The solid residue was slurried with Et₂O and the solid was collected by filtration and washed with Et₂O. The solid was purified by chromatography on SiO₂ (EtOAc/hexanes, 2:1) to give N-hydroxy-6-(4-(pentafluoro-λ⁶-sulfaneyl)benzamido)chromane-2-carboxamide (14c, 0.095 g, 35% 4-steps) as a white solid: mp 214-216° C.; IR (neat) 3282, 1641, 1523, 1498, 1422, 1263, 1217, 1091, 1079, 880, 870, 825, 773, 762, 665 cm⁻¹; ¹³C NMR (acetone-d₆, 150 MHz): δ167.6, 164.2, 156.0, 150.8, 139.8, 133.0, 129.2, 127.03, 127.00, 126.97, 123.0, 122.4, 120.8, 117.6, 75.6, 25.4, 24.4; ¹⁹F NMR (acetone-d₆, 470 MHz): δ62.2, 61.9; HRMS (ESI+) m/z calcd for C₁₇H₁₆O₄N₂F₅S [M+H] 439.0745, found 439.0744.

Methyl 6-(3,5-dichlorobenzamido)chromane-2-carboxylate (BB). A solution of the methyl 6-aminochromane-2-carboxylate (4, 0.306 g, 1.48 mmol) in CH₂Cl₂ (2.9 mL) was cooled to 0° C. and treated with pyridine (0.18 mL, 2.2 mmol), DMAP (0.053 g, 0.44 mmol) and 3,5-dichlorobenzoyl chloride (0.342 g, 0.813 mmol). After 10 min, the solution was warmed to room temperature. After 5 h, the reaction mixture was extracted with CH₂Cl₂, washed with 0.5M HCl, brine, dried (Na₂SO₄), filtered, and concentrated to give methyl 6-(3,5-dichlorobenzamido)chromane-2-carboxylate (BB, 0.577 g, crude-contained residual solvent) as a light pink solid: HRMS (ESI+) m/z calcd for C₁₈H₁₆O₄NCl₂ [M+H] 38510.04, found 380.0449.

Methyl (S)-6-(3,5-dichlorobenzamido)chromane-2-carboxylate. The mixture was separated by semi-preparative HPLC (Phenomenox Lux® Cellulose-3, 10 mm×250 mm, 5 μM, 50/50 hexanes/iPrOH, 220 nM) to give methyl (S)-6-(3,5-dichlorobenzamido)chromane-2-carboxylate (S-12d, 200 mg): mp 157-162° C.; [α]_{D}=+25.3 (c 0.11, CH₂Cl₂); Chiral HPLC purity >99% (Phenomenox Lux® Cellulose-3.

(S)-6-(3,5-Dichlorobenzamido)chromane-2-carboxylic acid ((S)-13d-int). A solution of methyl (S)-6-(3,5-dichlorobenzamido)chromane-2-carboxylate ((S)-12d, 200 mg, 0.526 mmol) in THE (1.5 mL) and MeOH (1.5 mL) was treated with 1M LiGH (0.70 mL) at room temperature. After 4 h, the solution was concentrated and the residue was diluted with H₂O, acidified using 1M HCl, and extracted with EtOAc (2×). The combined organic layer was washed with brine, dried (Na₂SO₄), filtered and concentrated. The solid residue was diluted with Et₂O/hexanes (1/1) and (S)-6-(3,5-dichlorobenzamido)chromane-2-carboxylic acid was collected by filtration ((S)-13d-int, 0.170 g, 88%) as an off-white solid: [α]_{D}=+24.5 (c 0.14, MeOH); mp 209-212° C.; ¹H NMR (DMSO-d₆, 400 MHz): δ13.01 (s, 1H), 10.23 (s, 1H), 7.96 (s, 1H), 7.95 (s, 1H), 7.47 (d, J=2.4 Hz, 1H), 7.41 (dd, J=2.4, 8.8 Hz, 1H), 6.80 (d, J=8.8 Hz, 1H), 6.80 (d, J=8.8 Hz, 1H), 4.76 (dd, J=4.0, 4.0 Hz, 1H), 2.82-2.77 (m, 1H), 2.68-2.63 (m, 1H), 2.16-2.14 (m, 1H), 2.13-2.07 (m, 1H); ¹³C NMR (DMSO-d₆, 100 MHz): δ171.9, 162.1, 150.0, 138.1, 134.3, 131.2, 130.7, 126.3, 121.8, 121.4, 120.2, 116.2, 72.8, 23.8, 22.6; HRMS (LCMS ESI+) m/z calcd for C₁₇H₁₂O₄NCl₂ [M−H] 364.0138, found 364.0130.

(2S)-6-(3,5-Dichlorobenzamido)-N-((tetrahydro-2H-pyran-2-yl)oxy)chromane-2-carboxamide (73). To a solution of (S)-6-(3,5-dichlorobenzamido)chromane-2-carboxylic acid (0.112 g, 0.306 mmol) in DMF (1.5 mL) was added O-(tetrahydro-2H-pyran-2-yl)hydroxylamine (0.049 g, 0.42 mmol) in CH₂Cl₂ (1.0 mL) followed by T3P (0.220 g, 50% EtOAc soln) in CH₂Cl₂ (1.0 mL). To this solution was added DIPEA (0.050 mL) at room temperature. After 18 h, the solution was extracted with CH₂Cl₂, washed with satd. NaHCO₃, and brine. The aqueous was back extracted with EtOAc (2×). The combined organic layer was dried (Na₂SO₄), filtered and concentrated. The residue was purified by chromatography on SiO₂ (ISCO-Rf, 0-100% EtOAc/hexanes) to give (2S)-6-(3,5-dichlorobenzamido)-N-((tetrahydro-2H-pyran-2-yl)oxy)chromane-2-carboxamide (73, 0.099 g, 66%) as an off-white foam: ¹H NMR (CDCl₃, 400 MHz): δ9.21 (s, 1H), 8.27 (s, 1H), 7.72 (s, 1H), 7.71 (s, 1H), 7.48 (app t, J=1.6 Hz, 1H), 7.39-7.37 (m, 1H), 7.29-7.27 (m, 1H), 6.83 (app t, J=7.6 Hz, 1H), 4.99 (s, 0.5H), 4.94 (s, 0.5H), 4.59-4.55 (m, 1H), 3.99-3.95 (m, 1H), 3.64-3.59 (m, 1H), 2.83-2.72 (m, 2H), 2.41-2.37 (m, 1H), 2.05-1.99 (m, 1H), 1.79-1.75 (m, 3H), 1.66-1.58 (m, 3H); HRMS (LCMS ESI+) m/z calcd for C₂₂H₂₂O₅N₂Cl₂Na [M+Na] 487.0798, found 487.0798.

(S)-6-(3,5-Dichlorobenzamido)-N-hydroxychromane-2-carboxamide. A solution of (2S)-6-(3,5-dichlorobenzamido)-N-((tetrahydro-2H-pyran-2-yl)oxy)chromane-2-carboxamide (0.099 g, 0.21 mmol) in MeOH (3.0 mL) was treated with Amberlyst-15 (0.016 g) at room temperature. After 2 d, the solution was filtered through Celite®, rinsed with MeOH and concentrated. The residue was slurried with Et₂O/hexanes (1/1) and the solid was collected by filtration and dried in vacuo to give (S)-6-(3,5-dichlorobenzamido)-N-hydroxychromane-2-carboxamide (0.049 g, 60%) as a light pink solid: mp 189-191° C.; [α]_{D}=+5.4 (c 0.13, MeOH); IR (neat) 3234, 1642, 1530, 1492, 1419, 1259, 1218, 1075, 865, 804 cm⁻¹; ¹H NMR (DMSO-d₆, 600 MHz): δ10.81 (s, 1H), 10.24 (s, 1H), 8.95 (s, 1H), 7.96 (s, 1H), 7.95 (s, 1H), 7.85 (d, J=1.8 Hz, 1H), 7.47 (d, J=1.8 Hz, 1H), 7.41 (dd, J=2.4, 8.4 Hz, 1H), 6.82 (d, J=9.0 Hz, 1H), 4.51 (dd, J=3.0, 3.0 Hz, 1H), 2.81-2.79 (m, 1H), 2.72-2.69 (m, 1H), 2.13-2.10 (m, 1H), 1.97-1.94 (m, 1H); ¹³C NMR (DMSO-d₆, 150 MHz): δ166.4, 162.1, 150.0, 138.1, 134.3, 131.4, 130.8, 126.4, 121.74, 121.72, 120.1, 116.5, 73.8, 24.3, 23.2; HRMS (LCMS ESI+) m/z calcd for C₁₇H₁₅O₄N₂Cl₂ [M+H] 381.0403, found 381.0400.

Methyl (S)-6-([1,1′-biphenyl]-4-carboxamido)chromane-2-carboxylate ((S)-12e). A solution of methyl (S)-6-aminochromane-2-carboxylate ((S)-4 (0.0726 g, 0.350 mmol) in CH₂Cl₂ (1.8 mL) was cooled to 0° C. and treated with pyridine (0.20 mL, 2.5 mmol) and DMAP (0.017 g, 0.14 mmol) followed by [1,1′-biphenyl]-4-carbonyl chloride (0.095 g, 0.438 mmol). After 5 min, the mixture was warmed to room temperature. After 11 h, the reaction mixture was extracted with CH₂Cl₂, washed with 0.5M HCl, satd. NaHCO₃, brine, dried (Na₂SO₄), filtered, and concentrated to give methyl (S)-6-([1,1′-biphenyl]-4-carboxamido)chromane-2-carboxylate ((S)-12e, 0.136 g, 98%) as a white solid: ¹H NMR (CDCl₃, 500 MHz): δ 7.97-7.91 (m, 2H), 7.73-7.68 (m, 3H), 7.65-7.61 (m, 2H), 7.56 (d, J=2.5 Hz, 1H), 7.51-7.45 (m, 2H), 7.43-7.37 (m, 1H), 7.21 (dd, J=8.8, 2.9 Hz, 1H), 6.94 (d, J=8.7 Hz, 1H), 4.75 (dd, J=7.3, 3.7 Hz, 1H), 3.81 (s, 3H), 2.89-2.80 (m, 2H), 2.31-2.20 (m, 2H); HRMS (ESI+) m/z calcd for C₂₄H₂₂NO₄ [M+H]388.1543, found 388.1539.

(S)-6-([1,1′-Biphenyl]-4-carboxamido)-N-hydroxychromane-2-carboxamide ((S)-14e). A solution of methyl (S)-6-([1,1′-biphenyl]-4-carboxamido)chromane-2-carboxylate ((S)-12e, 0.125 g, 0.25 mmol) in THE (1.5 mL) and MeOH (1.5 mL) was treated with LiGH monohydrate (0.0135 g, 0.355 mmol) in water (1.0 mL) at room temperature. After 1 h, the mixture was concentrated, and azeotroped with PhMe (2×) and the residue was used without further purification. The residue was dissolved in DMF (1.0 mL) and treated with O-(tetrahydro-2H-pyran-2-yl)hydroxylamine (0.100 g, 0.854 mmol). The mixture was cooled to 0° C., and treated with T3P (0.280 mL, 0.470 mmol, 50% EtOAc) and TEA (0.200 mL, 1.43 mmol). After 30 min, the mixture was warmed to room temperature. After 3 h, the mixture was extracted with EtOAc, washed with 0.5M HCl, brine, dried (Na₂SO₄), filtered through SiO₂ plug, and concentrated. To this crude product (0.118 g) in MeOH (5.0 mL) was added Amberlyst-15 (0.037 g, 172 mmol, washed with MeOH) at room temperature. After 24 h, additional Amberlyst-15 (0.022 g, 103 mmol) was added. After 22 h, the reaction mixture was filtered through Celite®, rinsed with MeOH, and concentrated. The residue was purified by trituration from hexanes/EtOAc (5:1) to give (S)-6-([1,1′-biphenyl]-4-carboxamido)-N-hydroxychromane-2-carboxamide ((S)-14e, 0.0672 g, 54%, 3-steps) as a light grey solid: mp decomposed 191° C.; [α]_{D}=+20.1 (c 0.30, DMSO); IR (CH₂Cl₂) 3259, 1639, 1527, 1494, 1422, 1220 cm⁻¹; ¹H NMR (DMSO-d₆, 500 MHz): 10.79 (s, 1H), 10.11 (s, 1H), 8.93 (s, 1H), 8.04 (d, J=8.4 Hz, 2H), 7.78 (d, J=8.4 Hz, 2H), 7.76 (d, J=7.3 Hz, 2H), 7.53-7.49 (m, 3H), 7.46 (dd, J=8.8, 2.4 Hz, 1H), 7.42 (app t, J=7.3 Hz, 1H), 6.82 (d, J=8.8 Hz, 1H), 4.51 (dd, J=9.0, 3.0 Hz, 1H), 2.87-2.79 (m, 1H), 2.74-2.69 (m, 1H), 2.15-2.10 (m, 1H), 1.99-1.93 (m, 1H); ¹³C NMR (DMSO-d₆, 125 MHz) δ 166.4, 164.6, 149.6, 142.9, 139.1, 133.8, 132.0, 129.0, 128.2, 128.1, 126.9, 126.5, 121.62, 121.57, 120.1, 116.4, 73.8, 24.3, 23.3; HRMS (ESI+) m/z calcd for C₂₃H₂₁O₄N₂ [M+H] 389.1496, found 389.1494.

Methyl 6-(3-(3-(pentafluoro-λ⁶-sulfaneyl)benzyl)ureido)chromane-2-carboxylate (12f). To a solution of methyl 6-aminochromane-2-carboxylate (4, 150 mg, 0.72 mmol) in CH₂Cl₂ (3.0 mL) was added carbonyl diimidazole (146 mg, 0.90 mmol) at room temperature. After 16 h, (3-(pentafluoro-λ⁶-sulfaneyl)phenyl)methanamine (200 mg, 0.86 mmol) in CH₂Cl₂ (2.0 ml), was added. The reaction mixture was extracted with CH₂Cl₂ and washed with H₂O. The aqueous layer was back extracted with CH₂Cl₂ (3×). The combined organic layer was dried (Na₂SO₄), filtered and concentrated. The residue was purified by chromatography on SiO₂ (ISCO-Rf, 0-50% EtOAc/hexanes) to give methyl 6-(3-(3-(pentafluoro-λ⁶-sulfaneyl)benzyl)ureido)chromane-2-carboxylate (12f, 70 mg, 21%) as a gray oil: ¹H NMR (CDCl₃, 400 MHz): δ 7.70-7.64 (m, 2H), 7.52-7.41 (m, 2H), 7.04 (d, J=2.4 Hz, 1H), 7.00-6.97 (m, 1H), 6.93 (d, J=8.4 Hz, 1H), 6.11 (br s, 1H), 5.02 (t, J=7.1 Hz, 1H), 4.77 (dd, J=7.4, 4.2 Hz, 1H), 4.51 (d, J=6.0 Hz, 2H), 3.81 (s, 3H), 2.90-2.68 (m, 2H), 2.34-2.16 (m, 2H).

N-Hydroxy-6-(3-(3-(pentafluoro-λ⁶-sulfaneyl)benzyl)ureido)chromane-2-carboxamide (14f). To an ice-cooled solution of hydroxylamine hydrochloride (56.0 mg,

0.80 mmol) in methanol (2.0 mL) was added powdered KOH (54 mg, 0.96 mmol) portion wise and the resulting mixture was stirred at room temperature. After 1 h, the precipitate was removed by filtration and the filtrate was added dropwise to an ice-cooled solution of methyl 6-(3-(3-(pentafluoro-16-sulfaneyl)benzyl)ureido)chromane-2-carboxylate (12f, 15.0 mg, 0.032 mmol) in methanol (2.0 mL). An additional amount of KOH (18.0 mg, 0.32 mmol) was added to the reaction solution After 1 h, 1M HCl was added dropwise to adjust pH of 7, and the solid was collected by filtration, washed with H₂O then hexanes and dried in vacuo. The solid was purified by reverse phase chromatography on C18 (ISCO-Rf, 5-100% MeOH/H₂O) to give N-hydroxy-6-(3-(3-(pentafluoro-λ⁶-sulfaneyl)benzyl)ureido)chromane-2-carboxamide as an off-white solid (14f, 12.0 mg): ¹H NMR (DMSO-d₆, 400 MHz): 10.75 (br s, 1H), 8.91 (br s, 1H), 8.41 (br s, 1H), 7.83-7.80 (m, 1H), 7.80-7.76 (m, 1H), 7.63-7.56 (m, 2H), 7.14 (d, J=2.4 Hz, 1H), 7.06 (dd, J=8.8, 2.5 Hz, 1H), 6.71 (d, J=9.1 Hz, 1H), 6.68 (t, J=6.2 Hz, 1H), 4.45 (dd, J=9.3, 2.3 Hz, 1H), 4.38 (d, J=5.8 Hz, 2H), 2.82-2.71 (m, 1H), 2.70-2.61 (m, 1H), 2.14-2.05 (m, 1H), 1.98-1.85 (m, 1H); HRMS (ESI+) m/z calcd for C₁₈H₁₉O₄N₃F₅S [M+H] 468.1011, found 468.1012.

Methyl 6-(3-(4-fluorophenyl)ureido)chromane-2-carboxylate (12g). To a solution of methyl 6-aminochromane-2-carboxylate (4, 150 mg, 0.72 mmol) in CH₂Cl₂ (2.5 mL) was added carbonyl diimidazole (146 mg, 0.90 mmol) at room temperature. After 16 h, 4-fluoroaniline (86.0 mg, 0.86 mmol) in CH₂Cl₂ (2.0 ml), was added. After 16 h, the reaction mixture was extracted with CH₂Cl₂ and washed with H₂O. The aqueous layer was back extracted with CH₂Cl₂ (3×). The combined organic layer was dried (Na₂SO₄), filtered and concentrated. The residue was purified chromatography on SiO₂ (ISCO-Rf, 0-80% EtOAc/hexanes) to give methyl 6-(3-(4-fluorophenyl)ureido)chromane-2-carboxylate (12g, 100 mg, 76%) as an off white solid: ¹H NMR (DMSO-d₆, 600 MHz): δ 8.59 (s, 1H), 8.38 (s, 1H), 7.43 (dd, J=9.1, 4.8 Hz, 2H), 7.17 (d, J=2.3 Hz, 1H), 7.13-7.05 (m, 3H), 6.74 (d, J=8.7 Hz, 1H), 4.85 (dd, J=7.1, 4.2 Hz, 1H), 3.69 (s, 3H), 2.76 (ddd, J=16.6, 6.8, 3.8 Hz, 1H), 2.60 (dt, J=16.6, 6.1 Hz, 1H), 2.19-2.11 (m, 1H), 2.09-1.98 (m, 1H); ¹³C NMR (DMSO-d₆, 150 MHz): δ 170.9, 158.0, 156.4, 155.7, 152.8, 148.3, 136.2, 132.5, 121.5, 119.8, 119.7, 118.5, 116.3, 115.3, 115.1, 106.3, 100.8, 99.5, 97.4, 72.8, 52.1, 23.9, 22.6.

Methyl 6-(3-(4-methoxybenzyl)ureido)chromane-2-carboxylate (12h). To a solution of methyl 6-aminochromane-2-carboxylate (4, 150 mg, 0.72 mmol) in CH₂Cl₂ (2.5 mL) was added carbonyl diimidazole (146 mg, 0.90 mmol) at room temperature. After 16 h (4-methoxyphenyl)methanamine (119.0 mg, 0.86 mmol) in CH₂Cl₂ (2.0 ml) was added. After 16 h, the reaction mixture was extracted with CH₂Cl₂ and washed with H₂O. The aqueous layer was back extracted with CH₂Cl₂ (3×). The combined organic layer was dried (Na₂SO₄), filtered and concentrated. The residue was purified by chromatography on SiO₂ (ISCO-Rf, 0-80% EtOAc/hexanes) to give methyl 6-(3-(4-methoxybenzyl)ureido)chromane-2-carboxylate (12 h, 115 mg, 43%) as yellow solid: ¹H NMR (CDCl₃, 600 MHz): δ 7.19 (d, J=8.2 Hz, 2H), 7.02 (br s, 1H), 6.88 (dd, J=8.2, 1.8 Hz, 1H), 6.86-6.82 (m, 3H), 6.30 (br s, 1H), 5.07 (t, J=5.2 Hz, 1H), 4.70 (dd, J=7.1, 4.2 Hz, 1H), 4.33 (d, J=5.5 Hz, 2H), 3.79 (s, 3H), 3.77 (s, 3H), 2.77 (ddd, J=16.6, 6.8, 3.8 Hz, 1H), 2.66 (dt, J=16.6, 6.1 Hz, 1H), 2.25-2.20 (m, 1H), 2.18-2.11 (m, 1H); ¹³C NMR (CDCl₃, 150 MHz): δ 171.2, 158.8, 156.4, 150.7, 131.0, 130.8, 128.8, 124.3, 122.7, 122.0, 117.5, 113.9, 73.7, 55.2, 52.4, 43.7, 24.3, 23.3.

N-Hydroxy-6-(3-(4-methoxybenzyl)ureido)chromane-2-carboxamide (14h). To an ice-cooled solution of hydroxylamine hydrochloride (543 mg, 7.75 mmol) in methanol (5.0 mL) was added powdered KOH (521 mg, 9.30 mmol) portion wise. After 1 h, the solid was removed by filtration and the filtrate was added dropwise to an ice-cooled solution of methyl 6-(3-(4-methoxybenzyl)ureido)chromane-2-carboxylate (12 h, 115 mg, 0.31 mmol) in MeOH/THF (2.0/2.0 mL). An additional amount of KOH (174 mg, 3.1 mmol) was then added. After 1 h, 1M HCl was added slowly to pH 7-8 and the solid was collected by filtration and washed with H₂O then hexanes and dried in vacuo to give N-hydroxy-6-(3-(4-methoxybenzyl)ureido)chromane-2-carboxamide (14 h, 80 mg, 70%) as a yellow solid: ¹H NMR (DMSO-d₆, 600 MHz): δ 10.74 (br s, 1H), 8.91 (s, 1H), 8.23 (s, 1H), 7.20 (d, J=8.4 Hz, 2H), 7.12 (d, J=2.4 Hz, 1H), 7.04 (dd, J=9.0, 2.4 Hz, 1H), 6.88 (d, J=8.4 Hz, 2H), 6.68 (d, J=9.0 Hz, 1H), 6.42 (t, J=6.0 Hz, 1H), 4.43 (dd, J=9.0, 2.4 Hz, 1H), 4.17 (d, J=6.0 Hz, 2H), 3.74 (s, 3H), 2.73 (t, J=16.6, 6.8, 3.8 Hz, 1H), 2.64 (dt, J=16.6, 6.1 Hz, 1H), 2.15-2.08 (m, 1H), 1.95-1.88 (m, 1H); ¹³C NMR (DMSO-d₆, 150 MHz): δ166.5, 158.1, 155.3, 147.9, 133.4, 132.4, 128.4, 121.6, 118.9, 117.6, 116.4, 113.6, 73.7, 55.0, 42.2, 24.4, 23.3; HRMS (ESI+) m/z [M−H] calcd for C₁₉H₂₀O₅N₃ 370.1398; found 370.1398.

Methyl 8-(3-(4-chloro-3-(trifluoromethyl)benzyl)ureido)chromane-2-carboxylate (12i). To a solution of methyl 6-aminochromane-2-carboxylate (4, 200 mg, 0.965 mmol) in CH₂Cl₂ (3.0 mL) was added carbonyl diimidazole (250 mg, 1.54 mmol) at room temperature. After 5 h, 4-chloro-3-(trifluoromethyl)benzylamine (243 mg, 1.16 mmol) was then added. After 6 h, the reaction mixture was extracted with CH₂Cl₂, washed with 0.25M HCl, and brine, dried (Na₂SO₄), filtered and concentrated. The residue was purified by chromatography on SiO₂ (4/1, hexanes/EtOAc) to give methyl 8-(3-(4-chloro-3-(trifluoromethyl)benzyl)ureido)chromane-2-carboxylate (12i, 0.290 g, 68%) as a white solid: 3; HRMS (ESI+) m/z [M+H] calcd for C₂₀H₁₉O₄N₂ClF₃ 443.0980; found 443.0976.

Lithium 8-(3-(4-chloro-3-(trifluoromethyl)benzyl)ureido)chromane-2-carboxylate (13i-int). To a solution of methyl 8-(3-(4-chloro-3-(trifluoromethyl)benzyl)ureido)chromane-2-carboxylate (12i, 0.209 g, 0.425 mmol) in THF/MeOH (3.0/1.5 mL) was added 1M LiOH (0.47 mL) at room temperature. After 15.5 h, the solution was concentrated and the residue was diluted with PhMe and concentrated (3×) to give lithium 8-(3-(4-chloro-3-(trifluoromethyl)benzyl)ureido)chromane-2-carboxylate (13i-int, crude) that was used without further purification: HRMS (ESI+) m/z [M+H] calcd for C₁₉H₁₇O₄N₂ClF₃ 429.0823; found 429.0819.

8-(3-(4-Chloro-3-(trifluoromethyl)benzyl)ureido)-N-((tetrahydro-2H-pyran-2-yl)oxy)chromane-2-carboxamide (13i). A solution of lithium 8-(3-(4-chloro-3-(trifluoromethyl)benzyl)ureido)chromane-2-carboxylate (13i-int, 0.185 g, 0.426 mmol, crude) in DMF (2.5 mL) was treated with O-(tetrahydro-2H-pyran-2-yl)hydroxylamine (0.055 g, 0.47 mmol) in DMF (1.5 mL) followed by T3P (0.304 mL, 0.511 mmol, 50% EtOAc soln) and DIPEA (0.08 mL, 0.51 mmol) at room temperature. After 3 h, the solution was extracted with EtOAc, washed with satd. NH₄Cl, 1M LiCl solution (3×), brine, dried (Na₂SO₄), filtered and concentrated. The residue was purified by chromatography on SiO₂ (2/1, hexanes/EtOAc) to give 8-(3-(4-chloro-3-(trifluoromethyl)benzyl)ureido)-N-((tetrahydro-2H-pyran-2-yl)oxy)chromane-2-carboxamide (13i, 0.079 g, 35%) as a white solid: HRMS (ESI+) m/z [M+Na] calcd for C₂₄H₂₅O₅N₃ClF₃Na 550.1327; found 550.1325.

8-(3-(4-Chloro-3-(trifluoromethyl)benzyl)ureido)-N-hydroxychromane-2-carboxamide (14i). To a solution of 8-(3-(4-chloro-3-(trifluoromethyl)benzyl)ureido)-N-((tetrahydro-2H-pyran-2-yl)oxy)chromane-2-carboxamide (13i, 0.079 g, 0.15 mmol) in MeOH (3.0 mL) was added Amberlyst-15 (0.006 g, 0.030 mmol, washed with MeOH). After 18 h, the reaction mixture was filtered through Celite®, rinsed with MeOH, and concentrated. The residue was slurried with Et₂O and the solid was collected by filtration to give 8-(3-(4-chloro-3-(trifluoromethyl)benzyl)ureido)-N-hydroxychromane-2-carboxamide (14i, 0.025 g, 38%) as a white solid: mp 195-197° C.; ¹³C NMR (DMSO-d₆, 150 MHz): δ 166.3, 155.5, 142.5, 140.8, 132.9, 131.6, 128.8, 128.0, 126.5, 126.3, 123.8, 122.5, 122.0, 121.7, 120.1, 117.6, 74.6, 41.9, 24.3, 23.0; HRMS (ESI+) m/z [M+H] calcd for C₁₉H₁₈O₄N₃ClF₃ 444.0932; found 444.0930.

Methyl 6-((((4-methoxybenzyl)oxy)carbonyl)amino)chromane-2-carboxylate (12j). To suspension of carbonyl diimidazole (0.122 g, 0.752 mmol) in 1,2,-dichloroethane (1.0 mL) was added methyl 6-aminochromane-2-carboxylate (0.110 g, 0.53 mmol) in 1,2-DCE (1.2 mL) at room temperature. The homogenous solution was heated at 50° C. overnight. The solution was concentrated to give crude product as amber oil that was used without further purification: HRMS (LCMS ESI+) m/z calcd for C₁₅H₁₆O₄N₃ [M+H] 302.1135, found 302.1133. To a solution of this crude product (0.160 g, 0.53 mmol-based on theoretical) in MeCN (3.0 mL) was added pyridine (0.10 mL, 1.22 mmol) followed by (4-methoxyphenyl)methanol (0.10 mL, 0.80 mmol) and DMAP (0.010 g, 0.081 mmol) at room temperature. The solution was heated at 50° C. After 3.5 h, the solution was extracted with EtOAc, washed with 0.5M HCl, brine, dried (Na₂SO₄), filtered and concentrated. The residue was purified by chromatography on SiO₂ (ISCO-Rf, 0-100% EtOAc/hexanes) to give methyl 6-((((4-methoxybenzyl)oxy)carbonyl)amino)chromane-2-carboxylate (12j, 0.207 g, contained (4-methoxyphenyl)methanol, 1.6/1 product/alcohol) that was used without further purification: HRMS (LCMS ESI+) m/z calcd for C₂₀H₂₁O₆Nal [M+Na]394.1261, found 394.1261.

6-((((4-Methoxybenzyl)oxy)carbonyl)amino)chromane-2-carboxylic acid (13j-int). A solution of methyl 6-((((4-methoxybenzyl)oxy)carbonyl)amino)chromane-2-carboxylate (0.207 g, crude, 1.6/1, prod/SM) in MeOH/THF (3.0 mL, 2/1) was treated with 1M LiGH (0.40 mL) at room temperature. After 5 h, the solution was concentrated and the residue was diluted with H₂O and extracted with EtOAc (2×). The aqueous was acidified with 3M HCl and extracted with EtOAc (2×), dried (Na₂SO₄), filtered and concentrated to give 6-((((4-methoxybenzyl)oxy)carbonyl)amino)chromane-2-carboxylic (13j-int, 0.060 g, approx. 90% purity) as a beige solid that was used without further purification: ¹H NMR (DMSO-d₆, 300 MHz): δ12.95 (s, 1H), 9.42 (s, 1H), 7.35 (d, J=8.7 Hz, 2H), 7.21-7.10 (m, 2H), 6.94 (d, J=8.7 Hz, 2H), 6.71 (d, J=8.7 Hz, 1H), 5.03 (s, 2H), 4.70 (dd, J=3.9, 6.9 Hz, 1H), 3.75 (s, 3H), 2.77-2.73 (m, 1H), 2.71-2.55 (m, 1H), 2.15-1.98 (m, 2H); HRMS (LCMS ESI+) m/z calcd for C₁₉H₁₈O₆N [M−H] 356.1129, found 356.1146.

4-Methoxybenzyl (2-(((tetrahydro-2H-pyran-2-yl)oxy)carbamoyl)chroman-6-yl)carbamate (13j). A solution of 6-((((4-methoxybenzyl)oxy)carbonyl)amino)chromane-2-carboxylic acid (13j-int, 0.060 g, approx. 90% purity) in CH₂Cl₂/DMF (2.0 mL, 3/1) was treated with O-(tetrahydro-2H-pyran-2-yl)hydroxylamine 0.023 g, 0.20 mmol) in CH₂Cl₂ (0.5 mL) followed by T3P (0.176 g, 50% EtOAc soln) then DIPEA (0.050 mL, 0.30 mmol) at room temperature. After 22 h, the solution was extracted with CH₂Cl₂, washed with 0.5M HCl, 1M LiCl, H₂O, dried (Na₂SO₄), filtered and concentrated. The residual oil was purified by chromatography on SiO₂ (ISCO-Rf, 0-10% MeOH/CH₂Cl₂) to give 4-methoxybenzyl (2-(((tetrahydro-2H-pyran-2-yl)oxy)carbamoyl)chroman-6-yl)carbamate (13j, 0.064 g 26%-3 steps) as a light yellow oil: ¹H NMR (CDCl₃, 400 MHz): δ9.18 (s, 0.5H), 9.17 (s, 0.5H), 7.35 (d, J=8.6 Hz, 2H), 7.18 (br s, 1H), 7.18 (br s, 1H), 7.08-7.05 (m, 1H), 6.91 (d, J=8.6 Hz, 2H), 6.81 (dd, J=7.1, 8.6 Hz, 1H), 6.65 (br s, 1H), 5.14 (s, 2H), 5.05 (app t, J=2.8 Hz, 0.5H), 4.98 (app t, J=3.5 Hz, 0.5H), 4.62-4.57 (m, 1H), 4.04-3.97 (m, 1H), 3.83 (s, 3H), 3.69-3.62 (m, 1H), 2.90-2.76 (m, 2H), 2.45-2.41 (m, 1H), 2.09-2.01 (m, 1H), 1.89-1.80 (m, 3H), 1.70-1.59 (m, 3H); HRMS (LCMS ESI+) m/z calcd for C₂₄H₂₈O₇N₂Na [M+Na] 479.1789, found 479.1788.

4-Methoxybenzyl (2-(hydroxycarbamoyl)chroman-6-yl)carbamate (14j). To a solution of 4-methoxybenzyl (2-(((tetrahydro-2H-pyran-2-yl)oxy)carbamoyl)chroman-6-yl)carbamate (13j, 0.074 g, 0.16 mmol) in MeOH (2.0 mL) was added Amberlyst-15 (0.021 g, washed with MeOH) at room temperature. After 24 h, the mixture was filtered through Celite®, rinsed with MeOH and concentrated. The solid residue was slurried with Et₂O and the solid was collected by filtration and dried in vacuo at 60° C. to give 4-methoxybenzyl (2-(hydroxycarbamoyl)chroman-6-yl)carbamate (14j, 0.026 g, 43%) as a light tan solid: mp 149-151° C.; IR (neat) 3294, 2892, 1689, 1634, 1612, 1528, 1517, 1492, 1425, 1241, 1220, 1175, 1079, 1056, 1030, 894, 871, 820, 807, 774 cm⁻¹; ¹H NMR (DMSO-d₆, 400 MHz): δ10.76 (s, 1H), 9.44 (s, 1H), 8.92 (d, J=1.6 Hz, 1H), 7.34 (d, J=8.6 Hz, 2H), 7.15-7.11 (m, 2H), 6.93 (d, J=8.6 Hz, 2H), 6.72 (d, J=8.7 Hz, 1H), 5.03 (s, 2H), 4.45 (dd, J=2.9, 8.9 Hz, 1H), 3.75 (s, 3H), 2.79-2.71 (m, 1H), 2.67-2.60 (m, 1H), 2.11-2.06 (m, 1H), 1.95-1.87 (m, 1H); ¹³C NMR (DMSO-d₆, 100 MHz): δ 166.5, 159.1, 153.6, 148.8, 131.94, 131.91, 130.1, 128.6, 121.7, 116.6, 113.8, 73.8, 65.4, 55.1, 24.4, 23.3; HRMS (LCMS ESI+) m/z calcd for C₁₉H₁₉O₆N₂ [M−H] 371.1238, found 371.1247.

Methyl 6-((((2-chlorobenzyl)oxy)carbonyl)amino)chromane-2-carboxylate (12k). To a solution of methyl 6-aminochromane-2-carboxylate (0.152 g, 0.733 mmol) in CH₂Cl₂ (1.5 mL) was added pyridine (0.20 mL, 2.4 mmol). The solution was cooled to 0° C. and treated dropwise with 2-chlorobenzyl carbonochloridate (0.255 g, 75% purity) in CH₂Cl₂ (1.0 mL). After 1 h, the solution extracted with CH₂Cl₂, washed with 0.5 M HCl, brine, dried (Na₂SO₄), filtered and concentrated. The residual oil was purified by chromatography on SiO₂ (ISCO-Rf, 0-100% EtOAc/hexanes) to give methyl 6-((((2-chlorobenzyl)oxy)carbonyl)amino)chromane-2-carboxylate as an amber oil (12k, 0.204 g, 74%): ¹H NMR (CDCl₃, 400 MHz): 37.41 (app t, J=4.9 Hz, 1H), 7.35 (dd, J=2.8, 4.4 Hz, 1H), 7.26-7.19 (m, overlapping signals, 2H), 7.17 (s, 1H), 6.95 (dd, J=2.6, 8.7 Hz, 1H), 6.81 (d, J=8.8 Hz, 1H), 6.53 (br s, 1H), 5.25 (s, 2H), 4.66 (dd, J=3.6, 7.5 Hz, 1H), 3.74 (s, 3H), 2.81-2.64 (m, 2H), 2.23-2.09 (m, 2H); HRMS (LCMS ESI+) m/z calcd for C₁₉H₁₉O₅NCl [M+H] 376.0946, found 376.0943.

6-((((2-Chlorobenzyl)oxy)carbonyl)amino)chromane-2-carboxylic acid (12k-int). To a solution of methyl 6-((((2-chlorobenzyl)oxy)carbonyl)amino)chromane-2-carboxylate (0.204 g, 0.543 mmol) in MeOH/THF (3.0 mL, 2/1) was added 1M LiGH (1.0 mL) at room temperature. After 5 h, the solution was concentrated, diluted with H₂O, acidified with 1M HCl and extracted with EtOAc (2×). The combined organic layer was washed with brine, dried (Na₂SO₄), filtered and concentrated to give 6-((((2-chlorobenzyl)oxy)carbonyl)amino)chromane-2-carboxylic acid (12k-int, 0.185 g, 94%) as an off-white solid: ¹H NMR (DMSO-d₆, 400 MHz): δ12.98 (s, 1H), 9.59 (s, 1H), 7.55 (dd, J=3.6, 5.6 Hz, 1H), 7.51 (dd, J=3.5, 5.3 Hz, 1H), 7.41-7.38 (m, 2H), 7.17 (s, 1H), 7.13 (d, J=8.8 Hz, 1H), 6.72 (d, J=8.7 Hz, 1H), 5.20 (s, 2H), 4.72 (dd, J=3.8, 6.6 Hz, 1H), 2.77-2.71 (m, 1H), 2.62-2.55 (m, 1H), 2.15-2.09 (m, 1H), 2.06-1.99 m, 1H); HRMS (LCMS ESI+) m/z calcd for C₁₈H₁₅O₅NCl [M−H] 360.0633, found 360.0649.

2-Chlorobenzyl (2-(((tetrahydro-2H-pyran-2-yl)oxy)carbamoyl)chroman-6-yl)carbamate (13k). To a solution of 6-((((2-chlorobenzyl)oxy)carbonyl)amino)chromane-2-carboxylic acid (12k-int, 0.185 g, 0.511 mmol) in CH₂Cl₂/DMF (2.0 mL, 1/1) was added 0-(tetrahydro-2H-pyran-2-yl)hydroxylamine (0.078 g, 0.67 mmol) in CH₂Cl₂ (0.5 mL) followed by T3P (0.394 g, 50% EtOAc) solution followed by DIPEA (0.10 mL, 0.61 mmol) at room temperature. After 6 h, the solution was extracted with CH₂Cl₂, washed with 0.5M HCl, 1M LiCl, H₂O, dried (Na₂SO₄), filtered and concentrated. The residual oil was purified by chromatography on SiO₂ (ISCO-Rf, 0-100% EtOAc/hexanes) to give 2-chlorobenzyl (2-(((tetrahydro-2H-pyran-2-yl)oxy)carbamoyl)chroman-6-yl)carbamate (13k, 0.215 g, 91%) as a light amber oil: ¹H NMR (CDCl₃, 400 MHz): δ9.16 (s, 0.5H), 9.15 (s, 0.5H), 7.46 (app t, J=4.9 Hz, 1H), 7.41-7.39 (m, 1H), 7.30-7.27 (m, overlapping signals, 2H), 7.20 (s, 1H), 7.08 (d, J=7.9 Hz, 1H), 6.81 (dd, J=7.2, 8.4 Hz, 1H), 6.73 (s, 1H), 5.30 (d, J=1.1 Hz, 2H), 5.03 (app t, J=2.8 Hz, 0.5H), 4.97 (app t, J=2.9 Hz, 0.5H), 4.60-4.56 (m, 1H), 4.02-3.97 (m, 1H), 3.69-3.61 (m, 1H), 2.83-2.74 (m, 2H), 2.44-2.39 (m, 1H), 2.07-2.01 (m, 1H), 1.85-1.79 (m, 3H), 1.62-1.58 (m, 3H); HRMS (LCMS ESI+) m/z calcd for C₂₃H₂₅O₆N₂ClNa [M+Na] 483.1293, found 483.1292.

2-Chlorobenzyl (2-(hydroxycarbamoyl)chroman-6-yl)carbamate (14k). To a solution of 2-chlorobenzyl (2-(((tetrahydro-2H-pyran-2-yl)oxy)carbamoyl)chroman-6-yl)carbamate (13k, 0.214 g, 0.464 mmol) in MeOH (2.0 mL) was added Amberlyst-15 (0.042 g, washed with MeOH) at room temperature. After 6 h, the mixture was diluted with EtOAc and concentrated. The residue was diluted with EtOAc, filtered through Celite®, rinsed with EtOAc and concentrated. The crude solid was slurried with Et₂O and collected by filtration and dried in vacuo at 60° C. to give 2-chlorobenzyl (2-(hydroxycarbamoyl)chroman-6-yl)carbamate (14k, 0.099 g, 57%) as a peach solid: mp 188-189° C.; IR (neat) 3280, 1690, 1636, 1528, 1425, 1257, 1239, 1218, 1064, 895, 874, 809, 747 680 cm⁻¹; ¹H NMR (DMSO-d₆, 600 MHz): δ10.75 (s, 1H), 9.58 (s, 1H), 8.90 (s, 1H), 7.54 (app t, J=5.2 Hz, 1H), 7.50 (dd, J=3.5, 5.3 Hz, 1H), 7.41-7.39 (m, 2H), 7.18 (s, 1H), 7.14 (d, J=8.3 Hz, 1H), 6.74 (d, J=8.8 Hz, 1H), 5.20 (s, 2H), 4.45 (dd, J=2.9, 8.9 Hz, 1H), 2.78-2.73 (m, 1H), 2.67-2.64 (m, 1H), 2.09-2.06 (m, 1H), 1.93-1.89 (m, 1H); ¹³C NMR (DMSO-d₆, 150 MHz): δ 166.4, 153.2, 148.9, 134.1, 132.6, 131.7, 130.2, 129.9, 129.3, 127.4, 121.8, 119.3, 117.9, 116.6, 73.7, 62.9, 24.3, 23.2; HRMS (LCMS ESI+) m/z calcd for C₁₈H₁₈O₅N₂Cl [M+H] 377.0899, found 377.0895.

Methyl 6-(((benzyloxy)carbonyl)amino)chromane-2-carboxylate (121). A solution of methyl 6-aminochromane-2-carboxylate (4, 0.200 g 0.965 mmol) in CH₂Cl₂ (6.0 mL) was cooled to 0° C. and treated with pyridine (0.084 mL, 1.1 mmol) followed by benzyl chloroformate (0.15 mL, 1.1 mmol) in CH₂Cl₂ (2.0 mL). After 10 min, the solution was warmed to room temperature. After 19 h, the reaction mixture was extracted with CH₂Cl₂, washed with H₂O, 0.1M HCl, brine (2×), dried (Na₂SO₄), filtered and concentrated to give methyl 6-(((benzyloxy)carbonyl)amino)chromane-2-carboxylate (121, 0.255 g, 77%) as a light brown solid.

Lithium 6-(((benzyloxy)carbonyl)amino)chromane-2-carboxylate (131-int). A solution of methyl 6-(((benzyloxy)carbonyl)amino)chromane-2-carboxylate (121, 0.255 g, 0.747 mmol, crude) in THF/MeOH (3.0/1.5 mL) was treated with 1 LiGH (0.78 mL) at room temperature. After 15.5 h, the solution was concentrated and the residue was diluted with PhMe and concentrated (3×) to give lithium 6-(((benzyloxy)carbonyl)amino)chromane-2-carboxylate (131-int, crude) that was used without further purification: HRMS (LCMS ESI+) m/z calcd for C₁₈H₁₈O₅N [M+H] 328.1179, found 328.1177.

Benzyl (2-(((tetrahydro-2H-pyran-2-yl)oxy)carbamoyl)chroman-6-yl)carbamate (131). A solution of lithium 6-(((benzyloxy)carbonyl)amino)chromane-2-carboxylate (131-int, 0.249 g, 0.747 mmol, crude) in DMF (3.0 mL) was treated with O-(tetrahydro-2H-pyran-2-yl)hydroxylamine (0.096 g, 0.822 mmol) in DMF (1.5 mL) followed by HATU (0.313 g, 0.822 mmol) and DIPEA (0.15 mL, 0.897 mmol) at room temperature. After 15.5 h, the solution was extracted with EtOAc, washed with 0.25M HCl (2×), 1M LiCl (3×), brine, dried (Na₂SO₄), filtered and concentrated. The residual oil was purified by chromatography on SiO₂ (2/1, hexanes:EtOAc) to give benzyl (2-(((tetrahydro-2H-pyran-2-yl)oxy)carbamoyl)chroman-6-yl)carbamate (131, 0.130 g, 41%) as a semi solid residue HRMS (LCMS ESI+) m/z calcd for C₂₃H₂₆O₆N₂Na [M+Na] 449.1683, found 449.1679.

Benzyl (2-(hydroxycarbamoyl)chroman-6-yl)carbamate (141). To a solution of benzyl (2-(((tetrahydro-2H-pyran-2-yl)oxy)carbamoyl)chroman-6-yl)carbamate (131, 0.130 g, 0.305 mmol) in MeOH (3.0 mL) was added Amberlyst-15 (0.013 g, 0.061 mmol, washed with MeOH) at room temperature. After 15 h, the reaction mixture was filtered through Celite®, washed with MeOH and concentrated. The solid residue was treated with Et₂O and the solid was collected by filtration to give benzyl (2-(hydroxycarbamoyl)chroman-6-yl)carbamate (141, 0.054 g, 52%) as a white solid: ¹H NMR (DMSO-d₆, 400 MHz): δ10.78 (d, J=1.6 Hz, 1H), 9.53 (s, 1H), 8.94 (s, 1H), 7.44-7.33 (m, overlapping signals, 5H), 7.18-7.13 (m, 2H), 6.75 (d, J=8.8 Hz, 1H), 5.13 (s, 2H), 4.47 (dd, J=2.8, 8.8 Hz, 1H), 2.79-2.73 (m, 1H), 2.68-2.63 (m, 1H), 2.12-2.08 (m, 1H), 1.95-1.89 (m, 1H); ¹³C NMR (DMSO-d₆, 100 MHz): δ166.9, 153.9, 149.3, 137.2, 132.4, 128.9, 128.53, 128.46, 122.3, 119.8, 118.4, 117.1, 74.2, 66.0, 24.8, 23.7; HRMS (LCMS ESI+) m/z calcd for C₁₈H₁₉O₅N₂ [M+H] 343.1288, found 343.1286.

Methyl 6-(naphthalene-1-sulfonamido)chromane-2-carboxylate (12m). A solution of 6-aminochromane-2-carboxylate (4, 150 mg, 0.72 mmol) in CH₂Cl₂ (10.0 mL) was cooled to 0° C. and treated with pyridine (146 mg, 1.85 mmol) followed by naphthalene-1-sulfonyl chloride (186 mg, 0.82 mmol). After 16 h, the reaction mixture was extracted with EtOAc and washed with H₂O.

The aqueous layer was back extracted with EtOAc (3×). The combined organic layer was washed with H₂O, brine, dried (Na₂SO₄), filtered and concentrated. The residue was purified by chromatography on SiO₂ (ISCO-Rf, 0-5% MeOH/CH₂Cl₂) to give methyl 6-(naphthalene-1-sulfonamido)chromane-2-carboxylate (12m, 220 mg, 75%) as a yellow oil: ¹H NMR (CDCl₃, 400 MHz): δ8.68 (d, J=8.0 Hz, 1H), 8.12 (d, J=7.6 Hz, 1H), 8.05 (d, J=8. Hz, 1H), 7.96 (d, J=8.0 Hz, 1H), 7.68 (d, J=8.8 Hz, 1H), 7.63 (d, J=8.4 Hz, 1H), 7.46 (app t, J=7.6 Hz, 1H), 6.70 (d, J=1.2 Hz, 1H), 6.64 (d, J=8.8 Hz, 2H), 6.51 (dd, J=8.1, 2.0 Hz, 1H), 4.66 (dd, J=7.2, 3.6 Hz, 1H), 3.78 (s, 3H), 2.65-2.60 (m, 2H), 2.18-2.11 (m, 2H); ¹³C NMR (CDCl₃, 100 MHz): δ171.7, 171.2, 151.3, 146.4, 139.8, 129.5, 123.7, 122.2, 122.1, 121.8, 117.6, 117.4, 115.8, 115.3, 73.7, 66.3, 66.2, 52.5, 52.4, 46.5, 46.4, 29.6, 24.8, 24.3, 23.5, 23.3.

N-Hydroxy-6-(naphthalene-1-sulfonamido)chromane-2-carboxamide (14m). To an ice-cooled solution of hydroxylamine hydrochloride (555 mg, 7.93 mmol) in methanol (5.0 mL) was added powdered KOH (520 mg, 9.30 mmol) portion wise. After 1 h, the solid was removed by filtration and the filtrate was added dropwise to an ice-cooled solution of methyl 6-(naphthalene-1-sulfonamido)chromane-2-carboxylate (12m, 100 mg, 0.28 mmol) in MeOH (5 mL). After 2 h, 1M HCl was added dropwise until pH 7-8 and the product was collected by filtration, washed with H₂O then hexanes and dried in vacuo. The solid was purified by chromatography on SiO₂ (0-5% MeOH/CH₂Cl₂) to give N-hydroxy-6-(naphthalene-1-sulfonamido)chromane-2-carboxamide (14m, 70.0 mg, 70%) as an off-white solid: ¹H NMR (DMSO-d₆, 600 MHz): δ10.73 (s, 1H), 10.30 (s, 1H), 8.88 (s, 1H), 8.70 (d, J=8.6 Hz, 1H), 8.20 (d, J=8.2 Hz, 1H), 8.11 (dd, J=7.2, 1.5 Hz, 1H), 8.07 (d, J=8 Hz, 1H), 7.71 (ddd, J=8.2, 6.7, 1.5 Hz, 1H), 7.67 (ddd, J=9.0, 7.9, 1.5 Hz, 1H), 7.58 (dd, J=8.9, 3.1 Hz, 1H), 6.70 (s, 1H), 6.68 (d, J=3.0 Hz, 1H), 6.58 (dt, J=7.8, 1.2 Hz, 1H), 4.40 (dd, J=9.0, 3.1 Hz, 1H), 2.62-2.52 (m, 2H), 1.98-1.96 (m, 1H), 1.81-1.76 (m, 1H); ¹³C NMR (DMSO-d₆, 150 MHz): δ 166.2, 150.1, 134.0, 133.6, 129.5, 128.9, 127.9, 127.5, 126.8, 124.4, 122.1, 121.8, 120.0, 116.9, 73.6, 23.9, 22.9; HRMS (LCMS ESI+) m/z calcd for C₂₀H₁₇O₅N₂S [M−H] 397.0853; found 397.0859.

Methyl 6-((3,5-dichlorophenyl)sulfonamido)chromane-2-carboxylate (12n). To a solution of 6-aminochromane-2-carboxylate (4, 0.194 g, 0.935 mmol) in THE (10 mL) was added triethylamine (0.17 mL, 1.20 mmol) and DMAP (0.0229 g, 0.187 mmol) followed by 3,5-dichlorobenzenesulfonyl chloride (0.285 g, 1.14 mmol) at room temperature. After 15 h, the reaction was extracted with EtOAc and washed with 1M HCl. The aqueous layer was back extracted with EtOAc (4×) and the combined organic layer was washed with brine, dried (Na₂SO₄), filtered and concentrated. The residue was purified by chromatography on SiO₂ (0-50% EtOAc/hexanes) to give methyl 6-((3,5-dichlorophenyl)sulfonamido)chromane-2-carboxylate (12n, 0.210 g, 40%) as a white powder: ¹H NMR (CDCl₃, 400 MHz): δ 7.56 (s, 2H), 7.51 (app t, J=1.8 Hz, 1H), 7.26 (s, 1H), 6.83-6.80 (m, 2H), 6.73 (s, 1H), 4.74 (dd, J=7.3, 3.7 Hz, 1H), 3.79 (s, 3H), 2.80-2.65 (m, 2H), 2.25-2.15 (m, 2H); HRMS (ESI+) m/z calcd for C₁₇H₁₅C₁₂NO₅SNa (M+Na) 437.9946, found 437.9935.

6-((3,5-Dichlorophenyl)sulfonamido)chromane-2-carboxylic acid (13n-int). A solution of methyl 6-((3,5-dichlorophenyl)sulfonamido)chromane-2-carboxylate (12n, 0.126 g, 0.302 mmol) in THF/MeOH (5 mL, 2:1) was treated with 1M LiGH (0.4 mL) at room temperature. After 16 h, the solution was treated with additional 1M LiGH (0.4 mL). After 1 h, the reaction mixture was extracted with EtOAc and H₂O. The aqueous layer was acidified with 0.5M HCl until pH 1 and extracted with EtOAc (2×). The combined organic layer was washed with brine, dried (Na₂SO₄), filtered and concentrated to give 6-((3,5-dichlorophenyl)sulfonamido)chromane-2-carboxylic acid (13n-int, 0.109 g, 89%) as a light pink powder: ¹H NMR (DMSO-d₆, 300 MHz): δ 13.00-12.98 (m, 1H), 10.08 (s, 1H), 7.95 (s, 1H), 7.63 (d, J=1.9 Hz, 2H), 6.78-6.70 (m, 3H), 4.75-4.71 (m, 1H), 3.32 (s, 3H), 2.74-2.52 (m, 2H), 2.08-1.97 (m, 2H); HRMS (ESI+) m/z calcd for C₁₆H₁₂Cl₂NO₅S (M−H) 399.9813, found 399.9788.

6-((3,5-Dichlorophenyl)sulfonamido)-N-((tetrahydro-2H-pyran-2-yl)oxy)chromane-2-carboxamide (13n). A solution of 6-((3,5-dichlorophenyl)sulfonamido)chromane-2-carboxylic acid (13n-int, 0.095 g, 0.24 mmol) in DMF (2.0 mL) was treated with O-(tetrahydro-2H-pyran-2-yl)hydroxylamine (30.5 mg, 0.260 mmol). To this solution was added T3P (0.174 mL, 0.274 mmol, 50% EtOAc soln) and DIPEA (0.050 mL, 0.30 mmol). After 28 h, the solution was extracted with EtOAc and washed with H₂O. The aqueous layer was back extracted with EOAc (3×). The combined organic layer was washed with brine, dried (Na₂SO₄), filtered and concentrated. The residue was purified by chromatography on SiO₂ (5-50% EtOAc/hexanes) to give 6-((3,5-dichlorophenyl)sulfonamido)-N-((tetrahydro-2H-pyran-2-yl)oxy)chromane-2-carboxamide (13n, 0.052 g, 39%) as a white foam: ¹H NMR (acetone-d₆, 300 MHz): δ 7.76 (s, 1H), 7.63 (dd, J=1.9, 0.8 Hz, 2H), 6.91 (app t, J=5.8 Hz, 2H), 6.74 (d, J=8.4 Hz, 1H), 4.98-4.94 (m, 1H), 4.61-4.55 (m, 1H), 3.52-3.44 (m, 2H), 2.82 (d, J=6.2 Hz, 2H), 2.27-2.21 (m, 1H), 1.77-1.60 (m, 3H), 1.60-1.49 (m, 3H); HRMS (ESI+) m/z calcd for C₂₁H₂₂Cl₂N₂O₆SNa (M+Na) 523.0473, found 523.0466.

6-((3,5-Dichlorophenyl)sulfonamido)-N-hydroxychromane-2-carboxamide (14n). A solution of 6-((3,5-dichlorophenyl)sulfonamido)-N-((tetrahydro-2H-pyran-2-yl)oxy)chromane-2-carboxamide (13n, 118 mg, 0.236 mmol) in MeOH (2 mL) was treated with Amberlyst-15 (25 mg, washed with MeOH) at room temperature. After 20 h, the solution was filtered through Celite® and concentrated. The residue was treated with Et₂O and the solid was collected by filtration to give 6-((3,5-dichlorophenyl)sulfonamido)-N-hydroxychromane-2-carboxamide (14n, 47.2 mg, 50%) as an off-white powder: mp 156-170° C.; ¹H NMR (acetone-d₆, 500 MHz): δ 10.39-10.36 (m, 1H), 8.88 (d, J=3.6 Hz, 1H), 8.03 (s, 1H), 7.76 (s, 1H), 7.63-7.63 (m, 2H), 6.93-6.90 (m, 2H), 6.76 (d, J=8.6 Hz, 1H), 4.62-4.59 (m, 1H), 2.84-2.67 (m, 2H), 2.28-2.23 (m, 1H), 2.00-1.92 (m, 1H); ¹³C NMR (acetone-d₆, 150 MHz): δ 167.4, 152.5, 143.8, 136.3, 133.2, 130.2, 126.5, 125.4, 123.9, 123.5, 118.3, 75.7, 25.3, 24.3; IR: 3264, 1632, 1568, 1490, 1396, 1169; HRMS (ESI+) m/z calcd for C₁₆H₁₅N₂O₅Cl₂S (M+H) 417.0073, found 417.0073.

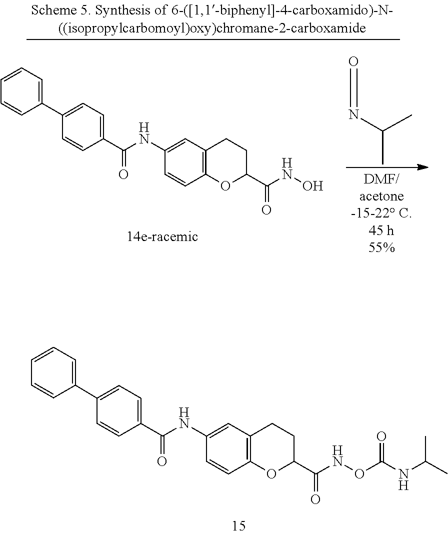

6-([1,1′-Biphenyl]-4-carboxamido)-N-((isopropylcarbamoyl)oxy)chromane-2-carboxamide (15). A solution of the 6-([1,1′-biphenyl]-4-carboxamido)-N-hydroxychromane-2-carboxamide (rac-14e, 0.0720 g, 0.185 mmol) in DMF/acetone (0.1/0.2 mL) was cooled to −15° C. The solution was treated with isopropyl isocyanate (20.0 μL, 0.204 mmol), and the mixture was warmed to room temperature. After 30 h, additional DMF/acetone (0.2/0.4 mL) was added followed by isopropyl isocyanate (20.0 mL, 0.204 mmol). After 15 h, the reaction mixture was concentrated, and H₂O was added and the solid precipitate was collected by filtration. The solid was triturated with hexanes/Et₂O (1:1) and dried in vacuo to give 6-([1,1′-biphenyl]-4-carboxamido)-N-((isopropylcarbamoyl)oxy)chromane-2-carboxamide (15, 0.048 g, 55%) as a beige solid: mp 200° C.-decomposed; IR (CH₂Cl₂) 3300, 2980, 1765, 1676, 1638, 1525, 1495, 1218 cm⁻¹; ¹H NMR (acetone-d₆, 500 MHz): δ 10.72 (s, 1H), 9.42 (s, 1H), 8.09 (d, J=8.0 Hz, 2H), 7.79 (d, J=8.5 Hz, 2H), 7.73 (d, J=7.5 Hz, 2H), 7.60-7.52 (m, 2H), 7.50 (t, J=8.0 Hz, 2H), 7.42 (t, J=7.0 Hz, 1H), 6.83 (d, J=8.5 Hz, 1H), 6.60 (s, 1H), 4.72 (dd, J=8.5, 3.0 Hz, 1H), 3.75-3.74 (m, 1H), 2.90-2.82 (m, 2H), 2.31-2.29 (m, 1H), 2.14-2.09 (m, 1H), 1.18-1.16 (m, 6H); ¹³C NMR (acetone-d₆, 125 MHz): δ 168.9, 165.6, 154.7, 150.4 144.7, 140.9, 135.2, 133.7, 129.88, 129.92, 128.88, 127.9, 127.7, 123.1, 122.4, 120.8, 117.5, 76.0, 44.6, 25.6, 24.4, 22.8; HRMS (ESI+) m/z calcd for C₂₇H₂₆O₅N₃ [M−H] 472.1867, found 472.1875.

### Part 2: 6-C Chromanes

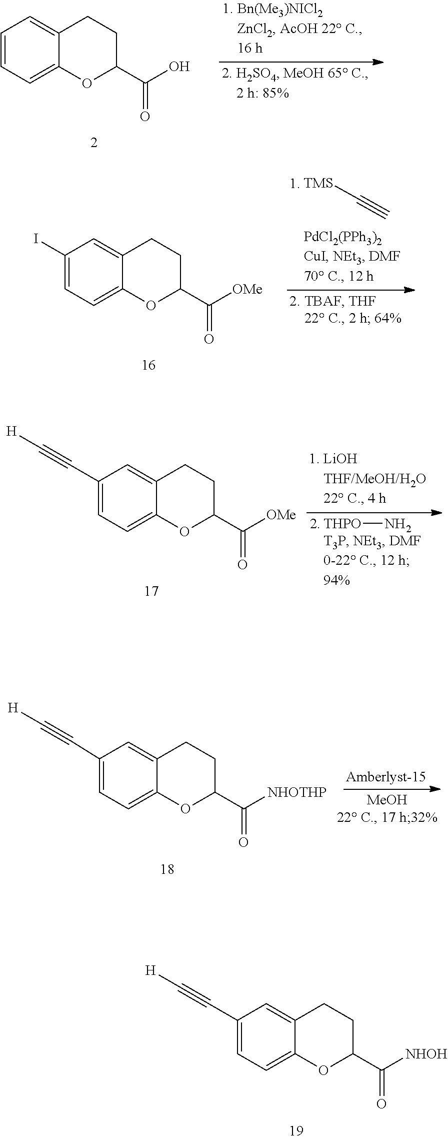

Methyl 6-iodochromane-2-carboxylate (16). Chromane-2-carboxylic acid (4.29 g, 21.7 mmol, 90% purity) in glacial AcOH (70.0 mL) was treated with ZnCl₂ (4.51 g, 33.1 mmol), followed by Bn(Me₃)NICl₂ (8.55 g, 24.5 mmol) at room temperature. After 16 h, the reaction mixture was extracted with CH₂Cl₂ and washed with H₂O. The aqueous layer was back extracted with CH₂Cl₂ (2×) and the combined organic layer was washed with H₂O (2×). The organic layer was concentrated to reduce volume (˜150 mL) then washed with 5% NaHSO₃ (2×), brine, dried (Na₂SO₄), filtered and concentrated to give a light beige/yellow solid (5.8 g). The solid was dissolved in MeOH (55 mL) and treated with conc. H₂SO₄ (15 drops) then heated at 50° C. After 2 h, the solution was concentrated, diluted with EtOAc (200 mL), washed with satd. NaHCO₃, brine, dried (Na₂SO₄), filtered and concentrated under reduced pressure to give a dark yellow/orange residue. The mixture was diluted with Et₂O/hexanes and concentrated to give methyl 6-iodochromane-2-carboxylate (16, 5.84 g, 85%) as a beige/yellow solid: ¹H NMR (CDCl₃, 300 MHz): δ 7.39 (dd, J=8.5, 2.2 Hz, 1H), 7.35-7.34 (m, 1H), 6.69 (d, J=8.5 Hz, 1H), 4.73 (dd, J=7.0, 4.0 Hz, 1H), 3.79 (s, 3H), 2.84-2.65 (m, 2H), 2.30-2.10 (m, 2H); HRMS (ESI+) m/z calcd for C₁₁H₁₁IO₃Na [M+Na] 340.9645, found 340.9643.

Methyl 6-ethynylchromane-2-carboxylate (17) Sonogashira: In 2×20 mL sealed tubes, a total amount of methyl 6-iodochromane-2-carboxylate (16, 3.49 g, 11.0 mmol) and trimethylsilylacetylene (122.0 mL, 84.9 mmol) in DMF (26 mL) was degassed via Ar sparging for 10 min. To this solution was added PdCl₂(PPh₃)₂ (0.0680 g, 0.221 mmol) and CuI (0.329 g, 1.73 mmol) and the mixture was sparged for 10 min. The mixture was then treated with TEA (5.0 mL, 35.6 mmol), sparged with Ar and the reaction vials were sealed. The mixture was heated at 80° C. for 12 h. The reaction mixtures were cooled to room temperature, combined, and extracted with EtOAc, washed with H₂O, and brine. The organic layer was dried (Na₂SO₄), filtered through Celite®, and concentrated. Deprotection: The resulting material (3.45 g, 11.98 mmol) in THF (35 mL) was treated with TBAF (1.0 M in THF, 13 mL) at room temperature. After 2 h, the reaction mixture was filtered through a pad of SiO₂ and rinsed with EtOAc and concentrated. The residue was purified by chromatography on SiO₂ (5-20% EtOAc/hexanes) to give methyl 6-ethynylchromane-2-carboxylate (17, 1.52 g, 64%) as a yellow solid: Mp 93-96° C.; IR (CH₂Cl₂) 3281, 2953, 2103, 1753, 1608, 1578, 1491, 1202, 1123 cm⁻¹; ¹H NMR (CDCl₃, 500 MHz): δ 7.25 (dd, J=8.5, 1.8 Hz, 1H), 7.19 (s, 1H), 6.86 (d, J=8.5 Hz, 1H), 4.76 (dd, J=7.2, 3.7 Hz, 1H), 3.79 (s, 3H), 2.97 (s, 1H), 2.82-2.76 (m, 1H), 2.74-2.68 (m, 1H), 2.30-2.23 (m, 1H), 2.21-2.15 (m, 1H); ¹³C NMR (CDCl₃, 125 MHz): δ 171.1, 154.1, 133.6, 131.8, 121.5, 117.2, 114.5, 83.7, 75.8, 74.0, 52.6, 24.4, 23.1; HRMS (ESI+) m/z calcd for C₁₃H₁₃O₃ [M+H] 217.0859, found 217.0860.

6-Ethynyl-N-((tetrahydro-2H-pyran-2-yl)oxy)chromane-2-carboxamide (18) To a solution of methyl 6-ethynylchromane-2-carboxylate (17, 0.351 g, 1.62 mmol) in THF (3.0 mL) and MeOH (3.0 mL) was added LiOH monohydrate (0.009 g, 2.10 mmol) in H₂O (3.0 mL) at room temperature. After 4 h, the solution was concentrated and the residue was azeotroped with toluene (2×). The crude residue was dissolved in DMF (4.0 mL) and treated with O-(tetrahydro-2H-pyran-2-yl)hydroxylamine (0.223 g, 1.90 mmol). The mixture was cooled to 0° C. and T3P (50%, 0.900 mL, 2.55 mmol, 50% EtOAc soln) and TEA (0.450 mL, 3.23 mmol) were added. The reaction mixture was slowly warmed to room temperature. After 12 h, the reaction mixture was extracted with EtOAc, washed with 0.5M HCl, brine, dried (Na₂SO₄), filtered, and concentrated. The residue was azeotroped with hexanes multiple times and the residue was dried in vacuo to give 6-ethynyl-N-((tetrahydro-2H-pyran-2-yl)oxy)chromane-2-carboxamide (18, 0.498 g, 94%, approx. 92% purity) as a thick orange/yellow foam: ¹H NMR (CDCl₃, 300 MHz): δ 9.10 (s, 1H), 7.27-7.24 (m, 1H), 7.23 (br s, 1H), 6.82 (dd, J=8.3, 5.0 Hz, 1H), 5.02 (app t, J=2.9 Hz, 0.5H), 4.96 (app t, J=2.9 Hz, 0.5H), 4.65-4.61 (m, 1H), 4.02-3.94 (m, 1H), 3.70-3.58 (m, 1H), 2.99 (s, 1H), 2.88-2.72 (m, 2H), 2.50-2.38 (m, 1H), 2.12-1.98 (m, 2H), 1.85-1.79 (m, 3H), 1.64-1.58 (m, 3H); RMS (ESI+) m/z calcd for C₁₇H₁₉NO₄Na [M+Na] 324.1206, found 324.1202.

Ethynyl-N-hydroxychromane-2-carboxamide (19). To a solution of 6-ethynyl-N-((tetrahydro-2H-pyran-2-yl)oxy)chromane-2-carboxamide (18, 0.153 g, 0.508 mmol) in MeOH (3.0 mL) was added Amberlyst-15 (0.027 g, washed with MeOH) at room temperature. After 20 h, the mixture was filtered through Celite®, rinsed with MeOH and concentrated. The residue was purified by chromatography on SiO₂ (0-100% EtOAc/hexanes) and the solid was further purified by trituration with EtOAc/hexanes to give 6-ethynyl-N-hydroxychromane-2-carboxamide (19, 0.035 g, 32%) as a white solid: mp 161-163° C.; IR (neat) 3335, 3305, 3266, 2981, 2842, 2105, 1662, 607, 1580, 1491, 1248 cm⁻¹; ¹H NMR (DMSO-d₆, 500 MHz): δ 10.82 (s, 1H), 8.96 (s, 1H), 7.22-7.18 (m, 2H), 6.81 (d, J=9.0 Hz, 1H), 4.56 (dd, J=8.7, 3.2 Hz, 1H), 3.98 (s, 1H), 2.80-2.73 (m, 1H), 2.71-2.66 (m, 1H), 2.14-2.08 (m, 1H), 1.96-1.88 (m, 1H); ¹³C NMR (DMSO-d₆, 100 MHz): δ 166.1, 153.9, 133.0, 130.8, 122.5, 117.0, 113.5, 83.6, 79.0, 74.0, 24.0, 22.6; HRMS (ESI+) m/z calcd for C₁₂H₁₀O₃N [M−H] 216.0655, found 216.0668.

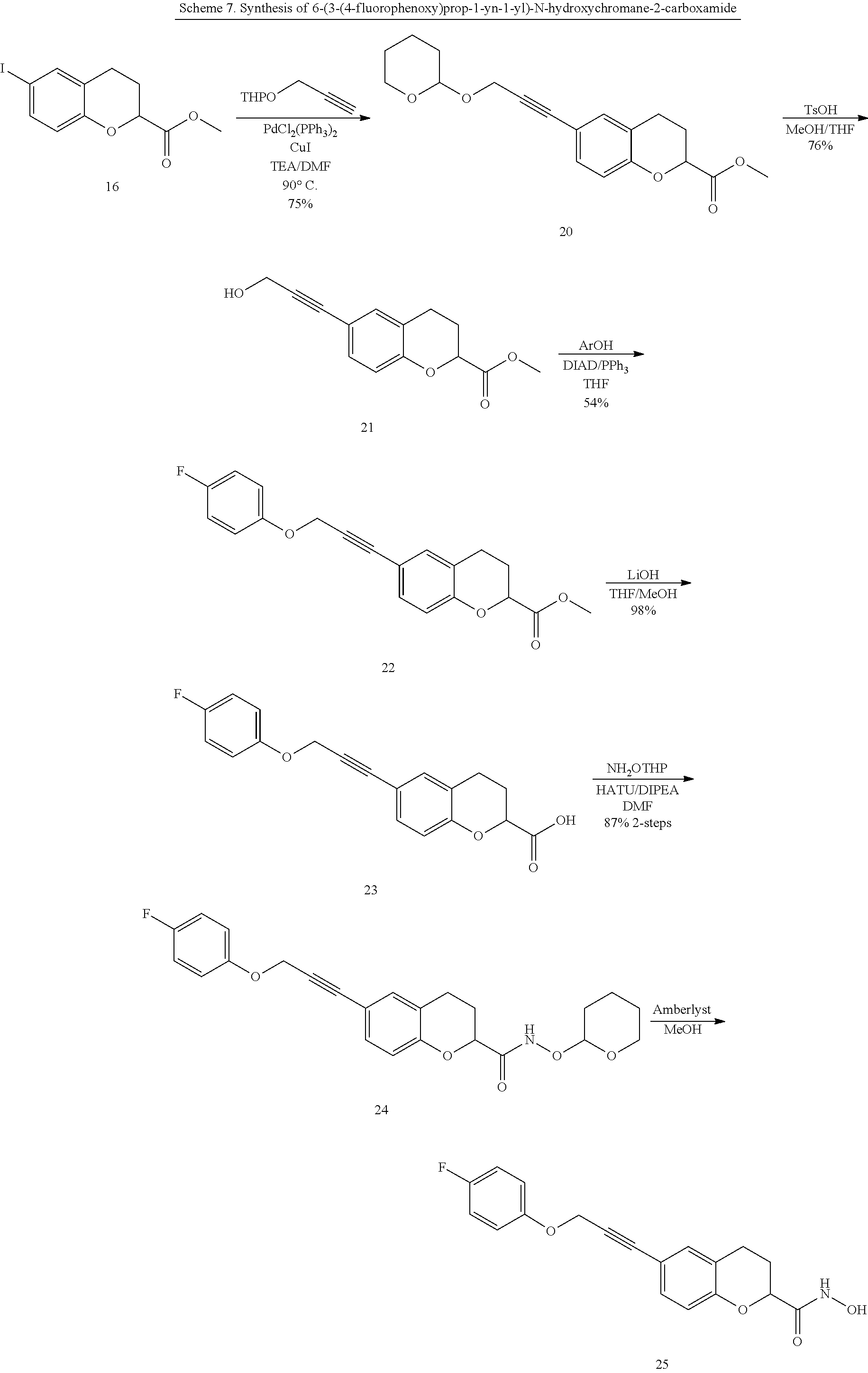

Methyl 6-(3-((tetrahydro-2H-pyran-2-yl)oxy)prop-1-yn-1-yl)chromane-2-carboxylate (20). A solution of methyl 6-iodochromane-2-carboxylate (16, 0.175 g, 0.55 mmol), 2-(prop-2-yn-1-yloxy)tetrahydro-2H-pyran (0.193 g, 1.38 mmol) in DMF (1.5 mL) was degassed by N₂ bubbling (5 min). To this solution was added CuI (0.018 g, 0.09 mmol) followed by PdCl₂(PPh₃)₂ (0.055 g, 0.078 mmol). The reaction mixture was degassed by N₂ bubbling (5 min) and treated with TEA (0.40 mL). The solution was degassed by N₂ bubbling (5 min) and the reaction vial was sealed and submerged into a preheated oil bath at 90° C. After 2 h, the reaction mixture was cooled to room temperature, extracted with EtOAc, washed with brine, dried (Na₂SO₄), filtered and concentrated. The residual oil was purified by flash chromatography on SiO₂ (0-20% EtOAc/hexanes) to give methyl 6-(3-((tetrahydro-2H-pyran-2-yl)oxy)prop-1-yn-1-yl)chromane-2-carboxylate (20, 0.136 g, 75%) as an amber oil: ¹H NMR (CDCl₃, 500 MHz): δ 7.22 (dd, J=1.5, 8.5 Hz, 1H), 7.18 (s, 1H), 6.87 (d, J=8.5 Hz, 1H), 4.91 (app t, J=3.5 Hz, 1H), 4.77 (dd, J=4.0, 4.0 Hz, 1H), 4.49 (q, J_{AB} 15.0 Hz, 2H), 3.92-3.88 (m, 1H), 3.81 (s, 3H), 3.59-3.56 (m, 1H), 2.79-2.75 (m, 1H), 2.74-2.72 (m, 1H), 2.27-2.23 (m, 1H), 2.22-2.20 (m, 1H), 1.86-1.82 (m, 1H), 1.80-1.75 (m, 1H), 1.69-1.55 (m, 6H).

Methyl 6-(3-hydroxyprop-1-yn-1-yl)chromane-2-carboxylate (21). To a solution of methyl 6-(3-((tetrahydro-2H-pyran-2-yl)oxy)prop-1-yn-1-yl)chromane-2-carboxylate (20, 0.196 g, 0.59 mmol) in MeOH/THF (4.0 mL, 3/1) was added p-toluenesulfonic acid monohydrate (0.103 g, 0.598 mmol) at room temperature. After 13 h, the solution was treated with NaHCO₃ and concentrated. The residue was extracted with EtOAc, washed with H₂O, brine, dried (Na₂SO₄), filtered and concentrated. The residue was purified by chromatography on SiO₂ (1/1 CH₂Cl₂/hexanes to 1/1 CH₂Cl₂/EtOAc) to give methyl 6-(3-hydroxyprop-1-yn-1-yl)chromane-2-carboxylate (21, 0.117 g, 76%) as a light yellow solid: mp 136-138° C.; IR (neat) 3481, 2938, 1734, 1493, 1440, 1391, 1354, 1296, 1216, 1127, 1095, 1060, 1033, 1018, 966, 892, 840, 793, 684 cm⁻¹; ¹H NMR (CDC₃, 300 MHz): δ7.18 (dd, J=1.8, 8.4 Hz, 1H), 7.13 (s, 1H), 6.85 (d, J=8.4 Hz, 1H), 4.74 (dd, J=3.9, 3.9 Hz, 1H), 4.54 (s, 2H), 3.79 (s, 3H), 2.82-2.67 (m, 2H), 2.29-2.16 (m, 2H); ¹³C NMR (CDCl₃, 150 MHz): δ171.1, 153.7, 133.0, 131.2, 121.3, 117.1, 114.8, 85.7, 85.5, 73.8, 52.5, 51.7, 24.3, 23.0; HRMS (ESI+) m/z calcd for C₁₄H₁₅O₄ [M+H] 247.0965, found 247.0974.

Methyl 6-(3-(4-fluorophenoxy)prop-1-yn-1-yl)chromane-2-carboxylate (22). To a solution of methyl 6-(3-hydroxyprop-1-yn-1-yl)chromane-2-carboxylate (21, 0.073 g, 0.30 mmol), 4-fluorophenol (0.052 g, 0.46 mmol), PPh₃ (0.107 g, 0.40 mmol) in THE (2.5 mL) was added DIAD (0.070 mL, 0.33 mmol) at 0° C. After 10 min, the solution was warmed to room temperature. After 4 h, the solution was extracted with EtOAc, washed with brine, dried (Na₂SO₄), filtered and concentrated. The residual oil was purified by chromatography on SiO₂ (hexanes to 4/1 hex/EtOAc) to give a mixture of product and SM (4-fluorophenol). The mixture was extracted with CH₂Cl₂, washed with 0.5M NaOH, brine, dried (Na₂SO₄), filtered and concentrated to give methyl 6-(3-(4-fluorophenoxy)prop-1-yn-1-yl)chromane-2-carboxylate (22, 0.054 g, 54%) as a light yellow oil: ¹H NMR (CDCl₃, 400 MHz): δ7.20 (dd, J=2.0, 8.4 Hz, 1H), 7.13 (s, 1H), 6.99-6.96 (m, 4H), 6.85 (d, J=8.4 Hz, 1H), 4.85 (s, 2H), 4.75 (dd, J=3.6, 3.6 Hz, 1H), 3.79 (s, 3H), 2.81-2.66 (m, 2H), 2.28-2.20 (m, 2H); ¹³C NMR (CDCl₃, 150 MHz): δ171.0, 157.6 (J_{CF}=239 Hz), 153.9, 153.8 (J_{CF}=2 Hz), 133.1, 131.3, 121.3, 117.1, 116.2 (J_{CF}=9 Hz), 115.8 (J_{CF}=22 Hz), 114.4, 87.2, 82.2, 73.9, 57.4, 52.5, 24.2, 22.9; ¹⁹F NMR (CDCl₃, 376 MHz): δ−123.2; HRMS (LCMS ESI+) m/z calcd for C₂₀H₁₅O₄F [M+H] 341.1184, found 341.1181.

Lithium 6-(3-(4-fluorophenoxy)prop-1-yn-1-yl)chromane-2-carboxylate (23). A solution of methyl 6-(3-(4-fluorophenoxy)prop-1-yn-1-yl)chromane-2-carboxylate (22, 0.054 g, 0.16 mmol) in THF/MeOH (2.0 mL, 1/3) was treated with 1M LiGH (0.2 mL) at room temperature and kept at this temperature overnight. The solution was concentrated and the residue was treated with PhMe and concentrated (3×) to give lithium 6-(3-(4-fluorophenoxy)prop-1-yn-1-yl)chromane-2-carboxylate (23) that was used without further purification: HRMS (LCMS ESI+) m/z calcd for C₁₉H₁₆O₄F [M+H] 327.1027, found 327.1026.

6-(3-(4-Fluorophenoxy)prop-1-yn-1-yl)-N-((tetrahydro-2H-pyran-2-yl)oxy)chromane-2-carboxamide (24). A solution of O-(tetrahydro-2H-pyran-2-yl)hydroxylamine (0.026 g, 0.22 mmol) in DMF (2.0 mL) was added to lithium 6-(3-(4-fluorophenoxy)prop-1-yn-1-yl)chromane-2-carboxylate (23, 0.053, 0.16 mmol, crude). To this solution was added HATU (0.063 g, 0.16 mmol) followed by DIPEA (0.050 mL, 0.30 mmol) at room temperature. After 24 h, the solution was extracted with EtOAc, washed with brine, dried (Na₂SO₄), filtered and concentrated. The residual oil was purified by chromatography on SiO₂ (ISCO-Rf, 0-100% EtOAc/hexanes) to give 6-(3-(4-fluorophenoxy)prop-1-yn-1-yl)-N-((tetrahydro-2H-pyran-2-yl)oxy)chromane-2-carboxamide (24, 0.059 g, 87%) as an off-white foamy solid: ¹H NMR (CDCl₃, 400 MHz): δ9.12 (s, 1H), 7.18 (dd, J=1.6, 8.4 Hz, 1H), 7.15 (s, 1H), 6.99-6.94 (m, 4H), 6.82-6.79 (m, 1H), 01-4.95 (m, 1H), 4.84 (s, 2H), 4.63-4.59 (m, 1 H), 3.99-3.94 (m, 1H), 3.67-3.59 (m, 1H), 2.87-2.74 (m, 2H), 2.42-2.40 (m, 1H), 2.07-2.03 (m, 1H), 1.82-1.78 (m, 3H), 1.59-1.54 (m, 3H); HRMS (LCMS ESI+) m/z calcd for C₂₄H₂₅O₅NF [M+H] 426.1711, found 426.1709.

6-(3-(4-Fluorophenoxy)prop-1-yn-1-yl)-N-hydroxychromane-2-carboxamide (25). To a solution of 6-(3-(4-fluorophenoxy)prop-1-yn-1-yl)-N-((tetrahydro-2H-pyran-2-yl)oxy)chromane-2-carboxamide (24, 0.101 g, 0.237 mmol) in MeOH (2.5 mL) was added Amberlyst-15 (0.018 g, washed with MeOH) at room temperature. After 24 h, the mixture was filtered through Celite®, rinsed with MeOH and concentrated. The residue was treated with Et₂O and concentrated (2×). The residue was slurried with Et₂O and the solid was collected by filtration and dried in vacuo to give 6-(3-(4-fluorophenoxy)prop-1-yn-1-yl)-N-hydroxychromane-2-carboxamide (25, 0.048 g, 59%) as an off-white solid: mp 149-151° C.; IR (neat) 3178, 1650, 1508, 1492, 1378, 1233, 1204, 1099, 1079, 1023, 1009, 995, 906, 889, 827, 793, 768 cm⁻¹; ¹H NMR (DMSO-d₆, 600 MHz): δ10.83 (s, 1H), 8.97 (s, 1H), 7.17-7.04 (m, 4H), 7.04 (d, J=2.4 Hz, 1H), 7.03 (s, 1H), 6.81 (d, J=9.0 Hz, 1H), 4.98 (s, 2H), 4.56 (dd, J=3.0, 3.0 Hz, 1H), 2.76-2.66 (m, 2H), 2.12-2.09 (m, 1H), 1.94-1.90 (m, 1H); ¹³C NMR (DMSO-d₆, 150 MHz): δ 166.1, 156.8 (J_{CF}=236 Hz), 154.0, 153.7, 132.8, 130.6, 122.5, 117.1, 116.3 (J_{CF}=9 Hz), 115.8 (J_{CF}=24 Hz), 113.2, 86.6, 83.1, 56.7, 23.9, 22.6; ¹⁹F NMR (DMSO-d₆, 470 MHz): δ−123.3; HRMS (ESI+) m/z calcd for C₁₉H₁₇O₄NF [M+H] 342.1136, found 342.1134; ELS purity (100%).

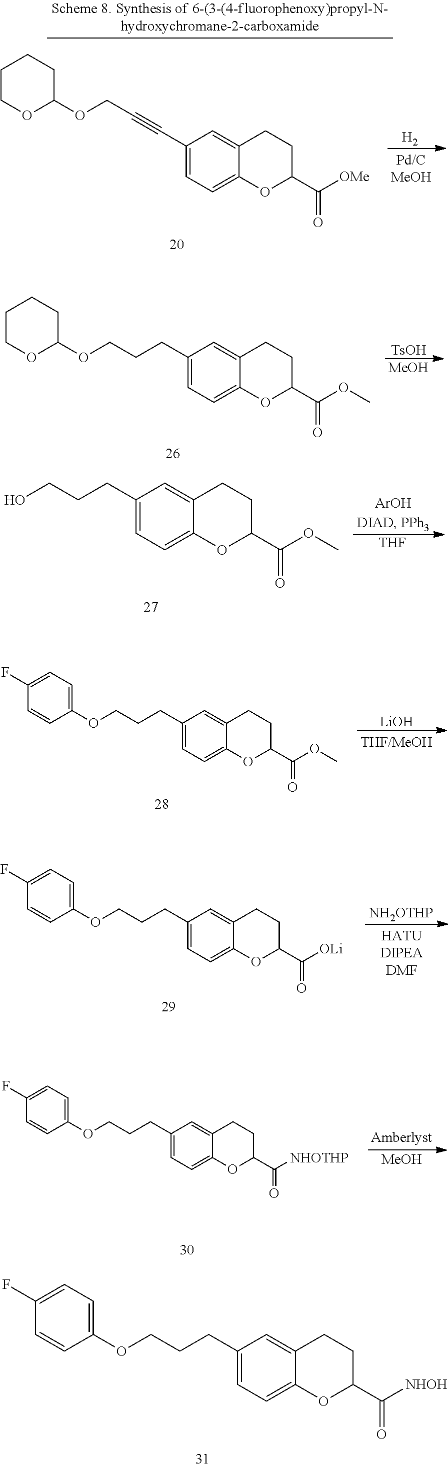

Methyl 6-(3-((tetrahydro-2H-pyran-2-yl)oxy)propyl)chromane-2-carboxylate (26). A solution of methyl 6-(3-((tetrahydro-2H-pyran-2-yl)oxy)prop-1-yn-1-yl)chromane-2-carboxylate (20, 0.207 g, 0.626 mmol) in MeOH (5.0 mL) was evacuated and purged with N₂ (2×). The solution was treated with Pd/C (10%, 0.092 g) and evacuated and purged with H₂ (2×) and kept under H₂ (1 atm-balloon). After 4 h, the solution was filtered through Celite®, rinsed with MeOH and concentrated to give methyl 6-(3-((tetrahydro-2H-pyran-2-yl)oxy)propyl)chromane-2-carboxylate (26, 0.188 g, 90%) as a colorless oil and was used without further purification: ¹H NMR (CDCl₃, 400 MHz): δ 6.93 (d, J=8.0 Hz, 1H), 6.86-6.83 (m, 2H), 4.69 (dd, J=3.2, 7.6 Hz, 1H), 4.57 (app t, J=2.4 Hz, 1H), 3.88-3.84 (m, 1H), 3.79 (s, 3H), 3.76-3.72 (m, 1H), 3.52-3.46 (m, 1H), 3.42-3.36 (m, 1H), 2.81-2.69 (m, 2H), 2.62-2.57 (m, 2H), 2.29-2.24 (m, 1H), 2.23-2.15 (m, 1H), 1.91-1.82 (m, 2H), 1.74-1/70 (m, 2H), 1.69-1.52 (m, 4H); HRMS (ESI+) m/z calcd for C₁₉H₂₆O₅Na [M+Na] 357.1672, found 357.1669.

Methyl 6-(3-hydroxypropyl)chromane-2-carboxylate (27). A solution of 6-(3-((tetrahydro-2H-pyran-2-yl)oxy)prop-1-yn-1-yl)chromane-2-carboxylate (26, 0.188 g, 0.56 mmol) in MeOH (5.0 mL) was treated with p-toluenesulfonic acid monohydrate (0.112 g, 0.577 mmol) at room temperature. After 18 h, the solution was treated with NaHCO₃ and concentrated.

The residue was extracted with EtOAc, washed with brine, dried (Na₂SO₄), filtered and concentrated to give methyl 6-(3-hydroxypropyl)chromane-2-carboxylate (27, 0.136 g, 97%) as a colorless oil that was used without further purification. A solution of methyl 6-(3-((tetrahydro-2H-pyran-2-yl)oxy)prop-1-yn-1-yl)chromane-2-carboxylate (26, 0.165 g, 0.499 mmol) in MeOH (5.0 mL) was evacuated and purged with N₂ (2×). The solution was treated with Pd/C (10%, 0.055 g) and evacuated and purged with H₂ (2×) and kept under H₂ (1-atm balloon). After 1 d, the mixture was filtered through Celite®, rinsed with MeOH and concentrated to give methyl 6-(3-hydroxypropyl)chromane-2-carboxylate (27, 0.105 g, 84%) as a colorless oil: ¹H NMR (CDCl₃, 300 MHz): δ 6.93 (d, J=8.1 Hz, 1H), 6.85-6.82 (m, 2H), 4.70 (dd, J=3.3, 3.3 Hz, 1H), 3.78 (s, 3H), 3.64 (t, J=6.3 Hz, 2H), 2.80-2.71 (m, 2H), 2.59 (t, J=7.2 Hz, 2H), 2.26-2.14 (m, 2H), 1.96 (br s, 1H), 1.88-1.81 (m, 2H); ¹³C NMR (CDCl₃, 125 MHz): δ 151.5, 134.1, 129.1, 127.5, 120.9, 116.7, 73.7, 62.2, 52.3, 34.3, 31.1, 24.7, 23.3; HRMS (ESI+) m/z calcd for C₁₄H₁₉O₄ [M+H] 251.1278, found 251.1277.

Methyl 6-(3-(4-fluorophenoxy)propyl)chromane-2-carboxylate (28). A solution of methyl 6-(3-hydroxypropyl)chromane-2-carboxylate methyl 6-(3-hydroxypropyl)chromane-2-carboxylate (27, 0.136 g, 0.54 mmol), 4-fluorophenol (0.063 g, 0.56 mmol), PPh₃ (0.145 g, 0.55 mmol) in THE (1.0 mL) was cooled to 0° C. and treated with DIAD (0.12 mL, 0.57 mmol). After 10 min, the solution was warmed to room temperature and kept at this temperature overnight.

The solution was extracted with EtOAc, washed with satd. NaHCO₃, brine, dried (Na₂SO₄), filtered and concentrated. The residual oil was purified by chromatography on SiO₂ (ISCO-Rf, 0-100% EtOAc/hexanes) followed by flash chromatography on SiO₂ (hexanes to 2/1 hexanes/Et₂O) to give methyl 6-(3-(4-fluorophenoxy)propyl)chromane-2-carboxylate (28, g, approx. 90% purity, that contained some 4-fluorophenol SM) and was used without further purification: HRMS (ESI+) m/z calcd for C₂₀H₂₁O₄FNa [M+Na] 367.1316, found 367.1313.

Lithium 6-(3-(4-fluorophenoxy)propyl)chromane-2-carboxylate (29). A solution of methyl 6-(3-(4-fluorophenoxy)propyl)chromane-2-carboxylate (28, 0.061 g, 0.16 mmol, 90% purity) in THF/MeOH (3 mL, 1/2) was treated with 1M LiGH (0.20 mL) at room temperature. After 18 h, the solution was concentrated and the residue was treated with PhMe and concentrated (3×). The crude lithium 6-(3-(4-fluorophenoxy)propyl)chromane-2-carboxylate (29) was used without further purification: HRMS (ESI+) m/z calcd for C₁₉H₁₈O₄F [M−H] 329.1184, found 329.1188.

6-(3-(4-Fluorophenoxy)propyl)-N-((tetrahydro-2H-pyran-2-yl)oxy)chromane-2-carboxamide (30). The lithium 6-(3-(4-fluorophenoxy)propyl)chromane-2-carboxylate (29, 0.054 g, crude) was treated with O-(tetrahydro-2H-pyran-2-yl)hydroxylamine (0.024 g, 0.20 mmol) in DMF (2.0 mL) followed by HATU (0.067 g, 0.17 mmol) and DIPEA (0.050 mL, 0.30 mmol) at room temperature. After 5 h, the solution was extracted with EtOAc, washed with brine, dried (Na₂SO₄), filtered and concentrated. The residual oil was purified by chromatography on SiO₂ (ISCO-Rf, 0-100% EtOAc/hexanes) to give 6-(3-(4-fluorophenoxy)propyl)-N-((tetrahydro-2H-pyran-2-yl)oxy)chromane-2-carboxamide (30, 0.055 g, 80% 2-steps) as a colorless oil: ¹H NMR (CDCl₃, 400 MHz): δ 9.13 (s, 1H), 6.95-6.93 (m, 3H), 6.82-6.81 (m, 1H), 6.83-6.77 (m, 3H), 5.03-4.96 (m, 1H), 4.62-4.57 (m, 1H), 3.98-3.91 (m, 1H), 3.90-3.88 (m, 2H), 2.85-2.75 (m, 3H), 2.72-2.68 (m, 3H), 2.42-2.40 (m, 1H), 2.06-2.03 (m, 2H), 2.01-1.79 (m, 3H), 1.65-1.55 (m, 5H); ¹⁹F NMR (CDCl₃, 376 MHz): δ−124.2; HRMS (ESI+) m/z calcd for C₂₄H₂₅O₅NFNa [M+Na] 452.1844, found 452.1843.

6-(3-(4-Fluorophenoxy)propyl)-N-hydroxychromane-2-carboxamide (31). A solution of 6-(3-(4-fluorophenoxy)propyl)-N-((tetrahydro-2H-pyran-2-yl)oxy)chromane-2-carboxamide (30, 0.055 g, 0.13 mmol) in MeOH (2.0 mL) was treated with Amberlyst-15 (0.020 g, washed with MeOH) at room temperature. After 16 h, additional Amberlyst-15 was added (0.010 g) and the solution was heated to reflux for 2 h. The solution was cooled to room temperature, filtered through Celite®, rinsed with MeOH and concentrated. The solid residue was slurried with Et₂O (2×) and the product was collected by filtration and dried in vacuo at 50° C. to give 6-(3-(4-fluorophenoxy)propyl)-N-hydroxychromane-2-carboxamide (31, 0.019 g, 43%) as an off-white solid: mp 145-147° C.; IR (neat) 3275, 2918, 1637, 1544, 1507, 1495, 1239, 1207, 1095, 1073, 1049, 1010, 896, 827, 799, 757 cm⁻¹; ¹H NMR (DMSO-d₆, 600 MHz): δ 10.74 (s, 1H), 8.90 (s, 1H), 7.11-7.08 (m, 2H), 6.94-6.90 (m, 4H), 6.72 (d, J=8.4 Hz, 1H), 4.46 (dd, J=3.0, 3.0 Hz, 1H), 3.91 (t, J=6.0 Hz, 2H), 2.75-2.72 (m, 1H), 2.67-2.60 (m, 3H), 2.10-2.07 (m, 1H), 1.96-1.92 (m, 3H); ¹³C NMR (DMSO-d₆, 150 MHz): δ156.4 (J_{CF}=236 Hz), 154.9, 151.5, 133.1, 129.1, 127.0, 121.5, 116.4, 115.7 (J_{CF}=22 Hz, 115.6, 73.8, 67.3, 30.6, 30.5, 24.3, 23.0; HRMS (ESI+) m/z calcd for C₁₉H₁₉O₄NF [M−H] 344.1293, found 344.1290.

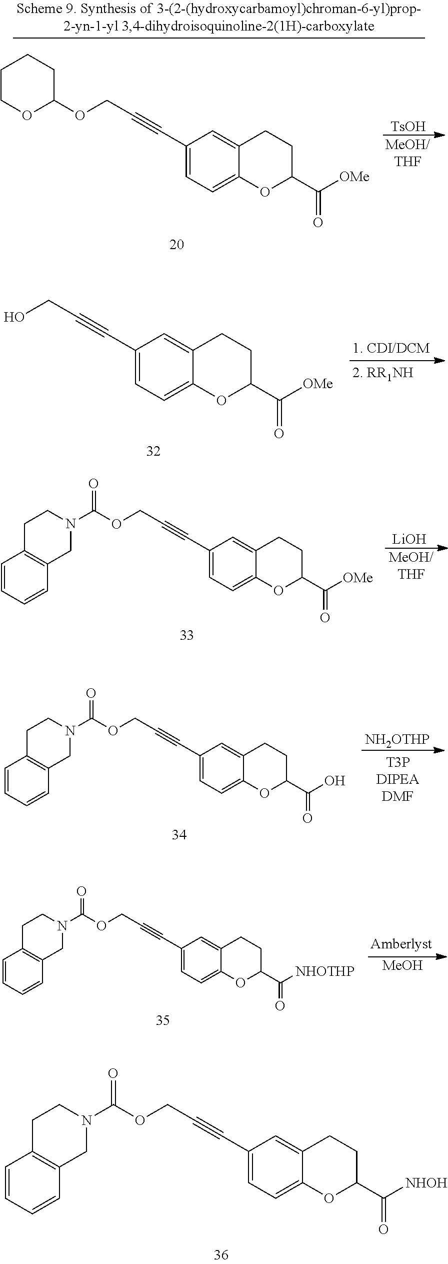

Methyl 6-(3-hydroxyprop-1-yn-1-yl)chromane-2-carboxylate (32). To a solution of methyl 6-(3-((tetrahydro-2H-pyran-2-yl)oxy)prop-1-yn-1-yl)chromane-2-carboxylate (20, 0.178 g, 0.539 mmol) in MeOH/THF (3.0 mL, 3/1) was added p-toluenesulfonic acid monohydrate (0.048 g, 0.25 mmol) at room temperature. After 23 h, the solution was treated with NaHCO₃ and concentrated. The residue was extracted with EtOAc, washed with H₂O, brine, dried (Na₂SO₄), filtered and concentrated. The residue was purified by chromatography on SiO₂ (1/1 hexanes/CH₂Cl₂ to 1/1 EtOAc/CH₂Cl₂) to give methyl 6-(3-hydroxyprop-1-yn-1-yl)chromane-2-carboxylate (32, 0.104 g, 78%) as a light yellow solid: ¹H NMR (CDCl₃, 400 MHz): δ 7.18 (dd, J=2.0, 8.4 Hz, 1H), 7.13 (s, 1H), 6.84 (d, J=8.4 Hz, 1H), 4.74 (dd, J=3.6, 3.6 Hz, 1H), 4.45 (s, 2H), 3.78 (s, 3H), 2.82-2.67 (m, 2H), 2.28-2.16 (m, 2H).

3-(2-(Methoxycarbonyl)chroman-6-yl)prop-2-yn-1-yl 3,4-dihydroisoquinoline-2(1H)-carboxylate (33). A solution of methyl 6-(3-hydroxyprop-1-yn-1-yl)chromane-2-carboxylate (32, 0.104 g, 0.422 mmol) in CH₂Cl₂ (2.5 mL) was treated with carbonyl diimidazole (0.069 g, 0.42 mmol) at room temperature. After 3 h, tetrahydroisoquinoline (0.068 g, 0.51 mmol) in CH₂Cl₂ (0.5 mL) was added dropwise. After 19 h, the solution was extracted with CH₂Cl₂, washed with 0.5M HCl, brine, dried (Na₂SO₄), filtered and concentrated. The residual oil was purified by chromatography on SiO₂ (ISCO-Rf, 0-100% EtOAc/hexanes) to give 3-(2-(methoxycarbonyl)chroman-6-yl)prop-2-yn-1-yl 3,4-dihydroisoquinoline-2(1H)-carboxylate (33, 0.139 g, 81%) as a light yellow oil: ¹H NMR (CDCl₃, 400 MHz): δ 7.25 (dd, J=2.0, 8.4 Hz, 1H), 7.22-7.18 (m, 3H), 7.17-7.14 (m, 2H), 6.88 (d, J=8.4 Hz, 1H), 4.99 (s, 2H), 4.77 (dd, J=4.0, 4.0 Hz, 1H), 4.68 (br s, 2H), 3.81 (s, 3H), 3.75 (t, J=6.0 Hz, 2H), 2.89 (t, J=5.6 Hz, 2H), 2.84-2.69 (m, 2H), 2.32-2.17 (m, 2H); HRMS (ESI+) m/z calcd for C₂₄H₂₃O₅NNa [M+Na]428.1468, found 428.1465.

6-(3-((1,2,3,4-Tetrahydroisoquinoline-2-carbonyl)oxy)prop-1-yn-1-yl)chromane-2-carboxylic acid (34). A solution of 3-(2-(methoxycarbonyl)chroman-6-yl)prop-2-yn-1-yl 3,4-dihydroisoquinoline-2(1H)-carboxylate (33, 0.139 g, 0.343 mmol) in MeOH/THF (3.0 mL, 2/1) was treated with 1M LiGH (0.50 mL) at room temperature. After 2.5 h, the solution was concentrated and the residue was acidified by 2M HCl, extracted with EtOAc, washed with brine, dried (Na₂SO₄), filtered and concentrated to give 6-(3-((1,2,3,4-tetrahydroisoquinoline-2-carbonyl)oxy)prop-1-yn-1-yl)chromane-2-carboxylic (34, 0.110 g, 82%) as a light yellow oil that was used without further purification: HRMS (LCMS ESI+) m/z calcd for C₂₃H₂₀O₅N [M−H]390.1336, found 390.1346.

3-(2-(((Tetrahydro-2H-pyran-2-yl)oxy)carbamoyl)chroman-6-yl)prop-2-yn-1-yl 3,4-dihydroiso-quinoline-2(1H)-carboxylate (35) To a solution of 6-(3-((1,2,3,4-tetrahydroisoquinoline-2-carbonyl)oxy)prop-1-yn-1-yl)chromane-2-carboxylic acid (34, 0.11 g, 0.28 mmol) in DMF (1.0 mL) was added O-(tetrahydro-2H-pyran-2-yl)hydroxylamine (0.049 g, 0.42 mmol) followed by T3P (0.252 g, 50% EtOAc) in DMF (1.0 mL) followed by DIPEA (0.10 mL, 0.60 mmol) at room temperature. After 9 h, the solution was extracted with EtOAc, washed with 0.5M HCl, brine, dried (Na₂SO₄), filtered and concentrated. The residual oil was extracted with CH₂Cl₂, washed with 0.5M LiCl, dried (Na₂SO₄), filtered and concentrated to give 3-(2-(((tetrahydro-2H-pyran-2-yl)oxy)carbamoyl)chroman-6-yl)prop-2-yn-1-yl 3,4-dihydroiso-quinoline-2(1H)-carboxylate (35, 0.114 g, 79%, small amount of residual solvent) as a light yellow oil that was used without further purification: ¹H NMR (CDCl₃, 500 MHz): δ9.12 (s, 0.5H), 9.11 (s, 0.5H), 7.23-7.11 (m, 6H), 6.81 (app t, J=8.5 Hz, 1H), 5.01 (br s, 0.5H), 4.95 (br s, overlapping, 0.5H), 4.95 (s, 2H), 4.66-4.61 (m, 3H), 3.98 (app t, J=9.5 Hz, 1H), 3.73 (t, J=6.0 Hz, 2H), 3.67-3.60 (m, 1H), 2.88-2.76 (m, 4H), 2.45-2.39 (m, 1H), 2.09-2.02 (m, 1H), 1.92-1.79 (m, 3H), 1.67-1.59 (m, 3H); HRMS (ESI+) m/z calcd for C₂₈H₃₀O₆N₂Na [M+Na] 513, 1996, found 513.1993.

3-(2-(Hydroxycarbamoyl)chroman-6-yl)prop-2-yn-1-yl 3,4-dihydroisoquinoline-2(1H)-carboxylate (36). A solution of 3-(2-(((tetrahydro-2H-pyran-2-yl)oxy)carbamoyl)chroman-6-yl)prop-2-yn-1-yl 3,4-dihydroiso-quinoline-2(1H)-carboxylate (35, 0.114 g, 0.22 mmol) in MeOH (3.0 mL) was treated with Amberlyst-15 (0.076 g) at room temperature. After 22 h, the mixture was filtered through Celite®, rinsed with MeOH and concentrated. The residue was purified by chromatography on SiO₂ (ISCO-Rf, 0-10% MeOH/CH₂Cl₂) to give 3-(2-(hydroxycarbamoyl)chroman-6-yl)prop-2-yn-1-yl 3,4-dihydroisoquinoline-2(1H)-carboxylate (36, 0.069 g, 77%) as a white foamy solid: mp 90-92° C.; IR (neat) 3268, 2926, 1669, 1608, 1492, 132, 1340, 1293, 1225, 1117, 1091, 1003, 931, 867, 824, 749 cm⁻¹; ¹H NMR (DMSO-d₆, 600 MHz): δ10.83 (s, 1H), 8.96 (s, 1H), 7.19-7.17 (m, 6H), 6.81 (d, J=8.4 Hz, 1H), 4.94 (s, 2H), 4.60-4.56 (m, overlapping signals, 3H), 3.62 (br s, 2H), 2.82-2.80 (m, 2H), 2.75-2.68 (m, 2H), 2.12-2.09 (m, 1H), 1.93-1.90 (m, 1H); ¹³C NMR (DMSO-d₆, 150 MHz): δ166.1, 154.1, 154.0, 134.3, 133.0, 130.7, 128.6, 126.4, 126.3, 126.2, 122.5, 117.1, 113.2, 85.6, 83.2, 74.9, 53.3, 45.3, 41.4, 28.2, 23.9, 22.6; HRMS (ESI+) m/z calcd for C₂₃H₂₁O₅N₂ [M−H] 405.1445, found 405.1458; ELS purity (100%).

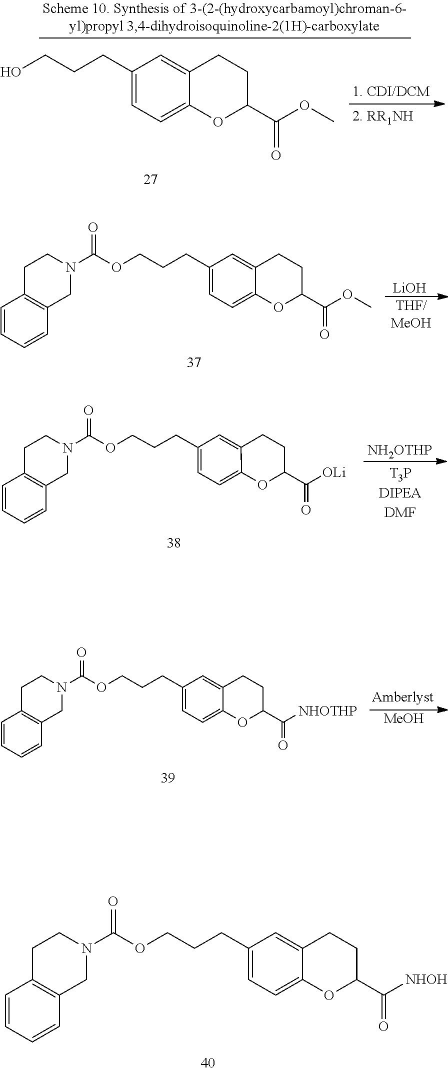

3-(2-(Methoxycarbonyl)chroman-6-yl)propyl 3,4-dihydroisoquinoline-2(1H)-carboxylate (37). A solution of methyl 6-(3-hydroxypropyl)chromane-2-carboxylate (27, 0.082 g, 0.31 mmol) in CH₂Cl₂ (1.5 mL) was treated with carbonyl diimidazole (0.052 g, 0.31 mmol) at room temperature. After 4 h, the solution was treated with 1,2,3,4-tetrahydroisoquinoline (0.053 g, 0.40 mmol) in CH₂Cl₂ (0.5 mL) at room temperature. After 19 h, the solution was extracted with CH₂Cl₂, washed with brine, dried (Na₂SO₄), filtered and concentrated. The residual oil was purified by chromatography on SiO₂ (ISCO-Rf, 0-100% EtOAc/hexanes) to give 3-(2-(methoxycarbonyl)chroman-6-yl)propyl 3,4-dihydroisoquinoline-2(1H)-carboxylate (37, 0.089 g, 70%) as a colorless oil: IR (neat) 2949, 2850, 1755, 1736, 1692, 1497, 1428, 1295, 1200, 1120, 1094, 1015, 930, 821, 746 cm⁻¹; ¹H NMR (CDCl₃, 400 MHz): δ7.20-7.17 (m, 2H), 7.15-7.12 (m, 2H), 6.96-6.93 (m, 1H), 6.86-6.84 (m, 2H), 4.69-4.68 (m, 1H), 4.62-4.59 (m, 2H), 4.15 (t, J=6.8 Hz, 2H), 3.79 (s, 3H), 3.71-3.68 (m, 2H), 2.85 (t, J=5.2 Hz, 2H), 2.79-2.72 (m, 2H), 2.62 (t, J=7.2 Hz, 2H), 2.26-2.24 (m, 1H), 2.20-2.13 (m, 1H), 1.95 (app pent, J=6.4 Hz, 2H); ¹³C NMR (CDCl₃, 150 MHz): δ171.4, 155.7, 151.6, 133.7, 129.1, 128.8, 128.5, 127.5, 126.4, 126.3, 126.26, 126.18, 120.9, 116.8, 73.7, 64.9, 52.4, 45.6, 41.5, 41.2, 31.4, 30.7, 28.8, 24.7, 23.4; HRMS (ESI+) m/z calcd for C₂₄H₂₈O₅N [M+H] 410.1962, found 410.1959.

Lithium 6-(3-((1,2,3,4-tetrahydroisoquinoline-2-carbonyl)oxy)propyl)chromane-2-carboxylate (38). A solution of 3-(2-(methoxycarbonyl)chroman-6-yl)propyl 3,4-dihydroisoquinoline-2(1H)-carboxylate (37, 0.089 g, 0.22 mmol) in MeOH/THF (2.0 mL, 3/1) was treated with 1M LiGH (0.30 mL) at room temperature. After 17 h, the solution was concentrated and the residue was treated with PhMe and concentrated (3×) to give lithium 6-(3-((1,2,3,4-tetrahydroisoquinoline-2-carbonyl)oxy)propyl)chromane-2-carboxylate (38) that was used without further purification.

3-(2-(((Tetrahydro-2H-pyran-2-yl)oxy)carbamoyl)chroman-6-yl)propyl 3,4-dihydroisoquinoline-2(1H)-carboxylate (39). A mixture of lithium 6-(3-((1,2,3,4-tetrahydroisoquinoline-2-carbonyl)oxy)propyl)chromane-2-carboxylate (38, 0.088 g, crude), 0-(tetrahydro-2H-pyran-2-yl)hydroxylamine (0.039 g, 0.33 mmol) in DMF (2.0 mL) was treated with T3P (0.236 g, 0.37 mmol, 50% EtOAc soln) in DMF (1.0 mL) followed by DIPEA (0.10 mL, 0.60 mmol) at room temperature. After 2 d, the solution was extracted with EtOAc, washed with brine, dried (Na₂SO₄), filtered and concentrated. The residual oil was purified by chromatography on SiO₂ (ISCO-Rf, 0-100% EtOAc/hexanes) to give semi-pure product. The material was repurified by chromatography on SiO₂ (0-10% MeOH/CH₂Cl₂) to give 3-(2-(((tetrahydro-2H-pyran-2-yl)oxy)carbamoyl)chroman-6-yl)propyl 3,4-dihydroisoquinoline-2(1H)-carboxylate (39, g, 76%, approx. 90% purity) as a white foamy solid: ¹H NMR (CDCl₃, 500 MHz): δ9.18 (s, 1H), 7.18-7.09 (m, 4H), 6.93 (d, J=8.2 Hz, 1H), 6.87 (s, 1H), 6.78 (app t, J=7.6 Hz, 1H), 5.02 (br s, 0.5H), 4.96 (br s, 0.5H), 4.58-4.55 (m, 3H), 4.14 (t, J=6.5 Hz, 2H), 4.01-3.96 (m, 1H), 3.67-3.62 (m, 3H), 2.84-2.72 (m, 4H), 2.61 (t, J=7.5 Hz, 2H), 2.40-2.37 (m, 1H), 1.97-1.91 (m, 3H), 1.88-1.79 (m, 3H), 1.64-1.57 (m, 3H); HRMS (ESI+) m/z calcd for C₂₈H₃₄O₆N₂Na [M+Na] 517.2309, found 517.2306.

3-(2-(Hydroxycarbamoyl)chroman-6-yl)propyl 3,4-dihydroisoquinoline-2(1H)-carboxylate (40). A solution of 3-(2-(((tetrahydro-2H-pyran-2-yl)oxy)carbamoyl)chroman-6-yl)propyl 3,4-dihydroisoquinoline-2(1H)-carboxylate (39, 0.111 g, approx. 90% purity) in MeOH (2.0 mL) was treated with Amberlyst-15 (0.022 g, washed with MeOH) at room temperature and kept at this temperature overnight. The solution was heated to reflux for 1 h, cooled to room temperature, filtered through Celite®, rinsed with MeOH and concentrated. The residual oil was purified by chromatography on SiO₂ (ISCO-Rf, 0-10% MeOH/CH₂Cl₂) to give recovered SM (0.091 g) that was re-subjected to the reaction conditions: MeOH (2.0 mL) and Amberlyst-15 (0.029 g, washed with MeOH). After 15 h, additional Amberlyst-15 (0.034 g) was added. After 14 h, the mixture was filtered through Celite®, rinsed with MeOH and concentrated. The residual oil was purified by chromatography on SiO₂ (ISCO-Rf, 0-10% MeOH/CH₂Cl₂) to give 3-(2-(hydroxycarbamoyl)chroman-6-yl)propyl 3,4-dihydroisoquinoline-2(1H)-carboxylate (40, 0.048 g, 58%) as a hard sticky solid: mp 82-84° C.; IR (neat) 3218, 2920, 1665, 1495, 1426, 1296, 1219, 1121, 1097, 1006, 928, 813, 745 cm⁻¹; ¹H NMR (DMSO-d₆, 600 MHz): δ10.75 (s, 1H), 8.92 (s, 1H), 7.18-7.16 (m, 4H), 6.92-6.88 (m, 2H), 6.72 (d, J=8.4 Hz, 1H), 4.52 (br s, 2H), 4.45 (d, J=7.2 Hz, 1H), 4.02 (t, J=6.0 Hz, 2H), 3.58 (t, J=5.4 Hz, 2H), 2.80-2.73 (m, 3H), 2.66-2.63 (m, 1H), 2.55 (t, J=7.2 Hz, 2H), 2.09-2.07 (m, 1H), 1.91-1.86 (m, 3H); ¹³C NMR (DMSO-d₆, 150 MHz): δ166.5, 154.8, 151.5, 134.4, 133.0, 129.1, 128.6, 127.0, 126.4, 126.2, 126.1, 121.5, 116.4, 73.8, 64.3, 45.2, 40.0, 30.7, 30.3, 24.4, 23.1; HRMS (ESI+) m/z calcd for C₂₃H₂₇O₅N₂Na [M+H] 411.1914, found 411.1914; ELS purity (99.5%).

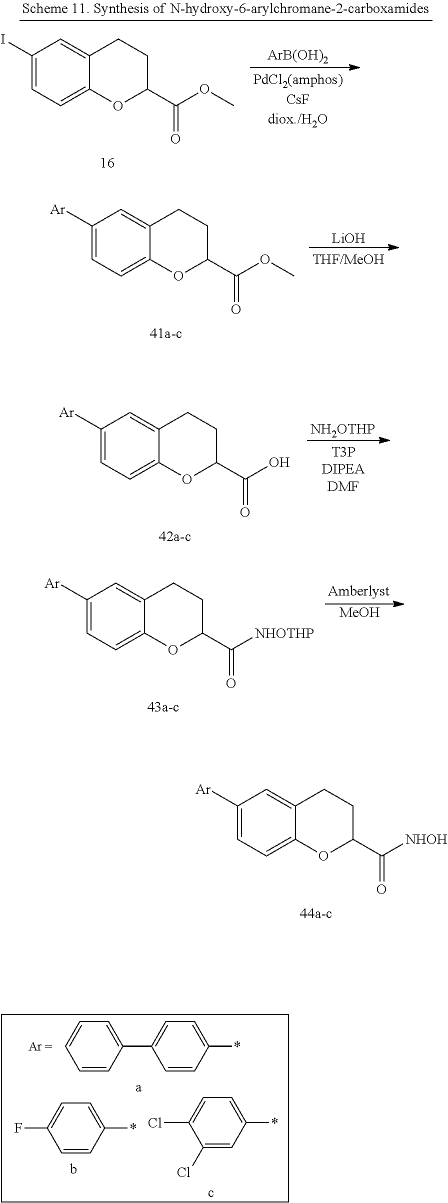

Methyl 6-([1,1′-biphenyl]-4-yl)chromane-2-carboxylate (41a). A solution of methyl 6-iodochromane-2-carboxylate (16, 0.178 g, 0.560 mmol), 4-biphenylboronic acid (0.182 g, 0.919 mmol) in dioxane/H₂O (1.2 mL/0.2 mL) was degassed by N₂ bubbling (5 min). To this solution was added PdCl₂ (amphos) (0.028 g, 0.039 mmol) followed by CsF (0.178 g, 1.16 mmol). The mixture was degassed by N₂ bubbling (5 min). The reaction vial was sealed and heated at 90° C. After 1.5 h, the solution was cooled to room temperature, extracted with EtOAc, washed with 0.5M NaOH, brine, dried (Na₂SO₄), filtered and concentrated. The residue was absorbed onto Celite® and purified by chromatography on SiO₂ (ISCO-Rf, 0-100% EtOAc/hexanes) to give methyl 6-([1,1′-biphenyl]-4-yl)chromane-2-carboxylate (41a, 0.060 g, 31%) as an off-white solid: ¹H NMR (CDCl₃, 300 MHz): δ7.66-7.59 (m, 6H), 7.48-7.32 (m, 5H), 7.03 (d, J=8.4 Hz, 1H), 4.79 (dd, J=3.9, 3.9 Hz, 1H), 3.82 (s, 3H), 2.92-2.84 (m, 2H), 2.34-2.23 (m, 2H).

Methyl 6-(4-fluorophenyl)chromane-2-carboxylate (41b). A solution of methyl 6-iodochromane-2-carboxylate (16, 0.056 g, 0.18 mmol), 4-fluorophenylboronic acid (0.04 g, 0.31 mmol) in dioxane/H₂O (0.6 mL/0.1 mL) was degassed by N₂ bubbling (5 min). To this solution was added PdCl₂ (amphos) (0.011 g, 0.015 mmol) followed by CsF (0.064 g, 0.42 mmol). The mixture was degassed by N₂ bubbling (5 min). The reaction vial was sealed and placed into preheated sand bath at 90° C. After 1 h, the solution was cooled to room temperature, extracted with EtOAc, washed with satd. NH₄Cl, brine, dried (Na₂SO₄), filtered and concentrated. The residue was absorbed onto Celite® and purified by chromatography on SiO₂ (ISCO-Rf, 0-100% EtOAc/hexanes) to give semi-pure product. The residual oil was extracted with CH₂Cl₂, washed with satd. NaHCO₃, brine, dried (Na₂SO₄), filtered and concentrated to give product methyl 6-(4-fluorophenyl)chromane-2-carboxylate (41b, 0.040 g, 71%, 90% purity) that was used without further purification: ¹H NMR (CDCl₃, 400 MHz): δ7.48-7.45 (m, 2H), 7.30 (dd, J=2.4, 8.4 Hz, 1H), 7.20 (d, J=2.0 Hz, 1H), 7.11-7.06 (m, 2H), 7.00 (d, J=8.4 Hz, 1H), 4.78 (dd, J=3.6, 3.6 Hz, 1H), 3.81 (s, 3H), 2.92-2.77 (m, 2H), 2.35-2.19 (m, 2H); ¹⁹F NMR (CDCl₃, 376 MHz): δ−116.7.

Methyl 6-(3,4-dichlorophenyl)chromane-2-carboxylate (41c). A solution of methyl 6-iodochromane-2-carboxylate (16, 0.140 g, 0.440 mmol), 3,4-dichloro-phenylboronic acid (0.133 g, 0.697 mmol) in dioxane/H₂O (1.2 mL/0.2 mL) was degassed by N₂ bubbling (5 min). To this solution was added PdCl₂ (amphos) (0.021 g, 0.029 mmol) followed by CsF (0.148 g, 0.96 mmol). The mixture was degassed by N₂ bubbling (5 min). The reaction vial was sealed and placed into preheated sand bath at 90° C. After 1.5 h, the solution was cooled to room temperature, extracted with EtOAc, washed with 0.5M NaOH, brine, dried (Na₂SO₄), filtered and concentrated. The residue was absorbed onto SiO₂ and purified by chromatography on SiO₂ (ISCO-Rf, 0-100% EtOAc/hexanes) to give methyl 6-(3,4-dichlorophenyl)chromane-2-carboxylate (41c, 0.094 g, 63%) as an amber oil that was used without further purification: ¹H NMR (CDCl₃, 400 MHz): δ 7.60 (d, J=2.0 Hz, 1H), 7.45 (d, J=8.4 Hz, 1H), 7.35-7.29 (m, 2H), 7.22 (s, 1H), 7.00 (d, J=8.4 Hz, 1H), 4.79 (dd, J=3.6, 3.6 Hz, 1H), 3.81 (s, 3H), 2.91-2.77 (m, 2H), 2.34-2.21 (m, 2H); HRMS (ESI+) m/z calcd for C₁₇H₁₄O₃Cl₂Na [M+Na] 359.0212, found 359.0210.

6-([1,1′-Biphenyl]-4-yl)chromane-2-carboxylic acid (42a). To a suspension of methyl 6-([1,1′-biphenyl]-4-yl)chromane-2-carboxylate (41a, 0.060 g, 0.17 mmol) in THF/MeOH (4.0 mL, 1/1) was added 1M LiGH (0.25 mL) at room temperature. Additional THE (1.0 mL) was added after 30 min. After approx. 2 h, the mixture became homogeneous. After 22 h, the solution was concentrated and the residue was diluted with H₂O and acidified with 2M HCl, extracted with EtOAc, washed with brine, dried (Na₂SO₄), filtered and concentrated to give 6-([1,1′-biphenyl]-4-yl)chromane-2-carboxylic acid (42a, 0.055 g, 86%, contained small amount of residual solvent) as an off-white solid that was used without further purification: HRMS (ESI+) m/z calcd for C₂₂H₁₇O₃ [M−H] 329.1172, found 329.1141.

6-(4-Fluorophenyl)chromane-2-carboxylic acid (42b). To a solution of methyl 6-(4-fluorophenyl)chromane-2-carboxylate (41b, 0.128 g, 90% purity, 0.40 mmol) in MeOH/THF (3.0 mL, 2/1) was added 1M LiGH (0.60 mL) at room temperature. After 2 d, the solution was concentrated. The residue was acidified with 2M HCl, extracted with EtOAc, washed with brine, dried (Na₂SO₄), filtered and concentrated to give 6-(4-fluorophenyl)chromane-2-carboxylic acid (42b, 0.104 g, 95%) as a beige solid that was used without further purification: HRMS (ESI+) m/z calcd for C₁₆H₁₂O₃F [M−H] 271.0765, found 271.0777.

6-(3,4-Dichlorophenyl)chromane-2-carboxylic acid (42c). To a solution of methyl 6-(3,4-dichlorophenyl)chromane-2-carboxylate (41c, 0.093 g, 0.28 mmol) in MeOH/THF (3.0 mL, 2/1) was added 1M LiGH (0.30 mL) at room temperature. After 21 h, the solution was concentrated and the residue was diluted with H₂O and acidified with 3M HCl, extracted with EtOAc (2×). The combined organic layer was washed with brine, dried (Na₂SO₄), filtered and concentrated to give crude 6-(3,4-dichlorophenyl)chromane-2-carboxylic acid (42c) as amber oil that was used without further purification: HRMS (ESI+) m/z calcd for C₁₆H₁₁O₃Cl₂ [M−H]321.0080, found 321.0063.

6-([1,1′-Biphenyl]-4-yl)-N-((tetrahydro-2H-pyran-2-yl)oxy)chromane-2-carboxamide (43a). To a solution of 6-([1,1′-biphenyl]-4-yl)chromane-2-carboxylic acid (42a, 0.055 g, 0.17 mmol) in DMF (1.5 mL) was added O-(tetrahydro-2H-pyran-2-yl)hydroxylamine (0.037 g, 0.32 mmol) followed by T3P (0.152 g, 50% EtOAc soln) and DIPEA (0.05 mL, 0.30 mmol) at room temperature. After 4 h, the solution was extracted with EtOAc, washed with 0.5M HCl, 1M LiCl (2×), dried (Na₂SO₄), filtered and concentrated to give 6-([1,1′-biphenyl]-4-yl)-N-((tetrahydro-2H-pyran-2-yl)oxy)chromane-2-carboxamide (43a, 0.068 g, 95%) as an off-white solid that was used without further purification: ¹H NMR (CDCl₃, 500 MHz): δ9.18 (s, 1H), 7.66-7.60 (m, 6H), 7.47-7.40 (m, 3H), 7.37-7.35 (m, 2H), 6.98 (app t, J=8.0 Hz, 1H), 5.06-4.98 (m, 1H), 4.69-4.66 (m, 1H), 4.03-3.99 (m, 1H), 3.70-3.62 (m, 1H), 2.97-2.87 (m, 2H), 2.50-2.48 (m, 1H), 2.12-2.10 (m, 1H), 1.88-1.82 (m, 3H), 1.61-1.58 (m, 3H); HRMS (LCMS ESI+) m/z calcd for C₂₇H₂₇O₄NNa [M+Na] 452.1832, found 452.1830.

6-(4-Fluorophenyl)-N-((tetrahydro-2H-pyran-2-yl)oxy)chromane-2-carboxamide (43b). A solution of 6-(4-fluorophenyl)chromane-2-carboxylic acid (42b, 0.104 g, 0.38 mmol) was treated with O-(tetrahydro-2H-pyran-2-yl)hydroxylamine (0.063 g, 0.54 mmol) in DMF (1.5 mL) followed by T3P (0.369 g, 0.58 mmol, 50% EtOAc soln). The solution was then treated with DIPEA (0.10 mL, 0.60 mmol) at room temperature. After 21 h, the solution was extracted with EtOAc, washed with 0.5M HCl, brine, dried (Na₂SO₄), filtered and concentrated. The residual oil was then extracted with CH₂Cl₂, washed with 0.5M LiCl, brine, dried (Na₂SO₄), filtered and concentrated to give 6-(4-fluorophenyl)-N-((tetrahydro-2H-pyran-2-yl)oxy)chromane-2-carboxamide (43b, 0.135 g, 90%) as a beige solid that contained small amount of residual solvent and was used without further purification: ¹H NMR (CDCl₃, 500 MHz): δ9.21 (s, 1H), 7.45 (dd, J=5.4, 8.6 Hz, 2H), 7.28 (dd, J=1.9, 6.5 Hz, 1H), 7.23 (s, 1H), 7.08 (app t, J=8.6 Hz, 2H), 6.94 (app t, J=8.2 Hz, 1H), 5.04 (br s, 0.5H), 4.98 (br s, 0.5H), 4.67-4.63 (m, 1H), 4.02-3.98 (m, 1H), 3.68-3.61 (m, 1H), 2.94-2.85 (m, 2H), 2.49-2.43 (m, 1H), 2.15-2.03 (m, 1H), 1.85-1.81 (m, 3H), 1.62-1.59 (m, 3H); HRMS (LCMS ESI+) m/z calcd for C₂₁H₂₂O₄NFNa [M+Na] 394.1425, found 394.1422.

6-(3,4-Dichlorophenyl)-N-((tetrahydro-2H-pyran-2-yl)oxy)chromane-2-carboxamide (43c). To a solution of 6-(3,4-dichlorophenyl)chromane-2-carboxylic acid (42c, 0.090 g, crude) in DMF (2.0 mL) was added O-(tetrahydro-2H-pyran-2-yl)hydroxylamine (0.041 g, 0.35 mmol) followed by T3P (0.276 g, 50% EtOAc soln) followed by TEA (0.10 mL, 0.71 mmol) at room temperature. After 19 h, the solution was extracted with EtOAc, washed with 0.5M HCl, 1M LiCl, dried (Na₂SO₄), filtered and concentrated. The residual oil was purified by chromatography on SiO₂(ISCO-Rf, 0-100% EtOAc/hexanes) to give 6-(3,4-dichlorophenyl)-N-((tetrahydro-2H-pyran-2-yl)oxy)chromane-2-carboxamide (43c, 0.094 g, 80%) as an off-white foam: ¹H NMR (CDCl₃, 400 MHz): δ9.19 (s, 1H), 7.58 (d, J=1.6 Hz, 1H), 7.44 (d, J=8.4 Hz, 1H), 7.34-7.26 (m, 2H), 7.23 (s, 1H), 6.94 (app t, J=7.2 Hz, 1H), 5.04 (br s, 0.5H), 4.98 (br s, 0.5H), 4.67-4.63 (m, 1H), 4.03-3.97 (m, 1H), 3.69-3.61 (m, 1H), 2.93-2.82 (m, 2H), 2.47-2.43 (m, 1H), 2.17-2.02 (m, 1H), 1.85-1.80 (m, 3H), 1.64-1.59 (m, 3H); HRMS (LCMS ESI+) m/z calcd for C₂₁H₂₁O₄NCl₂Na [M+Na] 444.0740, found 444.0737.

6-([1,1′-Biphenyl]-4-yl)-N-hydroxychromane-2-carboxamide (44a). To a solution of 6-([1,1′-biphenyl]-4-yl)-N-((tetrahydro-2H-pyran-2-yl)oxy)chromane-2-carboxamide (43a, 0.068 g, 0.16 mmol) in MeOH (2.0 mL) and THE (1.0 mL) was added Amberlyst-15 (0.032 g) at room temperature. The solution became heterogeneous overnight. The mixture was concentrated and the mixture was diluted with EtOAc and the solid was collected by filtration and dried in vacuo at 100° C. to give 6-([1,1′-biphenyl]-4-yl)-N-hydroxychromane-2-carboxamide (44a, 0.041 g, 75%) as a white solid: mp 218-219° C.; IR (neat) 3264, 1677, 1645, 1528, 1480, 1276, 1239, 1169, 1128, 1073, 1031, 1005, 907, 844, 818, 765, 695 cm⁻¹; ¹H NMR (DMSO-d₆, 600 MHz): δ10.83 (s, 1H), 8.96 (s, 1H), 7.72-7.69 (m, 6H), 7.49-7.44 (m, 4H), 7.37 (app t, J=7.2 Hz, 1H), 6.93 (d, J=8.4 Hz, 1H), 4.58 (dd, J=3.0, 3.0 Hz, 1H), 2.88-2.85 (m, 1H), 2.84-2.78 (m, 1H), 2.17-2.14 (m, 1H), 2.01-1.98 (m, 1H); ¹³C NMR (DMSO-d₆, 150 MHz): δ166.4, 153.1, 139.7, 138.9, 138.3, 131.8, 128.9, 127.5, 127.0, 126.6, 126.4, 125.3, 122.3, 117.1, 73.9, 24.2, 23.1; HRMS (ESI+) m/z calcd for C₂₂H₁₈O₃N [M−H] 344.1281, found 344.1277.

6-(4-Fluorophenyl)-N-hydroxychromane-2-carboxamide (44b). To a solution of 6-(4-fluorophenyl)-N-((tetrahydro-2H-pyran-2-yl)oxy)chromane-2-carboxamide (43b, 0.135 g, 0.345 mmol) in MeOH (2.5 mL) and THE (0.20 mL) was added Amberlyst-15 (0.085 g, washed with MeOH) at room temperature. After 15 h, the mixture was filtered through Celite®, rinsed with MeOH and concentrated. The solid residue was slurried with Et₂O and the solid was collected by filtration and dried in vacuo at 100° C. to give 6-(4-fluorophenyl)-N-hydroxychromane-2-carboxamide (44b, 0.070 g, 71%) as an off-white solid: mp 177-179° C.; IR (neat) 3233, 1641, 1486, 1289, 1230, 1129, 1071, 1028, 1013, 998, 905, 863, 840, 815, 667 cm⁻¹; ¹H NMR (DMSO-d₆, 600 MHz): δ10.83 (s, 1H), 8.96 (s, 1H), 7.62 (dd, J=5.4, 8.4 Hz, 2H), 7.38 (d, J=1.8 Hz, 1H), 7.36 (d, J=6.0 Hz, 1H), 7.24 (app t, J=9.0 Hz, 2H), 6.90 (d, J=8.4 Hz, 1H), 4.56 (dd, J=3.0, 3.0 Hz, 1H), 2.85-2.81 (m, 1H), 2.79-2.75 (m, 1H), 2.16-2.12 (m, 1H), 2.00-1.95 (m, 1H); ¹³C NMR (DMSO-d₆, 150 MHz): 3166.4, 161.4 (J_{CF}=243 Hz), 153.0, 136.4 (J_{CF}=3 Hz), 131.4, 128.1, 127.8 (J_{CF}=48 Hz), 125.4, 122.3, 117.1, 115.5 (J_{CF}=21 Hz), 73.9, 24.3, 23.1; ¹⁹FNMR (DMSO-d₆, 470 MHz): δ−116.7; HRMS (LCMS ESI+) m/z calcd for C₁₆H₁₃O₃NF [M−H] 286.0874, found 286.0886.

6-(3,4-Dichlorophenyl)-N-hydroxychromane-2-carboxamide (44c). To a solution of 6-(3,4-dichlorophenyl)-N-((tetrahydro-2H-pyran-2-yl)oxy)chromane-2-carboxamide (43c, 0.093 g, 0.22 mmol) in MeOH (3.0 mL) was added Amberlyst-15 (0.042 g, washed with MeOH) at room temperature. After 15 h, the mixture was filtered through Celite®, rinsed with MeOH and concentrated. The residue was purified by chromatography on SiO₂ (ISCO-Rf, 0-100% EtOAc/hexanes) to give 6-(3,4-dichlorophenyl)-N-hydroxychromane-2-carboxamide (44c, 0.042 g, 56%) as an off-white foamy solid: mp 89-91° C.; IR (neat) 3260, 2922, 1663, 1501, 1464, 1372, 1233, 1128, 1097, 1076, 1026, 1006, 908, 868, 810, 784, 679 cm⁻¹; ¹H NMR (DMSO-d₆, 600 MHz): δ 10.83 (s, 1H), 8.96 (s, 1H), 7.87 (d, J=1.8 Hz, 1H), 7.65 (d, J=8.4 Hz, 1H), 7.61 (dd, J=1.8, 8.4 Hz, 1H), 7.47-7.46 (m, overlapping signals, 2H), 6.91 (d, J=9.0 Hz, 1H), 4.58 (dd, J=3.6, 3.6 Hz, 1H), 2.85-2.80 (m, 1H), 2.79-2.76 (m, 1H), 2.16-2.13 (m, 1H), 2.01-1.96 (m, 1H); ¹³C NMR (DMSO-d₆, 150 MHz): δ 166.3, 153.7, 140.5, 131.6, 130.8, 129.6, 129.2, 127.9, 127.8, 126.2, 125.6, 122.5, 117.2, 73.9, 24.2, 22.9; HRMS (ESI+) m/z calcd for C₁₆H₁₂O₃NCl₂ [M−H] 336.0189, found 336.0199.

### Part 3: 7-O Chromanes

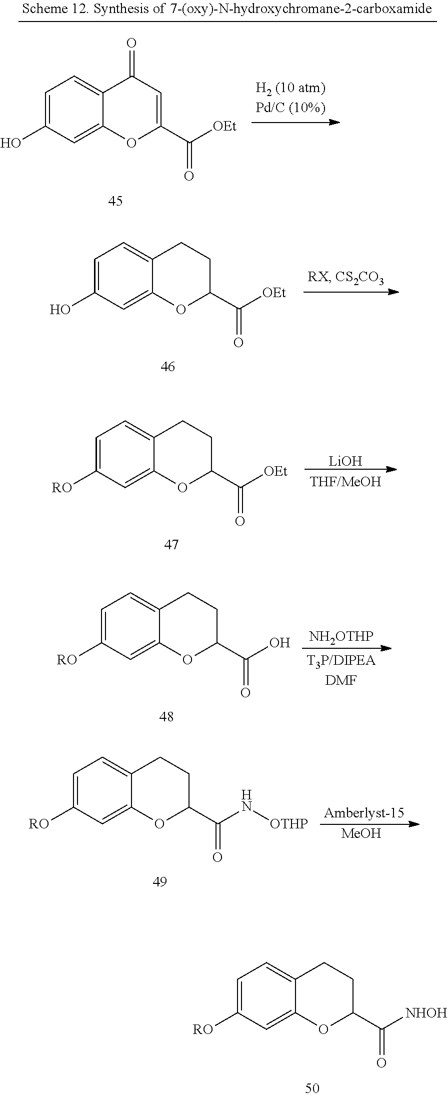

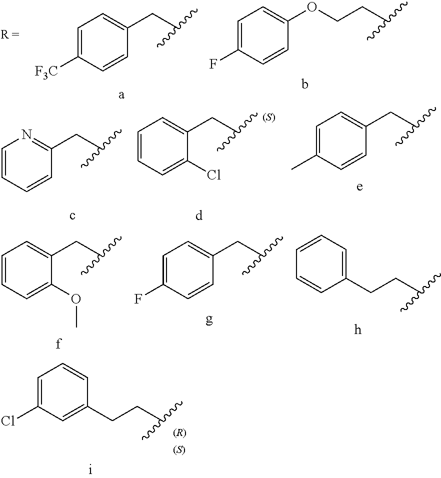

Ethyl 7-hydroxychromane-2-carboxylate (46). A solution of ethyl 7-hydroxy-4-oxo-4H-chromene-2-carboxylate (0.202 g, 0.863 mmol) in EtOH/THF (3 mL, 1:1) was evacuated and purged with N₂ (2×). To this solution was added 10% Pd/C (0.056 g, 0.061 mmol) and the mixture was evacuated and purged with H₂. The reaction mixture was kept under H₂ (1 atm-balloon) for 16 h. The reaction mixture was filtered through Celite®, rinsed with EtOAc and concentrated to give ethyl 7-hydroxychromane-2-carboxylate (46, 0.189 g, 98%) as a grey powder: ¹H NMR (CDCl₃, 500 MHz): δ 6.90 (d, J=8.2 Hz, 1H), 6.46 (d, J=2.5 Hz, 1H), 6.41 (dd, J=8.2, 2.5 Hz, 1H), 4.71 (dd, J=7.5, 3.6 Hz, 2H), 4.28 (qd, J=7.1, 1.0 Hz, 2H), 2.78-2.67 (m, 2H), 2.27-2.07 (m, 2H), 1.33-1.28 (m, 3H).

Ethyl 7-((4-(trifluoromethyl)benzyl)oxy)chromane-2-carboxylate (47a). To a solution of ethyl 7-hydroxychromane-2-carboxylate (46, 0.250 g, 1.12 mmol) and Cs₂CO₃ (0.746 g, 2.27 mmol) in DMF (2.5 mL) was added 4-trifluoromethylbenzyl bromide (0.531 g, 2.18 mmol) at room temperature. After 1 h, the reaction mixture was extracted with EtOAc washed with brine (2×), dried (Na₂SO₄), filtered, concentrated and azeotroped with heptanes (2×). The resulting oil was purified by chromatography on SiO₂, (0-20%, EtOAc/hexanes) to give ethyl 7-((4-(trifluoromethyl)benzyl)oxy)chromane-2-carboxylate (47a, 0.404 g, 94%) as a white solid: ¹H NMR (CDCl₃, 400 MHz): δ 7.64 (dd, J=10.1, 6.1 Hz, 2H), 7.56-7.52 (m, 2H), 6.96 (d, J=8.2 Hz, 1H), 6.58-6.54 (m, 2H), 5.10 (s, 2H), 4.71 (dd, J=7.5, 3.6 Hz, 1H), 4.31-4.25 (m, 2H), 2.80-2.68 (m, 2H), 2.29-2.17 (m, 2H), 1.33-1.27 (m, 3H); ¹⁹F NMR (CDCl₃, 471 MHz): δ−62.6.

7-((4-(Trifluoromethyl)benzyl)oxy)chromane-2-carboxylic acid (48a). A solution of ethyl 7-((4-(trifluoromethyl)benzyl)oxy)chromane-2-carboxylate (47a, 400 mg, 1.05 mmol) in THF/MeOH (2 mL, 1/1) was treated with 1M LiGH (1.26 mL) at room temperature. After 1 h, the solution was diluted with EtOAc and 0.5M HCl was added. The mixture was extracted with EtOAc, washed with brine, dried (Na₂SO₄), filtered, concentrated and azeotroped with CH₂Cl₂ and dried in vacuo to give 7-((4-(trifluoromethyl)benzyl)oxy)chromane-2-carboxylic acid (48a, 382 mg, quant.) as a white solid: ¹H NMR (DMSO-d₆, 400 MHz): δ 13.03 (dt, J=3.9, 1.4 Hz, 1H), 7.76-7.74 (m, 2H), 7.64 (d, J=8.0 Hz, 2H), 6.94 (d, J=8.3 Hz, 1H), 6.51 (dd, J=8.4, 2.6 Hz, 1H), 6.46 (d, J=2.5 Hz, 1H), 5.18 (s, 2H), 4.74-4.72 (m, 1H), 2.67-2.55 (m, 2H), 2.10-2.01 (m, 3H); HRMS (ESI+) m/z calcd for C₁₈H₁₄O₄F₃ [M−H] 351.0844, found 351.0852.

N-((Tetrahydro-2H-pyran-2-yl)oxy)-7-((4-(trifluoromethyl)benzyl)oxy)chromane-2-carboxamide (49a). A solution of 7-((4-(trifluoromethyl)benzyl)oxy)chromane-2-carboxylic acid (48a, 370 mg, 1.05 mmol) in DMF (2.5 mL) was treated with O-(tetrahydro-2H-pyran-2-yl)hydroxylamine (156 mg, 1.28 mmol). To this solution was added DIPEA (0.210 mL, 1.26 mmol) followed by T3P (0.750 mL, 1.26 mmol, 50% EtOAc soln). After 2 h, the solution was extracted with EtOAc and washed with H₂O. The aqueous layer was back extracted with EtOAc (3×). The combined organic layer was washed with brine, dried (Na₂SO₄), filtered and concentrated. The resulting oil was purified by chromatography on SiO₂ (0-30%, EtOAc/hexanes) to give N-((tetrahydro-2H-pyran-2-yl)oxy)-7-((4-(trifluoromethyl)benzyl)oxy)chromane-2-carboxamide (49a, 314 mg, 0.694 mmol, 66%) as a white foamy solid: ¹H NMR (CDCl₃, 400 MHz): δ 9.09 (s, 2H), 7.66-7.64 (m, 2H), 7.55-7.53 (m, 2H), 6.98-6.96 (m, 1H), 6.57-6.55 (m, 1H), 6.51-6.48 (m, 1H), 5.03-4.95 (m, 1H), 5.03-4.95 (m, 2H), 4.63-4.58 (m, 1H), 4.02-3.95 (m, 2H), 3.69-3.59 (m, 2H), 2.82-2.70 (m, 3H), 2.44-2.38 (m, 2H), 2.10-1.99 (m, 2H), 1.90-1.89 (m, 4H); ¹⁹F NMR (CDCl₃, 376 MHz): δ−62.6; HRMS (ESI+) m/z calcd for C₂₃H₂₄NO₅F₃Na [M+Na] 474.1504, found 474.1496.

N-Hydroxy-7-((4-(trifluoromethyl)benzyl)oxy)chromane-2-carboxamide (50a). A solution of N-((tetrahydro-2H-pyran-2-yl)oxy)-7-((4-(trifluoromethyl)benzyl)oxy)chromane-2-carboxamide (49a, 310 mg, 0.687 mmol) in MeOH (5 mL) was treated with Amberlyst-15 (60 mg, washed with MeOH) at room temperature. After 12 h, the solution was filtered through Celite®, rinsed with MeOH and concentrated. The solid residue was triturated with ether, sonicated and the resulting solid was collected by filtration and dried in vacuo to give N-hydroxy-7-((4-(trifluoromethyl)benzyl)oxy)chromane-2-carboxamide (50a, 0.131 g, 52%) as a white solid: mp 148-151° C.; IR (neat) 2851, 1636, 1329, 1111, 1093, 1067 cm⁻¹; ¹H NMR (DMSO-d₆, 400 MHz): δ 10.75 (s, 1H), 8.92 (d, J=1.6 Hz, 1H), 7.76-7.74 (m, 2H), 7.64 (d, J=8.0 Hz, 2H), 6.96 (d, J=8.4 Hz, 1H), 6.53 (dd, J=8.3, 2.6 Hz, 1H), 6.48 (d, J=2.5 Hz, 1H), 5.17 (s, 2H), 4.48 (dd, J=8.9, 3.0 Hz, 1H), 3.32 (s, 1H), 2.70-2.60 (m, 2H), 2.11-1.87 (m, 2H); ¹³C NMR (DMSO-d₆, 100 MHz): δ 166.3, 157.2, 154.0, 142.2, 129.9, 128.4, 128.0, 127.8, 125.37, 125.34, 125.30, 122.9, 114.4, 107.8, 102.8, 73.8, 68.2, 24.4, 22.3; ¹⁹F NMR (CDC₃, 376 MHz): δ−62.6; HRMS (ESI+) m/z calcd for C₁₈H₂₁NO₆Na [M−H] 366.0953, found 366.0961.

N-Hydroxy-7-((4-(trifluoromethyl)benzyl)oxy)chromane-2-carboxamide (50a). A solution of N-((tetrahydro-2H-pyran-2-yl)oxy)-7-((4-(trifluoromethyl)benzyl)oxy)chromane-2-carboxamide (49a, 310 mg, 0.687 mmol) in MeOH (5 mL) was treated with Amberlyst-15 (60 mg, washed with MeOH) at room temperature. After 12 h, the solution was filtered through Celite®, rinsed with MeOH and concentrated. The solid residue was triturated with ether, sonicated and the resulting solid was collected by filtration and dried in vacuo to give N-hydroxy-7-((4-(trifluoromethyl)benzyl)oxy)chromane-2-carboxamide (50a, 0.131 g, 52%) as a white solid: mp 148-151° C.; IR (neat) 2851, 1636, 1329, 1111, 1093, 1067 cm⁻¹; ¹H NMR (DMSO-d₆, 400 MHz): δ 10.75 (s, 1H), 8.92 (d, J=1.6 Hz, 1H), 7.76-7.74 (m, 2H), 7.64 (d, J=8.0 Hz, 2H), 6.96 (d, J=8.4 Hz, 1H), 6.53 (dd, J=8.3, 2.6 Hz, 1H), 6.48 (d, J=2.5 Hz, 1H), 5.17 (s, 2H), 4.48 (dd, J=8.9, 3.0 Hz, 1H), 3.32 (s, 1H), 2.70-2.60 (m, 2H), 2.11-1.87 (m, 2H); ¹³C NMR (DMSO-d₆, 100 MHz): δ 166.3, 157.2, 154.0, 142.2, 129.9, 128.4, 128.0, 127.8, 125.37, 125.34, 125.30, 122.9, 114.4, 107.8, 102.8, 73.8, 68.2, 24.4, 22.3; ¹⁹F NMR (CDCl₃, 376 MHz): δ−62.6; HRMS (ESI+) m/z calcd for C₁₈H₂₁NO₆Na [M−H] 366.0953, found 366.0961.

Ethyl 7-(2-(4-fluorophenoxy)ethoxy)chromane-2-carboxylate (47b). To a solution of ethyl 7-hydroxychromane-2-carboxylate (46, 0.182 g, 0.819 mmol) in DMF (1.0 mL) was added Cs₂CO₃ (0.534 g, 1.62 mmol) at room temperature. After 30 min, a solution of 1-(2-bromoethoxy)-4-fluorobenzene (0.183 g, 0.901 mmol) in DMF (1 mL) was added. After 5 h, the reaction mixture was extracted with EtOAc, washed with brine (2×), dried (Na₂SO₄), filtered, concentrated and azeotroped with heptanes (2×). The resulting oil was absorbed onto Celite® and purified by chromatography on SiO₂ (0-20%, EtOAc/hexanes) to give ethyl 7-(2-(4-fluorophenoxy)ethoxy)chromane-2-carboxylate (47b, 180 mg, 61%) as a white solid: ¹H NMR (CDCl₃, 500 MHz): δ 6.97 (t, J=8.7 Hz, 1H), 6.90 (td, J=12.2, 5.3 Hz, 2H), 6.54-6.50 (m, 1H), 4.69 (dd, J=7.6, 3.5 Hz, 1H), 4.27-4.26 (m, 4H), 2.77-2.67 (m, 2H), 2.26-2.14 (m, 2H), 1.29 (t, J=8.0 Hz, 3H); ¹⁹F NMR (CDCl₃, 470 MHz): δ−123.6.

7-(2-(4-Fluorophenoxy)ethoxy)-N-((tetrahydro-2H-pyran-2-yl)oxy)chromane-2-carboxamide (49b). To a solution of ethyl 7-(2-(4-fluorophenoxy)ethoxy)chromane-2-carboxylate (47b, 180 mg, 0.499 mmol) in THF/MeOH (2 mL, 3:1) was added 1M LiGH (0.55 mL) at room temperature. After 20 min, the solution was concentrated and the residue was azeotroped with PhMe (3×) and concentrated to give a white powder. The crude residue was dissolved in DMF (1 mL) and was treated with O-(tetrahydro-2H-pyran-2-yl)hydroxylamine (80.2 mg, 0.657 mmol). To this solution was added DIPEA (0.105 mL, 0.629 mmol) followed by T3P (0.360 mL, 0.605 mmol, 50% EtOAc soln) at room temperature. After 7 h, the reaction mixture was extracted with EtOAc and washed with H₂O. The aqueous layer was back extracted with EtOAc (3×). The combined organic layer was washed with brine, dried (Na₂SO₄), filtered and concentrated. The resulting oil was purified by chromatography on SiO₂ (0-50% EtOAc/hexanes) to give 7-(2-(4-fluorophenoxy)ethoxy)-N-((tetrahydro-2H-pyran-2-yl)oxy)chromane-2-carboxamide (49b, 125 mg, 58%) as a white solid: ¹H NMR (CDCl₃, 500 MHz): δ 9.09 (s, 2H), 7.00-6.96 (m, 3H), 6.89 (dd, J=9.1, 4.3 Hz, 2H), 6.55 (dd, J=8.4, 2.5 Hz, 1H), 6.48 (dd, J=8.8, 2.5 Hz, 1H), 5.03-4.96 (m, 1H), 4.62-4.59 (m, 2H), 4.27 (s, 4H), 4.01-3.97 (m, 1H), 3.69-3.61 (m, 2H), 2.81-2.74 (m, 3H), 2.45-2.38 (m, 1H), 2.10-2.01 (m, 2H), 1.86-1.81 (m, 3H), 1.60-1.60 (m, 3H); ¹⁹F NMR (CDCl₃, 470 MHz): δ−123.5; HRMS (ESI+) m/z calcd for C₂₃H₂₆FNO₆Na [M+Na] 454.1642, found 454.1634.

7-(2-(4-Fluorophenoxy)ethoxy)-N-hydroxychromane-2-carboxamide (50b). A solution of 7-(2-(4-fluorophenoxy)ethoxy)-N-((tetrahydro-2H-pyran-2-yl)oxy)chromane-2-carboxamide (49b, 123 mg, 0.285 mmol) in MeOH (2 mL) was treated with Amberlyst-15 (60 mg, washed with MeOH) at room temperature. After 23 h, the solution was filtered through Celite®, rinsed with MeOH and concentrated. The solid residue was triturated with Et₂O, sonicated and the resulting solid was collected by filtration and dried in vacuo to give 7-(2-(4-fluorophenoxy)ethoxy)-N-hydroxychromane-2-carboxamide (50b, 0.071 g, 72%) as a white powder: mp 144-147; IR (neat) 3174, 2929, 1649, 1507, 1067, 826 cm⁻¹; ¹H NMR (DMSO-d₆, 400 MHz): δ 10.77 (d, J=1.6 Hz, 1H), 8.94 (d, J=1.6 Hz, 1H), 7.13 (t, J=8.9 Hz, 2H), 6.98 (dt, J=11.7, 7.5 Hz, 3H), 6.50-6.44 (m, 2H), 4.50 (dd, J=8.8, 3.1 Hz, 1H), 4.24 (dd, J=13.7, 4.7 Hz, 4H), 2.70-2.62 (m, 2H), 2.10-1.89 (m, 2H); ¹³C NMR (DMSO-d₆, 100 MHz): δ 166.4, 157.8, 157.5, 155.4, 154.67, 154.65, 153.9, 130.0, 116.00, 115.84, 115.77, 115.76, 114.1, 107.6, 73.8, 66.8, 66.3, 24.5, 22.3; ¹⁹F NMR (DMSO-d₆, 470 MHz): δ−123.8; HRMS (ESI+) m/z calcd for C₁₈H₁₇FNO₅ [M−H] 346.1091, found 346.1080.

Ethyl 7-(pyridin-2-ylmethoxy)chromane-2-carboxylate (47c). To a solution of ethyl 7-hydroxychromane-2-carboxylate (46, 0.402 g, 1.81 mmol) in DMF (4.0 mL) was added Cs₂CO₃ (1.80 g, 5.47 mmol) followed by bromomethyl pyridine hydrobromide (0.704 g, 2.73 mmol) at room temperature. After 2 h, the reaction mixture was extracted with EtOAc, washed with brine (2×), dried (Na₂SO₄), filtered, concentrated and azeotroped with heptanes (2×). The resulting oil was absorbed onto Celite® and purified by chromatography on SiO₂ (0-30%, EtOAc/hexanes) to give ethyl 7-(pyridin-2-ylmethoxy)chromane-2-carboxylate (47c, 429 mg, 76%) as a light yellow oil: ¹H NMR (CDCl₃, 500 MHz): δ 8.58 (d, J=4.6 Hz, 1H), 7.70 (td, J=7.7, 1.7 Hz, 1H), 7.50 (d, J=7.8 Hz, 1H), 7.21 (dd, J=7.2, 5.2 Hz, 1H), 6.93 (d, J=8.3 Hz, 1H), 6.59 (d, J=2.5 Hz, 1H), 6.59-6.54 (m, 2H), 6.55 (dd, J=8.3, 2.6 Hz, 1H), 5.16 (s, 2H), 4.68 (dd, J=7.6, 3.5 Hz, 1H), 2.77-2.68 (m, 2H), 2.25-2.15 (m, 2H), 1.29 (dd, J=8.9, 5.5 Hz, 3H).

7-(Pyridin-2-ylmethoxy)-N-((tetrahydro-2H-pyran-2-yl)oxy)chromane-2-carboxamide (49c). A solution of ethyl 7-(pyridin-2-ylmethoxy)chromane-2-carboxylate (47c, 425 mg, 1.36 mmol) in THF/MeOH (3 mL, 3:1) was treated with 1M LiGH (1.49 mL) at room temperature. After 20 min, the solution was concentrated and the residue was azeotroped with PhMe (3×) and concentrated to give a light yellow powder that was used without further purification. The crude product was dissolved in DMF (3 mL) and treated with O-(tetrahydro-2H-pyran-2-yl)hydroxylamine (214 mg, 1.75 mmol). To this solution was added DIPEA (0.290 mL, 1.74 mmol) followed by T3P (1.10 mL, 1.85 mmol, 50% EOAc soln). After 3 h, the solution was extracted with EtOAc and washed with H₂O, brine (2×), dried (Na₂SO₄), filtered, concentrated and azeotroped with CH₂Cl₂ (2×). The resulting oil was purified by chromatography on SiO₂ (0-75% EtOAc/hexanes) to give 7-(pyridin-2-ylmethoxy)-N-((tetrahydro-2H-pyran-2-yl)oxy)chromane-2-carboxamide (49c, 244 mg, 39%) as a colorless oil: ¹H NMR (CDCl₃, 500 MHz): δ 9.09 (d, J=0.2 Hz, 1H), 8.60 (d, J=4.5 Hz, 1H), 7.71 (td, J=7.7, 1.7 Hz, 1H), 7.50 (d, J=7.8 Hz, 1H), 7.24-7.21 (m, 1H), 6.96 (d, J=8.4 Hz, 1H), 6.59 (dd, J=8.4, 2.6 Hz, 1H), 6.52 (dd, J=9.5, 2.5 Hz, 1H), 5.03-4.96 (m, 1H), 4.60 (ddd, J=9.7, 7.4, 2.6 Hz, 1H), 4.60 (ddd, J=9.7, 7.4, 2.6 Hz, 2H), 4.02-3.95 (m, 1H), 3.69-3.61 (m, 2H), 2.82-2.70 (m, 2H), 2.45-2.38 (m, 1H), 2.10-2.00 (m, 2H), 1.89-1.78 (m, 3H), 1.68-1.54 (m, 4H).

N-Hydroxy-7-(pyridin-2-ylmethoxy)chromane-2-carboxamide (50c). A solution of 7-(pyridin-2-ylmethoxy)-N-((tetrahydro-2H-pyran-2-yl)oxy)chromane-2-carboxamide (49c, 244 mg, 0.559 mmol) in MeOH/THF (2.0/0.5 mL) was treated with Amberlyst-15 (60 mg, washed with MeOH) at room temperature. The mixture was heated to reflux for 5 h and cooled to room temperature overnight. After 16 h, the mixture was heated to reflux for an additional 16 h. The solution was cooled to room temperature and filtered through Celite®, rinsed with MeOH and concentrated. The solid residue was triturated with Et₂O, sonicated and the solid was collected by filtration, and dried in vacuo to give N-hydroxy-7-(pyridin-2-ylmethoxy)chromane-2-carboxamide (50c, 101 mg, 60%) as a white solid: mp 125-128° C.; IR (neat) 3190, 2847, 1661, 1622, 1506, 1164 cm⁻¹; ¹H NMR (DMSO-d₆, 500 MHz): δ 10.76 (s, 1H), 8.92 (d, J=1.6 Hz, 1H), 8.57-8.56 (m, 1H), 7.82 (td, J=7.7, 1.7 Hz, 1H), 7.47 (d, J=7.8 Hz, 1H), 7.35-7.32 (m, 1H), 6.95 (d, J=8.4 Hz, 1H), 6.53 (dd, J=8.3, 2.6 Hz, 1H), 6.48 (d, J=2.5 Hz, 1H), 5.11 (s, 2H), 4.48 (dd, J=9.0, 3.0 Hz, 1H), 2.69-2.62 (m, 2H), 2.10-2.06 (m, 1H), 1.90-1.87 (m, 1H); ¹³C NMR (DMSO-d₆, 100 MHz): δ 166.4, 157.3, 156.8, 153.9, 149.1, 137.0, 130.0, 122.9, 121.5, 114.3, 107.8, 102.7, 73.9, 70.2, 24.5, 22.4; HRMS (ESI+) m/z calcd for C₁₆H₁₅N₂O₄ [M−H] 299.1032, found 299.1022.

Ethyl (S)-7-((2-chlorobenzyl)oxy)chromane-2-carboxylate (47d). To a solution of ethyl (S)-7-((2-chlorobenzyl)oxy)chromane-2-carboxylate (0.120 g, 0.54 mmol) in DMF (2.5 mL) was added Cs₂CO₃ (0.35 g, 1.08 mmol) followed by 2-chlorobenzyl bromide (0.14 mL, 1.08 mmol) at room temperature. After 1 h, the reaction mixture was extracted with EtOAc, washed with brine (2×), dried (MgSO₄), filtered, concentrated and azeotroped with heptanes (2×). The resulting oil was absorbed onto Celite® and purified by chromatography on SiO₂ (0-20%, EtOAc/hexanes) to give ethyl (S)-7-((2-chlorobenzyl)oxy)chromane-2-carboxylate (47d, 165 mg, 88%) as a colorless oil: ¹H NMR (CDCl₃, 500 MHz): δ7.57 (d, J=6.67 Hz, 1H), 7.41 (d, J=5.28 Hz, 1H), 7.31-7.26 (m, 2H), 6.96 (d, J=8.3 Hz, 1H), 6.63-6.57 (m, 2H), 5.15 (s, 2H), 4.73-4.70 (m, 1H), 4.30-4.28 (m, 2H), 2.86-2.66 (m, 2H), 2.34-2.12 (m, 2H), 1.32 (t, J=7.12 Hz, 3H).

(S)-7-((2-Chlorobenzyl)oxy)chromane-2-carboxylic acid (48d). A solution of ethyl (S)-7-((2-chlorobenzyl)oxy)chromane-2-carboxylate (47d, 0.160 g, 0.46 mmol, crude) in THF/MeOH (1.5/0.75 mL) was treated with 1 LiGH (0.48 mL) at room temperature. After 2 h, the solution was concentrated, diluted with H₂O, acidified with 1M HCl and extracted with EtOAc (2×). The combined organic layer was washed with brine, dried (Na₂SO₄), filtered and concentrated to give (S)-7-((2-chlorobenzyl)oxy)chromane-2-carboxylic acid (48d, 0.12 g, 82%) as a beige solid that was used without further purification.

(2S)-7-((2-Chlorobenzyl)oxy)-N-((tetrahydro-2H-pyran-2-yl)oxy)chromane-2-carboxamide (49d). To a solution of (S)-7-((2-chlorobenzyl)oxy)chromane-2-carboxylic (48d, 0.14 g, 0.44 mmol, crude) in CH₂Cl₂/DMF (2.0/0.2 mL) was added O-(tetrahydro-2H-pyran-2-yl)hydroxylamine (0.077 g, 0.66 mmol) in CH₂Cl₂ (1.0 mL). The solution was cooled to 0° C. and treated with T3P (0.419 g, 50% EtOAc soln) followed by DIPEA (0.09 mL, 0.57 mmol). The reaction mixture was slowly warmed to room temperature. After 4.5 h, the reaction mixture was extracted with CH₂Cl₂, washed with 0.2M HCl, 1M LiCl, H₂O, dried (Na₂SO₄), filtered and concentrated to give (2S)-7-((2-chlorobenzyl)oxy)-N-((tetrahydro-2H-pyran-2-yl)oxy)chromane-2-carboxamide (49d, 0.133 g, 85%) as a foamy solid: ¹H NMR (CDCl₃, 300 MHz): 39.13 (s, 1H), 7.58-7.55 (m, 1H), 7.44-7.41 (m, 1H), 7.29-7.26 (m, 2H), 7.00 (d, J=8.3 Hz, 1H), 6.63-6.53 (m, 2H), 5.16 (s, 2H), 4.66-4.60 (m, 1H), 4.05-3.98 (m, 1H), 3.73-3.63 (m, 1H), 2.87-2.71 (m, 2H), 2.50-2.38 (m, 1H), 2.16-1.96 (m, 1H), 1.94-1.82 (m, 3H), 1.70-1.55 (m, 3H).

(S)-7-((2-Chlorobenzyl)oxy)-N-hydroxychromane-2-carboxamide (50d). A solution of (2S)-7-((2-chlorobenzyl)oxy)-N-((tetrahydro-2H-pyran-2-yl)oxy)chromane-2-carboxamide (49d, 0.133 g, 0.32 mmol) in MeOH (3.0 mL) was treated with Amberlyst-15 (0.076 g, washed with MeOH) at room temperature. After 22 h, the mixture was filtered through Celite®, rinsed with MeOH and concentrated. The residue was slurried with Et₂O and the solid was collected by filtration and dried in vacuo at 90° C. to give (S)-7-((2-chlorobenzyl)oxy)-N-hydroxychromane-2-carboxamide (50d, 0.029 g, 27%) as a white solid: mp 141-147° C.; IR (neat) 3271, 2910, 1647, 1622, 1505, 1441, 1300, 1180, 1165, 1093, 849, 830, 790, 747, 693 cm⁻¹; ¹H NMR (DMSO-d₆, 600 MHz): δ10.78 (s, 1H), 8.93 (s, 1H), 7.55 (dd, J=4.8, 9.0 Hz, 1H), 7.51 (d, J=2.4 Hz, 1H), 7.38 (dd, J=1.8, 4.2 Hz, 2H), 6.96 (d, J=8.4 Hz, 1H), 6.53 (app t, J=2.4 Hz, 1H), 6.48 (d, J=1.8 Hz, 1H), 5.10 (s, 2H), 4.50 (dd, J=3.0, 9.0 Hz, 1H) 2.72-2.62 (m, 2H), 2.09-2.07 (m, 1H), 1.93-1.90 (m, 1H); ¹³C NMR (DMSO-d₆, 150 MHz): δ166.3, 157.3, 153.9, 134.4, 132.4, 129.9, 129.8, 129.7, 129.3, 127.3, 114.4, 107.7, 102.6, 73.8, 66.7, 24.4, 22.3; HRMS (ESI+) m/z calcd for C₁₇H₁₆ClNO₄ [M+H] 334.0684, found 332.0693.

Ethyl 7-((4-methylbenzyl)oxy)chromane-2-carboxylate (47e). To a solution of ethyl 7-hydroxychromane-2-carboxylate (46, 0.150 g, 0.675 mmol) in DMF (1.5 mL) was added Cs₂CO₃ (0.440 g, 1.35 mmol) followed by 4-methylbenzyl bromide (0.137 g, 1.35 mmol) at room temperature. After 1 h, the reaction mixture was extracted with EtOAc and washed with H₂O. The aqueous layer was back extracted with EtOAc (2×). The combined organic layer was washed with water, brine, dried (Na₂SO₄), filtered and concentrated. The residue was purified by chromatography on SiO₂ (ISCO-Rf, 0-10% EtOAc/hexanes) to give ethyl 7-((4-methylbenzyl)oxy)chromane-2-carboxylate as an oil (47e, 0.162 g, 73%): IR (neat) 3024, 2978, 2928, 2858, 1751, 1731, 1620, 1582, 1503, 1463, 1432, 1376, 1337, 1295, 1251, 1194, 1153, 1125, 1111, 1016, 963, 938, 831, 800, 773, 747, 734, 701, 660, 627, cm⁻¹; ¹H NMR (CDCl₃, 400 MHz): δ7.30 (d, J=8.0 Hz, 2H), 7.18 (d, J=7.6 Hz, 2H), 6.91 (d, J=8.4 Hz, 1H), 6.58 (d, J=2.0 Hz, 1H), 6.53 (dd, J=2.4, 8.4 Hz, 1H), 4.97 (s, 2H), 4.68 (dd, J=3.2, 7.6 Hz, 1H), 4.26 (q, J=6.8 Hz, 2H), 2.81-2.64 (m, 2H), 2.35 (s, 3H), 2.30-2.21 (m, 1H), 2.20-2.11 (m, 1H), 2.20-2.11 (m, 1H), 1.29 (t, J=7.2 Hz, 3H).

7-((4-Methylbenzyl)oxy)chromane-2-carboxylic acid (48e). A solution of ethyl 7-((4-methylbenzyl)oxy)chromane-2-carboxylate (47e, 0.160 g, 0.490 mmol) in EtOH/THF (4 mL, 3:1) was treated with 1 M LiGH (0.7 mL) at room temperature. After 2 h, the reaction mixture was acidified with 1M HCl and extracted with EtOAc. The aqueous layer was back extracted with EtOAc. The combined organic layer was dried (Na₂SO₄), filtered and concentrated to give 7-((4-methylbenzyl)oxy)chromane-2-carboxylic acid (48e, 0.104 g, 71%) as a light pink solid: IR (neat) 3046, 3013, 2969, 2943, 2918, 2858, 2778, 2614, 1702, 1620, 1583, 1508, 1455, 1438, 1343, 1298, 1213, 1196, 1161, 1133, 1013, 938, 916, 905, 839, 770, 702, 686, 630 cm⁻¹; ¹H NMR (DMSO-d₆, 400 MHz): δ7.29 (d, J=8.0 Hz, 2H), 7.18 (d, J=8.0 Hz, 2H), 6.92 (d, J=8.0 Hz, 1H), 6.48 (dd, J=2.4, 8.0 Hz, 1H), 6.42 (d, J=2.4 Hz, 1H), 4.99 (s, 2H), 4.73 (dd, J=3.6, 5.6 Hz, 1H), 2.72-2.66 (m, 1H), 2.56-2.54 (m, 1H), 2.30 (s, 3H), 2.15-2.10 (m, 1H), 2.09-1.98 (m, 1H).

7-((4-Methylbenzyl)oxy)-N-((tetrahydro-2H-pyran-2-yl)oxy)chromane-2-carboxamide (49e). A solution of 7-((4-methylbenzyl)oxy)chromane-2-carboxylic acid (48e, 0.102 g, 0.342 mmol) in DMF/CH₂Cl₂ (0.4 mL, 1:1) was cooled to 0° C. and treated with 0-(tetrahydro-2H-pyran-2-yl)hydroxylamine (0.056 g, 0.478 mmol) in CH₂Cl₂ (0.3 mL). To this solution was added T3P (0.261 g, 0.410 mmol, 50% CH₃CN soln) in CH₂Cl₂ (0.3 mL) followed by DIPEA (0.062 mL, 0.376 mmol). The solution was slowly warmed to room temperature. After 22 h, additional 0-(tetrahydro-2H-pyran-2-yl)hydroxylamine (0.056 g, 0.478 mmol) in CH₂Cl₂ (0.3 mL), T3P (0.261 g, 0.410 mmol) in CH₂Cl₂ (0.5 mL), and DIPEA (0.062 mL, 0.376 mmol) were added. The reaction mixture was warmed to 50° C. for 1 h. The reaction mixture was extracted with CH₂Cl₂ and washed with H₂O, 0.25 M HCl (2×), 1M LiCl (2×), and H₂O (2×), dried (Na₂SO₄), filtered and concentrated. The residue was purified by chromatography on SiO₂ (ISCO-Rf, 0-100% EtOAc/hexanes) to give 7-((4-methylbenzyl)oxy)-N-((tetrahydro-2H-pyran-2-yl)oxy)chromane-2-carboxamide (49e, 0.088 g, 65%) as an oil: ¹H NMR (CDCl₃, 600 MHz): δ 9.10 (s, 1H), 7.31 (d, J=7.8 Hz, 2H), 7.19 (d, J=7.8 Hz, 2H), 6.95 (d, J=7.8 Hz, 1H), 6.56 (dd, J=2.4, 8.4 Hz, 1H), 6.50 (dd, J=2.4, 12.0 Hz, 1H), 5.03 (app t, J=3.0 Hz, 0.5H), 4.98 (s, 2H), 4.96 (app t, J=3.0 Hz, 0.5H), 4.60 (td, J=3.0, 10.2 Hz, 1H), 4.01-3.96 (m, 1H), 3.69-3.61 (m, 1H), 2.82-2.70 (m, 2H), 2.44-2.39 (m, 1H), 2.36 (s, 3H), 2.10-1.98 (m, 1H), 1.89-1.76 (m, 3H), 1.70-1.50 (m, 3H).

N-Hydroxy-7-((4-methylbenzyl)oxy)chromane-2-carboxamide (50e). A solution of 7-((4-methylbenzyl)oxy)-N-((tetrahydro-2H-pyran-2-yl)oxy)chromane-2-carboxamide (49e, 0.083 g, 0.209 mmol) in CH₂Cl₂/MeOH (2 mL, 1:1) was treated with Amberlyst-15 (0.036 g, washed with MeOH) at room temperature. After 13 h, the reaction was heated at 45° C. for 2 h. The reaction mixture was cooled to room temperature and filtered through Celite®, rinsed with CH₂Cl₂/MeOH, and concentrated. The solid residue is triturated with Et₂O:hexanes (1:1) and the solid was collected by filtration and dried to give N-hydroxy-7-((4-methylbenzyl)oxy)chromane-2-carboxamide (50e, 0.050 g, 76%) as a tan solid: IR (neat) 3329, 3270, 3151, 3065, 2955, 2852, 1621, 1584, 1537, 1505, 1432, 1384, 1317, 1258, 1181, 1162, 1103, 1043, 1004, 960, 903, 831, 772, 749, 694, 676 cm⁻¹; ¹H NMR (DMSO-d₆, 600 MHz): δ10.76 (s, 1H), 8.93 (s, 1H), 7.29 (d, J=7.8 Hz, 2H), 7.18 (d, J=7.8 Hz, 2H), 6.93 (d, J=8.4 Hz, 1H), 6.50 (dd, J=2.4, 8.4 Hz, 1H), 6.45 (d, J=2.4 Hz, 1H), 4.98 (s, 2H), 4.47 (dd, J=3.0, 9.0 Hz, 1H), 2.72-2.66 (m, 1H), 2.63-2.59 (m, 1H), 2.30 (s, 3H), 2.09-2.05 (m, 1H), 1.92-1.86 (m, 1H); ¹³C NMR (DMSO-d₆, 150 MHz): δ 166.3, 157.5, 153.9, 136.9, 134.1, 129.8, 128.9, 127.6, 114.0, 107.9, 102.7, 100.7, 73.8, 69.0, 24.4, 22.3, 20.7; HRMS (ESI+) m/z calcd for C₁₈H₁₈NO₄ [M−H]312.1230, found 312.1224.

Ethyl 7-((3-methoxybenzyl)oxy)chromane-2-carboxylate (47f). A solution of ethyl 7-hydroxychromane-2-carboxylate (46, 0.150 g, 0.675 mmol) in DMF (1.5 mL) was treated with Cs₂CO₃ (0.440 g, 1.35 mmol) followed by 3-methoxybenzyl bromide (0.189 mL, 1.35 mmol) at room temperature. After 4 h, the reaction mixture was extracted with EtOAc and washed with H₂O. The aqueous layer was back extracted with EtOAc. The combined organic layer was washed with brine, dried (Na₂SO₄), filtered and concentrated. The residue was purified by chromatography on SiO₂ (ISCO-Rf, 0-20% EtOAc/hexanes) to give ethyl 7-((3-methoxybenzyl)oxy)chromane-2-carboxylate (47f, 0.171 g, 74%) as a colorless oil: IR (neat) 3063, 2976, 2934, 1750, 1731, 1619, 1603, 1503, 1457, 1375, 1262, 1193, 1125, 1111, 932, 50, 832, 770, 690, 625 cm⁻¹; ¹H NMR (CDCl₃, 600 MHz): 37.28 (app t, J=7.8 Hz, 1H), 6.99 (d, J=7.8 Hz, 1H), 6.97 (d, J=1.8 Hz, 1H), 6.91 (d, J=8.4 Hz, 1H), 6.84 (dd, J=2.4, 8.4 Hz, 1H), 6.57 (d, J=2.4 Hz, 1H), 6.54 (dd, J=2.4, 8.4 Hz, 1H), 4.99 (s, 2H), 4.68 (dd, J=3.0 Hz, 1H), 4.28-4.23 (m, 2H), 3.81 (s, 3H), 2.78-2.74 (m, 1H), 2.70-2.65 (m, 1H), 2.27-2.22 (m, 1H), 2.18-2.12 (m, 1H), 1.29 (t, J=7.2 Hz, 3H).

7-((3-Methoxybenzyl)oxy)chromane-2-carboxylic acid (48f). A solution of ethyl 7-((3-methoxybenzyl)oxy)chromane-2-carboxylate (47f, 0.163 g, 0.476 mmol) in EtOH/THF (4 mL, 3/1) was treated with 1M LiGH (0.7 mL) at room temperature. After 3 h, the reaction mixture was cooled to 0° C. and acidified with 1 M HCl to pH 2. This solution was extracted with EtOAc and washed with H₂O. The aqueous layer was back extracted with EtOAc (4×). The combined organic layer was washed with brine, dried (Na₂SO₄), filtered and concentrated to give 7-((3-methoxybenzyl)oxy)chromane-2-carboxylic acid (48f, 0.146g, 98%-contains residual solvent) as a white solid: ¹H NMR (DMSO-d₆, 600 MHz): δ13.05 (br s, 1H), 7.29 (app t, J=8.4 Hz, 1H), 6.98 (br s, 1H), 6.98 (d, J=1.8 Hz, 1H), 6.93 (d, J=8.4 Hz, 1H), 6.88-6.87 (m, 1H), 6.49 (dd, J=2.4, 8.4 Hz, 1H), 6.43 (d, J=2.4 Hz, 1H), 5.02 (s, 2H), 4.72 (dd, J=3.6, 6.0 Hz, 1H), 3.75 (s, 3H), 2.71-2.66 (m, 1H), 2.57-2.53 (m, 1H), 2.12-2.07 (m, 1H), 2.04-1.99 (m, 1H).

7-((3-Methoxybenzyl)oxy)-N-((tetrahydro-2H-pyran-2-yl)oxy)chromane-2-carboxamide (49f). A solution of 7-((3-methoxybenzyl)oxy)chromane-2-carboxylic acid (48f, 0.139 g, 0.442 mmol) in DMF/CH₂Cl₂ (0.4 mL, 1/1) was cooled to 0° C. and treated with O-(tetrahydro-2H-pyran-2-yl)hydroxylamine (0.085 g, 0.726 mmol) in CH₂Cl₂ (0.3 mL). To this solution was added T3P (0.360 g, 0.565 mmol, 50% CH₃CN soln) in CH₂Cl₂ (0.3 mL) followed by DIPEA (0.100 mL, 0.574 mmol). The solution was stirred at 0° C. for 10 min and then warmed to room temperature. After 4.5 h, additional 0-(tetrahydro-2H-pyran-2-yl)hydroxylamine (0.085 g, 0.726 mmol), T3P (0.415 g, 0.652 mmol, 50% CH₃CN soln), and DIPEA (0.100 mL, 0.574 mmol) were added. After 2 h, the reaction mixture was extracted with CH₂Cl₂ and washed with water. The aqueous layer was back extracted thoroughly with CH₂Cl_{2.} The combined organic layer was washed with 0.25M HCl (2×), 1M LiCl (2×), and H₂O (2×), dried (Na₂SO₄), filtered and concentrated. The residue obtained was purified by chromatography on SiO₂ (ISCO-Rf, 0-50% EtOAc/hexanes) to give 7-((3-methoxybenzyl)oxy)-N-((tetrahydro-2H-pyran-2-yl)oxy)chromane-2-carboxamide (49f, 0.122 g, 76%-contains residual solvent) as a colorless oil: ¹H NMR (CDCl₃, 600 MHz): δ9.10 (s, 1H), 7.30 (app t, J=7.8 Hz, 1H), 7.00 (d, J=7.8 Hz, 1H), 6.98 (d, J=8.4 Hz, 1H), 6.96 (d, J=8.4 Hz, 1H), 6.86 (dd, J=2.4, 8.4 Hz, 1H), 6.57 (dd, J=2.4, 8.4 Hz, 1H), 6.51 (dd, J=3.0, 12.6 Hz, 1H), 5.03 (app t, J=2.4 Hz, 0.5H), 5.01 (s, 2H), 4.97 (app t, J=3.0 Hz, 0.5H), 4.60 (td, J=2.4, 10.2 Hz, 1H), 4.01-3.97 (m, 1H), 3.82 (s, 3H), 3.68-3.63 (m, 1H), 2.84-2.74 (m, 1H), 2.76-2.71 (m, 1H), 2.45-2.38 (m, 1H), 2.11-1.98 (m, 1H), 1.87-1.80 (m, 3H), 1.68-1.58 (m, 3H).

N-Hydroxy-7-((3-methoxybenzyl)oxy)chromane-2-carboxamide (50f). A solution of 7-((3-methoxybenzyl)oxy)-N-((tetrahydro-2H-pyran-2-yl)oxy)chromane-2-carboxamide (49f, 0.115 g, 0.278 mmol) in CH₂Cl₂/MeOH (2 mL, 1/1) was treated with Amberlyst-15 (0.050 g, washed with MeOH) at room temperature. After 16 h, the reaction mixture was filtered through Celite®, rinsed with CH₂Cl₂/MeOH and concentrated. The solid residue was triturated with Et₂O:hexanes (1:2.5) collected by filtration and dried in vacuo to give N-hydroxy-7-((3-methoxybenzyl)oxy)chromane-2-carboxamide (50f, 0.060 g, 66%) as a light orange solid: IR (neat) 3285, 3083, 2949, 2848, 1619, 1584, 1537, 1508, 1452, 1374, 1282, 1203, 1164, 1152, 1128, 1102, 1040, 961, 901, 872, 766, 748, 699, 627 cm⁻¹; ¹H NMR (DMSO-d₆, 600 MHz): δ10.76 (s, 1H), 8.93 (d, J=1.2, Hz, 1H), 7.29 (app t, J=7.8 Hz, 1H), 6.98 (br s, 1H), 6.97 (br, 1H), 6.94 (d, J=8.4 Hz, 1H), 6.88 (dd, J=1.2, 7.2 Hz, 1H), 6.51 (dd, J=2.4, 8.4 Hz, 1H), 6.46 (d, J=2.4 Hz, 1H), 5.01 (s, 2H), 4.47 (dd, J=3.0, 9.0 Hz, 1H), 3.75 (s, 3H), 2.72-2.67 (m, 1H), 2.64-2.59 (m, 1H), 2.10-2.05 (m, 1H), 1.93-1.87 (m, 1H); ¹³C NMR (DMSO-d₆, 125 MHz): δ166.4, 159.3, 157.5, 153.9, 138.8, 129.9, 129.6, 119.5, 114.1, 113.1, 113.0, 107.9, 102.7, 73.8, 69.0, 55.1, 24.5, 22.4; HRMS (ESI+) m/z calcd for C₁₈H₁₈NO₅ [M−H]328.1179, found 328.1190.

Ethyl 7-((4-fluorobenzyl)oxy)chromane-2-carboxylate (47g). A solution of ethyl 7-hydroxychromane-2-carboxylate (46, 0.150 g, 0.675 mmol) in DMF/MeCN (5 mL, 1:1) was treated with Cs₂CO₃ (0.168 mL, 1.349 mmol) followed by 4-fluorobenzyl bromide (0.137 g, 1.350 mmol) and the reaction mixture was heated at 80° C. After 3 h, the reaction mixture was extracted with EtOAc and washed with H₂O. The aqueous layer was back extracted with EtOAc. The combined organic layer was washed with brine, dried (Na₂SO₄), filtered and concentrated. The residue obtained was purified by chromatography on SiO₂ (ISCO-Rf, 0-50% EtOAc/hexanes) to give ethyl 7-((4-fluorobenzyl)oxy)chromane-2-carboxylate (47g, 0.195 g, 87%) as an oil: ¹H NMR (CDCl₃, 600 MHz): δ 7.38 (dd, J=5.4, 9.0 Hz, 2H), 7.06 (app t, J=8.4 Hz, 2H), 6.92 (d, J=8.4 Hz, 1H), 6.56 (d, J=3.0 Hz, 1H), 6.52 (dd, J=3.0, 8.4 Hz, 1H), 4.97 (s, 2H), 4.69 (dd, J=3.6, 7.8 Hz, 1H), 4.28-4.24 (m, 2H), 2.80-2.75 (m, 1H), 2.69-2.66 (m, 1H), 2.28-2.23 (m, 1H), 2.19-2.13 (m, 1H), 1.30 (t, J=7.2 Hz, 3H).

7-((4-Fluorobenzyl)oxy)chromane-2-carboxylic acid (48g). A solution of ethyl 7-((4-fluorobenzyl)oxy)chromane-2-carboxylate (47g, 0.186 g, 0.563 mmol) in EtOH/THF (4 mL, 3:1) was treated with 1M LiGH (0.7 mL) at room temperature. After 1.5 h, the reaction mixture was cooled to 0° C. and 1M HCl was slowly added until pH 2. The mixture was extracted with EtOAc and washed with H₂O. The aqueous layer was back extracted with EtOAc. The combined organic layer was dried (Na₂SO₄), filtered and concentrated to give 7-((4-fluorobenzyl)oxy)chromane-2-carboxylic acid (48g, 0.166 g, 97%) as a solid residue that was used without further purification: IR (neat) 3067, 3013, 2974, 2917, 2673, 1695, 1618, 1602, 1582, 1467, 1426, 1319, 1264, 1223, 1197, 1130, 1040, 953, 905, 834, 787, 663 cm⁻¹; ¹H NMR (DMSO-d₆, 600 MHz): δ12.96 (br s, 1H), 7.47 (app t, J=5.4, 8.4 Hz, 2H), 7.21 (app t, J=8.4 Hz, 2H), 6.93 (d, J=8.4H, 1H), 6.50 (dd, J=2.4, 8.4 Hz, 1H), 6.45 (d, J=2.4 Hz, 1H), 5.03 (s, 2H), 4.74 (dd, J=3.6, 6.6 Hz, 1H), 2.72-2.67 (m, 1H), 2.57-2.52 (m, 1H), 2.13-2.08 (m, 1H), 2.05-1.99 (m, 1H).

7-((4-Fluorobenzyl)oxy)-N-((tetrahydro-2H-pyran-2-yl)oxy)chromane-2-carboxamide (49g). A solution of 7-((4-fluorobenzyl)oxy)chromane-2-carboxylic acid (48g, 0.163 g, 0.539 mmol) in DMF/CH₂Cl₂ (1 mL, 1:1) was cooled to 0° C. and treated with 0-(tetrahydro-2H-pyran-2-yl)hydroxylamine (0.127 g, 1.08 mmol) in CH₂Cl₂ (0.3 mL). To this solution was added T3P (0.688 g, 1.08 mmol, 50% CH₃CN soln) in CH₂Cl₂ (0.4 mL) followed by DIPEA (0.190 mL, 1.08 mmol). The solution was slowly warmed to room temperature overnight. After 14 h, the reaction was extracted with CH₂Cl₂ and washed with H₂O, 0.25M HCl (2×), 1M LiCl (3×), and H₂O (2×), brine, then dried (Na₂SO₄), filtered and concentrated. The residue was purified by chromatography on SiO₂ (ISCO-Rf, 0-40% EtOAc/CH₂Cl₂) to give 7-((4-fluorobenzyl)oxy)-N-((tetrahydro-2H-pyran-2-yl)oxy)chromane-2-carboxamide (49g, 0.156 g, 72%) as a white solid: IR (neat) 3140, 2944, 1689, 1660, 1601, 1583, 1467, 1426, 1353, 1304, 1258, 1173, 1120, 1063, 1008, 970, 907, 856, 792, 734, 668 cm⁻¹; ¹H NMR (CDCl₃, 600 MHz): δ 9.09 (br s, 1H), 7.39 (dd, J=5.4, 8.4 Hz, 2H), 7.07 (app t, J=8.4 Hz, 2H), 6.96 (d, J=8.4 Hz, 1H), 6.56 (dd, J=3.0, 8.4 Hz, 1H), 6.50 (dd, J=2.4, 11.4 Hz, 1H), 5.02 (app t, J=3.0 Hz, 0.5H), 4.99 (s, 2H), 4.96 (app t, J=3.0 Hz, 0.5H), 4.62-4.59 (m, 1H), 4.01-3.96 (m, 1H), 3.69-3.63 (m, 1H), 2.84-2.71 (m, 2H), 2.46-2.39 (m, 1H), 2.10-1.99 (m, 1H), 1.89-1.81 (m, 3H), 1.68-1.60 (m, 3H).

7-((4-Fluorobenzyl)oxy)-N-hydroxychromane-2-carboxamide (50g). A solution of 7-((4-fluorobenzyl)oxy)-N-((tetrahydro-2H-pyran-2-yl)oxy)chromane-2-carboxamide (49g, 0.156 g, 0.389 mmol) in CH₂Cl₂/MeOH (2 mL, 1:1) was treated with Amberlyst-15 (0.069 g, washed with MeOH) at room temperature. After 5 h, the reaction mixture was heated to 45° C. for 1 h. The reaction mixture was cooled to room temperature and filtered through Celite®, rinsed with CH₂Cl₂/MeOH, and concentrated. The solid was triturated with Et₂O/hexanes (1:2) and the solid was collected by filtration and dried to give 7-((4-fluorobenzyl)oxy)-N-hydroxychromane-2-carboxamide (50g, 0.090 g, 73%) as a tan solid: IR (neat) 3160, 2952, 2853, 1648, 1620, 1583, 1544, 1459, 1417, 1340, 1296, 1231, 1179, 1115, 1072, 1020, 959, 876, 826, 755, 699, 621 cm¹; 1H NMR (DMSO-d₆, 600 MHz): δ10.76 (s, 1H), 8.94 (s, 1H), 7.46 (dd, J=5.4, 8.4 Hz, 2H), 7.21 (app t, J=8.4 Hz, 2H), 6.94 (d, J=8.4 Hz, 1H), 6.51 (dd, J=2.4, 8.4 Hz, 1H), 6.47 (d, J=2.4 Hz, 1H), 5.02 (s, 2H), 4.47 (dd, J=3.0, 9.0 Hz, 1H), 2.72-2.67 (m, 1H), 2.61 (dt, J=4.8, 10.8 Hz, 1H), 2.10-2.05 (m, 1H), 1.93-1.83 (m, 1H); ¹³C NMR (DMSO-d₆, 150 MHz): 3166.3, 161.7 (J_{CF}=241 Hz), 157.4, 153.9, 133.4 (J_{CF}=3 Hz), 129.8, 129.74, 129.69, 115.2 (J_{CF}=20 Hz), 114.2, 107.8, 102.7, 73.8, 68.4, 24.4, 22.3; ¹⁹F NMR (DMSO-d₆, 376 MHz): δ−114.6; HRMS (ESI+) m/z calcd for C₁₇H₁₅NO₄F [M−H] 316.0980, found 316.0987.

Ethyl 7-phenethoxychromane-2-carboxylate (47h). A solution of ethyl 7-hydroxychromane-2-carboxylate (46, 0.100 g, 0.45 mmol) in MeCN (5 mL) was treated with K₂CO₃ (0.125 g, 0.904 mmol) followed by 2-bromoethyl benzene (0.126 mL, 0.922 mmol) and the reaction mixture was heated at 65° C. After 8.5 h, the reaction mixture was cooled to room temperature. After 16 h, the reaction mixture was extracted with EtOAc and washed with H₂O. The aqueous layer was back extracted with EtOAc (4×). The combined organic layer was washed with brine, dried (Na₂SO₄), filtered and concentrated. The residue was purified by chromatography on SiO₂ (ISCO-Rf, 0-50% EtOAc/hexanes) to give ethyl 7-phenethoxychromane-2-carboxylate (47 h, 0.093 g, 63%) as an oil: ¹H NMR (CDCl₃, 600 MHz): δ 7.32-7.27 (m, 3H), 7.24-7.22 (m, 1H), 6.90 (d, J=8.4 Hz 1H), 6.51 (d, J=3.0 Hz, 1H), 6.46 (dd, J=2.4, 8.4 Hz, 1H), 4.68 (dd, J=3.6, 7.8 Hz, 1H), 4.27-4.23 (m, 2H), 4.15-4.11 (m, 2H), 3.07 (t, J=7.2 Hz, 2H), 2.79-2.74 (m, 1H), 2.70-2.65 (m, 1H), 2.28-2.23 (m, 1H), 2.18-2.12 (m, 1H), 1.29 (t, J=7.2 Hz, 3H).

Phenethoxychromane-2-carboxylic acid (48h). A solution of ethyl 7-phenethoxychromane-2-carboxylate (47 h, 0.158 g, 0.486 mmol) in EtOH/THF (4 mL, 3/1) was treated with 1M LiGH (0.7 mL) at room temperature. After 2 h, the reaction mixture was cooled to 0° C. and acidified with 1 M HCl to pH 2. The solution was extracted with EtOAc and washed with H₂O. The aqueous layer was back extracted with EtOAc (4×). The combined organic layer was washed with brine, dried (Na₂SO₄), filtered and concentrated to give 7-phenethoxychromane-2-carboxylic acid (48 h, 0.142 g, 98%) as a white solid: ¹H NMR (DMSO-d₆, 600 MHz): δ 12.98 (br s, 1H), 7.31-7.29 (m, overlapping signals, 4H), 7.23-7.20 (m, 1H), 6.92 (d, J=7.8 Hz, 1H), 6.42 (dd, J=3.0, 8.4 Hz, 1H), 6.37 (d, J=2.4 Hz, 1H), 4.74 (dd, J=4.2, 6.6 Hz, 1H), 4.16-4.10 (m, 2H), 3.00 (t, J=7.2 Hz, 2H), 2.71-2.66 (m, 1H), 2.57-2.52 (m, 1H), 2.13-2.08 (m, 1H), 2.05-1.99 (m, 1H).

7-Phenethoxy-N-((tetrahydro-2H-pyran-2-yl)oxy)chromane-2-carboxamide (49h). A solution of 7-phenethoxychromane-2-carboxylic acid (48 h, 0.140 g, 0.47 mmol) in DMF/CH₂Cl₂ (2 mL, 1/1) was cooled to 0° C. and treated with O-(tetrahydro-2H-pyran-2-yl)hydroxylamine (0.110 g, 0.94 mmol) in CH₂Cl₂ (0.3 mL). To this solution was added T3P (0.600 g, 0.94 mmol, 50% CH₃CN soln) in CH₂Cl₂ (0.4 mL) followed by DIPEA (0.163 mL, 0.94 mmol). After 10 min, the reaction mixture was slowly warmed to room temperature. After 13 h, the reaction mixture was extracted with CH₂Cl₂ and washed with H₂O. The aqueous layer was back extracted with CH₂Cl₂ and the combined organic layer was washed with 0.25 M HCl (2×), 1M LiCl (3×), and water (2×), dried (Na₂SO₄), filtered and concentrated. The residue was purified by chromatography on SiO₂ (ISCO-Rf, 0-25% EtOAc/CH₂Cl₂) to give 7-phenethoxy-N-((tetrahydro-2H-pyran-2-yl)oxy)chromane-2-carboxamide (49 h, 0.159 g, 85%) as a light yellow oil: IR (neat) 3225, 3026, 2869, 1681, 1620, 1504, 1470, 1431, 1338, 1256, 1177, 1110, 1022, 896, 815, 698 cm⁻¹; ¹H NMR (CDCl₃, 600 MHz): δ 9.09 (s, 1H), 7.33-7.27 (m, overlapping signals, 4H), 7.26-7.23 (m, 1H), 6.94 (d, J=8.4 Hz, 1H), 6.50 (dd, J=2.4, 8.4 Hz, 1H), 6.43 (dd, J=2.4, 10.2 Hz, 1H), 5.02 (app t, J=3.0 Hz, 0.5H), 4.96 (app t, J=3.0 Hz, 0.5H), 4.61-4.58 (m, 1H), 4.13 (t, J=6.6 Hz, 2H), 4.00-3.96 (m, 1H), 3.69-3.61 (m, 1H), 3.08 (t, J=6.6 Hz, 2H), 2.83-2.70 (m, 2H), 2.45-2.38 (m, 1H), 2.09-1.98 (m, 1H), 1.89-1.78 (m, 3H), 1.69-1.60 (m, 3H).

N-Hydroxy-7-phenethoxychromane-2-carboxamide (50h). A solution of 7-phenethoxy-N-((tetrahydro-2H-pyran-2-yl)oxy)chromane-2-carboxamide (49 h, 0.154 g, 0.388 mmol) in CH₂Cl₂/MeOH (4 mL, 1/1) was treated with Amberlyst-15 (0.045 g, washed with MeOH) at room temperature. After 14 h, additional Amberlyst-15 (0.020 g) were added. After 2.5 h, the reaction mixture was filtered through Celite®, rinsed with CH₂Cl₂/MeOH and concentrated. The solid residue was triturated with Et₂O:hexanes (1:2.5) and the solid was collected by filtration and dried in vacuo to give N-hydroxy-7-phenethoxychromane-2-carboxamide (50 h, 0.086 g, 71%) as a tan solid: IR (neat) 3281, 3084, 2952, 1621, 1583, 1537, 1464, 1384, 1316, 1271, 1202, 1163, 1075, 1006, 902, 858, 789, 720, 694 cm⁻¹; ¹H NMR (DMSO-d₆, 600 MHz): 310.74 (br s, 1H), 8.92 (br s, 1H), 7.31-7.28 (m, overlapping signals, 4H), 7.24-7.20 (m, 1H), 6.93 (d, J=8.4 Hz, 1H), 6.44 (dd, J=2.4, 8.4 Hz, 1H), 6.41 (d, J=3.0 Hz, 1H), 4.48 (dd, J=3.0, 9.0 Hz, 1H), 4.13-4.10 (m, 2H), 3.00 (t, J=7.2 Hz, 2H), 2.71-2.66 (m, 1H), 2.61 (dt, J=5.4, 10.8 Hz, 1H), 2.10-2.06 (m, 1H), 1.92-1.86 (m, 1H); ¹³C NMR (DMSO-d₆, 150 MHz): δ166.4, 157.5, 153.8, 138.4, 129.8, 128.9, 128.3, 126.2, 113.9, 107.6, 102.3, 73.8, 68.1, 34.8, 24.4, 22.3; HRMS (ESI+) m/z calcd for C₁₈H₁₈NO₄ [M−H] 312.1230, found 312.1239.

Ethyl 7-((4-chlorobenzyl)oxy)chromane-2-carboxylate (47i). A solution of ethyl 7-hydroxychromane-2-carboxylate (46, 0.150g, 0.674 mmol) in DMF/MeCN (5 mL, 1:1) was treated with Cs₂CO₃ (0.478g, 1.46 mmol) followed by 4-chlorobenzyl bromide (0.287 g, 1.39 mmol). The reaction mixture was heated at 85° C. for 1 h. The mixture was cooled to room temperature and extracted with EtOAc, washed with H₂O, brine (2×), 1M LiCl, H₂O, dried (Na₂SO₄), filtered and concentrated. The residue was purified by chromatography on SiO₂ (ISCO-Rf, 0-20% EtOAc/hexanes) to give ethyl 7-((4-chlorobenzyl)oxy)chromane-2-carboxylate (47i, 0.219 g, 94%) as a yellow oil: ¹H NMR (CDCl₃, 600 MHz): δ 7.34 (br s, 4H), 6.92 (d, J=8.4 Hz, 1H), 6.55 (d, J=2.4 Hz, 1H), 6.51 (dd, J=3.0, 8.4 Hz, 1H), 4.97 (d, J=1.2 Hz, 2H), 4.68 (dd, J=3.6, 7.8 Hz, 1H), 4.27-4.23 (m, 2H), 2.79-2.74 (m, 1H), 2.70-2.65 (m, 1H), 2.28-2.23 (m, 1H), 2.18-2.12 (m, 1H), 1.30 (t, J=7.2 Hz, 3H).

7-((4-Chlorobenzyl)oxy)chromane-2-carboxylic acid (48i). A solution of ethyl 7-((4-chlorobenzyl)oxy)chromane-2-carboxylate (47i, 0.187 g, 0.540 mmol) in EtOH/THF (4 mL, 3/1) was treated with 1M LiGH (0.700 mL) at room temperature. After 2 h, the solution was cooled to 0° C. and acidified with 1M HCl to pH 1-2. The mixture was extracted with EtOAc and washed with H₂O. The aqueous layer was back extracted with EtOAc. The combined organic layer was washed with brine, dried (Na₂SO₄), filtered and concentrated to give 7-((4-chlorobenzyl)oxy)chromane-2-carboxylic acid (48i, 170 mg, 99%) as a white solid: ¹H NMR (DMSO-d₆, 300 MHz): δ 12.95 (br s, 1H), 7.44 (br s, 4H), 6.93 (d, J=8.4 Hz, 1H), 6.49 (dd, J=2.7, 8.4 Hz, 1H), 6.44 (d, J=2.4 Hz, 1H), 5.05 (s, 2H), 4.74 (dd, J=3.9, 6.3 Hz, 1H), 2.74-2.65 (m, 1H), 2.59-2.55 (m, 1H), 2.16-1.98 (m, 2H). [0283] 7-((4-Chlorobenzyl)oxy)-N-((tetrahydro-2H-pyran-2-yl)oxy)chromane-2-carboxamide (49i). A solution of 7-((4-chlorobenzyl)oxy)chromane-2-carboxylic acid (48i, 0.165 g, 0.520 mmol) in DMF/CH₂Cl₂ (0.6 mL, 1/1) was cooled to 0° C. and treated with 0-(tetrahydro-2H-pyran-2-yl)hydroxylamine (0.125 g, 1.07 mmol) in CH₂Cl₂ (0.3 mL). To this solution was added T3P (0.680 g, 1.07 mmol, 50% CH₃CN soln) in CH₂Cl₂ (0.3 mL) followed by DIPEA (0.190 mL, 1.09 mmol). The solution was stirred at 0° C. for 10 min and then warmed to room temperature. After 15 h, the reaction mixture was extracted with CH₂Cl₂ and washed with H₂O. The aqueous layer was back extracted with CH₂Cl₂ (2×). The combined organic layer was washed with 0.25M HCl (2×), 1M LiCi (2×), dried (Na₂SO₄), filtered and concentrated. The solid was purified by chromatography on SiO₂ (ISCO-Rf, 0-60% EtOAc/hexanes) to give 7-((4-chlorobenzyl)oxy)-N-((tetrahydro-2H-pyran-2-yl)oxy)chromane-2-carboxamide (49i, 0.96 g, 44%-contained small amount of residual solvent) as a white solid: ¹H NMR (DMSO-d₆, 600 MHz): δ 11.27 (s, 1H), 7.44 (s, 4H), 6.95 (d, J=8.4 Hz, 1H), 6.52 (dd, J=3.0, 8.4 Hz, 1H), 6.48 (dd, J=2.4, 6.0 Hz, 1H), 5.05 (s, 2H), 4.90-4.89 (m, 1H), 4.52-4.49 (m, 1H), 4.03-3.98 (m, 1H), 3.51-3.48 (m, 1H), 2.73-2.68 (m, 1H), 2.65-2.61 (m, 1H), 2.10-2.06 (m, 1H), 1.91-1.86 (m, 1H), 1.68-1.65 (m, 3H), 1.54-1.50 (m, 3H).

7-((4-Chlorobenzyl)oxy)-N-hydroxychromane-2-carboxamide (50i). A solution of 7-((4-chlorobenzyl)oxy)-N-((tetrahydro-2H-pyran-2-yl)oxy)chromane-2-carboxamide (49i, 0.087 g, 0.208 mmol) in MeOH/CH₂Cl₂ (2.6 mL, 1/1) was treated with Amberlyst-15 (0.040 g, washed with MeOH) at room temperature. After 24 h, the reaction mixture was filtered through Celite®, rinsed with MeOH/CH₂Cl₂ (1:1), and concentrated. The residue was triturated with Et₂O/hexanes (1:1) and the solid was collected by filtration and dried in vacuo to give 7-((4-chlorobenzyl)oxy)-N-hydroxychromane-2-carboxamide (50i, 0.048 g, 69%) as a pink solid: IR: δ278, 3065, 2949, 2848, 1621, 1585, 1505, 1454, 1379, 1316, 1278, 1204, 1161, 1127, 1090, 1017, 940, 874, 806, 761, 693, 664 cm⁻¹; ¹H NMR (DMSO-d₆, 600 MHz): δ10.76 (s, 1H), 8.93 (s, 1H), 7.44 (s, 4H), 6.94 (d, J=8.4 Hz, 1H), 6.51 (dd, J=2.4, 8.4 Hz, 1H), 6.46 (d, J=2.4 Hz, 1H), 5.04 (s, 2H), 4.47 (dd, J=3.0, 9.0 Hz, 1H), 2.72-2.67 (m, 1H), 2.62 (dt, J=4.8, 10.8 Hz, 1H), 2.09-2.05 (m, 1H), 1.92-1.86 (m, 1H); ¹³C NMR (DMSO-d₆, 100 MHz): δ166.3, 157.2, 153.9, 136.2, 132.2, 129.9, 129.3, 128.4, 114.2, 107.8, 102.7, 73.8, 68.2, 24.4, 22.3; HRMS (ESI+) m/z calcd for C₁₇H₁₅NO₄Cl [M−H] 332.0684, found 332.0705.

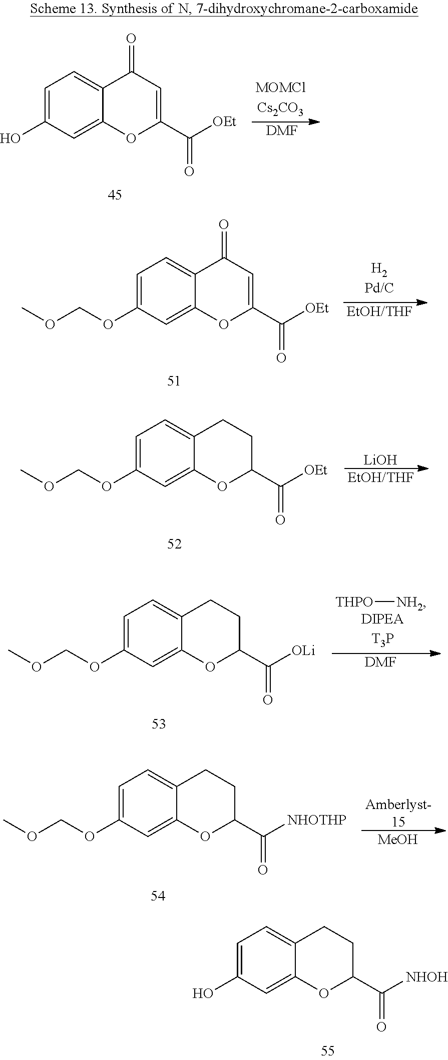

Ethyl 7-(methoxymethoxy)-4-oxo-4H-chromene-2-carboxylate (51). To a solution of ethyl 7-hydroxy-4-oxo-4H-chromene-2-carboxylate (45, 0.238 g, 1.02 mmol) in DMF (2.5 mL) was added Cs₂CO₃ (0.671 g, 2.06 mmol) at room temperature. To this orange colored mixture was added MOMCI (0.14 mL, 1.57 mmol) at room temperature. After 30 min, the mixture was extracted with EtOAc, washed with brine (2×), dried (Na₂SO₄), filtered and concentrated to give ethyl 7-(methoxymethoxy)-4-oxo-4H-chromene-2-carboxylate (51, 0.279 g, 98%) as a light yellow solid that was used without further purification: mp 104-106° C.; ¹H NMR (CDCl₃, 500 MHz): 38.01 (d, J=8.8 Hz, 1H), 7.14 (d, J=2.2 Hz, 1H), 7.01 (dd, J=2.2, 8.8 Hz, 1H), 6.98 (s, 1H), 5.20 (d, J=3.6 Hz, 2H), 4.38 (q, J=7.2 Hz, 2H), 3.44 (s, 3H), 1.36 (t, J=7.2 Hz, 3H); ¹³C NMR (CDCl₃, 125 MHz): 3177.4, 162.2, 160.3, 157.3, 151.9, 126.9, 118.9, 116.2, 114.7, 103.4, 94.3, 62.7, 56.3, 13.9; HRMS (ESI+) m/z calcd for C₁₄H₁₅O₆ [M+H] 279.0863, found 279.0862.

Ethyl 7-(methoxymethoxy)chromane-2-carboxylate (52). A solution of ethyl 7-(methoxymethoxy)-4-oxo-4H-chromene-2-carboxylate (51, 0.279 g, 1.00 mmol) in EtOH/THF (5 mL, 4/1) was evacuated and purged with N₂ (2×). To this solution was added 10% Pd/C (0.031 g) and the mixture was evacuated and purged with H₂ (2×). The reaction mixture was kept under H₂ (1 atm-balloon) overnight. The mixture was filtered through Celite®, rinsed with EtOAc and concentrated to give ethyl 7-(methoxymethoxy)chromane-2-carboxylate (52, 0.267 g, 99%) as a light yellow oil that was used without further purification: ¹H NMR (CDCl₃, 500 MHz): 36.90 (d, J=8.3 Hz, 1H), 6.64 (d, J=2.2 Hz, 1H), 6.55 (dd, J=2.4, 8.4 Hz, 1H), 5.09 (q, J_{AB}=6.7 Hz, 2H), 4.66 (dd, J=3.5, 3.5 Hz, 1H), 4.23 (q, J=7.1 Hz, 2H), 3.43 (s, 3H), 2.77-2.71 (m, 1H), 2.69-2.63 (m, 1H), 2.25-2.19 (m, 1H), 2.16-2.09 (m, 1H), 1.27 (t, J=7.2 Hz, 3H); ¹³C NMR (CDCl₃, 100 MHz): δ170.6, 156.5, 153.9, 129.6, 114.5, 109.3, 104.5, 94.3, 73.6, 61.2, 55.7, 24.5, 22.5, 14.0; HRMS (ESI+) m/z calcd for C₁₄H₁₉O₅[M+H] 267.1227, found 267.1224.

Lithium 7-(methoxymethoxy)chromane-2-carboxylate (53). To a solution of ethyl 7-(methoxymethoxy)chromane-2-carboxylate (52, 0.265 g, 0.995 mmol) in EtOH/THF (4.0 mL, 3/1) was added 1M LiGH (1.0 mL) at room temperature. After 2 d, the solution was concentrated and the residue was diluted with PhMe and concentrated (2×) to give lithium 7-(methoxymethoxy)chromane-2-carboxylate (53) that was used without further purification: HRMS (ESI+) m/z calcd for C₁₂H₁₃O₅ [M−H] 237.0757, found 237.0760.

7-(Methoxymethoxy)-N-((tetrahydro-2H-pyran-2-yl)oxy)chromane-2-carboxamide (54). A solution of lithium 7-(methoxymethoxy)chromane-2-carboxylate (53, 0.242 g, 0.99 mmol, crude), 0-(tetrahydro-2H-pyran-2-yl)hydroxylamine (0.153 g, 1.31 mmol) in DMF (3.0 mL) was treated with T3P (0.756 g, 50% EtOAc soln) in DMF (1.0 mL) followed by DIPEA (0.20 mL, 1.20 mmol) at room temperature. After 22 h, the solution was extracted with EtOAc, washed with 0.5M HCl, brine, dried (Na₂SO₄), filtered and concentrated to give 7-(methoxymethoxy)-N-((tetrahydro-2H-pyran-2-yl)oxy)chromane-2-carboxamide (54, 0.155 g, 46%) as a light yellow oil that was used without further purification: ¹H NMR (CDCl₃, 400 MHz): δ9.16 (s, 1H), 6.94 (d, J=8.8 Hz, 1H), 6.61-6.58 (m, overlapping signals, 2H), 5.12 (s, 2H), 5.02-5.01 (m, 0.5H), 4.97-4.96 (m, 0.5H), 4.60-4.56 (m, 1H), 4.02-3.96 (m, 1H), 3.72-3.58 (m, 1H), 3.46 (s, 3H), 2.79-2.69 (m, 2H), 2.45-2.38 (s, 1H), 2.06-1.98 (m, 1H), 1.96-1.79 (m, 3H), 1.67-1.58 (m, 3H); HRMS (LCMS ESI+) m/z calcd for C₁₇H₂₃O₆NNa [M+Na] 360.1418, found 360.1415.

N,7-Dihydroxychromane-2-carboxamide (55). To a solution of 7-(methoxymethoxy)-N-((tetrahydro-2H-pyran-2-yl)oxy)chromane-2-carboxamide (54, 0.155 g, 0.46 mmol) in MeOH (2.5 mL) was added Amberlyst-15 (0.051 g, washed with MeOH) at room temperature. After 16 h, the solution was filtered through Celite®, rinsed with MeOH and concentrated. The solid residue was slurried with Et₂O and the off-white solid was collected by filtration and dried in vacuo at 100° C. to give N,7-dihydroxychromane-2-carboxamide (55, 0.069 g, 72%) as an off-white solid: mp 174-176° C.; IR (neat) 3346, 2896, 1685, 1621, 1597 1536, 1504, 1460, 1359, 1337, 1303, 1195, 1153, 1120, 1099, 1068, 1056, 1000, 958, 899, 837, 818, 778 cm⁻¹; ¹H NMR (DMSO-d₆, 600 MHz): 310.75 (s, 1H), 9.18 (s, 1H), 8.91 (s, 1H), 6.80 (d, J=8.4 Hz, 1H), 6.27 (dd, J=2.4, 8.4 Hz, 1H), 6.24 (d, J=1.8 Hz, 1H), 4.43 (dd, J=2.4, 2.4 Hz, 1H), 2.65-2.58 (m, 1H), 2.7-2.54 (m, 1H), 2.07-2.05 (m, 1H), 1.90-1.85 (m, 1H); ¹³C NMR (DMSO-d₆, 150 MHz): δ166.5, 156.4, 153.8, 129.7, 112.1, 108.3, 103.1, 73.8, 24.7, 22.4; HRMS (ESI+) m/z calcd for C₁₀H₁₀O₄N [M−H] 208.0604, found 208.0608.

### Part 4: 7-C Chromanes

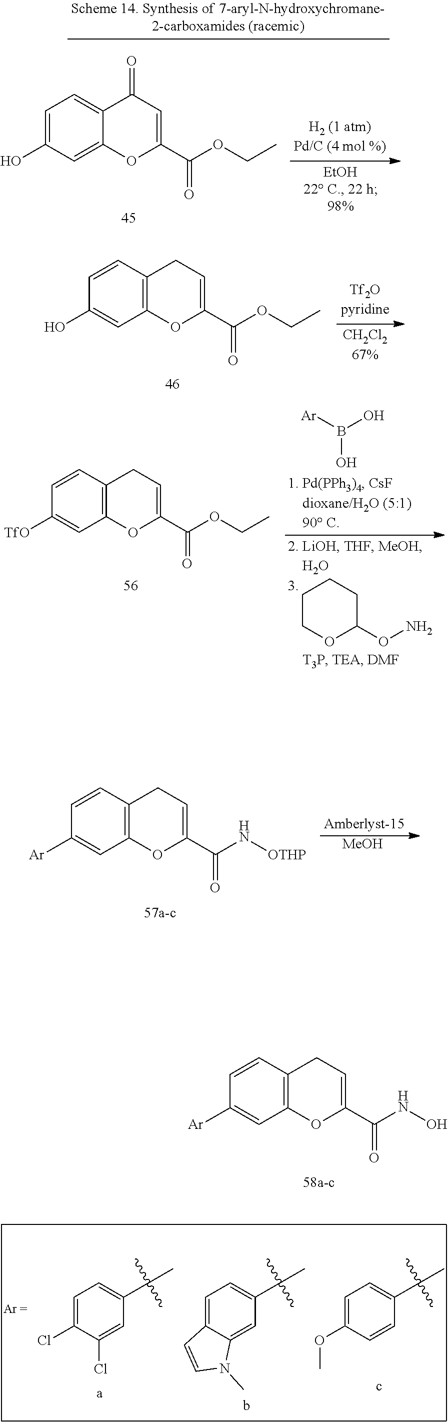

Ethyl 7-hydroxychromane-2-carboxylate (46). A solution of ethyl 7-hydroxy-4-oxo-4H-chromene-2-carboxylate (45, 2.01 g, 8.59 mmol) in EtOH (18 mL) was evacuated and purged with N₂ (2×). The solution was treated with Pd/C (10%, 0.320 g, 0.301 mmol) and evacuated and purged with H₂ (2×) and kept under H₂ (1 atm-balloon). After 17 h, the mixture was filtered through Celite® and rinsed with EtOAc. The filtrate was concentrated to give ethyl 7-hydroxychromane-2-carboxylate (46, 1.86 g, 98%) as a grey solid: ¹H NMR (CDCl₃, 500 MHz): δ 6.87 (d, J=8.2 Hz, 1H), 6.46 (d, J=2.5 Hz, 1H), 6.39, (dd, J=8.2, 2.5 Hz, 1H), 5.07 (s, 1H), 4.69 (dd, J=7.5, 3.6 Hz, 1H), 4.26 (q, J=7.2 Hz, 2H), 2.79-2.73 (m, 1H), 2.69-2.64 (m, 1H), 2.28-2.22 (m, 1H), 2.19-2.12 (m, 1H), 1.29 (t, J=7.2 Hz, 3H).

Ethyl 7-(((trifluoromethyl)sulfonyl)oxy)chromane-2-carboxylate (56). A solution of ethyl 7-hydroxychromane-2-carboxylate (46, 1.85 g, 3.32 mmol) in CH₂Cl₂ (20 mL) was cooled to 0° C. and treated with pyridine (1.30 mL, 16.1 mmol) followed by trifluoromethanesulfonic anhydride (2.00 mL, 11.9 mmol) dropwise. After 5 min, the mixture was warmed to room temperature. After 3 h, the mixture was extracted with Et₂O and washed with 10% HCl, satd. NaHCO₃, brine, dried (Na₂SO₄), filtered through a pad of SiO₂ and rinsed with hexanes:EtOAc (2:1) to give ethyl 7-(((trifluoromethyl)sulfonyl)oxy)chromane-2-carboxylate (56, 2.64 g, 90%) as a yellow oil: IR(CH₂Cl₂) 2942, 1753, 1612, 1597, 1494, 1421, 1206, 1106 cm⁻¹; ¹H NMR (CDCl₃, 500 MHz): δ 7.08 (d, J=8.5 Hz, 1H), 6.87 (d, J=2.5 Hz, 1H), 6.79, (dd, J=8.4, 2.5 Hz, 1H), 4.75 (dd, J=7.1, 5.3 Hz, 1H), 4.25 (q, J=7.2 Hz, 2H), 2.86-2.80 (m, 1H), 2.78-2.72 (m, 1H), 2.31-2.25 (m, 1H), 2.24-2.17 (m, 1H), 1.29 (t, J=7.3 Hz, 3H); ¹³C NMR (CDCl₃, 125 MHz): δ 170.3, 154.5, 148.5, 130.6, 121.9, 118.9 (q, J=321 Hz), 113.6, 110.3, 73.9, 61.8, 24.1, 22.9, 14.3; ¹⁹F NMR (CDCl₃, 470 MHz): δ−72.9; HRMS (ESI+) m/z calcd for C₁₃H₁₄F₃O₆S [M+H] 355.0458, found 355.0458.

7-(3,4-Dichlorophenyl)-N-((tetrahydro-2H-pyran-2-yl)oxy)chromane-2-carboxamide (57a). In a N₂-filled glove box, a microwave vial was charged with Pd(PPh₃)₄ (0.041 g, 0.036 mmol) and CsF (0.232 g, 1.53 mmol). 3,4-Dichlorophenylboronic acid (0.143 g, 0.747 mmol) and ethyl 7-(((trifluoromethyl)sulfonyl)oxy)chromane-2-carboxylate (7, 0.245 g, 0.692 mmol) in degassed dioxane/H₂O (5:1, 8.2/1.8 mL) were added to the reaction vial, and the mixture was sparged with N₂ for 15 min. The vial was sealed and heated at 90° C. for 7 h. The reaction mixture was extracted with EtOAc and satd. NaHCO₃: satd. NaCl (1:1). The aqueous layer was back extracted with EtOAc (2×). The combined organic layer was dried (Na₂SO₄), filtered through a pad of SiO₂, rinsed with EtOAc, and concentrated to afford a crude yellow oil (0.323 g) that was used with no further purification: ¹H NMR (300 MHz; CDCl₃) δ 7.65 (d, J=2.0 Hz, 1H), 7.47 (d, J=8.4 Hz, 1H), 7.39 (dd, J=8.4, 2.1 Hz, 1H), 7.14-7.10 (m, 2H), 7.05 (dd, J=7.7, 1.8 Hz, 1H), 4.76 (dd, J=7.4, 3.8 Hz, 1H), 4.27 (q, J=7.3 Hz, 2H), 2.93-2.73 (m, 2H), 2.37-2.16 (m, 2H), 1.31 (t, J=7.1 Hz, 3H). The oil was dissolved in THE (2.0 mL) and MeOH (2.0 mL). The mixture was treated with LiOH monohydrate (0.0581 g, 1.38 mmol) in H₂O (2.0 mL) at room temperature. After 1.5 h, the solution was concentrated and the residue was azeotroped with PhMe (2×). The solid residue (0.228 g, 0.692 mmol) was dissolved in DMF (1.5 mL) and treated with O-(tetrahydro-2H-pyran-2-yl)hydroxylamine (0.146 g, 1.25 mmol). The mixture was cooled to 0° C. and treated with TEA (0.100 mL, 0.717 mmol) followed by T3P (0.630 mL, 1.06 mmol, 50% EtOAc soln). The mixture was warmed to room temperature. After 15 h, the mixture was extracted with EtOAc, washed with 0.5 M HCl, brine, dried (Na₂SO₄), filtered through a pad of SiO₂, rinsed with EtOAc, and concentrated to give 7-(3,4-dichlorophenyl)-N-((tetrahydro-2H-pyran-2-yl)oxy)chromane-2-carboxamide (57a, 0.174 g, 59%-3 steps) as a yellow oil: ¹H NMR (CDCl₃, 300 MHz): δ 9.14 (s, 1H), 7.65 (d, J=3.5 Hz, 1H), 7.51-7.48 (m, 1H), 7.40-7.37 (m, 1H), 7.16-7.07 (m, 3H), 5.04 (br s, 0.5H), 4.98 (br s, 0.5H), 4.68-4.64 (m, 1H), 4.03-3.96 (m, 1H), 3.70-3.61 (m, 2H), 2.91-2.84 (m, 2H), 2.55-2.43 (m, 1H), 2.18-2.04 (m, 1H), 1.86-1.59 (m, 6H).

7-(1-Methyl-1H-indol-5-yl)-N-((tetrahydro-2H-pyran-2-yl)oxy)chromane-2-carboxamide (57b). In a N₂-filled glove box, a microwave vial was charged with Pd(PPh₃)₄ (0.041 g, 0.036 mmol) and CsF (0.232 g, 1.53 mmol). N-Methylindole-5-boronic acid (0.133 g, 0.760 mmol) and ethyl 7-(((trifluoromethyl)sulfonyl)oxy)chromane-2-carboxylate (7, 0.245 g, 0.692 mmol) in degassed dioxane/H₂O (5:1, 8.2 mL/1.8 mL) were added to the vial, and the mixture was sparged with N₂ for 15 min. The vial was sealed and heated at 90° C. for 7 h. The reaction mixture was extracted with EtOAc and satd. NaHCO₃: satd. NaCl (1:1). The aqueous layer was back extracted with EtOAc (2×). The combined organic layer was dried (Na₂SO₄), filtered through a pad of SiO₂, rinsed with EtOAc, and concentrated to give a crude light-yellow semi solid (0.213 g) that was used with no further purification: ¹H NMR (300 MHz; CDCl₃) δ 7.82 (d, J=1.2 Hz, 1H), 7.47 (dd, J=8.5, 1.6 Hz, 1H), 7.36 (d, J=8.5 Hz, 1H), 7.26 (bs, 1H), 7.19 (dd, J=7.8, 1.8 Hz, 1H), 7.09 (d, J=7.8 Hz, 1H), 7.07 (d, J=3.1 Hz, 1H), 6.52 (d, J=3.1 Hz, 1H), 4.75 (dd, J=7.6, 3.6 Hz, 1H), 4.28 (q, J=7.1 Hz, 2H), 3.81 (s, 3H), 2.93-2.75 (m, 2H), 2.36-2.29 (m, 1H), 2.37-2.17 (m, 1H), 1.31 (t, J=7.1 Hz, 3H). The oil was dissolved in THE (2.0 mL) and MeOH (2.0 mL). The mixture was treated with LiOH monohydrate (0.037 g, 1.54 mmol) in H₂O (2.0 mL) at room temperature. After 1.5 h, the solution was concentrated and the residue was azeotroped with PhMe (2×). The solid (0.217 g, 0.692 mmol) was dissolved in DMF (1.5 mL) and treated with O-(tetrahydro-2H-pyran-2-yl)hydroxylamine (0.146 g, 1.25 mmol). The mixture was cooled to 0° C. and treated with TEA (0.300 mL, 1.08 mmol) followed by T3P (0.600 mL, 1.01 mmol, 50% EtOAc soln). The mixture was warmed to room temperature. After 24 h, additional T3P (0.700 mL, 2.35 mmol) and O-(tetrahydro-2H-pyran-2-yl)hydroxylamine (0.125 g, 2.35 mmol) was added. After 40 h, the mixture was extracted with EtOAc, washed with 0.5M HCl, brine, dried (Na₂SO₄), filtered through SiO₂, rinsed with EtOAc, and concentrated to give 7-(1-methyl-1H-indol-5-yl)-N-((tetrahydro-2H-pyran-2-yl)oxy)chromane-2-carboxamide (58c, 0.094 g, 30%-3 steps, approx. 90% purity) as a yellow oil: ¹H NMR (CD₃OD, 400 MHz): δ 7.76 (s, 1H), 7.44-7.39 (m, 2H), 7.20-7.10 (m, 5H), 6.46 (s, 1H), 4.99 (br s, 1H), 4.64-4.60 (m, 1H), 4.10-4.07 (m, 2H), 4.00-3.87 (m, 1H), 3.82 (s, 3H), 3.62-3.51 (m, 1H), 2.96-2.80 (m, 2H), 2.33-2.31 (m, 1H), 2.13-2.04 (m, 1H), 1.91-1.49 (6H).

7-(4-Methoxyphenyl)-N-((tetrahydro-2H-pyran-2-yl)oxy)chromane-2-carboxamide (57c). In a N₂-filled glove box, a microwave vial was charged with Pd(PPh₃)₄ (0.0410 g, 0.0355 mmol) and CsF (0.236 g, 1.55 mmol). 4-Methoxyphenylboronic acid (0.164 g, 0.999 mmol) and ethyl 7-(((trifluoromethyl)sulfonyl)oxy)chromane-2-carboxylate (7, 0.250 g, 0.705 mmol) in degassed dioxane/H₂O (5:1, 9 mL/1.8 mL) were added to the vial, and the mixture was sparged with N₂ for 15 min. The vial was sealed and heated at 90° C. for 13 h. The reaction mixture was extracted with EtOAc and satd. NaHCO₃: satd. NaCl (1:1). The aqueous layer was back extracted with EtOAc (2×). The combined organic layer was dried (Na₂SO₄), filtered through a pad of SiO₂, rinsed with EtOAc, and concentrated to afford a crude oil (˜0.260 g) that was used with no further purification: ¹H NMR (300 MHz; CDCl₃) δ 7.51 (d, J=8.8 Hz, 2H), 7.16 (bs, 1H), 7.09-7.07 (m, 2H), 6.95 (d, J=8.8 Hz, 2H), 4.74 (dd, J=7.5, 3.6 Hz, 1H), 4.28 (q, J=7.1 Hz, 2H), 3.84 (s, 3H), 2.93-2.73 (m, 2H), 2.36-2.15 (m, 2H), 1.31 (t, J=7.1 Hz, 3H). The oil was dissolved in THE (2.0 mL) and MeOH (2.0 mL). The mixture was treated with LiOH monohydrate (0.0371 g, 0.884 mmol) in H₂O (2.0 mL) at room temperature. After 1.5 h, the solution was concentrated and the residue was azeotroped with PhMe (2×). The solid (0.214 g, 0.736 mmol) was dissolved in DMF (3.5 mL) and treated with O-(tetrahydro-2H-pyran-2-yl)hydroxylamine (0.166 g, 1.42 mmol). The mixture was cooled to 0° C. and treated with TEA (0.200 mL, 1.43 mmol) and T3P (50%, 0.660 mL, 1.11 mmol, 50% EtOAc soln). The mixture was warmed to room temperature. After 13 h, the mixture was extracted with EtOAc, washed with 0.5M HCl, brine, dried (Na₂SO₄), filtered through SiO₂, and concentrated to give 7-(4-methoxyphenyl)-N-((tetrahydro-2H-pyran-2-yl)oxy)chromane-2-carboxamide (57c, 0.150 g, 53%-3 steps) as a yellow oil: ¹H NMR (CDCl₃, 300 MHz): δ 9.15 (s, 1H), 7.50 (d, J=14.7 Hz, 2H), 7.11-7.07 (m, 3H), 6.97 (d, J=14.7 Hz, 2H), 5.05 (br s, 0.5H), 4.98 (br s, 0.5H), 4.69-4.64 (m, 1H), 4.02-3.95 (m, 1H), 3.85 (s, 3H), 3.69-3.60 (m, 1H), 3.50-3.48 (m, 1H), 2.96-2.79 (m, 2H), 2.53-2.44 (m, 1H), 2.17-2.05 (m, 1H), 1.90-1.80 (m, 3H), 1.70-1.16 (m, 3H).

7-(3,4-Dichlorophenyl)-N-hydroxychromane-2-carboxamide (58a). To a solution of 7-(3,4-dichlorophenyl)-N-((tetrahydro-2H-pyran-2-yl)oxy)chromane-2-carboxamide (57a, 0.170 g, 0.403 mmol) in MeOH (5.0 mL) was added Amberlyst-15 (0.040 g, 199 mmol, washed with MeOH) at room temperature. After 44 h, the reaction mixture was filtered through Celite®, rinsed with MeOH, and concentrated. The residue was triturated with hexanes/EtOAc (5:1) to give 7-(3,4-dichlorophenyl)-N-hydroxychromane-2-carboxamide (58a, 0.055 g, 39%) as a tan solid: mp 167-169° C.; IR (CH₂Cl₂) 3167, 2893, 1671, 1550, 1470, 1308 cm⁻¹; ¹H NMR (DMSO-d₆, 500 MHz): δ 10.78 (s, 1H), 8.96 (s, 1H), 7.86 (d, J=1.8 Hz, 1H), 7.70 (d, J=8.4 Hz, 1H), 7.62 (dd, J=8.4, 1.8 Hz, 1H), 7.21-7.16 (m, 3H), 4.60 (dd, J=8.3, 3.0 Hz, 1H), 2.85-2.78 (m, 1H), 2.76-2.70 (m, 1H), 2.15-2.12 (m, 1H), 2.02-1.95 (m, 1H); ¹³C NMR (DMSO-d₆, 125 MHz): δ 166.3, 153.6, 140.4, 136.5, 131.6, 130.9, 130.1, 130.0, 128.1, 126.5, 122.3, 118.7, 114.8, 73.8, 24.0, 22.6; HRMS (ESI+) m/z calcd for C₁₆H₁₂O₃NCl₂ [M−H] 336.0189, found 336.0198.

N-Hydroxy-7-(1-methyl-1H-indol-5-yl)chromane-2-carboxamide (58b). To a solution of 7-(1-methyl-1H-indol-5-yl)-N-((tetrahydro-2H-pyran-2-yl)oxy)chromane-2-carboxamide (57b, 0.094 g, 0.208 mmol, 90% purity) in MeOH (3.0 mL) was added Amberlyst-15 (0.029g, 134 mmol, washed with MeOH) at room temperature. After 24 h, the reaction mixture was filtered through Celite®, rinsed with MeOH, and concentrated. The residue was triturated with hexanes:EtOAc (5:1) to give N-hydroxy-7-(1-methyl-1H-indol-5-yl)chromane-2-carboxamide (58b, 0.027 g, 40%) as a grey-brown solid: mp 155-158° C.; IR (CH₂Cl₂) 3207, 2922, 1668, 1618, 1483, 1422, 1212 cm⁻¹; ¹H NMR (DMSO-d₆, 500 MHz): δ 10.80 (s, 1H), 8.94 (s, 1H), 7.76 (s, 1H), 7.49 (d, J=8.5 Hz, 1H), 7.40 (dd, J=8.5, 1.2 Hz, 1H), 7.34 (d, J=2.9 Hz, 1H), 7.17-7.10 (m, 3H), 6.46 (d, J=2.8 Hz, 1H), 4.58 (dd, J=8.5, 2.8 Hz, 1H), 3.81 (s, 3H), 2.84-2.78 (m, 1H), 2.75-2.70 (m, 1H), 2.17-2.13 (m, 1H), 2.02-1.95 (m, 1H); ¹³C NMR (DMSO-d₆, 125 MHz): δ 166.5, 153.5, 140.8, 135.9, 131.1, 130.3, 129.7, 128.5, 120.1, 119.8, 118.8, 118.0, 114.6, 110.0, 100.7, 73.9, 32.5, 24.3, 22.6; HRMS (ESI+) m/z calcd for C₁₉H₁₉O₃N₂ [M+H] 323.1390, found 323.1390.

N-Hydroxy-7-(4-methoxyphenyl)chromane-2-carboxamide (58c). To a solution of 7-(4-methoxyphenyl)-N-((tetrahydro-2H-pyran-2-yl)oxy)chromane-2-carboxamide (57c, 0.150 g, 0.391 mmol) in MeOH (5.0 mL) was added Amberlyst-15 (0.060 g, 283 mmol, washed with MeOH) at room temperature. After 16 h, the reaction mixture was filtered through Celite®, rinsed with MeOH, and concentrated. The residue was triturated with hexanes:EtOAc (5:1) to give N-hydroxy-7-(4-methoxyphenyl)chromane-2-carboxamide (58c, 0.078 g, 67%) as a tan-colored solid: mp 181-183° C.; IR (CH₂Cl₂) 3321, 2913, 2166, 1679, 1607, 1492, 1248 cm⁻¹; ¹H NMR (DMSO-d₆, 500 MHz): δ 10.79 (s, 1H), 8.94 (s, 1H), 7.54 (d, J=8.7 Hz, 2H), 7.09-7.06 (m, 3H), 7.00 (d, J=8.7 Hz, 2H), 4.57 (dd, J=8.6, 3.1 Hz, 1H), 3.78 (s, 3H), 2.82-2.77 (m, 1H), 2.74-2.68 (m, 1H), 2.16-2.10 (m, 1H), 2.01-1.94 (m, 1H); ¹³C NMR (DMSO-d₆, 125 MHz): δ 166.4, 158.8, 153.6, 139.0, 132.2, 129.9, 127.4, 120.4, 118.3, 114.3, 114.1, 73.9, 55.1, 24.3, 22.6; HRMS (ESI+) m/z calcd for C₁₇H₁₈O₄N [M−H] 300.1230, found 300.1230.

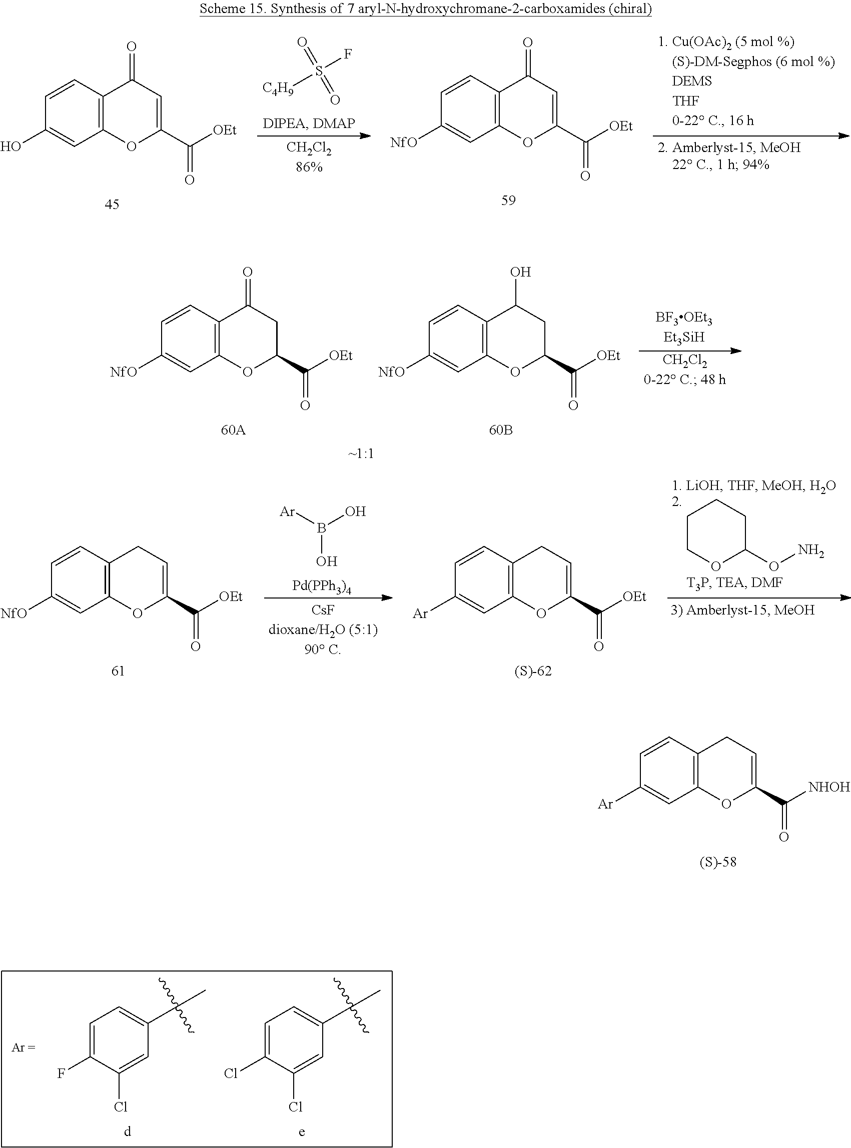

Ethyl 4-oxo-7-(((perfluorobutyl)sulfonyl)oxy)-4H-chromene-2-carboxylatecarboxylate (59). A solution of ethyl 7-hydroxy-4-oxo-4H-chromene-2-carboxylate (46, 5.00 g, 21.4 mmol) in CH₂Cl₂ (60 mL) was treated with DMAP (0.284 g, 2.33 mmol) and DIPEA (5.20 mL, 29.9 mmol) at room temperature. The solution was cooled to 0° C. and 1,1,2,2,3,3,4,4,4-nonafluorobutane-1-sulfonyl fluoride (92%, 5.00 mL, 25.6 mmol) was added dropwise. After 30 min, the mixture was warmed to rt. After 21 h, the reaction mixture was extracted CH₂Cl₂ and washed with H₂O. The aqueous layer was back extracted with CH₂Cl₂. The combined organic layer was washed with 0.5M HCl, brine, dried (Na₂SO₄), filtered, and concentrated. The crude mixture was filtered through a SiO₂ pad, rinsed with hexanes/EtOAc (2:1) and concentrated to give ethyl 4-oxo-7-(((perfluorobutyl)sulfonyl)oxy)-4H-chromene-2-carboxylatecarboxylate (59, 9.44 g, 86%) as a beige solid: mp 125-128° C.; IR (CH₂Cl₂) 3092, 3044, 1745, 1656, 1614, 1429, 1234, 1191, 1142, 1119 cm; ¹H NMR (CDCl₃, 400 MHz): δ 8.31 (d, J=8.9 Hz, 1H), 7.61 (d, J=2.3 Hz, 1H), 7.38 (dd, J=8.9, 2.3 Hz, 1H), 7.15 (s, 1H), 4.48 (q, J=7.1 Hz, 2H), 1.45 (t, J=7.1 Hz, 3H); ¹³C NMR (CDCl₃, 100 MHz): δ 177.1, 160.1, 156.3, 153.2, 153.0, 128.6, 124.1, 119.6, 115.4, 112.3, 63.5, 14.2; ¹⁹F NMR (CDCl₃, 376 MHz): δ−80.5 (t, J=9.6 Hz, 3F), −108.3 (t, J=13.5 \tilde{H}z, 2F), −120.7 (br 2 s, 2F), −125.68-−125.76 (m, 2F); HRMS (ESI+) m/z calcd for C₁₆H₁₀F₉O₇S [M+H] 516.9998, found 516.9991.

Ethyl (S)-4-oxo-7-(((perfluorobutyl)sulfonyl)oxy)chromane-2-carboxylate (60A) and ethyl (2S)-4-hydroxy-7-(((perfluorobutyl)sulfonyl)oxy)chromane-2-carboxylate (60B). In a three-neck flask equipped with an addition funnel, N₂ sparging line, and an outlet needle, a bondi-blue solution of Cu(OAc)₂ (Strem, 0.0748 g, 0.412 mmol) in freshly distilled THE (50 mL) was stirred under an atmosphere of N₂ until a homogeneous green-blue solution was obtained (ca. 15 min). To this solution was added (S)-DM-Segphos (0.357 g, 0.494 mmol) and the mixture was stirred for 15 min at rt, cooled to 0° C. and treated dropwise with diethoxymethylsilane (1.98 mL, 12.3 mmol) over a period of 5 min. The reaction mixture was stirred for an additional 30 min at 0° C. at which time the solution turned from blue-green to yellow. A solution of ethyl 4-oxo-7-(((perfluorobutyl)sulfonyl)oxy)-4H-chromene-2-carboxylatecarboxylate (59, 4.25 g, 8.23 mmol) in dry THE (35 mL) was added dropwise. The solution was stirred at 0° C. for 10 min and warmed to room temperature. After 5 h, the brown reaction mixture was cooled to 0° C. and treated with satd. NH₄Cl under vigorous stirring for 15 min. The mixture was extracted with EtOAc and washed with satd. NaCl, satd. NaHCO₃, dried (Na₂SO₄), and concentrated. The residue was diluted with hexanes/EtOAc (1:3) and passed through a SiO₂ pad (packed with hexanes) and rinsed with hexanes/EtOAc (1:3) to give a solid residue. This solid residue was re-subjected to the reaction conditions described above using THE (75 mL) to solubilize the solid material. The reaction was stirred for 12 h, and worked up as described above. The obtained crude material (6.5 g) was dissolved in MeOH (100 mL) and treated with Amberlyst-15 (0.288 g, washed with MeOH) at room temperature. After 2 h, the reaction mixture was filtered through Celite® and concentrated. The residue was purified by chromatography on SiO₂ (22-66% EtOAc/hexanes) to give ethyl (S)-4-oxo-7-(((perfluorobutyl)sulfonyl)oxy)chromane-2-carboxylate (60A, 2.28 g, 50%-94% purity-contains small amount Segphos impurity) as a yellow solid and ethyl (2S)-4-hydroxy-7-(((perfluorobutyl)sulfonyl)oxy)chromane-2-carboxylate (60B, 1.88 g, 44%) as a white solid: 60A: ¹H NMR (CDCl₃, 300 MHz): δ 7.98 (d, J=8.7 Hz, 1H), 7.08 (d, J=2.3 Hz, 1H), 7.00 (dd, J=8.7, 2.3 Hz, 1H), 5.14 (dd, J=8.2, 5.6 Hz, 1H), 4.27 (q, J=7.1 Hz, 2H), 3.11 (d, J=5.6 Hz, 1H), 3.10 (d, J=8.2 Hz, 1H), 1.28 (t, J=7.1 Hz, 3H); HRMS (ESI+) m/z calcd for C₁₆H₁₂F₉O₇S [M+H] 519.0155, found 519.0155; 60B: ¹H NMR (CDCl₃, 300 MHz): δ 7.41 (d, J=8.3 Hz, 1H), 6.93-6.89 (m, 2H), 4.90 (app t, J=5.0 Hz, 1H), 4.85 (app t, J=4.5 Hz, 1H), 4.23 (q, J=7.1 Hz, 2H), 2.54-2.39 (m, 2H), 1.28 (t, J=7.1 Hz, 3H); HRMS (ESI+) m/z calcd for C₁₆H₁₃F₉O₇SNa [M+Na] 543.0130, found 543.0131.

Ethyl (S)-7-(((perfluorobutyl)sulfonyl)oxy)chromane-2-carboxylate (61). A solution of ethyl (2S)-4-hydroxy-7-(((perfluorobutyl)sulfonyl)oxy)chromane-2-carboxylate (60B, 1.70 g, 3.27 mmol) in CH₂Cl₂ (12 mL) was treated with triethylsilane (1.67 mL, 10.5 mmol) at room temperature. The reaction mixture was then cooled to 0° C., and BF₃·OEt₂ (1.21 mL, 9.80 mmol) was added. After 10 min, the mixture was warmed to room temperature. After 48 h, the reaction mixture was extracted with CH₂Cl₂ and washed with H₂O. The organic layer was washed with brine (20 mL) and treated with SiO₂ and Na₂SO₄ and the mixture was stirred for 20 min. The suspension was filtered through a pad of SiO₂ and concentrated. The residue was purified by chromatography on SiO₂ (18% EtOAc/hexanes) to give ethyl (S)-7-(((perfluorobutyl)sulfonyl)oxy)chromane-2-carboxylate (61, 1.25 g, 76%) as a colorless oil: [α]_{D}=−7.9 (c 0.34, CH₂Cl₂); IR (CH₂Cl₂) 2986, 1753, 1612, 1597, 1494, 1422, 1236, 1197, 1143, 1104 cm⁻¹; ¹H NMR (CDCl₃, 500 MHz): δ 7.08 (d, J=8.5 Hz, 1H), 6.88 (d, J=2.5 Hz, 1H), 6.81 (dd, J=8.5, 2.5 Hz, 1H), 4.75 (dd, J=7.1, 3.4 Hz, 1H), 4.29-4.23 (m, 2H), 2.87-2.81 (m, 1H), 2.78-2.72 (m, 1H), 2.32-2.26 (m, 1H), 2.24-2.17 (m, 1H), 1.29 (t, J=7.1 Hz, 3H); ¹³C NMR (CDCl₃, 125 MHz): δ 170.3, 154.5, 148.7, 130.6, 121.9, 113.7, 110.3, 74.0, 61.8, 24.1, 22.9, 14.3; ¹⁹F NMR (CDCl₃, 470 MHz): δ−80.6 (t, J=10.1 Hz, 3F), −108.9 (t, J=13.6 Hz, 3F), −120.8-−120.90 (m, 2F), −125.76-−125.84 (m, 2F); HRMS (ESI+) m/z calcd for C₁₆H₁₃F₉O₆SNa [M+Na] 527.0181, found 527.0182.

Ethyl (S)-7-(3-chloro-4-fluorophenyl)chromane-2-carboxylate (S)-62d. In a N₂-filled glove box, a microwave vial was charged with Pd(PPh₃)₄ (0.0332 g, 0.0288 mmol) and CsF (0.192 g, 1.27 mmol). A solution of ethyl (S)-7-(((perfluorobutyl)sulfonyl)oxy)chromane-2-carboxylate (61, 0.290 g, 0.575 mmol) in dioxane/H₂O (5:1, 5.5 mL/1.1 mL) was sparged with N₂ (15 min) and added to the reaction vial, followed by the (3-chloro-4-fluorophenyl)boronic acid (0.108 g, 0.621 mmol). After additional sparging with N₂ (5 min), the vial was sealed, and heated at 90° C. for 15 h. The reaction mixture was extracted with EtOAc and washed with satd.

NaHCO₃:satd. NaCl (1:1). The aqueous layer was back extracted with EtOAc (2×). The combined organic layer was dried (Na₂SO₄), filtered, and concentrated. The residue was purified by chromatography on SiO₂ (0-18% EtOAc/hexanes) to give ethyl (S)-7-(3-chloro-4-fluorophenyl)chromane-2-carboxylate ((S)-62d, 0.138 g, 70%) as a yellow oil: [α]_{D}=−28.5 (c 0.14, CH₂Cl₂); IR (CH₂Cl₂) 2979, 2937, 1752, 1566, 1488, 1190 cm⁻¹; ¹H NMR (CDCl₃, 500 MHz): δ 7.59 (dd, J=7.0, 2.2 Hz, 1H), 7.41 (ddd, J=8.6, 4.5, 2.3 Hz 1H), 7.17 (app t, J=8.7 Hz, 1H), 7.11-7.09 (m, 2H), 7.03 (dd, J=7.8, 1.7 Hz, 1H), 4.76 (dd, J=7.4, 7.3 Hz, 1H), 4.27 (q, J=7.1 Hz, 2H), 2.90-2.84 (m, 1H), 2.81-2.76 (m, 1H), 2.34-2.28 (m, 1H), 2.25-2.17 (m, 1H), 1.31 (t, J=7.1 Hz, 3H); ¹³C NMR (CDCl₃, 125 MHz): δ 170.9, 157.74 (d, J_{CF}=249 Hz), 154.0, 138.7, 138.11 (d, J_{CF}=4.13 Hz), 130.1, 129.2, 126.7 (d, J_{CF}=7.1 Hz), 121.32 (d, J_{CF}=17.7 Hz), 121.1, 119.5, 116.9 (d, J_{CF}=21.2 Hz), 115.5, 74.0, 61.6, 24.7, 23.2, 14.4; ¹⁹F NMR (CDCl₃, 471 MHz): δ−118.2; HRMS (ESI+) m/z calcd for C₁₈H₁₇ClFO₃ [M+H] 335.0845, found 335.0844.

Ethyl (S)-7-(3,4-dichlorophenyl)chromane-2-carboxylate (S)-62e. In a N₂-filled glove box, a microwave vial was charged with Pd(PPh₃)₄ (0.0300 g, 0.0260 mmol) and CsF (0.174 g, 1.15 mmol). A solution of ethyl (S)-7-(((perfluorobutyl)sulfonyl)oxy)chromane-2-carboxylate (61, 0.260 g, 0.516 mmol) in dioxane/H₂O (4.4:1, 5.3 mL/1.2 mL) was sparged with N₂ (15 min) and added to the reaction vial followed by the (3,4-dichlorophenyl)boronic acid (0.106 g, 0.556 mmol). After additional sparging with N₂ (5 min), the vial was sealed, and heated at 90° C. for 16 h. The reaction mixture was extracted with EtOAc washed with satd. NaHCO₃/satd. NaCl (1:1).

The aqueous layer was back extracted with EtOAc (2×). The combined organic layer was dried (Na₂SO₄), filtered, and concentrated. The residue was purified by chromatography on SiO₂ (0-18% EtOAc/hexanes) to give ethyl (S)-7-(3,4-dichlorophenyl)chromane-2-carboxylate ((S)-62e, 0.158 g, 87%) as a yellow oil: [α]_{D}=−45.3 (c 0.25, CH₂Cl₂); IR (CH₂Cl₂) 2979, 2934, 2850, 1751, 1469, 1187, 1130 cm⁻¹; ¹H NMR (CDCl₃, 500 MHz): δ 7.65 (d, J=2.1 Hz, 1H), 7.47 (d, J=8.3 Hz, 1H), 7.39 (dd, J=8.3, 2.1 Hz, 1H), 7.14-7.09 (m, 2H), 7.05 (dd, J=7.9, 1.8 Hz, 1H), 4.76 (dd, J=7.2, 3.8 Hz, 1H), 4.23 (q, J=7.1 Hz, 2H), 2.93-2.73 (m, 2H), 2.37-2.16 (m, 2H), 1.31 (t, J=7.1 Hz, 3H); ¹³C NMR (CDCl₃, 126 MHz): δ 170.9, 154.0, 140.9, 138.4, 132.9, 131.4, 130.8, 130.2, 128.9, 126.3, 121.5, 119.4, 115.4, 74.0, 61.6, 24.7, 23.2, 14.4; HRMS (ESI+) m/z calcd for C₁₈H₁₇Cl₂O₃ [M+H] 351.0549, found 351.0548.

(S)-7-(3-Chloro-4-fluorophenyl)-N-hydroxychromane-2-carboxamide (S)-58d. A solution of the ethyl (S)-7-(3-chloro-4-fluorophenyl)chromane-2-carboxylate ((S)-62d, 0.115 g, 0.344 mmol) in THF (1.5 mL) and MeOH (1.5 mL) was treated with LiGH monohydrate (0.0159 g, 0.378 mmol) in H₂O (1.0 mL) at room temperature. After 1 h, the solution was concentrated, and azeotroped with PhMe (2×). The residue was dissolved in DMF (0.6 mL) and treated with 0-(tetrahydro-2H-pyran-2-yl)hydroxylamine (0.120 g, 1.02 mmol). The mixture was cooled to 0° C. and treated with T3P (0.300 mL, 0.504 mmol, 50% EtOAc soln) and TEA (0.100 mL, 0.717 mmol). After 30 min, the mixture was warmed to room temperature. After 4 h, the reaction mixture was extracted with EtOAc, washed with 0.5M HCl, brine, dried (Na₂SO₄), filtered through a SiO₂ plug, and concentrated. The orange residue (0.127 g) was dissolved in MeOH (6.0 mL) and treated with Amberlyst-15 (0.027 g, 126.3 mmol, washed with MeOH) at room temperature under. After 20 h, the reaction mixture was filtered through Celite®, rinsed with MeOH, and concentrated. The residue was purified by trituration with hexanes/EtOAc (5:1) to give (S)-7-(3-chloro-4-fluorophenyl)-N-hydroxychromane-2-carboxamide ((S)-58d, 0.079 g, 74%) as a white solid: [α]_{D}=−7.7 (c 0.12, DMSO); mp 173-174° C.; IR (CH₂Cl₂) 3261, 2919, 1655, 1488, 1213 cm⁻¹; ¹H NMR (DMSO-d₆, 500 MHz): δ 10.78 (s, 1H), 8.96 (s, 1H) 7.81 (dd, J=7.1, 2.3 Hz, 1H), 7.63 (ddd, J=8.6, 4.7, 2.3 Hz, 1H), 7.48 (app t, J=8.9 Hz, 1H), 7.18-7.13 (m, 3H), 4.59 (dd, J=8.5, 3.2 Hz, 1H), 2.84-2.78 (m, 1H), 2.75-2.70 (m, 1H), 2.16-2.10 (m, 1H), 2.02-1.95 (m, 1H); ^{1 13}C NMR (DMSO-d₆, 125 MHz): δ 166.3, 156.7 (d, J_{C-F}=247 Hz), 153.6, 137.7 (d, J_{C-F}=3.6 Hz), 136.8, 130.0, 128.3, 126.9 (d, J_{C-F}=7.3 Hz), 121.8, 119.9 (d, J_{C-F}=17.9 Hz), 118.8, 117.3 (d, J_{C-F}=20.9 Hz), 114.8, 73.8, 24.1, 22.6; ¹⁹F NMR (DMSO-d₆, 470 MHz): δ−118.8; HRMS (ESI+)

m/z calcd for C₁₆H₁₂O₃NClF [M−H] 320.0484, found 320.0492; ELS purity (100%).

(S)-7-(3,4-Dichlorophenyl)-N-hydroxychromane-2-carboxamide (S)-58e. A solution of ethyl (S)-7-(3,4-dichlorophenyl)chromane-2-carboxylate ((S)-62e, 0.109 g, 0.310 mmol) in THF (2 mL) and MeOH (2 mL) was treated with LiGH monohydrate (0.0143 g, 0.341 mmol) in H₂O (1.5 mL) at room temperature. After 2 h, the solution was concentrated, and azeotroped with PhMe (2×). The residue was dissolved in DMF (1.0 mL) and treated with 0-(tetrahydro-2H-pyran-2-yl)hydroxylamine (0.115 g, 0.982 mmol). The mixture was cooled to 0° C., and treated with T3P (0.300 mL, 0.504 mmol, 50% EtOAc soln) and TEA (0.100 mL, 0.717 mmol). After 30 min, the mixture was warmed to room temperature. After 18 h, the reaction mixture was extracted with EtOAc, washed with 0.5M HCl, brine, dried (Na₂SO₄), filtered through a SiO₂ plug, and concentrated. The yellow oil (0.149 g) was dissolved in MeOH (3.0 mL) and treated with Amberlyst-15 (0.030 g, 105.8 mmol, washed with MeOH) at room temperature. After 7 h, the reaction mixture was filtered through Celite®, rinsed with MeOH, and concentrated. The residue was purified by trituration with hexanes/EtOAc (5:1) followed by a MeOH (1 mL) rinse to give (S)-7-(3,4-dichlorophenyl)-N-hydroxychromane-2-carboxamide ((S)-58e, 0.029 g, 34%) as a peach/beige solid: [α]_{D}=−25.4 (c 0.05, DMSO); mp 134-136° C.; IR (CH₂Cl₂) 3252, 2928, 1651, 1550, 1469, 1130 cm⁻¹; ¹H NMR (DMSO-d₆, 500 MHz): δ 10.78 (s, 1H), 8.96 (s, 1H), 7.87 (d, J=2.1 Hz, 1H), 7.70 (d, J=8.4 Hz, 1H), 7.62 (dd, J=8.4, 2.1 Hz, 1H), 7.20 (dd, J=7.9, 1.7 Hz, 1H), 7.17-7.16 (m, 2H), 4.60 (dd, J=8.5, 3.2 Hz, 1H), 2.85-2.78 (m, 1H), 2.76-2.70 (m, 1H), 2.16-2.11 (m, 1H), 2.02-1.95 (m, 1H); ¹³C NMR (DMSO-d₆, 100 MHz): δ 166.3, 153.7, 140.4, 136.5, 131.6, 131.0, 130.1, 130.0, 128.1, 126.6, 122.3, 118.8, 114.8, 73.8, 24.1, 22.6; HRMS (ESI+) m/z calcd for C₁₆H₁₂O₃NCl₂ [M−H] 336.0189, found 336.0201.

### Part 5: Substitutions at the Chromane Pyrano Group

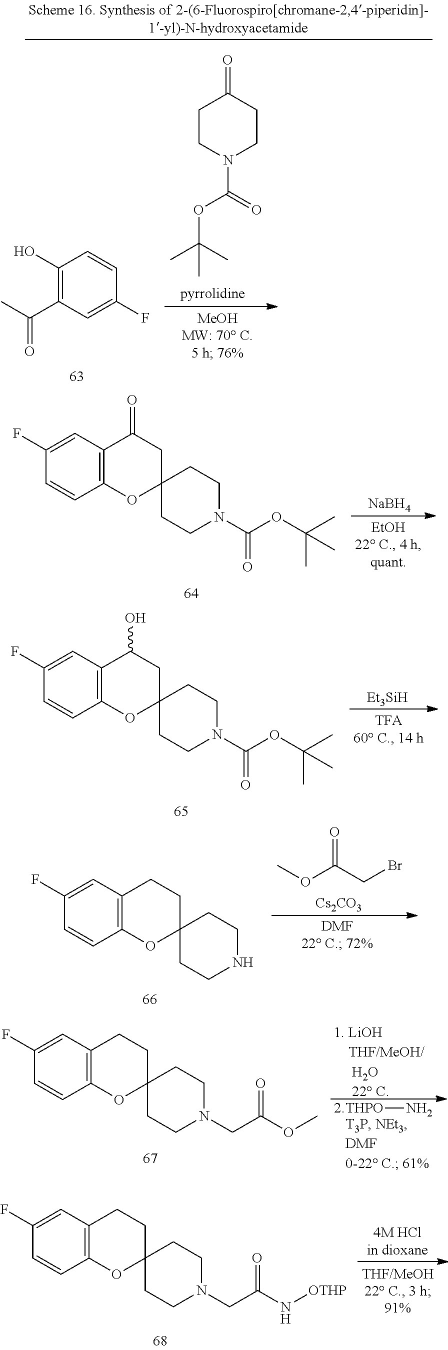

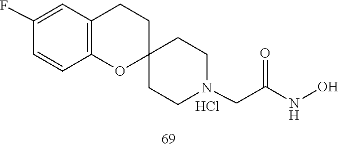 [SMILES: O=C(CN1CCC2(CCc3cc(F)ccc3O2)CC1)NO ; Cl]

tert-Butyl 6-fluoro-4-oxospiro[chromane-2,4′-piperidine]-1′-carboxylate (64). A mixture of 1-(5-fluoro-2-hydroxyphenyl)ethan-1-one (0.992 g, 6.31 mmol), tert-butyl 4-oxopiperidine-1-carboxylate (1.28 g, 6.36 mmol), and pyrrolidine (1.00 mL, 12.2 mmol) in MeOH (4 mL) was subjected to microwave irradiation at 70° C. for 5 h. The solution was concentrated and the mixture was purified by chromatography on SiO₂ (0-100% EtOAc/hexanes) to give tert-butyl 6-fluoro-4-oxospiro[chromane-2,4′-piperidine]-1′-carboxylate (64, 1.62 g, 76%) as a yellow solid: ¹H NMR (CDCl₃, 500 MHz): δ 7.50 (dd, J=8.0, 3.0 Hz, 1H), 7.21 (ddd, J=9.0, 8.2, 3.0 Hz, 1H), 6.95 (dd, J=9.0, 4.0 Hz, 1H), 3.86 (br s, 2H), 3.19 (br s, 2H), 2.70 (s, 2H), 2.00 (d, J=13.2 Hz, 2H), 1.59 (td, J=13.0, 3.9 Hz, 2H), 1.45 (s, 9H); ¹³C NMR (CDCl₃, 125 MHz): δ 190.9, 157.3 (d, J_{CF}=242.7 Hz), 155.3, 154.8, 123.9 (d, J_{CF}=24.4 Hz), 121.3 (d, J_{CF}=6.4 Hz), 120.0 (d, J_{CF}=7.3 Hz), 111.9 (d, J_{CF}=23.9 Hz) 79.97, 78.26, 77.16, 47.9, 39.2, 34.0; ¹⁹F NMR (CDCl₃, 470 MHz): δ−121.6; HRMS (ESI+) m/z calcd for C₁₃H₁₅O₂NF [M+H-Boc]236.1086, found 236.1078.

tert-Butyl-6-fluoro-4-hydroxyspiro[chromane-2,4′-piperidine]-1′-carboxylate (65). A solution of tert-butyl 6-fluoro-4-oxospiro[chromane-2,4′-piperidine]-1′-carboxylate (64, 0.310 g, 0.923 mmol) in EtOH (2.5 mL) was treated with NaBH₄ (0.045 mg, 1.19 mmol) at room temperature portion wise. Upon completion of reaction, the mixture was diluted with H₂O and extracted with EtOAc (3×). The combined organic layer was washed with H₂O, brine, dried (Na₂SO₄), filtered, and concentrated to give tert-butyl-6-fluoro-4-hydroxyspiro[chromane-2,4′-piperidine]-1′-carboxylate (65, 0.312 g, quant.) as a white solid: ¹H NMR (CDCl₃, 300 MHz): δ 7.15 (dd, J=9.1, 3.0 Hz, 1H), 6.90 (td, J=8.4, 3.1 Hz, 1H), 6.79 (dd, J=9.0, 4.7 Hz, 1H), 4.84 (m, 1H), 3.85 (br s, 2H), 3.28-3.10 (m, 2H), 2.15 (dd, J=13.6, 6.1 Hz, 1H), 1.94-1.60 (m, 5H), 1.46 (s, 9H); ¹⁹F NMR (CDCl₃, 471 MHz): δ−123.1; HRMS (ESI+) m/z calcd for C₁₈H₂₄FNO₄Na [M+Na] 360.1582, found 360.1581.

6-Fluorospirochromane-2,4′-piperidine (66). Triethylsilane (0.700 mL, 4.38 mmol) was added to a solution of tert-butyl-6-fluoro-4-hydroxyspiro[chromane-2,4′-piperidine]-1′-carboxylate (65, 0.259 g, 0.919 mmol) in TFA (3.3 mL). The reaction mixture was heated to 60° C. for 14 h. The mixture was concentrated and the residue was extracted with EtOAc and washed with 0.5M HCl. The aqueous was treated with 2.5M NaOH to pH 14 and extracted with EtOAc (2×). The combined organic layer was dried (Na₂SO₄), filtered and concentrated to give 6-fluorospirochromane-2,4′-piperidine (66, 0.132 g, 65%) as a beige solid: mp 119-121° C.; IR (neat) 2939, 1554, 1491, 1432, 1218 cm⁻¹; ¹H NMR (CDCl₃, 500 MHz): δ 6.81-6.73 (m, 3H), 3.03 (app t, J=11.0 Hz, 2H), 2.86-2.84 (m, 2H), 2.75 (app t, J=6.8 Hz, 2H), 1.80-1.74 (m, 5H), 1.53 (app td, J=12.4, 4.2 Hz, 2H); ¹³C NMR (CDCl₃, 126 MHz): δ 156.7 (d, J_{CF}=238 Hz), 149.5, 122.6 (d, J_{CF}=7.3 Hz), 118.2 (d, J_{CF}=8.0 Hz), 115.3 (d, J_{CF}=22.6 Hz), 114.1 (d, J_{CF}=7.3 Hz), 73.2, 42.2, 35.7, 32.0, 21.7; ¹⁹F NMR (CDCl₃, 470 MHz): δ−125.0; HRMS (ESI+) m/z calcd for C₁₃H₁₇ONF [M+H] 222.1289, found 222.1286.

Methyl 2-(6-fluorospiro[chromane-2,4′-piperidin]-1′-yl)acetate (67). A solution of 6-fluorospirochromane-2,4′-piperidine (66, 0.225 g, 1.02 mmol) in anhydrous DMF (4 mL) was treated with Cs₂CO₃ (0.562 g, 1.73 mmol). The mixture was then treated with methyl-2-bromoacetate (100.0 mL, 1.03 mmol) dropwise at room temperature. After 1 h, the reaction mixture was extracted with EtOAc and washed with brine. The aqueous layer was back extracted with EtOAc (2×). The combined organic layer was dried (Na₂SO₄), filtered, and concentrated to give methyl 2-(6-fluorospiro[chromane-2,4′-piperidin]-1′-yl)acetate (67, 0.207 g, 69%) as a yellow oil that crystallized upon standing: ¹H NMR (CDCl₃, 500 MHz): δ 6.81-6.73 (m, 3H), 3.73 (s, 3H), 3.26 (s, 2H), 2.76-2.71 (m, 4H), 2.55 (td, J=11.5, 3.0 Hz, 2H), 1.83-1.71 (m, 6H); ¹⁹F NMR (CDCl₃, 470 MHz): δ−124.9; HRMS (ESI+) m/z calcd for C₁₆H₂₁FNO₃ [M+H] 294.1500, found 294.1494.

2-(6-Fluorospiro[chromane-2,4′-piperidin]-1′-yl)-N-((tetrahydro-2H-pyran-2-yl)oxy)acetamide (68). A solution of methyl 2-(6-fluorospiro[chromane-2,4′-piperidin]-1′-yl)acetate (67, 0.207 g, 0.706 mmol) in THE (1.5 mL) and MeOH (1.5 mL) was treated with LiGH monohydrate (0.0369 g, 0.879 mmol) in H₂O (1.5 mL) at room temperature. After 1 h, the solution was concentrated and the residue was azeotroped with PhMe (2×). The crude residue was dissolved in DMF (3 mL) and treated with O-(tetrahydro-2H-pyran-2-yl)hydroxylamine (0.101 g, 0.864 mmol). The mixture was cooled to 0° C. and T3P (0.650 mL, 0.919 mmol, 50% EtOAc soln) and TEA (0.200 mL, 1.43 mmol) were added. The reaction mixture was warmed to room temperature. After 16 h, the reaction mixture was extracted with EtOAc and washed with 1M NaOH. The aqueous layer was back extracted with EtOAc. The combined organic layer was dried (Na₂SO₄), filtered, and concentrated. The residue was purified by chromatography on SiO₂ (100% EtOAc to 5% MeOH/CH₂Cl₂) to give 2-(6-fluorospiro[chromane-2,4′-piperidin]-1′-yl)-N-((tetrahydro-2H-pyran-2-yl)oxy)acetamide (68, 0.163 g, 61%) as a white foam: ¹H NMR (CDCl₃, 500 MHz): δ 9.50 (s, 1H), 6.80-6.74 (m, 3H), 4.97 (s, 1H), 4.00-3.98 (m, 1H), 3.66-3.63 (m, 1H), 3.14 (s, 2H), 2.75 (t, J=7.0 Hz, 2H), 2.65-2.57 (m, 4H), 1.84-1.56 (m, 12H); ¹⁹F NMR (CDCl₃, 470 MHz): δ−124.7; HRMS (ESI+) m/z calcd for C₂₀H₂₈FN₂O₄ [M+H] 379.2028, found 379.2027.

2-(6-Fluorospiro[chromane-2,4′-piperidin]-1′-yl)-N-hydroxyacetamide hydrogen chloride salt (69). A solution of 2-(6-fluorospiro[chromane-2,4′-piperidin]-1′-yl)-N-((tetrahydro-2H-pyran-2-yl)oxy)acetamide (68, 0.160 g, 0.423 mmol) in Et₂O (4.55 mL) and MeOH (0.25 mL) was treated with 4M HCl in dioxane (0.250 mL, 1.00 mmol). After 2 h, the white solid was collected by filtration and dried in vacuo to give 2-(6-fluorospiro[chromane-2,4′-piperidin]-1′-yl)-N-hydroxyacetamide hydrogen chloride salt (69, 0.128 g, 91%) as a white solid: mp 200-203° C.; IR (neat) 3038, 2852, 1677, 1490, 1432, 1367, 1211 cm⁻¹; ¹H NMR (DMSO-d₆, 500 MHz; 100° C.): δ 6.93-6.88 (m, 2H), 6.83-6.81 (m, 1H), 3.87 (br s, 2H), 3.38-3.36 (m, 4H), 2.78 (app t, J=6.8 Hz, 2H), 2.11-2.01 (m, 2H), 1.97-1.94 (m, 2H), 1.86 (app t, J=6.8 Hz, 2H); ¹³C NMR (101 MHz; 25° C., rotamers, DMSO-d₆): δ 166.8, 160.7, 156.1 (d, J_{CF}=236.7 Hz), 148.3, 122.9 (d, J_{CF}=7.1 Hz), 118.0 (d, J_{CF}=7.7 Hz), 115.4 (d, J_{CF}=22.8 Hz), 114.0 (d, J_{CF}=23.3 Hz), 70.0, 54.3, 48.4, 47.9, 30.8, 30.4, 20.8; ¹⁹F NMR (DMSO-d₆, 376 MHz; rotamers): δ−124.92, −123.94, −124.13; HRMS (ESI+) m/z calcd for C₁₅H₁₈O₃N₂F [M−H] 293.1296, found 293.1307.

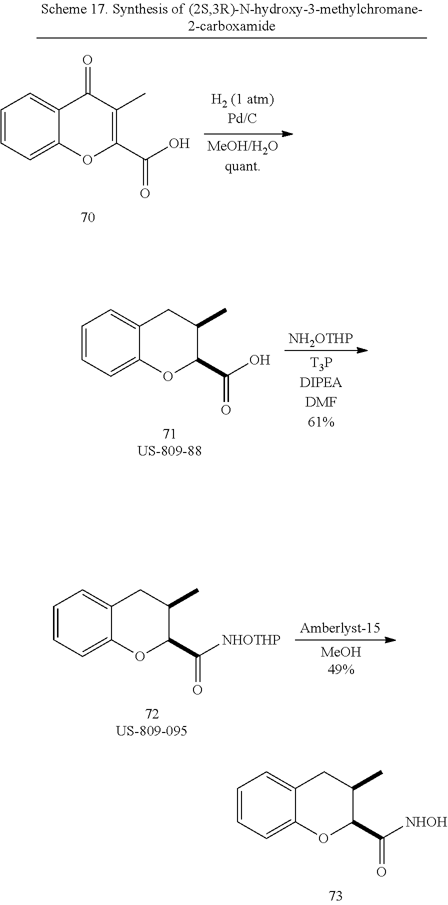

(rel)-(2S,3R)-3-Methylchromane-2-carboxylic acid (71). A solution of 3-methyl-4-oxo-4H-chromene-2-carboxylic acid (70, 0.250 g, 1.16 mmol) in MeOH/H₂O (3 mL, 2.7/0.3) was evacuated and purged with N₂ (2×). To this solution was added 10% Pd/C (60.7 mg, 0.057 mmol) and the mixture was evacuated and purged with H₂ (2×). The reaction mixture was kept under H₂ (1 atm-balloon) overnight. After 16 h, the reaction mixture was transferred to a Parr reactor, and was stirred under H₂ (10 bar) for 26 h. Additional Pd/C (73.7 mg, 0.0692 mmol) was added to the mixture, and the reaction mixture was stirred under H₂ (10 bar) for another 24 h. The mixture was filtered through Celite®, rinsed with EtOAc and concentrated to give (rel)-(2S,3R)-3-methylchromane-2-carboxylic acid (71, 0.230 mg, quant.) as an off-white powder: ¹H NMR (CDCl₃, 500 MHz): δ 7.15 (app t, J=7.7 Hz, 1H), 7.08-7.07 (m, 1H), 6.95-6.92 (m, 2H), 4.68 (d, J=2.6 Hz, 1H), 3.50 (s, 3H), 3.17-3.13 (m, 1H), 2.70-2.67 (m, 1H), 2.59 (dd, J=16.4, 3.1 Hz, 1H), 1.04 (d, J=7.0 Hz, 3H); HRMS (ESI+) m/z calcd for C₁₁H₁₁O₃ [M−H] 191.0708, found 191.0699.

(rel)-(2S,3R)-3-Methyl-N-((tetrahydro-2H-pyran-2-yl)oxy)chromane-2-carboxamide (72). A solution of (rel)-(2S,3R)-3-methylchromane-2-carboxylic acid (71, 0.230 g, 1.20 mmol) in DMF (2.5 mL) was treated with O-(tetrahydro-2H-pyran-2-yl)hydroxylamine (0.182 g, 1.49 mmol). To the solution was added DIPEA (0.240 mL, 1.44 mmol) followed by T3P (0.860 mL, 1.44 mmol, 50% EtOAc soln) at room temperature. After 2 h, the reaction mixture was extracted with EtOAc and washed with H₂O, brine (2×), dried (Na₂SO₄), filtered and concentrated. The residue was purified by chromatography on SiO₂ (0-50% EtOAc/hexanes) to give (rel)-(2S,3R)-3-methyl-N-((tetrahydro-2H-pyran-2-yl)oxy)chromane-2-carboxamide (72, 0.214 g, 61%) as a colorless oil:

(rel)-(2S,3R)—N-Hydroxy-3-methylchromane-2-carboxamide (73). A solution of (rel)-(2S,3R)-3-methyl-N-((tetrahydro-2H-pyran-2-yl)oxy)chromane-2-carboxamide (72, 213 mg, 0.732 mmol) in MeOH (3 mL) was treated with Amberlyst-15 (0.040 g, washed with MeOH) at room temperature. After 17 h, the reaction mixture was filtered through Celite®, rinsed with MeOH and concentrated. The solid residue was treated with Et₂O, sonicated and the solid was collected by filtration and dried in vacuo to give (rel)-(2S,3R)—N-hydroxy-3-methylchromane-2-carboxamide (73, 0.075 g, 49%) as a white solid: mp 156-160° C.; IR (neat) 3312, 2933, 1634, 1611, 1540, 1486, 1454, 1349, 1228, 1119, 1089, 1057, 999, 914, 851, 794, 750, 711 cm⁻¹; ¹H NMR (acetone-d₆, 400 MHz): δ 10.47 (br s, 1H), 8.27 (d, J=2.3 Hz, 1H), 7.10 (dd, J=10.3, 7.8 Hz, 2H), 6.90-6.85 (m, 2H), 4.61 (d, J=2.3 Hz, 1H), 3.18-3.14 (m, 1H), 2.65-2.57 (m, 2H), 0.92 (d, J=6.9 Hz, 3H); ¹³C NMR (acetone-d₆, 100 MHz): δ 166.8, 153.7, 131.1, 128.0, 121.9, 121.6, 117.2, 78.3, 32.7, 28.7, 13.2; HRMS (ESI+) m/z calcd for C₁₁H₁₄NO₃ [M+H]208.0974, found 208.0967.

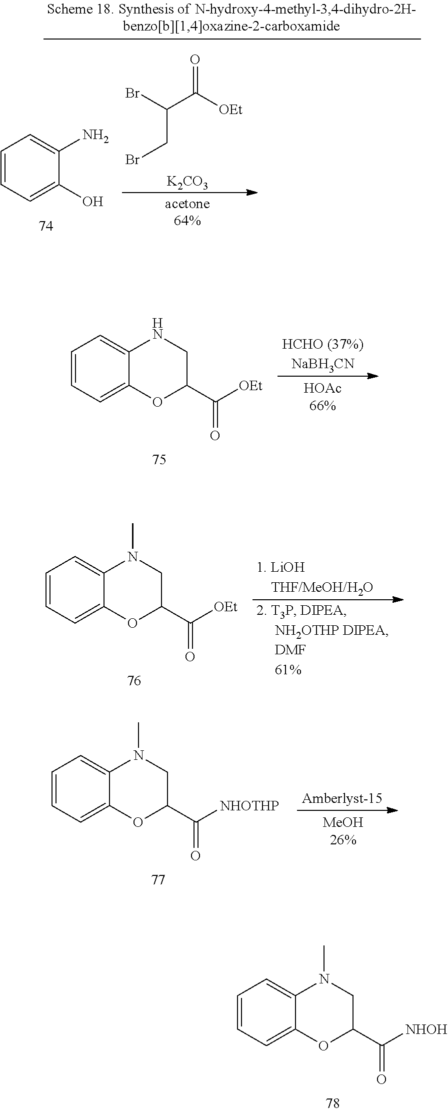

Ethyl 3,4-dihydro-2H-benzo[b][1,4]oxazine-2-carboxylate (75). A solution of 2-aminophenol (2.00 g, 18.1 mmol), ethyl-2,3-dibromopropanoate (3.40 mL, 22.7 mmol) and K₂CO₃ (7.17 g, 50.9 mmol) in acetone (8 mL) was heated to reflux for 45 min. The reaction mixture was extracted with CH₂Cl₂ and washed with brine. The aqueous layer was back extracted with CH₂Cl₂ (2×). The combined organic layer was washed with brine, dried (Na₂SO₄), filtered and concentrated. The residue was purified by chromatography on SiO₂ (ISCO-Rf, 0-20% EtOAc/hexanes) to give ethyl 3,4-dihydro-2H-benzo[b][1,4]oxazine-2-carboxylate (75, 2.42 g, 64%) as a red-brown oil: ¹H NMR (CDCl₃, 500 MHz): δ 6.93 (dd, J=8.0, 1.4 Hz, 1H), 6.79 (td, J=7.6, 1.5 Hz, 1H), 6.72 (ddd, J=7.9, 7.4, 1.6 Hz, 1H), 6.61 (dd, J=7.8, 1.6 Hz, 1H), 4.80 (dd, J=5.1, 3.2 Hz, 1H), 4.25 (t, J=7.0 Hz, 2H), 3.62-3.55 (m, 2H), 1.27 (t, J=7.1 Hz, 3H).

Ethyl 4-methyl-3,4-dihydro-2H-benzo[b][1,4]oxazine-2-carboxylate (76). To a solution of ethyl 3,4-dihydro-2H-benzo[b][1,4]oxazine-2-carboxylate (75, 0.401 g, 1.94 mmol) in AcOH (1.5 mL) was added 37% formaldehyde aqueous solution (0.58 mL, 7.79 mmol) at room temperature. After 10 min, the reaction mixture was cooled to 0° C. and treated with sodium cyanoborohydride (0.256 g, 3.87 mmol) portion wise over 10 min. The solution was warmed to room temperature. After 15 min, the reaction mixture was poured into ice water and extracted with EtOAc (3×). The combined organic layer was washed with brine, dried (Na₂SO₄), filtered and concentrated. The residue was purified by chromatography on SiO₂, (ISCO-Rf, 0-15% EtOAc/hexanes) to give ethyl 4-methyl-3,4-dihydro-2H-benzo[b][1,4]oxazine-2-carboxylate (76, 284 mg, 66%) as a yellow oil: ¹H NMR (CDCl₃, 300 MHz): δ 6.89 (ddd, J=19.5, 7.8, 1.5 Hz, 2H), 6.74-6.66 (m, 2H), 4.86 (t, J=4.2 Hz, 1H), 4.25 (q, J=7.1 Hz, 2H), 3.43-3.42 (m, 2H), 2.88 (s, 3H), 1.27 (t, J=7.1 Hz, 3H).

4-Methyl-N-((tetrahydro-2H-pyran-2-yl)oxy)-3,4-dihydro-2H-benzo[b][1,4]oxazine-2-carboxamide (77). To a solution of ethyl 4-methyl-3,4-dihydro-2H-benzo[b][1,4]oxazine-2-carboxylate (76, 283 mg, 1.28 mmol) in THF/EtOH (2.5 mL, 1:1) was added 1M LiGH (1.40 mL) at room temperature. After 40 min, the solution was concentrated and the residue was azeotroped with PhMe (3×) and concentrated to give a light yellow solid. This solid was dissolved in DMF (2.5 mL) and was treated with O-(tetrahydro-2H-pyran-2-yl)hydroxylamine (0.191 mg, 1.56 mmol) at room temperature. To this solution was added DIPEA (0.255 mL, 1.54 mmol) followed by T3P (0.92 mL, 1.54 mmol, 50% EtOAc soln). After 1.5 h, the solution was extracted with EtOAc and washed with H₂O, brine (2×), dried (Na₂SO₄), filtered and concentrated. The residue was purified by chromatography on SiO₂ (ISCO-Rf, 0-35% EtOAc/hexanes) to give 4-methyl-N-((tetrahydro-2H-pyran-2-yl)oxy)-3,4-dihydro-2H-benzo[b][1,4]oxazine-2-carboxamide (77, 245 mg, 65%-contains residual solvent) as a yellow oil: ¹H NMR (CDCl₃, 300 MHz): δ 9.09 (s, 1H), 6.96-6.87 (m, 2H), 6.73 (ddd, J=7.7, 6.3, 1.5 Hz, 2H), 5.05-4.94 (m, 1H), 4.90-4.86 (m, 1H), 4.13-3.93 (m, 1H), 3.58-3.50 (m, 2H), 3.48-3.35 (m, 1H), 2.90 (s, 3H), 1.87-1.81 (m, 3H), 1.64-1.55 (m, 3H); HRMS (ESI+) m/z calcd for C₁₅H₂₀N₂O₄Na [M+Na] 315.1321, found 315.1312.

N-Hydroxy-4-methyl-3,4-dihydro-2H-benzo[b][1,4]oxazine-2-carboxamide (78). A solution of 4-methyl-N-((tetrahydro-2H-pyran-2-yl)oxy)-3,4-dihydro-2H-benzo[b][1,4]oxazine-2-carboxamide (77, 240 mg, 0.821 mmol) in MeOH (2 mL) was treated with Amberlyst-15 (50 mg, washed with MeOH) at room temperature. After 19 h, the reaction mixture was filtered through Celite®, rinsed with MeOH and concentrated. The solid residue was triturated with Et₂O, sonicated and the solid was collected by filtration and dried in vacuo to give N-hydroxy-4-methyl-3,4-dihydro-2H-benzo[b][1,4]oxazine-2-carboxamide (78, 44 mg, 26%) as a light brown solid: mp 118-120° C.; IR (neat) 3149, 3060, 2817, 1654, 1503, 1261, 1214, 1084, 742 cm⁻¹; ¹H NMR (DMSO-d₆, 400 MHz): δ 10.78 (d, J=1.4 Hz, 1H), 8.97 (d, J=1.6 Hz, 1H), 6.81-6.76 (m, 2H), 6.69 (dd, J=8.0, 1.5 Hz, 1H), 6.62 (ddd, J=8.0, 7.2, 1.5 Hz, 1H), 4.70 (dd, J=6.3, 3.1 Hz, 1H), 3.32-3.23 (m, 2H), 2.80 (s, 3H); ¹³C NMR (DMSO-d₆, 100 MHz): δ 164.9, 142.9, 136.3, 121.5, 117.9, 115.9, 112.7, 72.7, 49.7, 38.2; HRMS (ESI+) m/z calcd for C₁₀H₁₃N₂O₃ [M+H] 209.0926, found 209.0921.

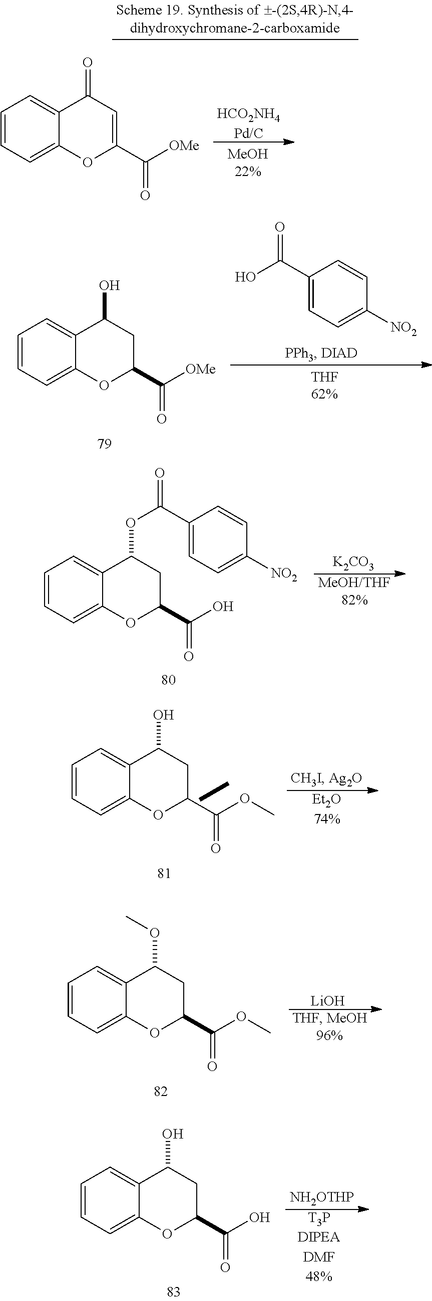

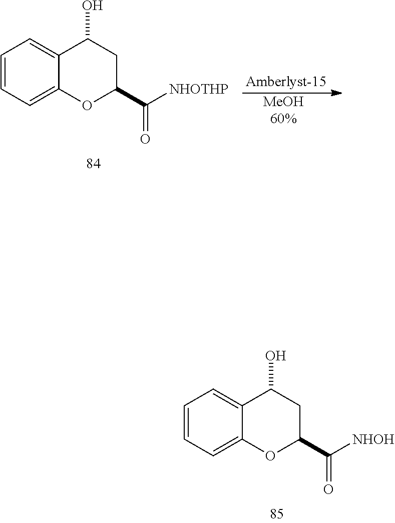

(rel)-Methyl (2S,4S)-4-hydroxychromane-2-carboxylate (79). To a solution of methyl 4-oxo-4H-chromene-2-carboxylate (5.50 g, 26.9 mmol) in MeOH (135 mL) was added ammonium formate (10.2 g, 161 mmol) and Pd/C (10%, 2.15 g, 2.02 mmol). The reaction mixture was heated at reflux for 1 h and then cooled to room temperature. After 12 h, the reaction mixture was filtered through Celite® and washed with methanol and concentrated. The residue was purified by chromatography on SiO₂ (10-30% EtOAc/hexanes) to give (rel)-methyl (2S,4S)-4-hydroxychromane-2-carboxylate (79, 3.6 g, 64%) as white solid: ¹H NMR (CDCl₃, 500 MHz): δ 7.30 (d, J=7.5 Hz, 1H), 7.27-7.24 (m, 1H), 6.99-6.96 (m, 2H), 4.90 (app t, J=5.0, 1H), 4.84 (app t, J=4.0 Hz, 1H), 3.76 (s, 3H), 2.52-2.47 (m, 1H), 2.44-2.39 (m, 1H), 1.76 (br s, 1H).

(rel)-Methyl-(2S,4R)-4-((4-nitrobenzoyl)oxy)chromane-2-carboxylate (80). To a solution of (rel)-methyl (2S,4S)-4-hydroxychromane-2-carboxylate (79, 0.057 g, 0.27 mmol), 4-nitrobenzoic acid (0.056 g, 0.33 mmol), PPh₃ (0.086 g, 0.32 mmol) in THE (0.75 mL) was added DIAD (0.070 mL, 0.33 mmol) at 0° C. After 1 h, the reaction was warmed to room temperature. After 2 d, the solution was concentrated and the residue was purified by chromatography on SiO₂ (ISCO-Rf, 0-100% EtOAc/hexanes) to give (rel)-methyl-(2S,4R)-4-((4-nitrobenzoyl)oxy)chromane-2-carboxylate (80, 0.089 g, 91%) as an off-white solid: mp 120-122° C.; IR (neat) 2956, 1719, 1608, 1585, 1525, 1486, 1461, 1343, 1263, 1214, 1098, 1058, 1002, 861, 839, 758, 717 cm⁻¹; ¹H NMR (CDCl₃, 400 MHz): δ8.27 (d, J=8.8 Hz, 2H), 8.20 (d, J=8.8 Hz, 2H), 7.40 (dd, J=1.2, 7.6 Hz, 1H), 7.35-7.30 (m, 1H), 7.06 (d, J=8.4 Hz, 1H), 7.00-6.97 (m, 1H), 6.27 (app t, J=2.8 Hz, 1H), 4.92 (dd, J=2.4, 12.4 Hz, 1H), 3.87 (s, 3H), 2.61 (app dt, J=2.8, 2.8 Hz, 1H), 2.43-2.35 (m, 1H); ¹³C NMR (CDCl₃, 100 MHz): 3170.2, 163.8, 153.9, 150.7, 135.2, 131.1 130.9, 130.7, 123.6, 121.6, 118.7, 117.6, 70.4 66.4, 52.7, 31.1.

(rel)-Methyl (2S,4R)-4-hydroxychromane-2-carboxylate (81). To a suspension of (rel)-methyl-(2S,4R)-4-((4-nitrobenzoyl)oxy)chromane-2-carboxylate (80, 1.33 g, 3.72 mmol) in MeOH (20 mL) and THE (3.0 mL) was added K₂CO₃ (0.524 g, 3.79 mmol) at room temperature.

After 10 min, the mixture was acidified with 3M HCl and extracted with EtOAc, washed with brine, dried (Na₂SO₄), filtered and concentrated. The residue was absorbed onto Celite® and purified by chromatography on SiO₂ (ISCO-Rf, 0-100% EtOAc/hexanes) followed by a second chromatography on SiO₂ (0-10% MeOH/CH₂Cl₂) to give (rel)-methyl (2S,4R)-4-hydroxychromane-2-carboxylate (81, 0.633 g, 82%) as a light yellow oil: IR (neat) 3405, 2954, 2917, 1737, 1611, 1584, 1485, 1460, 1284, 1225, 1180, 1092 1063, 1027, 850, 774 cm⁻¹; ¹H NMR (CDCl₃, 400 MHz): 37.30 (dd, J=1.6, 7.6 Hz, 1H), 7.28-7.24 (m, 1H), 7.02-6.95 (m, 2H), 4.88 (dd, J=2.4, 11.6 Hz, 1H), 4.84 (app t, J=3.2 Hz, 1H), 3.85 (s, 3H), 2.43-2.38 (m, 1H), 2.18-2.11 (m, 1H); ¹³C NMR (CDCl₃, 100 MHz): 3171.1, 153.1, 130.1, 129.5, 123.3, 121.4, 117.4, 70.0, 62.7, 52.5, 33.3; HRMS (ESI+) m/z calcd for C₁₁H₁₂O₄Na [M+Na] 231.0628, found 231.0627.

(rel)-Methyl (2S,4R)-4-methoxychromane-2-carboxylate (82). To a solution of (rel)-methyl (2S,4R)-4-hydroxychromane-2-carboxylate (81, 0.21 g, 1.0 mmol) in distilled Et₂O (6.7 mL) was added activated 3 Å molecular sieves (1.2 g), Ag₂O (0.93 g, 4.0 mmol) and CH₃I (0.75 mL, 12 mmol) subsequently. The resulting mixture was stirred at room temperature for 18 h. The reaction mixture was filtered through Celite® and concentrated. The residue was purified by chromatography on SiO₂ (2-10% EtOAc/hexanes) to give (rel)-methyl (2S,4R)-4-methoxychromane-2-carboxylate (82, 0.16 g, 74%) as colorless oil: ¹H NMR (CDCl₃, 500 MHz): δ7.24-7.22 (m, 2H), 7.00 (d, J=8.5 Hz, 1H), 6.95 (app t, J=7.5 Hz, 1H), 4.87 (dd, J=2.5, 12.5 Hz, 1H), 4.28 (app t, J=3.0 Hz, 1H), 3.85 (s, 3H), 3.46 (s, 3H), 2.51 (dt, J=3.0, 14.0 Hz, 1H), 2.07-2.04 (m, 1H); ¹³C NMR (CDCl₃, 125 MHz): δ171.2, 153.5, 130.5, 130.1, 120.8, 120.7, 117.4, 71.5, 70.2, 56.0, 52.4, 30.1.

(rel)-(2S,4R)-4-Methoxychromane-2-carboxylic acid (83). A solution of (rel)-methyl (2S,4R)-4-methoxychromane-2-carboxylate (82, 0.16 g, 0.72 mmol) in 2:1 THF/MeOH (9.0 mL, 2:1) was treated with 1M LiGH (0.80 mL) at room temperature. After 3 h, the reaction mixture was treated with 1M HCl and extracted with EtOAc (3×). The combined organic layer was dried (MgSO₄), filtered and concentrated to give (rel)-(2S,4R)-4-methoxychromane-2-carboxylic acid (83, 0.15 g, 96%) as colorless oil that was used without any further purification: ¹H NMR (CDCl₃, 500 MHz): 37.29-7.25 (m, 2H), 7.00 (d, J=7.5 Hz, 2H), 4.92 (dd, J=2.0, 12.0 Hz, 1H), 4.31 (app t, J=3.0 Hz, 1H), 3.47 (s, 3H), 2.64 (dt, J=2.5, 15.0 Hz, 1H), 2.07-2.01 (m, 1H).

(rel)-(2S,4R)-4-Methoxy-N-((tetrahydro-2H-pyran-2-yl)oxy)chromane-2-carboxamide (84). To a solution of (rel)-(2S,4R)-4-methoxychromane-2-carboxylic acid (83, 0.14 g, 0.68 mmol) in DMF (3.0 mL, 0.23 M) was added O-(tetrahydro-2H-pyran-2-yl)hydroxylamine (0.12 g, 1.0 mmol) followed by T3P (0.65 g, 1.0 mmol, 50% EtOAc soln) then DIPEA (0.19 mL, 1.2 mmol) at room temperature. After 15 h, the solution was extracted with CH₂Cl₂, washed with brine, dried (MgSO₄), filtered and concentrated. The residue was purified by chromatography on SiO₂ (50% EtOAc/hexanes) to give (rel)-(2S,4R)-4-methoxy-N-((tetrahydro-2H-pyran-2-yl)oxy)chromane-2-carboxamide (84, 0.11 g, 48%) as a white foamy solid: ¹H NMR (CDCl₃, 500 MHz): δ9.23 (s, 0.5H), 9.22 (s, 0.5H), 7.29-7.27 (m, 2H), 6.99 (app t, J=7.5 Hz, 1H), 6.95 (app t, J=8.0 Hz, 1H), 5.07 (app t, J=2.5 Hz, 0.5H), 5.02 (app t, J=3.0 Hz, 0.5H), 4.87-4.83 (m, 1H), 4.30 (dd, J=2.5, 5.5 Hz, 1H), 4.07-4.03 (m, 1H), 3.73-3.67 (m, 1H), 3.48 (s, 1.5H), 3.47 (s, 1.5H), 2.74 (dd, J=3.0, 6.0 Hz, 0.5H), 2.73 (dd, J=3.0, 6.0 Hz, 0.5H), 1.99-1.84 (m, 4H), 1.72-1.57 (m, 3H); HRMS (ESI+) m/z calcd for C₁₆H₂₁O₅NNa [M+Na] 330.1312, found 330.1309.

(rel)-(2S,4R)—N-Hydroxy-4-methoxychromane-2-carboxamide (85). A solution of (rel)-(2S,4R)-4-methoxy-N-((tetrahydro-2H-pyran-2-yl)oxy)chromane-2-carboxamide (84, 0.11 g, 0.34 mmol) in MeOH (2.3 mL) was treated with Amberlyst-15 (30 mg, washed with MeOH) at room temperature. After 15 h, the reaction mixture was filtered through Celite®, rinsed with MeOH and concentrated. The solid was triturated with Et₂O, collected by filtration and dried in vacuo to give (rel)-(2S,4R)—N-hydroxy-4-methoxychromane-2-carboxamide (85, 0.045 g, 60%) as a light pink solid: mp 102-104° C.; IR (neat) 3212, 2887, 1653, 1485, 1462, 1350, 1280, 1225, 1011, 885, 750 cm⁻¹; ¹H NMR (acetone-d₆, 500 MHz): δ10.50 (br s, 1H), 8.11 (s, 1H), 7.30 (dd, J=6.0, 7.5 Hz, 1H), 7.25 (app t, J=7.5 Hz, 1H), 6.96-6.91 (m, overlapping signals, 2H), 4.71 (dd, J=2.0, 12.0 Hz, 1H), 4.34 (app t, J=3.0 Hz, 1H), 3.43 (s, 3H), 2.54 (dt, J=2.5, 14.0 Hz, 1H), 1.96-1.90 (m, 1H); ¹³C NMR (acetone-d₆, 125 MHz): δ166.9, 153.3, 130.9, 129.7, 121.6, 120.4, 116.9, 71.2, 70.9, 55.2, 30.1; HRMS (ESI+) m/z calcd for C₁₁H₁₄O₄ [M+H] 224.0917, found 224.0918.

### Part 6: Unsubstituted Chromanes

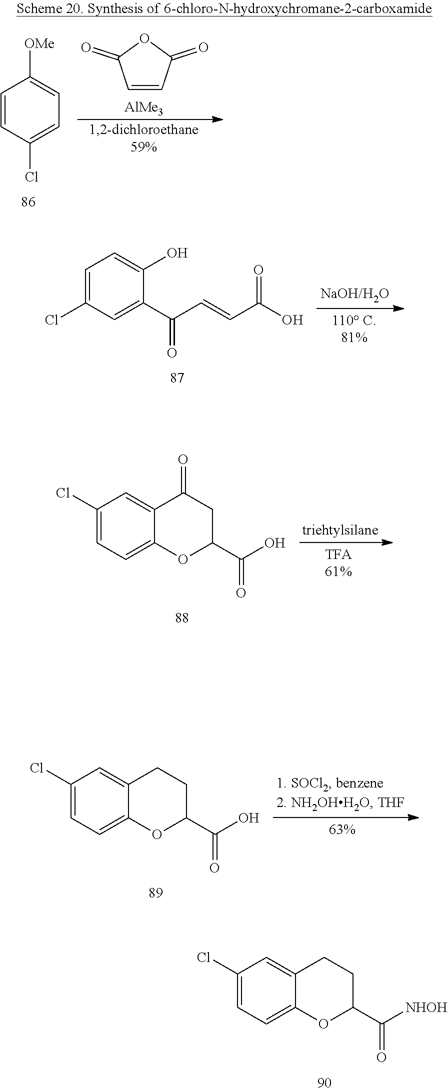

(E)-4-(5-Chloro-2-hydroxyphenyl)-4-oxobut-2-enoic acid (87). Maleic anhydride (1.04 g, 8.00 mmol) and anhydrous aluminum chloride (2.84 g, 10.6 mmol) were dissolved 1,2-dichloroethane (10 mL) by heating at 50° C. for 15 min. To this solution was added 4-chloroanisole (1.14 mL, 9.33 mmol) dropwise at 50° C. The mixture was refluxed for 1 h and poured into conc. HCl (4.3 mL) with ice (27.8 g). The resulting solids were collected by filtration, washed with H₂O, CH₂Cl₂ and dried in vacuo to give (E)-4-(5-chloro-2-hydroxyphenyl)-4-oxobut-2-enoic acid (87, 1.26 g, 59%) as a yellow solid: ¹H NMR (DMSO-d₆, 300 MHz): δ 13.12 (s, 1H), 11.31 (s, 1H), 7.87 (d, J=15.5 Hz, 1H), 7.68 (d, J=2.8 Hz, 1H), 7.53 (dd, J=8.8, 2.8 Hz, 1H), 7.05 (d, J=8.8 Hz, 1H), 6.62 (d, J=15.5 Hz, 1H); HRMS (ESI+) m/z calcd for C₁₀H₆ClO₄ [M−H] 224.9960, found 224.9936.

6-Chloro-4-oxochromane-2-carboxylic acid (88). (E)-4-(5-Chloro-2-hydroxyphenyl)-4-oxobut-2-enoic (87, 0.725 g, 3.20 mmol) was suspended in H₂O (21 mL) and thoroughly sonicated for 10 minutes. To this suspension was added 1M sodium hydroxide (3.36 mL, 3.36 mmol). The solution was heated to 100° C. for 1 h, and the mixture was cooled down to room temperature and acidified with conc. HCl until pH˜1. The solid was collected by filtration, washed with H₂O, brine and dried in vacuo to give 6-chloro-4-oxochromane-2-carboxylic acid (88, 0.586 g, 80%) as an orange solid: ¹H NMR (DMSO-d₆, 400 MHz): δ 13.56 (s, 1H), 7.68-7.59 (m, 2H), 7.18 (d, J=8.6 Hz, 1H), 5.36 (dd, J=7.3, 5.3 Hz, 1H), 3.14 (dd, J=17.1, 5.3 Hz, 1H), 2.99 (dd, J=17.1, 7.3 Hz, 1H); HRMS (ESI+) m/z calcd for C₁₀H₆ClO₄ [M−H] 224.9960, found 224.9936.

6-Chlorochromane-2-carboxylic acid (89). A solution of 6-chloro-4-oxochromane-2-carboxylic acid (88, 0.500 g, 2.21 mmol) in TFA (3.67 mL) was treated with triethylsilane (1.52 mL, 9.49 mmol). The solution was heated to reflux for 2 h and then cooled to room temperature. The solution was treated with NaOH (to basic pH) and was extracted with Et₂O (3×). The aqueous layer was then acidified with 6M HCl and extracted with Et₂O (3×). The combined organic layer was washed with brine, dried (Na₂SO₄), and concentrated. The solid was recrystallized from CHCl₃/hexanes to give 6-chlorochromane-2-carboxylic acid (89, 0.287 g, 61%) as an off-white solid: mp 165-166° C.; IR (neat) 2859, 1576, 1479, 1445, 1331, 1293, 1267, 1230, 1189, 1126, 1096, 1079, 1060, 1014, 957, 927, 904, 876, 843, 828, 779, 738, 710, 681. ¹H NMR (DMSO-d₆, 300 MHz): δ 13.06 (s, 1H), 7.12 (m, 2H), 6.82 (d, J=8.3 Hz, 1H), 4.80 (dd, J=6.1, 4.1 Hz, 1H), 2.79 (dt, J=16.9, 6.2 Hz, 1H), 2.69-2.55 (m, 1H), 2.22-1.96 (m, 2H); ¹³C NMR (DMSO-d₆, 100 MHz): δ 172.19, 152.72, 129.35, 127.51, 124.22, 124.16, 118.46, 73.30, 23.77, 22.67; HRMS (ESI+) m/z calcd for C₁₀H₈ClO₃ [M−H] 211.0167, found 211.0149.

6-Chloro-N-hydroxychromane-2-carboxamide (90). To a suspension of 6-chlorochromane-2-carboxylic acid (89, 0.150 g, 0.705 mmol) in dry benzene (2 mL) was added thionyl chloride (0.067 ml, 0.92 mmol). The reaction mixture was heated at reflux for 2.5 h. The solution was concentrated and treated with additional benzene and concentrated (2×). The residue was dissolved in THE (3 mL) and treated with hydroxylamine (50% aqueous soln, 0.300 mL, 6.36 mmol). The reaction mixture was concentrated and extracted with EtOAc and washed with phosphate (pH 7) buffer (2×), brine, dried (Na₂SO₄) and concentrated. The residue was triturated with CHCl₃ to give 6-chloro-N-hydroxychromane-2-carboxamide (90, 0.101 g, 63%) as a light peach solid: mp 160-162° C.; IR (neat) 3351, 2981, 2846, 1663, 1579, 1524, 1480, 1433, 1415, 1330, 1300, 1268, 1191, 1174, 1130, 1105, 1087, 1065, 1006, 970, 903, 877, 851, 824, 799, 755, 710, 672; ¹H NMR (DMSO-d₆, 400 MHz): δ 10.82 (s, 1H), 8.95 (s, 1H), 7.16-7.10 (m, 2H), 6.83 (d, J=8.4 Hz, 1H), 4.54 (dd, J=8.7, 3.2 Hz, 1H), 2.85-2.64 (m, 2H), 2.14-2.07 (m, 1H), 1.97-1.91 (m, 1H); ¹³C NMR (DMSO-d₆, 125 MHz): δ 166.13, 152.17, 128.79, 126.90, 124.07, 123.84, 118.30, 73.82, 23.80, 22.77; HRMS (ESI+) m/z calcd for C₁₀H₁₁ClNO₃ [M+H] 228.0422, found 228.0422.

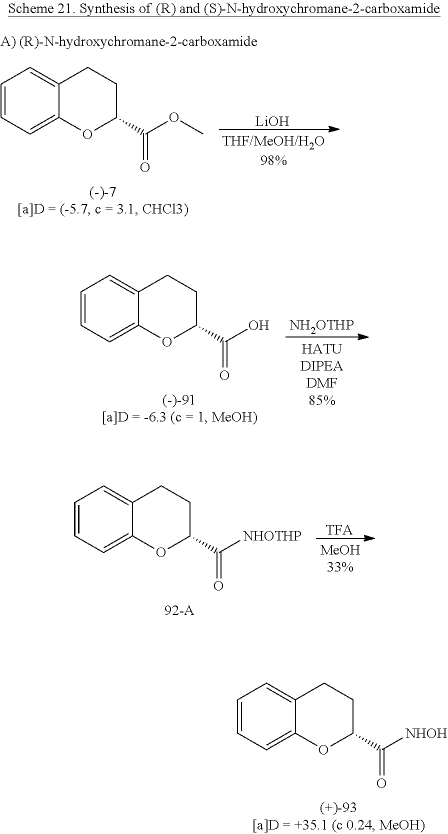

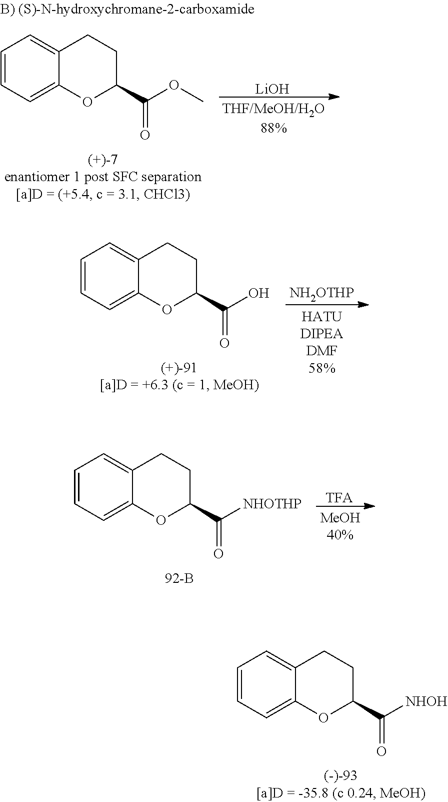

(R)-Chromane-2-carboxylic acid ((−)-91). A solution of methyl (R)-chromane-2-carboxylate (2R-8, 0.110 g, 0.57 mmol) in THF/MeOH (3.0 mL, 1/2) was treated with 1M LiGH (0.70 mL) at room temperature. After 7 h, the solution was concentrated. The residue was diluted with H₂O, acidified with 1M HCl, extracted with EtOAc (2×). The combined organic layer was washed with brine, dried (Na₂SO₄), filtered and concentrated to give product ((−)-91, 0.100 g, 98%) as a white solid: mp 77-79° C.; [α]_{D}=−6.3 (c 1, MeOH); ¹H NMR (CDCl₃, 400 MHz): δ 7.14 (app t, J=7.6 Hz, 1H), 7.05 (d, J=7.2 Hz, 1H), 6.93-6.88 (overlapping signals, 2H), 4.75 (dd, J=3.2, 3.2 Hz, 1H), 2.91-2.78 (overlapping signals, 2H), 2.40-2.36 (m, 1H), 2.35-2.16 (m, 1H).

(S)-Chromane-2-carboxylic acid ((+)-91). To a solution of methyl (S)-chromane-2-carboxylate (2S-8, 0.109 g, 0.57 mmol) in THE (2.0 mL) and MeOH (1.0 mL) was added 0.25M LiGH (2.4 mL) dropwise at room temperature. After 3 h, the solution was concentrated, diluted with H₂O, acidified with 1M HCl, and extracted with EtOAc (2×). The combined organic layer was washed with brine, dried (Na₂SO₄), filtered and concentrated. ¹H NMR indicates small amount of starting material remaining. The solid was dissolved in THE (0.5 mL), MeOH (0.5 mL) and treated with 0.25M LiGH (0.5 mL) at room temperature. After 6 h, the reaction was worked-up as described above to give (S)-chromane-2-carboxylic acid ((+)-91, 0.089 g, 88%) as a white solid: mp 75-77° C.; [α]_{D}=+6.3 (c 1.0, MeOH); ¹H NMR (CDCl₃, 500 MHz): δ9.69 (br s, 1H), 7.14 (app t, J=7.5 Hz, 1H), 7.05 (d, J=7.0 Hz, 1H), 6.94-6.89 (overlapping signals, 2H), 4.76 (dd, J=3.5, 3.5 Hz, 1H), 2.92-2.78 (overlapping signals, 2H), 2.40-2.36 (m, 1H), 2.35-2.22 (m, 1H).

(2R)—N-((Tetrahydro-2H-pyran-2-yl)oxy)chromane-2-carboxamide (92-A). To a solution of (R)-chromane-2-carboxylic acid ((−)-91, 0.100 g, 0.56 mmol) in DMF (2.0 mL) was added O-(tetrahydro-2H-pyran-2-yl)hydroxylamine (0.091 g, 0.78 mmol) followed by HATU (0.235 g, 0.61 mmol) and DIPEA (0.10 mL, 0.60 mmol) at 0° C. After 15 min, the solution was warmed to room temperature and kept at this temperature for 5 h. The solution was extracted with EtOAc, washed with 0.5M HCl, brine, dried (Na₂SO₄), filtered and concentrated. The residue was purified by chromatography on SiO₂ (ISCO-Rf, 0-10% MeOH/CH₂Cl₂) to give (2R)—N-((tetrahydro-2H-pyran-2-yl)oxy)chromane-2-carboxamide (92-A, 0.133 g, 85%) as an off-white solid residue: ¹H NMR (CDCl₃, 500 MHz): δ9.21 (s, 1H), 7.10 (app t, J=7.5 Hz, 1H), 7.05 (d, J=7.0 Hz, 1H), 6.90-6.85 (overlapping signals, 2H), 5.03-5.02 (m, 0.5H), 4.96-4.94 (m, 0.5H), 4.63-4.59 (m, 1H), 3.98-3.95 (m, 1H), 3.67-3.61 (m, 1H), 2.87-2.76 (m, 2H), 2.42-2.39 (m, 1H), 2.12-2.01 (m, 1H), 1.86-1.79 (m, 3H), 1.58-1.56 (m, 3H); ¹³C NMR (CDCl₃, 125 MHz): δ167.5, 167.4, 152.7, 152.6, 129.7, 129.6, 129.4, 127.5, 121.9, 121.8, 121.4, 120.9, 116.8, 116.6, 102.7, 102.6, 75.4, 62.6, 62.5, 27.92, 27.90, 25.1, 24.9, 24.8, 23.9, 23.7, 18.5; HRMS (LCMS ESI+) m/z calcd for C₁₅H₁₉O₄NNa [M+Na] 300.1206, found 300.1202.

(2S)—N-((Tetrahydro-2H-pyran-2-yl)oxy)chromane-2-carboxamide (92-B). To a solution of (S)-chromane-2-carboxylic acid ((+)-91, 0.084 g, 0.47 mmol) in DMF (2.0 mL) was added O-(tetrahydro-2H-pyran-2-yl)hydroxylamine (0.064 g, 0.54 mmol) followed by HATU (0.182 g, 0.47 mmol) and DIPEA (0.080 mL, 0.48 mmol) at room temperature. After 5 h, the solution was extracted with EtOAc, washed with 0.5M HCl, brine, dried (Na₂SO₄), filtered and concentrated. The residue was purified by chromatography on SiO₂ (ISCO-Rf, 0-100% EtOAc/hexanes) to give (2S)—N-((tetrahydro-2H-pyran-2-yl)oxy)chromane-2-carboxamide (92-B, 0.076 g, 58%) as an off-white solid residue: ¹H NMR (CDCl₃, 500 MHz): δ9.19 (s, 1H), 7.10 (app t, J=7.5 Hz, 1H), 7.05 (d, J=7.3 Hz, 1H), 6.90-6.84 (overlapping signals, 2H), 5.03-5.02 (m, 0.5H), 4.95-4.94 (m, 0.5H), 4.63-4.59 (m, 1H), 3.99-3.96 (m, 1H), 3.67-3.58 (m, 1H), 2.89-2.75 (m, 2H), 2.45-2.39 (m, 1H), 2.12-2.01 (m, 1H), 1.86-1.80 (m, 3H), 1.58-1.56 (m, 3H); ¹³C NMR (CDCl₃, 125 MHz): δ167.4, 167.3, 152.6, 152.5, 150.8, 129.7, 129.6, 129.1, 127.5, 121.9, 121.8, 121.3, 120.3, 116.6, 105.8, 102.6, 102.5, 75.4, 62.6, 62.5, 27.9, 27.8, 25.1, 24.9, 24.7, 23.9, 23.7, 18.4; HRMS (LCMS ESI+) m/z calcd for C₁₅H₁₉O₄NNa [M+Na] 300.1206, found 300.1203.

(R)—N-Hydroxychromane-2-carboxamide ((+)-93). To a solution of (2R)—N-((tetrahydro-2H-pyran-2-yl)oxy)chromane-2-carboxamide (92-A, 0.133 g, 0.48 mmol) in MeOH (3.5 mL) was added TFA (1.0 mL) at room temperature. After 3 h, the solution was concentrated and the residue was purified by chromatography on SiO₂ (ISCO-Rf, 0-100% EtOAc/hexanes) to give (R)—N-hydroxychromane-2-carboxamide ((+)-93, 0.031 g, 33%) as an off-white solid: [α]_{D}=+35.1 (c 0.24, MeOH); ¹H NMR (acetone-d₆, 500 MHz): δ 10.39 (s, 1H), 8.42 (br s, 1H), 7.04-7.00 (overlapping signals, 2H), 6.99-6.77 (overlapping signals, 2H), 4.58 (dd, J=2.0, 2.0 Hz, 1H), 2.83-2.77 (m, 1H), 2.72-2.67 (m, 1H), 2.25-2.21 (m, 1H), 1.99-1.96 (m, 1H); ¹³C NMR (acetone-d₆, 125 MHz): δ168.1, 154.2, 130.4, 128.1, 123.0, 121.6, 117.7, 75.6, 25.6, 24.3; HRMS (LCMS ESI+) m/z calcd for C₁₀H₁₂O₃N [M+H] 194.0812, found 194.0811.

(S)—N-Hydroxychromane-2-carboxamide ((−)-93). To a solution of (2S)—N-((tetrahydro-2H-pyran-2-yl)oxy)chromane-2-carboxamide (92-B, 0.076 g, 0.27 mmol) in MeOH (4.0 mL) was added TFA (1.0 mL, 13.4 mmol) at room temperature. After 2 h, the solution was concentrated. The residual oil was purified by chromatography on SiO₂ (ISCO-Rf, 0-100% EtOAc/hexanes) to give (S)—N-hydroxychromane-2-carboxamide ((−)-93, 0.021 g, 40%) as an off-white solid: [α]_{D}=−35.8 (c 0.24, MeOH); ¹H NMR (acetone-d₆, 600 MHz): δ10.41 (s, 1H), 8.45 (br s, 1H), 7.08-7.05 (overlapping signals, 2H), 6.84-6.82 (overlapping signals, 2H), 4.62 (dd, J=3.0, 3.0 Hz, 1H), 2.88-2.83 (m, 1H), 2.77-2.72 (m, 1H), 2.30-2.26 (m, 1H), 2.01-1.99 (m, 1H); ¹³C NMR (acetone-d₆, 150 MHz): δ168.7, 154.9, 131.2, 128.8, 123.7, 122.4, 118.4, 76.3, 26.3, 25.0; HRMS (LCMS ESI+) m/z calcd for C₁₀H₁₂O₃N [M+H] 194.0812, found 194.0811.

The activity of the compounds in Table 1 was measured, and is provided in Table 2.

TABLE 2

| | | HDAC | HDAC | HDAC | HDAC | HDAC | HDAC | HDAC | HDAC | HDAC | HDAC |
|---|---|---|---|---|---|---|---|---|---|---|---|
| | PanHDAC | 4 at | 4 at | 5 at | 5 at | 6 at | 6 at | 6 at | 7 at | 8 at | 8 at |
| Compound | at 1 uM | 1 uM | 5 uM | 200 nM | 1 uM | 50 nM | 100 nM | 200 nM | 1 uM | 200 nM | 1 uM |
| 1-1 | 0 | 15 | — | 0 | 71 | 33 | — | 53 | 19 | — | 1 |
| 1-2 | 0 | 0 | — | 20 | 67 | 5 | — | 77 | 15 | — | 92 |
| 1-3 | 0 | 7 | — | 33 | 75 | 46 | — | 87 | 20 | — | 7 |
| 1-4 | 0 | 14 | — | 0 | 69 | 47 | — | 62 | 17 | — | 0 |
| 1-5 | 0 | 14 | — | 0 | 67 | 29 | — | 74 | 18 | — | 0 |
| 1-6 | 0 | 4 | — | 11 | 60 | 41 | — | 71 | 19 | — | 0 |
| 1-7 | 0 | 18 | — | 10 | 71 | 35 | — | 77 | 22 | — | 8 |
| 1-8 | 0 | 14 | — | 11 | 63 | 0 | — | 19 | 19 | — | 0 |
| 1-9 | 0 | 0 | — | 22 | 56 | 50 | — | 88 | 19 | — | 0 |
| 1-10 | 0 | 0 | — | 0 | 81 | 42 | — | 39 | 19 | — | 2 |
| 1-11 | 3 | 18 | — | 0 | 73 | 25 | — | 65 | 24 | — | 0 |
| 1-12 | 0 | 0 | — | 17 | 49 | 28 | — | 43 | 20 | — | 0 |
| 1-13 | 0 | 12 | — | 0 | 68 | 23 | — | 36 | 19 | — | 0 |
| 1-14 | 21 | 16 | — | 13 | 70 | 0 | — | 99 | 18 | — | 2 |
| 1-15 | 0 | 17 | — | 11 | 68 | 26 | — | 57 | 12 | — | 0 |
| 1-16 | 0 | 26 | — | 3 | 51 | 73 | — | 61 | 34 | — | 5 |
| 1-17 | 0 | 10 | — | 0 | 69 | 40 | — | 78 | 18 | — | 62 |
| 1-18 | 0 | 15 | — | 23 | 50 | 68 | — | 85 | 22 | — | 2 |
| 1-19 | 0 | 13 | — | 19 | 70 | 27 | — | 81 | 23 | — | 0 |
| 1-20 | 0 | 13 | — | 4 | 76 | 32 | — | 68 | 0 | — | 5 |
| 1-21 | 0 | 13 | — | 16 | 72 | 73 | — | 84 | 19 | — | 0 |
| 1-22 | 0 | 18 | — | 17 | 72 | 0 | — | 70 | 53 | — | 0 |
| 1-23 | 0 | 0 | — | 17 | 75 | 50 | — | 79 | 19 | — | 2 |
| 1-24 | 0 | 16 | — | 16 | 58 | 23 | — | 79 | 14 | — | 0 |
| 1-25 | 0 | 0 | — | 0 | 69 | 0 | — | 84 | 17 | — | 1 |
| 1-26 | 0 | 17 | — | 0 | 66 | 20 | — | 69 | 19 | — | 0 |
| 1-27 | 0 | 13 | — | 19 | 61 | 31 | — | 77 | 15 | — | 0 |
| 1-28 | 0 | 17 | — | 0 | 72 | 44 | — | 75 | 14 | — | 0 |
| 1-29 | 0 | 5 | — | 0 | 72 | 37 | — | 47 | 18 | — | 0 |
| 1-30 | 0 | 17 | — | 9 | 69 | 29 | — | 44 | 21 | — | 5 |
| 1-31 | 0 | 13 | — | 29 | 70 | 41 | — | 39 | 0 | — | 0 |
| 1-32 | 14 | 47 | — | 14 | 75 | 70 | — | 52 | 24 | — | 0 |
| 1-33 | 0 | 35 | — | 7 | 68 | 32 | — | 71 | 25 | — | 3 |
| 1-34 | 0 | 16 | — | 13 | 68 | 13 | — | 66 | 18 | — | 0 |
| 1-35 | 0 | 5 | — | 8 | 70 | 0 | — | 74 | 20 | — | 0 |
| 1-36 | 0 | 18 | — | 34 | 66 | 6 | — | 33 | 19 | — | 1 |
| 1-37 | 6 | 14 | — | 26 | 66 | 2 | — | 78 | 13 | — | 0 |
| 1-38 | 0 | 16 | — | 34 | 71 | 21 | — | 50 | 0 | — | 0 |
| 1-39 | 0 | 16 | — | 3 | 79 | 34 | — | 62 | 21 | — | 0 |
| 1-40 | 0 | 6 | — | 0 | 68 | 30 | — | 67 | 17 | — | 0 |
| 1-41 | 0 | 67 | — | 41 | 83 | 55 | — | 87 | 27 | — | 1 |
| 1-42 | 0 | 0 | — | 28 | 74 | 14 | — | 71 | 19 | — | 0 |
| 1-43 | 0 | 20 | — | 58 | 72 | 34 | — | 68 | 22 | — | 0 |
| 1-44 | 0 | 17 | — | 9 | 57 | 29 | — | 83 | 19 | — | 22 |
| 1-45 | 0 | 16 | — | 17 | 54 | 0 | — | 80 | 17 | — | 9 |
| 1-46 | 0 | 16 | — | 5 | 66 | 48 | — | 64 | 11 | — | 0 |
| 1-47 | 0 | 19 | — | 0 | 64 | 53 | — | 76 | 19 | — | 60 |
| 1-48 | 0 | 0 | — | 21 | 68 | 0 | — | 79 | 20 | — | 5 |
| 1-49 | 0 | 14 | — | 27 | 66 | 0 | — | 84 | 19 | — | 0 |
| 1-50 | 0 | 15 | — | 37 | 69 | 48 | — | 86 | 19 | — | 0 |
| 1-51 | 8 | 31 | — | 3 | 74 | 64 | — | 76 | 22 | — | 0 |
| 1-52 | 0 | 33 | — | 7 | 63 | 0 | — | 80 | 24 | — | 0 |
| 1-53 | 0 | — | 49 | 17 | — | 25 | — | — | 0 | — | 0 |
| 1-54 | 0 | 0 | — | 20 | — | 39 | — | 95 | 0 | — | 83 |
| 1-55 | 0 | 8 | — | 0 | — | 44 | — | 92 | 22 | — | 27 |
| 1-56 | 0 | — | 32 | 23 | — | 13 | — | — | 89 | — | 42 |
| 1-57 | 0 | 15 | — | 24 | — | 7 | — | 92 | 20 | — | 75 |
| 1-58 | 0 | 12 | — | 11 | — | 31 | — | 89 | 24 | — | 77 |
| 1-59 | 0 | 0 | — | 0 | — | 0 | — | 92 | 11 | — | 82 |
| 1-60 | 0 | — | 35 | 22 | — | 33 | — | — | 8 | — | 51 |
| 1-61 | 0 | — | 33 | 35 | — | 41 | — | — | 80 | — | 0 |
| 1-62 | 0 | — | 53 | 12 | — | 32 | — | — | 0 | — | 0 |
| 1-63 | 0 | — | 19 | 22 | — | 45 | — | — | 12 | — | 37 |
| 1-64 | 3 | — | 36 | 38 | — | 47 | — | — | 34 | — | 49 |
| 1-65 | 0 | — | 40 | 23 | — | 17 | — | — | 29 | — | 18 |
| 1-66 | 0 | — | 31 | 14 | — | 31 | — | — | 0 | — | 0 |

### REFERENCES

1. Dolle, R. E.; Chu, G. U.S. Patent, 20050054630 A1, 2005.
2. Manchand, P. S.; Micheli, R. A.; Saposnik, S. J. Tetrahedron 1992, 48, 9391-9398.
3. Chaudhari, S. S.; Kadam, A. B.; Khairatkar-Joshi, N.; Mukhopadhyay, I.; Karnik, P. V.; Raghuram, A.; Rao, S. S.; Vaiyapuri, T. S.; Wale, D. P.; Bhosale, V. M.; Gudi, G. S.; Sangana, R. R.; Thomas, A. Biorg. Med. Chem. 2013, 21, 6542-6553.

### Embodiments

Embodiment P1. A compound represented by a structure of Formula (I):

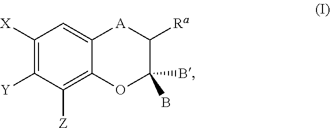

or a pharmaceutically acceptable salt, solvate, or prodrug thereof, wherein:
A is CR′R′, CR′OH, or NR′;
R′ is H or alkyl;
R^{a} is H or alkyl;
one of B and B′ is H or alkyl and the other is C(O)Q or B and B′ together form a cycloalkyl or heterocycle that is substituted by C(O)Q or (CH₂)₁₋₂C(O)Q;
Q is NHOH;
X is H, halo, or R¹;
Y is H, halo, or R¹;
Z is H, halo, or R¹;
each R¹ is independently D-E-G,
where D is a bond, —O—, —NR—, —OCONR—, —OCO—, —NRSO₂—, —NRCO—, —NRSO₂NR—, —NRCOO—, —NRCONR—, or —NRC(NR)NR—;
E is absent or is selected from a C₁-C₆ alkane, C₁-C₆ alkene, C₁-C₆ alkyne, or alkoxy; and
   G is an optionally substituted aryl or

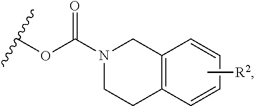

where R² is sleeted from optionally substituted alkyl, alkoxy, aryl, halo, SF₅, and
wherein at least one of X, Y and Z is not H.

Embodiment P2. The compound of embodiment P1, wherein A is CH₂.

Embodiment P3. The compound of embodiment P1 or P2, wherein B is C(O)Q and B′ is H.

Embodiment P4. The compound of any one of embodiments P1 to P3, wherein X is R¹, and is represented by the following:

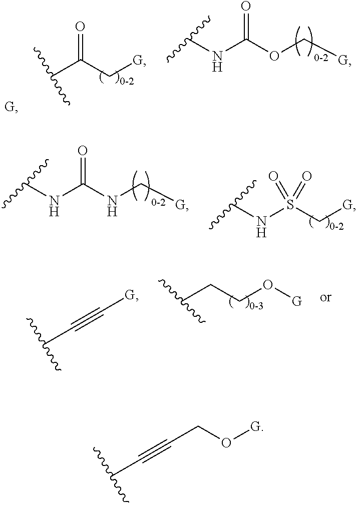

Embodiment P5. The compound of embodiment P4, wherein G is

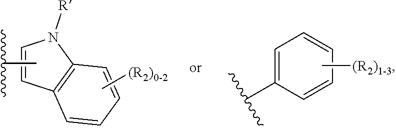

where R² is sleeted from optionally substituted alkyl, alkoxy, aryl, halo, SF₅, or two adjacent R² form a ring.

Embodiment P6. The compound of any one of embodiments P1 to P3, wherein Z is R¹, and is represented by the following:

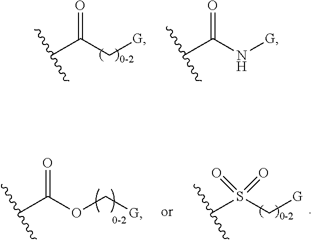

Embodiment P7. The compound of embodiment P6, wherein G is

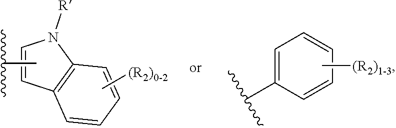

where R² is selected from optionally substituted alkyl, alkoxy, aryl, halo, SF₅, or two adjacent R² form a ring.

Embodiment P8. The compound of any one of embodiments P1 to P3, wherein Y is G or

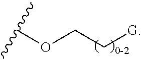

Embodiment P9. The compound of embodiment P8, wherein G is

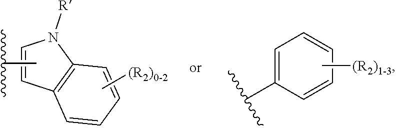

where R² is selected from optionally substituted alkyl, alkoxy, aryl, halo, SF₅, or two adjacent R² form a ring.

Embodiment P10. The compound of any one of embodiments P1, P2 and P4 to P9, wherein B and B′ are

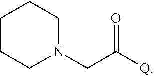

Embodiment P11. A compound selected from Table 1, or a pharmaceutically acceptable salt, solvate, or prodrug thereof.

Embodiment P12. A pharmaceutical composition comprising a compound of any one of embodiments P1 to P11 and at least one pharmaceutically acceptable excipient.

Embodiment P13. A method of inhibiting histone acetylation in a cell, comprising contacting the cell with a compound of any one of embodiments P1 to P11.

Embodiment P14. A method of treating a disease capable of treatment by inhibition of histone acetylation in a patient in need thereof, comprising administering a compound or composition of any one of embodiments P1 to P12.

Embodiment P15. The method of embodiment P14, wherein the disease is a malignancy.

Embodiment P16. The method of embodiment P15, wherein the malignancy is a hematologic malignancy.

Embodiment P17. The method of embodiment P14, wherein the disease is an infectious disease.

While the disclosure has been described with reference to the specific embodiments thereof, it should be understood by those skilled in the art that various changes may be made and equivalents may be substituted without departing from the true spirit and scope of the disclosure as defined by the appended claim(s). In addition, many modifications may be made to adapt a particular situation, material, composition of matter, method, operation or operations, to the objective, spirit and scope of the disclosure. All such modifications are intended to be within the scope of the claim(s) appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations are not a limitation of the disclosure.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A compound represented by a structure of Formula (II): 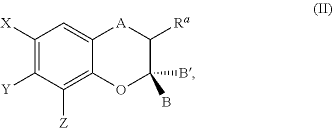
or a pharmaceutically acceptable salt, solvate, or prodrug thereof, wherein:
A is CR′R′, CR′OH, CR′OCH₃, or NR′;
each R′ is independently H or alkyl;
R^{a} is H or alkyl;
one of B and B′ is H or alkyl and the other is C(O)Q or B and B′ together form a cycloalkyl or heterocycle that is substituted by C(O)Q or (CH₂)₁₋₂C(O)Q;
Q is NHOH;
X is H, halo, or R¹;
Y is H, halo, or R¹;
Z is H, halo, or R¹;
each R¹ is independently D-E-G,
where D is a bond, —O—, —NR—, —OCONR—, —OCO—, —NRSO₂—, —NRCO—, —NRSO₂NR—, —NRCOO—, —NRCONR—, or —NRC(NR)NR—;
E is absent or is selected from an optionally substituted C₁-C₆ alkyl, C₁-C₆ alkenyl, C₁-C₆ alkynyl, or alkoxy; and
G is H, an optionally substituted aryl, an optionally substituted heteroaryl, 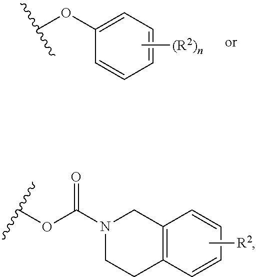
where each R² is independently selected from optionally substituted alkyl, alkoxy, aryl, halo, and SF₅;
each R is independently H or C₁-C₃ alkyl; and
n is 0, 1, 2, or 3; and
wherein at least one of X, Y and Z is not H.

2. The compound of claim 1, wherein A is CH₂.

3. The compound of claim 1, wherein B is C(O)Q and B′ is H.

4. The compound of claim 1, wherein X is R¹, and is represented by the following:
G

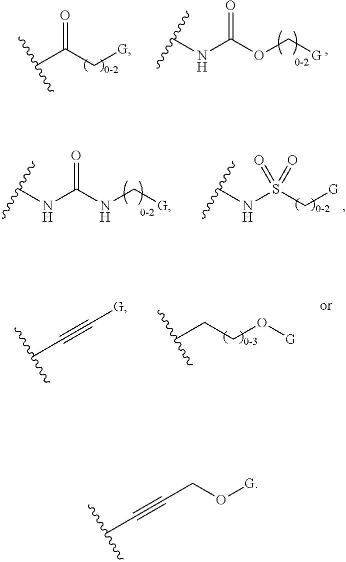

5. The compound of claim 4, wherein G is 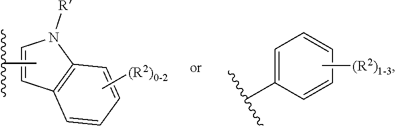
where each R² is independently selected from optionally substituted alkyl, alkoxy, aryl, halo, SF₅, or two adjacent R² form a ring.

6. The compound of claim 1, wherein Z is R¹, and is represented by the following:

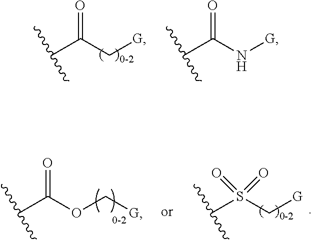

7. The compound of claim 6, wherein G is 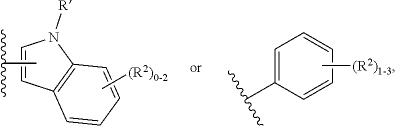
where each R² is independently selected from optionally substituted alkyl, alkoxy, aryl, halo, SF₅, or two adjacent R² form a ring.

8. The compound of claim 1, wherein Y is G or

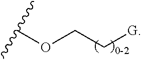

9. The compound of claim 8, wherein G is 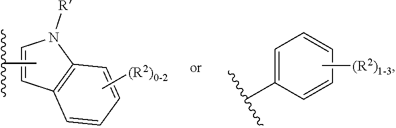
where each R² is independently selected from optionally substituted alkyl, alkoxy, aryl, halo, SF₅, or two adjacent R² form a ring.

10. The compound of claim 1, wherein G is phenyl optionally substituted with 1 to 5 substituents independently selected from F, Cl, CH₃, CF₃, OMe, SF₅, and phenyl.

11. The compound of claim 1, wherein G is heteroaryl optionally substituted with 1 to 5 substituents independently selected from F, Cl, CH₃, CF₃, OMe, and SF₅.

12. The compound of claim 1, wherein E is absent.

13. The compound of claim 1, wherein R^{a} is H.

14. The compound of claim 1, wherein each R² is independently selected from F, Cl, CH₃, CF₃, OMe, and SF₅.

15. The compound of claim 1, wherein R′ is H or CH₃.

16. The compound of claim 1, wherein R is H.

17. The compound of claim 1, wherein B and B′ together form a heterocycle having the following structure:

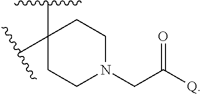

18. A compound selected from Table 1, or compound 50b, compound 50d, or compound 50e, or a pharmaceutically acceptable salt, solvate, or prodrug thereof.

19. A pharmaceutical composition comprising a compound of claim 1, and at least one pharmaceutically acceptable excipient.

20. A method of inhibiting histone acetylation in a cell, comprising contacting the cell with a compound of claim 1.

21. A method of treating a disease capable of treatment by inhibition of histone acetylation in a patient in need thereof, comprising administering a compound or composition of claim 1.

22. The method of claim 21, wherein the disease is a malignancy.

23. The method of claim 22, wherein the malignancy is a hematologic malignancy.

24. The method of claim 21, wherein the disease is an infectious disease.
